SEQUENCE LISTING (91 sequences)

SEQ ID NO: 1 - DNA, 38 bp, synthetic construct
  source 1..38: mol_type = other DNA; note = oriT_pMRC01
acaccacccaattttggagtggtgtgtaagtgcgcatt

SEQ ID NO: 2 - DNA, 38 bp, synthetic construct
  source 1..38: mol_type = other DNA; note = oriT_RSF1010
ccagtttctcgaagagaaaccggtaagtgcgccctccc

SEQ ID NO: 3 - DNA, 40 bp, synthetic construct
  source 1..40: mol_type = other DNA; note = oriT_pRS01
tccgtaagatgctatcatcttactatgcttgcaaaaggtc

SEQ ID NO: 4 - DNA, 41 bp, synthetic construct
  source 1..41: mol_type = other DNA; note = oriT_pMV158
cactttatgaatataaagtatagtgtgttatactttacatg

SEQ ID NO: 5 - DNA, 42 bp, synthetic construct
  source 1..42: mol_type = other DNA; note = oriT_pTF1
gcacgggtaatctcgaagagattactctaagtgcgcccttgc

SEQ ID NO: 6 - DNA, 45 bp, synthetic construct
  source 1..45: mol_type = other DNA; note = oriT_pSC101
gggcgcacgtttctgaacgaagtgaagaaagtctaagtgcgccct

SEQ ID NO: 7 - DNA, 51 bp, synthetic construct
  source 1..51: mol_type = other DNA; note = oriT_pBTK445
agcctttaaagcgaaaatagggtactccatgctcgctatatcatcctgaca

SEQ ID NO: 8 - DNA, 52 bp, synthetic construct
  source 1..52: mol_type = other DNA; note = oriT_pBBR1
ggtcacgactttgcgaagcaaagtctagtgagtatactcaagcattgagtgg

SEQ ID NO: 9 - DNA, 53 bp, synthetic construct
  source 1..53: mol_type = other DNA; note = oriT_R721
cacacgattgtaacatgaccggaacggtcttgtgtacaatcggtatcgtgcct

SEQ ID NO: 10 - DNA, 59 bp, synthetic construct
  source 1..59: mol_type = other DNA; note = oriT_pRmeGR4a
gcaggaaaacggcgtagcacatttttccgtatcctgcccctccacattgtaaggggatt

SEQ ID NO: 11 - DNA, 60 bp, synthetic construct
  source 1..60: mol_type = other DNA; note = oriT_ColE1
gggtgtcggggcgcagccctgacccagtcacgtagcgatagcggagtgtatactggctta

SEQ ID NO: 12 - DNA, 61 bp, synthetic construct
  source 1..61: mol_type = other DNA; note = oriT_pTiC58
ggatccaagggcgcaattatacgtcgctgacgcgacgccttgcgtagggggccaaacaggg

SEQ ID NO: 13 - DNA, 64 bp, synthetic construct
  source 1..64: mol_type = other DNA; note = oriT_pMdT1
aggtttcggggcgcagccctgaaccagtcacctagcgctagcggagtgtatactggcttagtat

SEQ ID NO: 14 - DNA, 71 bp, synthetic construct
  source 1..71: mol_type = other DNA; note = oriT_R1
agcaaatcagcaaaaacttgtttttgcgtggggtgtggtgcttttggtggtgagaaccaccaacctgttga

SEQ ID NO: 15 - DNA, 71 bp, synthetic construct
  source 1..71: mol_type = other DNA; note = oriT_Tn5520
cttattggggaattttcagcgatacggagtattgcggctcggaaaattccctaataagctacggtattttc

SEQ ID NO: 16 - DNA, 90 bp, synthetic construct
  source 1..90: mol_type = other DNA; note = oriT_QKH54
gtgaagatagttaaccggcttgccggttagctaacttcacctatcttgcccggctcttcgagccgtttaacgccaggtgagtatcgcata

SEQ ID NO: 17 - DNA, 92 bp, synthetic construct
  source 1..92: mol_type = other DNA; note = oriT_R64
ggggtgtcggggcgaagccctgaccagatggcaattgtaatagcgtcgcgtgtgacggtattacaattgcacatcctgtcccgtttttcggg

SEQ ID NO: 18 - DNA, 99 bp, synthetic construct
  source 1..99: mol_type = other DNA; note = oriT_R751
gaataagggacagtgaagatagataaccggctcgccggttagctaacttcacacatcctgcccgccttacggcgttaataacaccaaggaaagtctaca

SEQ ID NO: 19 - DNA, 814 bp, synthetic construct
  source 1..814: mol_type = other DNA; note = oriT_RP4
cgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggct

SEQ ID NO: 20 - DNA, 100 bp, synthetic construct
  source 1..100: mol_type = other DNA; note = oriT_pKL1
cggggtgtcggggtgaagccctgaccaagtggtaatcgtatcggcgtgcatgcgcggttatacgattacacatcctgtcccgatttctgaggcgttttaa

SEQ ID NO: 21 - DNA, 112 bp, synthetic construct
  source 1..112: mol_type = other DNA; note = oriT_RK2
ccggccagcctcgcagagcaggattcccgttgagcaccgccaggtgcgaataagggacagtgaagaaggaacacccgctcgcgggtgggcctacttcacctatcctgcccgg

SEQ ID NO: 22 - DNA, 160 bp, synthetic construct
  source 1..160: mol_type = other DNA; note = oriT_R1162
ggccagtttctcgaagagaaaccggtaaatgcgccctcccctacaaagtagggtcgggattgccgccgctgtgcctccatgatagcctacgagacagcacattaacaatggggtgtcaagatggttaaggggagcaacaaggcggcggatcggctggcca

SEQ ID NO: 23 - DNA, 174 bp, synthetic construct
  source 1..174: mol_type = other DNA; note = oriT_Tn4555
ccctcgggagagcccacaactacgtaagcggagcgtgtagttatagtgggctatatcaatggcaagccattgtctgcaaactccagcctacggcttccgctctcctccgtcagggaggtttttcatcatcgttgccgattggagatgcaccgaccagcacaaggtctaaatcgt

SEQ ID NO: 24 - DNA, 192 bp, synthetic construct
  source 1..192: mol_type = other DNA; note = oriT_pHT
ccaaagaattaatgcaaagagcataagggaaaactaatagcaccttcctaaaggaaggtggctaagttggctgtgccaactggttttctttcaaaatcacttcatattttttgctatcacaaaaaaatccattttcgacctattttcggtcataatatagtacctacttttggtcatagtttcgtccgtagt

SEQ ID NO: 25 - DNA, 199 bp, synthetic construct
  source 1..199: mol_type = other DNA; note = oriT_Tn4399
taaggtgattatgttgtttttcttcatctgttctatctgttttttagtgaataatccgattgatgtaatctgaaaagtccgtgaccatcgggagccgttcccctcatctttttgaggggcaagtggtcggggaatgtaatacgccgacattaacttgctatcctaaaaaagatgtgatttacggcttagatgccgaatc

SEQ ID NO: 26 - DNA, 216 bp, synthetic construct
  source 1..216: mol_type = other DNA; note = oriT_Tn916
aagcggaagtcgcaggtgtggactgatcttgctggctggtgtggcaatagccacgccagcacttaaccccccgtatctaacaggggggtacaaatcgacaggaaacagtcaaaaaaacattagaaaatcctttggttacaagggatttacaaaatttcagcgtatgtcaaatgggctttaaaagttgacatacggcctttttgattggagggattt

SEQ ID NO: 27 - DNA, 242 bp, synthetic construct
  source 1..242: mol_type = other DNA; note = oriT_pST12
ttgtgtgattatatcgcgtaccacttttcgactgttttaccgccggtattctgccgtctgacgctttgacgggtatttctgcctgacaatactgtcactgccaaaaaactgccgtgcctttgtcggtaattcgagcttgctgacaggacaggatgtgcaattgttataccgcgcatacatgcacgctattacaattaccctggtcagggcttcgccccgacaccccatgtcagatacggagc

SEQ ID NO: 28 - DNA, 268 bp, synthetic construct
  source 1..268: mol_type = other DNA; note = oriT_pCU1
gtattaccaaagtaataaagcaaactcattataaaacaatgagttattaggtgtttttaatacctaattattaccgaatattgacgctatttatttttttattttttaaatcagtgtgatagcgtgatttatgccgctgcgttaggtgtatagcaggttaagggataaaaaatcatcttttttggtaggagcgatctacgtaggttaaggactaactgactaaaaagcgttcaatattccgtattcatgcttgcatgaataccagtac

SEQ ID NO: 29 - DNA, 279 bp, synthetic construct
  source 1..279: mol_type = other DNA; note = oriT_pSU233
cgctagcagcgcccctgacggtatcctataaaaaaacacaccgcgccgctagcagcacccctaatataaaataatgttttttataaaaatagtcagtaccacccctacaaagcggtgtcggcgcgttgctgtagctgcgttaacgacgctgctttaaataaatcagatttaaacaatataaatccacaaatacaactcnatgatattaaagataaatcagcaaaaacttgtttttgcgtggggtgtggtgcttttggtggtgagaaccaccaacctgtt

SEQ ID NO: 30 - DNA, 290 bp, synthetic construct
  source 1..290: mol_type = other DNA; note = oriT_F
cgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcct

SEQ ID NO: 31 - DNA, 304 bp, synthetic construct
  source 1..304: mol_type = other DNA; note = oriT_pMAB01
tgcctcgcagagcaggatgacccgttgagcgcccccggcgcgaataagggacagtgaagatagataaccggctcgccggttagctaacttcacacatcctgcccgccttacggcgttaataacaccaaggaaagtctacaccagccattacgatttatccgcaactatcgcgctatcaggccgcaaaagcagcaacggatatagcgaaaaccgccacaatggcccataatgccgctatcgaagcgtgccaatgcacgccgatagcggactttttgcgtttccgtagcgccgcttagtagcgtta

SEQ ID NO: 32 - DNA, 402 bp, synthetic construct
  source 1..402: mol_type = other DNA; note = oriT_R388
ccgcctcgtcctccaaaagtgcctgcttttccgggcttagccgtacttggatggggtcgcctagtgccatgtcctctcccgtagtgttactgtagtggttcaatcctagcatttacaaggggttgcggcaatattgtagtggcataacactacacaggttttcgtccttggcgtggaagtcattgtaaatcaatgacttacgcgcaccgaaaggtgcgtattgtctatagcccagatttaaggataccaacccggcttttaaggacggaaaccatgcgataacgccagcgtgaccctaaagagggtcaaaactgctcccaatgcgctatgcgcattgggttatcgtgcagcaatgatgcaactataatgctatgatggtgctacaatgatgcagaaaatgag

SEQ ID NO: 33 - DNA, 456 bp, synthetic construct
  source 1..456: mol_type = other DNA; note = oriT_pS7a
ggatgatcaaacaaaatacgagagattttttgttcgttcatccatggttttagaaaaaagagggacgatttcggaagaagaaaatcgtctcttttttttcttctttttgtatgacaaaaagaaagatcttttgcccatttttattttttataaaatgggcaggtggcgtttgcgtaaagcaaatcgacacaatccaaaggggataaaaggggaaagtgaaacttcccccttttcaagccacattgtaatacaagaacgaagtgctttgtattacaatgtgatagcttgcagtatttatggttttatatggtctattttgttgtgaggattgtaaccgaatagggcgcaatacttattacaaaatcaatgacaaagggcgattgagaaatgagcgctggggcattttatctttgaggaagttcttgatggatcagaaaaatgtatcacaaatttaaa

SEQ ID NO: 34 - DNA, 456 bp, synthetic construct
  source 1..456: mol_type = other DNA; note = oriT_pS7b
tggatgatcaaacaaaatacgagagattttttgttcgttcatccatggttttagaaaaaagagggacgatttcggaagaagaaaatcgtctcttttttttcttctttttgtatgacaaaaagaaagatcttttgcccatttttattttttataaaatgggcaggtggcgtttgcgtaaagcaaatcgacacaatccaaaggggataaaaggggaaagtgaaacttcccccttttcaagccacattgtaatacaagaacgaagtgctttgtattacaatgtgatagcttgcagtatttatggttttatatttcctattttgttgtgaggattgtaaccgaatagggcgcaatgcttattacaaaatcaatgacaaagggcgagtgaggaatgagcgctgaggcattttatctttgaggaagttcttgatggatcagaaaaatgtatcacaaatttaa

SEQ ID NO: 35 - DNA, 697 bp, synthetic construct
  source 1..697: mol_type = other DNA; note = oriT_R702
ccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgacgagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctcc

SEQ ID NO: 36 - DNA, 475 bp, synthetic construct
  source 1..475: mol_type = other DNA; note = oriT_pMUR274
actccgatacgttgccctccgaacggtattcaaggtcgattttttgcgcttcggtcactcttaaactgatagatggcataggtttcctttgtgtaataccgatgtaatacatacaaatctagcatagatgcggcttaattccacatatgtaatacgttgtgtattacatattaaaacacaaattagaataatttgttttgttttcaagcatttacgatgaaaatcgtaattgcgtatggtgtatagccgttaagggataccataccacgccttttttaagggagaaaccggtgttacgtgcaagtgaatcgctcaaaaagcgttcacattcacacctttcatgcttgcatgaaaggaaacggacgggaattagacaaaaataagacacgatgagtaagttattgagacaagaaaaggacacaaataagacattttttagaaaaaaacattgacttgagactagaaatggacaata

SEQ ID NO: 37 - DNA, 550 bp, synthetic construct
  source 1..550: mol_type = other DNA; note = oriT_R100
ttactctggccataagataaaacctttcattattaagcaacgaacttttcactataaatatgcatatagtgtttacaagtaagaaagacactcctagcagcgcctctaggatcatcctataaaaaaatgcgatccggcgctaggggcgtccctaatatatatcaatgtttttcgtgaaaattgtcagtactgatcctaataagagtcgctatagggtcgtaacaggatcgccaacgactctctatttaataattcagaattattaaatataaatagcgtttgttaattacatgatttaaaacgtaaatcagcaaaaacttgtttttgcgtagtgtgtggtgcttttggtggtgagaaccaccaacctgttgagcctttttgtggagtgggttaaattatttacggataaagtcaccagaggtggaaaaatgaaaaaatggatgttagcaatctgcctgatgtttataaatgggatctgcgaagccgccgattgctttgatcttgcaggtcgggattacaaaatagacccggatttactaagaatgatatc

SEQ ID NO: 38 - DNA, 551 bp, synthetic construct
  source 1..551: mol_type = other DNA; note = oriT_pVCR94deltaX
gagcagagctatgtgtgacaagaagtatagagattacgaggtagccatcatggtcgatgtgaaccctttcgacagggttatgaatgaattgaaaagtcgtggccgcaagaacgctcacatcctgagcatcctccaattcgactggcctgcatcggaggccatcatcgagaagctgagctgctacatcacagacgggattaaggctaatcaggagcctgtgatttacccgatcattgaagaagctctgcatcgctacagccagctcgtgtttcatgagcagagagagaaatatgaagacccggccagaattggggcatttctggaaaccctgatcaccgaaacctgccgggcgttggaagtgcaaattgtcgatagtggcggtgattcatggtctgtcgattcaggagagtcgttctcactgtggctttcttcccatccaggagaactatccattaacccgcagccccatgaggatgagacctctttgcgtggcttgctgtatgagctcatcacctgtgagagcgtgaaaactgttttaaggagaaccgact

SEQ ID NO: 39 - DNA, 650 bp, synthetic construct
  source 1..650: mol_type = other DNA; note = oriT_R46
agcgccgcagataatctgaccgattacctcctgaaaccaggtctatataggccaaaaagttcatctgatacttttgcggttattattggcattcagtcctcacattgtgcatttcttaaacaaaagattgggatctaacaagctgaaatcttagtattaccaaagtaataaagcaaactcattataaaacaatgagttattaggtgtttttaatacctaattattaccgaatattgacgctatttatttttttattttttaaatcagtgtgatagcgtgatttatgccgctgcgttaggtgtatagcaggttaagggataaaaaatcatcttttttggtaggagcgatctacgtaggttaaggactaactgactaaaaagcgttcaatattccgtattcatgcttgcatgaataccagtacaacactattacaacaaaagtacatcaaaattacatcaaaagtacatcacttgaaggttgacagtacaacagaattacatcattatctggtactgaggtagccagtacaacaaaagtacatcaaaaatacatcataaatacatcagaaatacatcaaaattacatcattctaaatgagggtactatgaagcccaaaagtatcagggcggcacttcagttgatgttgccgg

SEQ ID NO: 40 - DNA, 36 bp, synthetic construct
  source 1..36: mol_type = other DNA; note = oriT_pGO1
cacgcgaacggaacgttcgcataagtgcgcccttac

SEQ ID NO: 41 - DNA, 38 bp, synthetic construct
  source 1..38: mol_type = other DNA; note = oriT_pIP501
atacgaagtaacgaagttactgcgtataagtgcgccct

SEQ ID NO: 42 - DNA, 392 bp, synthetic construct
  source 1..392: mol_type = other DNA; note = R6K
gatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcctttaaggtttaacggttgtggacaacaagccagggatgtaacgcactgagaagcccttagagcctctcaaagcaattttgagtgacacaggaacacttaacggctgacatgg

SEQ ID NO: 43 - DNA, 2222 bp, synthetic construct
  source 1..2222: mol_type = other DNA; note = RK2
gcgatgcaggtggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcttcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggactatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccggaattaattccccggatcgatccgtcgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccctacatggctctgctgtagtgagtgggttgcgctccggcagcggtcctgatcccccgcagaaaaaaaggatctcaagaagatcctttgatcttttctac

SEQ ID NO: 44 - DNA, 1522 bp, synthetic construct
  source 1..1522: mol_type = other DNA; note = pBBR1
ctaccggcgcggcagcgttacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtaggggg

SEQ ID NO: 45 - DNA, 1969 bp, synthetic construct
  source 1..1969: mol_type = other DNA; note = pRO1600
gataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggcatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggccgtgaaaggcaggccggtccgtggtggccacggcctctaggccagatccagcggcatctgggttagtcgagcgcgggccgcttcccatgtctcaccagggcgagcctgtttcgcgatctcagcatctgaaatcttcccggccttgcgcttcgctggggccttacccaccgccttggcgggcttcttcggtccaaaactgaacaacagatgtgtgaccttgcgcccggtctttcgctgcgcccactccacctgtagcgggctgtgctcgttgatctgcgtcacggctggatcaagcactcgcaacttgaagtccttgatcgagggataccggccttccagttgaaaccactttcgcagctggtcaatttctatttcgcgctggccgatgctgtcccattgcatgagcagctcgtaaagcctgatcgcgtgggtgctgtccatcttggccacgtcagccaaggcgtatttggtgaactgtttggtgagttccgtcaggtacggcagcatgtctttggtgaacctgagttctacacggccctcaccctcccggtagatgattgtttgcacccagccggtaatcatcacactcggtcttttccccttgccattgggctcttgggttaaccggacttcccgccgtttcaggcgcagggccgcttctttgagctggttgtaggaagattcgatagggacacccgccatcgtcgctatgtcctccgccgtcactgaatacatcacttcatcggtgacaggctcgctcctcttcacctggctaatacaggccagaacgatccgctgttcctgaacactgaggcgatacgcggcctcgaccagggcattgcttttgtaaaccattgggggtgaggccacgttcgacattccttgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggcgcctgagagg

SEQ ID NO: 46 - DNA, 3674 bp, synthetic construct
  source 1..3674: mol_type = other DNA; note = RSF1010
tcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgattcccacacattatacgagccggaagcataaagtgtaaagcctagatccgaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttag

SEQ ID NO: 47 - DNA, 2982 bp, synthetic construct
  source 1..2982: mol_type = other DNA; note = pAMbeta1
attctcccaagaattagaaatgagtagatcaaattattcacgaatagaatcaggaaaatcagatccaaccataaaaacactagaacaaattgcaaagttaactaactcaacgctagtagtggattttaatcccaaatgagccaacagaaccagagccagaaacagaatcagaacaagtaacattggatttagaaatggaagaagaaaaaagcaatgacttcgtgtgaataatgcacgaaatcgttgcttattttttttaaaagcggtatactagatataacgaaacaacgaactgaatagaaacgaaaaaagagccatgacacatttataaaatgtttgacgacattttataaatgcatagcccgataagattgccaaaccaacgcttatcagttagtcagatgaactcttccctcgtaagaagttatttaattaactttgtttgaagacggtatataaccgtactatcattatatagggaaatcagagagttttcaagtatctaagctactgaatttaagaattgttaagcaatcaatcggaaatcgtttgattgctttttttgtattcatttatagaaggtggagtttgtatgaatcatgatgaatgtaaaacttatataaaaaatagtttattggagataagaaaattagcaaatatctatacactagaaacgtttaagaaagagttagaaaagagaaatatctacttagaaacaaaatcagataagtatttttcttcggagggggaagattatatatataagttaatagaaaataacaaaataatttattcgattagtggaaaaaaattgacttataaaggaaaaaaatctttttcaaaacatgcaatattgaaacagttgaatgaaaaagcaaaccaagttaattaaacaacctattttataggatttataggaaaggagaacagctgaatgaatatcccttttgttgtagaaactgtgcttcatgacggcttgttaaagtacaaatttaaaaatagtaaaattcgctcaatcactaccaagccaggtaaaagcaaaggggctatttttgcgtatcgctcaaaatcaagcatgattggcggtcgtggtgttgttctgacttccgaggaagcgattcaagaaaatcaagatacatttacacattggacacccaacgtttatcgttatggaacgtatgcagacgaaaaccgttcatacacgaaaggacattctgaaaacaatttaagacaaatcaataccttctttattgattttgatattcacacggcaaaagaaactatttcagcaagcgatattttaacaaccgctattgatttaggttttatgcctactatgattatcaaatctgataaaggttatcaagcatattttgttttagaaacgccagtctatgtgacttcaaaatcagaatttaaatctgtcaaagcagccaaaataatttcgcaaaatatccgagaatattttggaaagtctttgccagttgatctaacgtgtaatcattttggtattgctcgcataccaagaacggacaatgtagaattttttgatcctaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttaatagggcgtaataacgtcatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagtgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaagtaaaaaaagaagaaaaagaaagctatataaaggcgctgacaaattcttttgacttagagcatacattcattcaagagactttaaacaagctagcagaacgccctaaaacggacacacaactcgatttgtttagctatgatacaggctgaaaataaaacccgcactatgccattacatttatatctatgatacgtgtttgttttttctttgctgtttagcgaatgattagcagaaatatacagagtaagattttaattaattattagggggagaaggagagagtagcccgaaaacttttagttggcttggactgaacgaagtgagggaaaggctactaaaacgtcgaggggcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgattacatggattggattagttcttgtggttacgtggtttttaactaaaagtagtgaatttttgatttttggtgtgtgtgtcttgttgttagtatttgctagtcaaagtgattaaatagaattctcatgtttgacagcttatcatcggagctccgatgataagctgtcaaacatgagaattcccg

SEQ ID NO: 48 - DNA, 4154 bp, synthetic construct
  source 1..4154: mol_type = other DNA; note = pLME106
gatccggcggaacttcacgtcctggcggtggagttggcgggcgcgttccagccgttcctccagcacggtgatccgggcctccagacgctcacgctcaccctgctccaggtgccgggtcaccgtcaccgtccgcaccggccgggcctcggcctgggcggcccggcgttcctcactggcccgcttccggcaatcgtcggaacaccacacccggggccgaccccgcccaccgtgggcctccaccggcgccccgcagtggggacacgcccgcagcgccgacgcatcctcatccaaggccatcaccgggtcggaatccatacccgaaaccatatcgtccggacgatgaactgcgccagacagctaagaatgcacgaggtgtgtctccgattctcaggaaacgctcagcattttccgagacgttcggcgcacgcacacacccccacaagaaccgacccgcccagcatccgccgacacgtcgatccgcacccgcgatgggctggccgaggccgactacgaccgctagtcagcacctgcgctgatctaccgtcgccctgaccgactctcccgtcgggattgtcgccggccgctgccagcatggacctgcggccccgccccctcgccctgcaactcgagggaggcggggccgtccaccccccacaccaccccgacaccgtgatgcgcccatgtcgcctaacgggttgcccgacctccccgacatcaagaaaacctgacaccgtcgccgcaagcgctacactgactactagtagtcaggaggtgcgtgatgaccatcgccacatcggtgaaactctccgaagagaccggccgcaaactcgatgaactagcccgggccaccgggcgatccaagtcctactacctgcgcgaggccatcgaggaccacatcgaccagatggtccacgactacgccatcgcccgactcgccgacgacgtgcgagccggccgggccgccacctacagcgccgacgaagtggaccagatccttggcctggacgattgagtacaccgaccccgccgtcaaagcactgcgcaaactcgaccgagcccaggcccgccgcatcaccgcctacatacgtgagctcaccggcctggacgatccccaccaacgcgggaaaggcctcaccgggcccctggccggactctggcgctaccgcgtcggggactaccggatcatctgcgacctgaacgccgaccgcctggccatcatcgccctgaccatcgagcaccgatcccaggcctaccgctgacacgcaaccccgcaccctcggccaagacgtcacacaccacccgccccaccgagcactgaggatgtcaactcgcccgagccggcctgccggccgtcttacgggttgtcttggcgggcggggtgtctttgccctggcccagcagccccacgatctcccgcagcgtgtcggcggtggcggcgtcccgggccgcctgacgctccgcctccgccctggcctgctcggctgcctgcgcccgatcctccgcggcggcggcctgctccctcgcctcggccagctcgccggtcagggcctcgacccgggcctgcacctgccccaggcgcgcctccgcctcctgctgcacctgctcggcccgggcctccgcctggtcccgggccgcctcggcctcggcccggtgctgatccgccagggccgcctcggccaccgcttcggcctgcccatccaccgcctgctcggcccgagccccgaactcctcgcgggccgcatcactcgcctgacgccacgccgccgcccacaccagacccaacggctccgacagatccggcggggccggcgtctggaccgacgccgagacgtcgcgcaggaaccccgccgcagcgtcggtggagcaccccgcctccgccttcaacgaccgcaccgtcacccgccgacccgcaccgctcaaccgcgcataggccgccgccaaccttgacccattcgactccatgacccaccctcccattctgtaccctgtacctgttcctaggtacgttcctaatgtacctcaccggatgcagaacccgcaacccccctcacactccccctgcacggggcccgccccctgcacccccgctgccgcgcccgctcctgcgtcgcggccttgcccctgcccaacgccgggccggcgggcagcccaccagaggctctgtgagacgtcggcgcccccgtccacctaccctaaagaccaaccggccgtggaaacgtctgtgaggagccttgtaggagttcccaggacaagccagcaaggccgggcctgacggcccggaaaggaagtcgctgcgctcctacgaagaagcccctctggggacccccagaccccggaactatctgatttggtttagcggcgtacttccgtcataccggaatttatggcatgctgtggtcatggcgacgacgacggtcgatgagcagtgggagcaggtgtggctgccccgctggcccctggcctccgacgacctggcagcgggcatctaccggatggcccgcccctcggcgctgggggtccgatacatcgaggtcaacccccaagccatcagcaacctcctcgtggtcgactgcgaccaccccgacgctgccatgcgcgccgtctgggaccgccacgactggctgcccaacgccatcgtcgagaaccccgacaacggccacgcccacgccgtgtgggccctggaagcagccatcccgcgcaccgagtacgcccaccgcaagcccatcgcctacgccgccgccgtcaccgagggcctgcgccgatccgtcgacggagacgcctcctacgccggcctgatcaccaagaaccccgaacaccccgcctggaacaccacctggtgcaccgaccacctctaccggctggccgagctcgacacccacctggatgccgccggcctcatgcccgccccctcctggcgacgcacccgccggcgcaaccccgtcggcctgggccgcaactgcgccatcttcgagaccgcccgcacctgggcctaccgcgacgcccgccgcatccgacaacgccacgaatacccgaccgccgaggactcggccgacctgcacgccgtcatcgcctccaccgtcgaggcgctcaacgccggctacagcgaacccctgccggcccgcgaggccgccggcatcgccgccagcatccaccgatggatcacccaccgtttctacggctggatcgactcccacaccgtcaacgaggccactttctccaccatccagagctacagaggacacaagggagccggcaaggctcgtcctcgtgcccgccgtgctgcttctatcaccgattgggaggcatgatggctgacgtccagcaccgcgtgaagcgtcggggcacggcccgcgaggccgcagaacgtgtaggggcctccatccgaaccgcccagcggtggacctccatcccccgtgaggaatggatcactcagaaggccgtcgagcgtgaggagatccgggcctacaagtacgacgaggggcacacgtggggcgagacctcgcgccacttcgggatcgcgaagaccaccgcccaggagcgggcccggcgggctcgaagggagcgggcggccgaagcggagaaggctgccgaggaggccgaggccgcgctgcgtccgacactcttcgagggccaggagcaaggttctgcatgagcaaccccgagtcctcgggtagaccgtctggcccgacgttaagcatggctgaagcggcccgtgcctgtggggtttcagtgtccacggtgaggcgtcaccgtgatgccctggtggcccacggtgctacccgtcatgacgcgtcatgggtgatacccctatcagcgttgatttcatgcggtttgatgccccgggtgacaccccctgatgccccgtcacccaataacgtggcgcctgccatgacgtcccacggtgacgcccccctgacgggggaagtccaagagctgcgcgagcgactggccaacgctgagcatcgagccgagctagcagtagaggttggggacgacgtctcggcgactccggagaacaccaagtcagggtctcatgagtgtgcgatagcttgagctgtctaccaatctggatatagctatatcggtcgtttgtgtctgattcgccagtgagccaacggcgggggcgacacgcggtggcgaaaccccctggcaga

SEQ ID NO: 49 - DNA, 22046 bp, synthetic construct
  source 1..22046: mol_type = other DNA; note = pTZC1
gcgagcacacttccctcacgctgactcgtcactccgacacgctcgctcgtgtccgtgaactggcgggcgacggcccgccagtcacccggatcgatgaccgccgcattagtcgaaacccacacgccatcctgggtcgcatcgggccgcgtgatgtacgccagcgccttcgccggcgtcgtcttgatctgccccatcttcacaacagccatcactccgcccccgcttccgttgcggcgtcgatggcccgttggacttgtcccagcagctcccgcgtggcacgcatctgctcaaccgtcaccacatcatcagtgttcacctgccgagcgatctggttgatgttgttcccgatcctcgacaactcggcgcgcagcggagccggatcgaacgccacgcgacgcacgacgatcttcccctcagtgagagcgcgccgcgcatagtcggcgaacgtcctcgcctccgcaagttccatacgccgttccacccgcttccattcagcatcactgagccacaagcccttgaagacccctcgcgaccgcttcgcgccctgctcagccatgcatccactcctaccgggtttgggcagagcccatagtgcccagggggccacgcaactgtgtaacagttgctatgcttgctaccccgtcagggtcagtctacaacctacggtctacatggcgctagaatgagaaacggatcgtccggctaaccggacgatccgagatcggaagggccgcccaggggcggcccacgatgatccttgtacggggtgtcccgcgctgcccgcgcgggacaccctaggggtcgccgtcggcggccccgcaccaagatgaaggagggcgtatggccaccaagtccgaccccgatctaatcgacgacctgctgcgccaagacgaggcggagctgcgggcgaagaagaagcggctgcgcgcctttcagacggctctggatgagcttcggcacgccagcgaggcagtggccacggccggtgccgccctcatcgctgccggcgatgtctcccgcgccgaggcgagcaaggtcttcaagctctccaagggagaacgcgccgccgcgttccctacccggcctcgctcagagtcgagcgtcgcggatgcggtcgatgagccgccgaaccccgtagacgatccatccgatgagtcggatgaacagcacaccgatctgggtcagtagccacaccgcgacgtggaagatcgcgctcagtccgtacatcgcgatgatgaggatgagcgcgagcacggcgatcacgccggcgatcatcaaccacgcgagccccatcgctcatcctcccttcaactctcggtgtctccatggtgccacttcgggatgactcttgtcccgatgccatcaggatggtgcgctccaggtcgccgtcaagggcgctgcgcgtcgctgcgcgatgccgcaagcggcacccttgaccgcgaccctccacacaccgattggcagttatcggaacgaaggggcgctctgggctcggacgagggctcggcgtaccgacgacgtgctcacaccgagcacggtggcgatatgcgcgttgctctgaccctgcgccttcatgcgcaaggcggcgtcggtccgctccggacccatgacggtgggacggccaccgactctgccctgggcccgcgcgtaggccaggccccgccgggtgttctcccggatcgtgtccacgcgcagctgggcgaacacggccatgatccccacaatggcctggcccatcgggctcgacgtgtcgatgctcaacgccggctccgtcaggctcctgatactcactccctgcccgatcaggtcatggacgatctcgatggccatgacctcgcttccggccagccggtccagcgcccggaacaccagcgtgtcgcccggacgcagatagtcgcggcacgccaaccactgcggccggtcggccgcacggctggactcgccgtggtccacgaacacacgctcggccccggccgcgcgcagctcggcctcctgcgcggccgggttctgctcgcgcgtggacacgcgcgcgtacccgacgatggtcatcttctcccccgccccacagggccgcctcggtgcggctcgtcgtcgccgcgctccgctcgacgacgttccacctcccgctcgatgcgctcgtgcaggccgctgcgggtactctcccgccgggctaggccgcgaatgctcgccgcccacgccttggtgcgcgtcttcacgtcgtcgagctgactggccgtcagaccgtagacgcgcacctcttcgggctggatgcgctccacgttgtcgaggttctgggcgaaccgtcctaggtcgaagccggcatcagcgttgcgggccaagttcagcagttccttgtcgctgtacctgccggaacgccggatcgcgtccacatcgagatagtcgcgggtctcggcccgcgagaacagggcacccaccttgttccctaccgcgtcctcgatggcaagcacgggcccgacctccagccgcacgggcgggtgagcccgccagtccacgcccaggtccatgtcggtactgcgcccttgcgcactgacgatggtgagctgggcgaacgtgtcctggcggcggcgggtctccacggtgtagccggccgcacgcagcgcggcgataatccggtcgagcgacgtgccgaatcgggcctgggcctgctggacggtgaacaggtccacgtcctctgtgggccggtcgatcagcccatgctcacggatcgcgcccgagccggccagggcgaagccggcatcgtcgccgacggcctccagggccaggcgcgtgatgcgccgctgctcctcctggtcaccgctcacacgggcacccgaagccgggggaaccgtccctcccacaagacgcggacgtgcgggtccatgttcaggatcggccacgtctcgatgagccgatctcggttcatcaagcggccctgctcgtcaaccgtcccctcggcgagcagcgcctggtaggccatacgacgccaacccaggttcgacacgtccacaccgagccgatcagcctgccagcgcacagagtgaggcaggtcgatgggcccgtcatagggcccacgcagctcgtcgagcgaagcgggcgcgtcatagggcttgacatcgcggaaccgcacacgagtcgctgccacctcagccatggcccgcctccttccatctcatcccagtttacccggccaggtcgaaaacggtctatcactggtttcggtttcggtgggggttttgaccgcccttccaccgccaacctacgcggattgtgaatgtgatcgctatatgtcgtggattcttgatcactctatggcgtgctggtctggtggaccgggaagacattgctcttcgcatggcaaacggtgccgtctttaggatttctctgcgccaggatccggaagggatggcacttcttctcctgccgacatcggtcggagcggctgatggtatcgcgatcagcttgcctttagcaccccacggccgtaaacagtgtggcccactgccccgcgtccaactgtttcggcagtgcgtgtgccggaacaccggcctcacgcaagacctgggcacaccgtcgcgcacgccgacgccccacaacccgggacacgatgtcggcgacgccacggcccttgccggtgaacacgtcatggacgaaggacgcgtaccgcgcacgctccgaccagtccacgagaggttcctcccggcgacgtatcaccaggacaccggcgtcgacggtggggcggggacggaaatgcgtggacggcacccgcccgtactgttcgaagtcgacccacggccaccactgggcagtcatcatcgtggcaccaccgacaccggcgcggcggcgcgcgacctcccactgcaccaggagcaccgcgtgggtccaccccggtgagtgaaggacgtgccgcaggatcgcggtggtcaggtggaacggcaggttccccaccaggacgtgcgggccgtccggaagcacaaaatcaaggacatcctgctcgtacaggtggacctccgggcgcagccgcttctccaaccaccggacagaggccgggtcgatctcgacggcggtcaacgatccaccggcagccaagacacgatcctgaaggggaaaggtcagcgccccgtgtcccgggccgatctcgatgaccgggacggaggcgttgacggggacgaggtcgacgatccgtctgatcgtcgcctcattgacgaggtagttctggccgttctcgtgacggcccttgttcggcctgtaggtaggcatggaaagacactccgcagcagatatcgtgctccgggcatgccgaaaaggccgcccggctggacaagctgagcggtgggtgtctctacctccgtggaacgtcccccgttagcgcacacacccaccggcggctccggggttccggagccgaccgaaggtggtgaaaattgcattcattgcacccatggggtgaaccataccacagcacgcggatgcctgacctgccctgtcccggtcatccaaaactgtcatgtggacctgacccggtcggtctgccgactggtgctggatacgccgccggggtcagattggtgggtgagtacctacgcctccacggaggtacggcagcaagcaacgtgcatgcgctggcagaaacaattctgtctgatgttctccctcggctcgggctaggtaatgagtagtagtacaaaactgtactgcccgttctctcttgtattgaaatgctaaaggtttacaagacatctacggcgaacgcactgaaacagggcgctcctgcgagaatcgacccgaaaactgtctcgtatacctgtctcaccgtaatgtgttccaccttcttccaatctggggtttggtgaggcatgatggtggtcatgagactgttgggctacacccgggtgtccaccgtcggtcaggatccgactcttcaacacgacgccttggtcaccgccggggttcaggaccgtgatgtcttcagcgatgtcacctccggggcgaaaaacgccactgagcgtccggggatgaagaagctcctcgcctacgctcaacccggtgacacggtggtggtgtggcgcatcgaccggctgggccggtccctactcgatgtactcaacacggtgaacctgttacgcgaacgagacgtgaaaatcaagtccgtctccgacggcatcgacccggagacctcctcgggccggttgatgctcggcatgctgggcaccctggctgagtacgaacgagaactgatcaccgaacgcgtcaacgccggcatcgccgcagcaaagtccaacggcacccgcttcggccgaccacctgtggatccagaggtggtcgaccgcaaactcgccatcgtcgccgaggaacgagccaaaggccgcagtgccgaagacgccgcgagcatggtcggctggtcacgggcgacactgtaccgccatctgcagggcgccaaacgacgacagtcagcactgcccgcctgacacggacacaatgaccagcgcgtgaggtgacggtgatggacgagatgcaacgctgggagatcctccggctccacatcgaagacgacatcaccctgaccgacctggcacaggccaccgacatcagcacccgaaccctatcccggtgggtagcccgataccgcgccgacggaatccgcgggctacgcaacaccacacgatccgacgccggagcccatcgcatatccgcggaactcgtcgcctacatcgaacaccttggtctcaccaagccacgcccatcgatcgccgccctgcatcgcctcgtgagctgtcgagcacaacaactatcgctgaaaccacccagctacgccaccgtgcgcagcatcatccaagcccttgacccggcgatggtcaccctcgcattggagggcccgacgtcctaccgagatcgacacgaactggtctaccggcaccgggctgaacaccccaacgccatctggcaggccgatcacacccaactcgacatcctcatccagaacccggacggcaccccgactcgcccctggctcaccatcatcatcgacgactactcccgggcagtgtgcggctacatggtcaccaccaccgcaccctcggcaatgaacaccgccctggcactacgccaggcgatctggcgaaaaacagaccccacctgggcgatgtgcggtattcccgacgtcctctacgtcgatcacggctccgacttcaccagtggccatatcacgtacaccacgacagcactgaagatccggatcatccactcgaccatcgcccgtccgcagggccgcggcaagatcgagcggttcttcagcaccgtcaacaccgaacttctcaccaccctgcccggccacctcgcccccggcgtccgcaacccacaccccgtactagacctgacgagcctggataccgccgtcggcgagttcatcagcagctacaaccagcgcacgcattcttcaatcaacaccagcccgaaagccgcgtggatcgggcaggggtggatccccagaatgccggagaaccttgaagaactcgacggactcctgctgcgggtctccacccaccgccgagtccagcgagacggcatccacttccaaggccagcgctacatcagcccgaccctggcaccttttgtcggccatgacgtcaccatccgctacgacccgcgggatctctccgagatccgggtctacgaccacgacacgttgctgtgcgtcgctgtcgatgaagaccaccccaaccagcgctacagcctggccgatatccaggccgctcgtcgacgccgacggcgtcaactacgtgccgggatcaacgagcgcatccccatccacgagccacgcccatcagaccttgcccctgtgaaccccgatgtgagcgccgaagcgccacggccgcgtggtcgtacgtctcgcctgcggacctatgaagaggacctgtcaccatgaaccgcgacttcatcgtcaccaaagagcaccgccgcttcgtcgagttcgccaacgcgatccgcaaagacgccaccatcggcatctgccacggtgatgcaggagtcggcaaaacacagtccgccagacgctatgcccactgggatgctctgggctcgttcattgacgactggggtccacgcagtgaatctgacctggccatctacgcgacggctcatcgggcgcgcaccgtgttctacacccctgaggtgcaaccgaagtaccggacgttgatccgtgacatcgaattttaccggggcaaactcgacgtctgcatcatggagcatctgatggccaccggacagcgggacaggctccacatgcgcagatccagtggcgagaagctcacccaactgatcattattgatgaggcagaacgtctgcctcccaccgccctggagatgctgcgcgacatccacgatcgtgacggtgtggcgatcatgttcatcggcatgcccggtattgaccagcgcttccggcactaccctcagttgttcagccggctggggttctcgcatcgctaccgtgccctgggcaaagacgagctgctgttcgtgctgaaccggcactggaggcgcctgggtagagaattgaacccggaggatttcacggatgcgcaggccatcgccgcgatcgagcggctgacccgcggcaatttccgtgtggtggagcgattgttcccacagatcaagcgagtgttgaagatcaacgagttggagaccatcaccgacgatgtgattgaagctgccgccagcaccctggtactcggccactgaccaggtcagtacgacacatagtgatcaaaaaagcaggccacatagcgatcacattcacagcggatcacacgacataccgccaaccggtgcaaaaacgatcgtttttgagacgcaccgcgagagcggagccgttagccgctcccggagcgtccacaacgcgtctcaaaaccggtcgtgtcgcaccctggttttacgcccgggcagtctgcttatgtgtgataaagaagcaatagaagtgcaaaaaattttgccgttcctatccgacacttggccattgtgtcggataggtcgggcggttattcgggcaagtcaatcttgccgacaaagctgtaataaatctcaatgtcctgcctgcgggtgccgttctcatcatagctgcactcatgcaccacaattttctcgatcatttcccgcaagagagtgggggtcagttcttcaaaggcaaggtgcttgcggacaatgcccataaatttctcggcgttgacggtagttgcctgtgacttgtccagttcggcttgcagggcggcggctctctttttcagctccgcttgctcggcttcgtagtcagccgacagttccatgaaacgctcatcgctgattttgccgtttacattgtcctcatacagccgcttgataatgcggctgatttcagaaatgcgttcctgcgcctgttcaagctgcttgatggctgcggcggtctttcgcttgccgccgatctcgttctgctggacaaaatgtaatccgcaggattaggagatagaagttccgttactttgggacgcactacctctctgtgaaattcattagattcgtcacccattgcattatcccaaaattgtgcgttctcctcccagatttttttactttcctctgttcccatgttctctcccactccccaaatttgcttttttgcttccattaaatcttccttactatattccattgttaccctccataacttctgattgttgccgtcttgacgattatgtatctttacattaccttctgaaacatatggcgcaccttgtccaggcggctgtttggacggcggggctggatgaccggctgaccgacagcggcctgatatcctttcagctctgtaaggcatacgctccgcccgttggtgtaaaaggccagatcagtacggtatgcctgtatacagcgggcgggaatctcgccagtaaagacaacttcatccttttttacctgggccgtttcgatggtggcacagtatttcggtgcatcatgataagccctggaaaggtattcctggggcgcatagaggatgaaggagagataaggttccagcagctgcgtccccgattccttcaatgcctgttccaatacaatcggggccaatgagcggaagtccgccggcgtgctgaccggactgtaataaagcccgtattcaaagcaaatcttacagtccgttacgttccagccgaacaagccctgctccagcccgtaacggataccatccctgacagcgttttgaaaactctggttcaagtatcccagcgaaacccggctctcgtattgtacaccggagccaagcgagagtggtgtaacagacagtcctatggatgcccaaaacgggttgggcggcacctcgatatggatggtgtggctggctgctttgagcggccgctccatataaatgacggagggttcctttaccactgtttcaagcttgtatttttccgacagcaaagcggaaacaacctccaactgcacccggcccaaaaaagaaagaatgatctcatgggtgatggaatccacttcgcaacgcaaaagcgggtcagtatccgcaagttgcgtaagagcgtccagcagccgttctctttgcgctgccgttttcggcgcaatcgtcgtccgcagcatggggagggggtcctcgcgccaccttttacgagggagccgggtttggtcccctaatacatcgtttaacctcacgctgtcgctgggaaggataacaatttcaccctgataagcggtgtctgtccgaacaatttcccctttggatggaatacgcatctctgtgattttcagcttttctctcccggccagggccaccgtatcccgcaggcgcagcgttccgctgtataaccgtagatagacacgccgctggccgcaatcggtgtactcaaccttgaaaacgctgccgcatagggcggcgcccccctgttccccaatcggttggaacagccctgtcaccgcatccatcaacggttgaatgccaaggccatttttggcgctgccatgatagactgggaacagggaggcgtcttgaacccgctgctgttcctcccgcgcaagtttttcccggctgattggttctcctgcgatatacttttccaataattcatcgttattttcgatgaccgcatcccatgcttctatgtcggtattttcctccaggactatttccggggacagcgacaccgtctgcttgatgataatatcggcggagagcttatcccgaacagactgaaccacgctctgcaaatcaacgccagcctggtcgatcttgttgataaagataacggtgggaatgttcattttccgcagggcatggaacagaatacgggtctgggcctgcacgccatctttagcggagatcaccaagatggccccatctaaaacagccaaagagcggtacacctccgccaaaaaatccatgtggccgggcgtatccacaatgttaactttacatctgtgccactggaaggaagtgactgccgcttgaatggtaatcccacgctgccgctccaaaaacatggtgtccgtcctcgttgtccctttttcgacgctccccggttctgaaatggctccgctggcatatagcaggctctccgtcaaggtcgtctttccagcgtctacatgggcaagaattccaatattgattattttcatgtgattgtcctccctttacagccccaaagggcataaaaatccccagcagtaaaatacttttaccactggggattataagttgcggacatacacatatacagcatacacctgtttgtgattgctgtttttggggatatgtcaaaattgataaggcaaaagtattcttaaattgggtacaaaaaactaagcccctacaaaaggagctatcataatcctttgttcccactatttgattatagttttatttaagaataccttgccgcatattttttactccttttctggattaaatcattgtatcacatcagttttaggaaagcaagtacctaaaagaaatttttcttccccttatatgtaacaatcataccggcttcctagcgttcagaatgttttctgctgtctgctgtggtgtttggttggaattgtccaaccaaaagccgatccgtggtgttgtctgcattttactaaatacaaattcaatgtatacagaaagatataaggagtgggagggattccgccgtagttggcattgtaggaaaatccaaaagtttagattttcccacaatgcttatcttttggtctttggttcggaatagtgtagtgctggcggtctatctcttgttttcggttgcttgcttccttaccgtacatgagcattcgcgcagtgcattcccgaccacgtccggcacggcacctcgaccgtctgcgccgcgctgaacattgcgacccgcttcgacaaactcgccatcacgtaccgcgccggcgtcaccctctgcgccatcctcacctgggtccgactattgggagacacgacctaggagaagaccggctcccacatccgctaccgcttcgtgggaggccgtggcccggctcccgccgggcatgaagaagggtccgctgaaggtttggttgacctcctacggcagcagctcagccagttctcgcagctgctcaaggatccgctcggcctggtcgggagcgacattcttgtccagctgcttgagctgacgctgcatcttctcgatgactgcgtcggaaccagcggactcgctgtggagcttcaggaagtgtacccaagcacgcgtctcgataccgaggatgggcacacgcatcgtcgactcaacgaacgaatccatgagggggcgacaccgaggcagtcgtccgaagttgccaccgccatcgttcaccggatgcaccgtcacgcggtcagccttcgacgggtacggcgtcgcgtcggccgggtactccagctccgcactgccatcggtctccttgatgttggtccaccagtagtcgcgccactcggcggcgtacttggcggactcgacccacggctcggccaccaccggcacgccgctgacggcgctgtcgaggtgccacggcacgcagcacaccaggtcccagtcggtgcaggcatcgggggagacctggtaccgcagcgaagggaccttctccttggccagcatccaccagcccttgagctcgatgccgatcgcgatgggctcaccctcggttccacgattcaccagacgcacgtcggggaaggcctgcgccgaccgctcgaaccggtaggtcgaccactcggagtccgggtcccagatctgccgaaggctgttcagcgtgcgcacgacctccagctcgatgcccgagcccaagaagctgttgaggctgaacaggtcggtggcgttgaccccgctgatctcgttcttcgactcgaactcgcccggaagggcttggagggccgcggagacccccttctggagcttggtatgcggatcgttgggatccagcaccgggcgggcaggacccgcacccacctgggcgctcgtcgccactgcatcaaccgacattgctctcctcctcggggttggcgtcgaagagggccacgagctcgtcgttggtctcggcctcctcggcggcgacgtcaagccgctcgtgcgcaagctccgcgaagtaggggtcacgctcggcaaggaaagcctgccgacgcagggccaccgcagccacggagcccgtgccaagcccgccgaagggctcccatacgacgtcgccctccttcgtgacggcgtgcaccaggcgctccatgagctccagtggcttctggttcagatgggtcgtgctggccttcgtgggcttgtacacgcgcggcgccgaccgacgcatcgagcccttcatacgctcgccgtcgtgcagcggggggcggctccacacgttggtcaggccatggacgtggtgccactggtggcgcatccggtcccactgcttcgcggtgaccgacgtcacaccgtcgagcgagaagtacgggcggccgctctccaggccgtgctgattgcagtacgcggccatggcggccacggcgactcccggcggccagtaccagagccagtcgttcgtcaggtacttgcgcgtggcggcgttcttcacgccgcaggcctcattggccaggtacatcggcagaccagagcggcgccactcgtgccgcagccactgctgggcatcgagaatgcccgcgtcggtcgccgcctcgaagcggcgctggtagaggacgcagacctccgtgacgacggggaagcggcggatggtgttgccgttgacgttgcccgcgatgtggctcagacccttgtcccagaccacggtctggacgtagtcccagccctgacgcttcaactcaggatgcaccgtggcccagcccacctccgtaccccagaaccacagcgccgtgcccggggcggcagccttcgtccactgctcgatgtgcggggcgtaccagtcaacgagcccttcctcgtcggtggtgtccccgtaaaaaccgcggacgccgtaggcgccgtcgctgatgatgcacgtcggcgacggccaggacgcgtaggcgtccgccacatcccccacgtgcaggtcgtagggcctcttcttctcggccatgctcccagcctctcataatcgatggttactggtcattcgcccggtccaggtacgtcaggccggcgcggcgtagcgcgtcggccggcgtgccgccgctccagtcgggggcggtcccggtcatcggcaggggcgtggccgaccgggctgtgttctccagctcgtcggccagttcgtcccactcgggcacgtcgaggacctggttgaccgagacctcgacgacgacgctcaccccgcgctcggccagcagccggcccacggtctccttgtacgcctcggcgtagtcggcctggtcctgctcccaccgggcctctacagcgtcagcggcgcgccgcgcctcgatgagggtggccgccagcggatgggcactctcggctgctcgctcctccacggccttgatgatctttccggcctcctcatacatcgcggtcacccggtcggggtcactgtcccactcgggtcccccgagctggggcatgtcggcctggagcgcggccagccggtcgcgctcctcggccgtggccacggcctcccagagtgcatcggccgccgcgtcgacgcggcggccgagttcttcggccgcgtcctcgtagagctggtagagcccgaagtcctcgaagacggcctccacgtcgagcgcgaggcgcaccggctcggtgcgccaggccagcaggtcccgatcatcggtgccggctgtcccctcgacgacctggcggatgagggcggcctcccacgatccgggccggccggccagcagctcgtcgacgctgccgatgttcgcggccacggccgtgagcacgtggcacgcgatccccgcgaagtcgtccggctccgtgtgctcgcttggggtgcccgctccgacgacgcgacgccggcgcgccgcgtcggtcagggccgtgatcgcctgggcgatcgggtccgggactgtctcggtcatgtctggctccttcagtgggtggtgatgccgggcttggggcctggcgtcacggcgtcgaggtcggggaacgggcggccgtcgttgtactcggcttccagccgggcgactttctcgttgacggcttcttggacgaactgccagtaggggcggatgccgagcttcgcctgcgtgtagaaccacgctcccctggcacgccggccttcctcgatggtgtgcttgaagctggtgcgcgtccatccttcgccggtcccggtctttttcgcggcctcgtgccgggcctcgtcaacgttgggcggctgcgccgcctcgacggcctggggcacgagggggttgctcaggttggtggtgcgccgctcgggccgcttgctcatttgatcgctcctttagtgatggtgtcgaggtgctggcggtagaggtcggccagctcggcggcgtccttgccgccccactggtccaggccggtggcagcctccagcgcgtcggcgatgacggcgcgcttcgggatgggcggatcaaggatggtcaggccctcgatcttctccaggtcttccagagcgcttcgcgcgccgatggtttgcgcctcgtactggttgacgatcaccccggccacggccaactgcgggttgtagtacttgcgcacgatggcgatcgtctgaagcagccgaccgagcccggcgatcgagtagaccttggcctgggtgacgatggccacccgctcggcggccactaggccgttgagggtgaggtgatccagcgatggcgggcagtcgatgagcaccaggtcgtagcggtccgcgacgctggccacggcctcgctcagccggtgctcgacgccgggggtctgcgtggtcagcagttcgttgcgaacgctggtcagcgcctcgttgggcggcgtcggggccacgtcgaggccatcccacacgccggggacgatgacgctctccagcgtctcggtactgcgttcactcagcgcgtcggccacgccgacgtcctcgggcgtgggcgtgtctttcgcggccgacatggtggcgttaccctgcgggtcgaggtcgatcaggagggtccgccggccctcggtgacggcggcgcgcgcgaagttgacggcggtggtcgtcttcccgacaccgcccttctggttgctgattgcgagggtcatgctcatgctgttggttccgttcgtgcgattggtgcggttcgtggtgttagtgccattcgttctgttcgcactaactagtgtagtcgcttcccgcctcgctgtcacggcgtcgaggacagtcgggagatcaagtccggccggaggccggcgcaccgtgggggctccggccggacttgttctcactgttcattctattcgttcctttcgttctaatggtgcgaatcgcaccattagaactagtcggtgatggcttcgatgatccggtgcgcggcggccagcacacggctggcgctcttgcggatgaggtcggcgtctcccttggaccatccggcgacgtagccgacgctgtaggcgctggtgtcgagtccaacgatgccggcgaccacgtgggcgacgctttcggcctcgacctcgcattgtccccggtgctcgtggtactcggtgggggtgatgtcggcgtgcatgagcgcgtgggcggcctcgtgaagggtcgtcttggcggcttgggcgggggagatatcggccgcgatcacgattcgcttgtcgtcgtggctggtgtagccgttgagtccggccccgagctggtcgtgctcgatggtccagccctggccggtgagccagtcggtcacggcctcggcgatgccggcggggtcgtcgccgctgagctggtgggcgtcggcggggttctcggggatcggctcggctccctcgatggggtcggtctgggcgaggtcgaagacggacacggggaagaatcgcgtgcggcgtcgctcggtctcctctccggtggtctcgtcctcgatcgtttcggtgacctcgcggccgccgaagattctgattccgcgctcacctttgcggacctgccggccgagcttctgccaggtgcggtaccccgcgacctgcgtcgcgttctcgcgctgggcgaggatcaggagcaagttgttcaggctgtagcggtggaacttcccggcgaaggcgaggaactgggcccatgcttccgaggtggccagggcctcaacctgctgggcgatggtctcgtgcagttcggccgcttcggtgcggcgctgctcgggggtcttgtgggtcttgatcttccgggccatgatgttggttcctttcgtgtgattggtgcggttcgtgcgaactgcctgtcttccctcacgtttttttgccattgaaggcactggaagtgccgccagggagggagcccggagtgcaagggaccgtggaataccggactgagcgcagcgagggaggatatgccgcgaaagcccttgcgcgtagggcggacgaccgtatgctgccgaaggcttcaatggcaaaaacgtgcgccgtaggcgcatgctgccggccgcgcagcggccgtccttatgatgacggtccgccagggccgcctggggagggtcggcctgggggccggctcgttgtggtcggctgtccacctgtgggcgggccagccggtcgggagctgggccgtccacaggtgcgtcaggcggtcccctctacggagcggccctgtggggcgttctgcgggccgtgacccccttccgcgtgtggttgcctgggtggggttgagttcggccgtttcggggcgttgtatggcgtcgtttttcgtggtcgatcatggctgccgcctgtggacggcccttcggcccgcccacagttgccttcgaaaccgggtggccgggttccggcgtggttgggacgagggttgcccccggcggtcaggcggcagtgacgaggctctgatgaaggtggtcgagtgctccggcggcgatcgcttcgcgggccgctcgatggtcagcgcggccgtctggtcggcgcgggtagggggggtagatgtcccagggtcggccgaacgtgagcgcaatctgtcccgtgggctggtggcgtcgtttgtggcggcctggtgtggtcatccattccagttcggcctgccaccattcccacagatcgcgctcggccttgtagcggccttctcgtgcgtcgagacgcccctcgacgccgagccgtcgggccgcgaggtcgcgcagctcgggccgggttcgccgccagccggtgggcgtggccacgatgagaccggcgtaggcgagtcggtcgagtaggggggtgatctccgtagtggtgacctgggggtgattgagcctggcgtagaggtttcctgcggcgaggcccagggcgtgggggttggtgaaggtgtcgtggcgggcgaggtctaggcggtgggtgagttcgtccaggaggaggtttctccatcggctgcccgcccctgcggggcgtgcagccccttgtgaccgggcatggtctgcatccatgtggatagcggtctggggctcgattgtccaggtggtggcgtgcctgccggtgcccgcctgggcctggctgatccacccgtcgccggccaggcgcaggagggcggtgcgggcggtctctcggccgatgccggcgagcagggcgaggcggcgcacgtctgcttcgacggctgcgctgacggcttggagggcgagcaggcacagggcgtcgagcacgcgccggtcggccggccctcctccgcgtgtccagcgccctggtgccgcatcggcgcggcgctgcacctggtcgacggtggcggcgatcgcttcggctcgggggtcgaaggtgggatcgtcgcctgcctggcgcgcgtgggtggccacgaacctgacggcggcctgccacactcggccgagcgcggcttcgctgctttgggctccgtgtgtggggcgcgcctggcgtgcacggcctccgtggcgtgcgcgctgggtgcgggcgtgctccatgccgggtgccgtggacacgagggcggccgcgtcgcggtagtgccagtgggcggcggctgcaccgagcaggatcacgtagaggacgcgggaggcgtcctcactggcggcggcgtcttcgtcgacggcagcgcggctcttggcgggcatggcacggcggggtccgggcagccacggccgcccgtcctcgtcgacgggcacgcgcagctcggctgcgtgggggagcgaggtcaccgtggccacgtcgccgaactcctcattgagtcgggtcgccagggccacgagctgggctggtgtggtggtcgggtgtgtgagggtggcgagatttccggcgatcacgcgggatgctccaccgtggcggtggggcgtgccgggggggcgcaagcatccggtggtcgggttgagcaggggagtggggtccaggctggtggcgagaccctggagggagtgcgtcaggtggtgcacgagctgcgggtcggccgtctcggccagggcgatccacacgtggcggccgccggtggggccggactcgcagatgacgtgctcgatgccggcctggtcgagcaccgtggcgatcgccgtggcatcccgctctgcttgggcggctccgccctcgtgggcgtcgaggtcgaggccgatgtagcggtagcggcgctgcgcgtccgtcaggtacatggcccacgggccggccggggccggccccgcgacgggcacctgcgtcgggtaggcgttcacctggtcgccgctggcggcgcgcacctgcgggcgcgggctcaacgtgcgtgtgagacgccacgccgcgccgatatgcgtagcgtcacggttatcggcatctgttgctatcatgtacgtgttccttccggggaaagacgcaacgatccctccttcggttggtgggcccctgccaagggccctaggtgtttgggtgttgcatcgggtgccgactgccaaaccggcttccgtacttggttcgcttgatctggttgtgttggggaactcagcagatcaggcgcttactcttctgacccccgccctgccaagggcgggggtctgttcatgtctatgcggtgcccgtagggtcctcctcgatgctcagttccggttgggtgctatctggggttggaagctcccatccctgtggccagccgtccgggcctgggacaccagagcggatcgcagcttcaaggactgcccactggggggcacccttggcgcgcgcgtaggcgtcgagcttggccttgacgtccggcgcaacgaagatgtgaagcgctacggtaggcgctccacggtcgcgacggaatcggctggtcatgacagtcatcatataagggcggctgtacatgtacacgggccgacacgccggcgtgtcgcgacttcttttcagatatcagagtcctcgggcgacggcggcggctgcgcgcagccatgcgcgttgtgtgctggggcgcaggctgtcccattggaggatgccgtccacgatggcggggtcgtgggggatcgtgacgacttcgcgggcgaggtcctggtagccggtgacgatgttggtgatgtcggtcttgctggcgcgcgggtcggcttgggtgaccacggcgacggcgttgtccgcgagctggcgtgagtgttggtcgcggccgcgcagcgcgtcgaggaggagcgcgccggcttcggcgtggtctgcgcgggtggtggtgggcacgacgatctggtcggcgtgttcgatggcttccagccacacaggatcggattcgtcgttgccggtgtcgatgaagatgaggcggtagtacttgccgacgacgttccagatgaggtcgacgtcgtgaggggtgacgcgctggtcggcggcgagctggatgggctggctgcgcagcacgtcgaaccggtcggcggtctggtggtgaacgtagtgcgccagatcggccgactgggctccggtgcccaacagtcgttcggtttggggaaggaggtcgaggacggtggcctcgtggggtccctgttcggtccgccatcccagggtgcccctggtttggttggcgtcccaggcgaccacgcccgcgccgccataacgcgcgaacacagcactgagtagcaccgtggtgggggttttgcctgccccgcccttcccgttggccacgatgatcgtcctggggccgggccagtgctgggagacggcgtgcacgtcgtcccgttccgcgcgctcctgctggctcgggttcatgcgcagcccgagccgggtggcaacgccgcgccagccctgcgtggcgggctgctcgatctcggtggctttgaggaatgaatggcggctctggacctcgcgcctgctgggcaggtgcgcgatggggctgtcggccgagcgtgggagagtgtgtgcctgggtgtgagggctgtcctggcgtggggttgggctgtcatagaccggggccgccgtggcggccgcggcctcggcgggatcgtggagttcgccgtcgggggtgacgatgacggtccacacgccgtcggggtcggaggcggtgagcgtggcgctttcgccggcggtggctgcgcgctcctgggcgatcgcgacgacctgggcgcgggcgtcttcgaggctggtggcggtgatcttctgcggcgtgccgtcgatcgtgacgatggcgctgccgtcgcgggctgtggtggcctctatgtccatggggatgctccttcgttcagctttgggtggcctgggtctgcatggctgtgatcgcccactggccgttctcttgggtcgcggtgacccatgcgtcccaggccacgtcgtcgggggcgtctgagccgtctcggggcgtggtcttgacgaggtagtggacgatccggtgcgcgtcgccgtcttggtcggggtcgagtccttcctcgacaatctcgcggatggtgacggtggtgtaggagtcatgctgcgcggcctgtgtgaactgggcctggcccttcgcggtgtcggggtcgtagtcggcggccgcttggcgtagcgcgtcggttaggtagatcgccgcccgttgggtggcgtaggcgctggtcttgtcgaccgtggtgtcccaggtggcggccgtggccacgtaggcgcggatgacggcctcggggtcggtgcggtcgacggtgctggggttgggcaggtcggcgagccaggtgggcttgcccccggggatgggtgtgccgtcgggtgcctgcccgattgtggggctggtcgtggccggcgtgctggtggcgctcgtctgatccggtgccggggtgtcgttggctgtcgtgtgggcgcagccggtgatggcggccagagtgatggtgccgatgacgagggcggcggcacgccggtgggtgcgcctcattggattctcctgtagttgctcggtgagccgtagatgggctcgaagttgatgccatcaatgtggttgtcggcgctgaccatccagccgttgccggcgtagatggcgacgtggtaggccggcgtgccgtagaagatgaggtcgccgaccttgagttcgccgcgctcgatgccggtgccgacctgctgctgctgggcggctgtgcggggcaggctgatgccgaggcggcggtagacggcgctggtgagcccggagcagtc

SEQ ID NO: 50 - DNA, 1615 bp, synthetic construct
  source 1..1615: mol_type = other DNA; note = pBC1
ccccgatggtttttcccccgacggctcgcctttgggaagtttgaagggcgcaggttccacacgaaaaaccacaaggctttttttgagtaataaaaaagcggacggcgagggcgtgaaaaaaacccgatgaacccttgaaaataaagggcgggacgggttttttcttatcttgataatatatagaaacaatgagattttaaaaaaagccctcaaacccttgataatactgggtttgtggcgtttttttggggtggataaataaaaaccctctgtgttatggttatgttgactagacaaaaccatacagagggctttacgcctttctgtatccagaaaggagttattaaattttatgcacgttaatcatagcattaaaacgtcaaatctatcaaatatcgaatttttgcaagataaaacgaaaacaggaaaagagagagattggaaaggcaagaaacaacggtctttgctgacagcggaacacttcgaggtagcagggctgactagcaaagcggaacagtgcgagagtgtgctgacacgttggtgtttaagcgaactgccgaagggttaaaactatatcaagcatggttctgtaaagtgaggttatgcccgatgtgcaattggcgaagatcgctgaaaatagcttaccagaataaaagggttgtagaggcggttaatcaacgtgagaacgttcagtggctattcctaacccttaccgtccgcaacacgagccctgagagccttccagagacgatttcagccatgtttgaggggtttaataggctgacgaagtacaaagcctttaaaacgtctgtaaagggctattttagggctttagaggtcacaaagaatagagaccctcatagtgaatggtttggcacgtatcaccctcattttcacgttctgctgtgtgttccatccagctatttcaagaaaaaagaattatacataaccgaacaagaatggactgacctttggaaaaaggctatgaagttggattacacgccgattgtccacgtgcaaagggtaaaacccaaagaacagcttgaggacatggaaacctatgaagaacagcttaaaaacgccattagggaacaaaatgcgattttagaagtctctaaatatccggtcaaagatacggacgtcattaaagggaataaggtcacggccgaaaatgtggaaaccgttttggcgttagacaacgccttggcaaataagcggttaatcgggtatggcggtcttttgaaacaggttcacaaggaattaaaccttggagatccggaagatggagatttagttcatgtttcggaagaggatgaaatcgctaatggtgcatttgaggtcatggcgaaatggcatatcggttttagagattattggattcaaaaatagcaggagagaaaactcctgcttttttattttttccgaagttattggcgaaagcaaactttttatcgagcgaagcgaaccctattgaatacctgcatggcaaggtatgtaaatgggcactctgtgatttttggatacaaaatagactctagcgagccgattttatgacgcagcaaaaaacgtagtctttttgcgttggagagcccttcaagtaaactgaccaaggtgg

SEQ ID NO: 51 - DNA, 1854 bp, synthetic construct
  source 1..1854: mol_type = other DNA; note = pEP2
atggtaaatctgcgcagacagccctgtgcagctgaaacgcggttacgtatagcttgccatatgtctagccatacgtaaccgcaggtaaaaggcatatttttcgcgtgtcatggctagtaaataacaccggtgtcatttagagtcagggaaagacaatgaaaaacgaagaaagccaccgggcggcaacccgatgactttcgcttatcacccagcacacacctgggagaaatcacggtcatgagtttacagactcatgcgcagaatgcgcacactaaaacacctacccgcgtcgagcgcgaccgtggtggactggacaacaccccagcatctgccagtgaccgcgaccttttacgcgatcatctaggccgcgatgtactccacggttcagtcacacgagactttaaaaaggcctatcgacgcaacgctgacggcacgaactcgccgcgtatgtatcgcttcgagactgatgctttaggacggtgcgagtacgccatgctcaccaccaagcagtacgccgccgtcctggtcgtagacgttgaccaagtaggtaccgcaggcggtgaccccgcagacttaaacccgtacgtccgcgacgtggtgcgctcactgattactcatagcgtcgggccagcctgggtgggtattaacccaactaacggcaaagcccagttcatatggcttattgaccctgtctacgctgaccgtaacggtaaatctgcgcagatgaagcttcttgcagcaaccacgcgtgtgctgggtgagcttttagaccatgacccgcacttttcccaccgctttagccgcaacccgttctacacaggcaaagcccctaccgcttatcgttggtataggcagcacaaccgggtgatgcgccttggagacttgataaagcaggtaagggatatggcaggacacgaccagttcaaccccaccccacgccagcaattcagctctggccgcgaacttatcaacgcggtcaagacccgccgtgaagaagcccaagcattcaaagcactcgcccaggacgtagacgcggaaatcgccggtggtctcgaccagtatgacccggaacttatcgacggtgtgcgtgtgctctggattgtccaaggaaccgcagcacgcgacgaaacagcctttagacatgcgcttaagactggccaccgcttgcgccagcaaggccaacgcctgacagacgcagcaatcatcgacgcctatgagcacgcctacaacgtcgcacacacccacggcggtgcaggccgcgacaacgagatgccacccatgcgcgaccgccaaaccatggcaaggcgcgtgcgcgggtatgtcgcccaatccaagagcgagacctacagcggctctaacgcaccaggtaaagccaccagcagcgagcggaaagccttggccacgatgggacgcagaggcggacaaaaagccgcacaacgctggaaaacagaccccgagggcaaatatgcgcaagcacaaaggtcgaagcttgaaaagacgcaccgtaagaaaaaggctcaaggacgatctacgaagtcccgtattagccaaatggtgaacgatcagtatttccagacagggacagttcccacgtgggctgaaataggggcagaggtaggagtctctcgcgccacggttgctaggcatgtcgcggagctaaagaagagcggtgactatccggacgtttaaggggtctcataccgtaagcaatatacggttcccctgccgttaggcagttagataaaacctcacttgaagaaaaccttgaggggcagggcagcttatatgcttcaaagcatgacttcctctgttctcctagacctcgcaaccctccgccataacctcaccgaattc

SEQ ID NO: 52 - DNA, 2178 bp, synthetic construct
  source 1..2178: mol_type = other DNA; note = pWVO1
cgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagccccctgacgaaagtcgaagggggtttttattttggtttgatgttgcgattaatagcaatacaattgcaataaacaaaatgatcgatgctgtttggcaaaaaaagaaaaagtgattaatttatattttatttatggcgctaatttattacggctttttttgttgtcggctagccgattctgatacatttttttaagcacaaaaaccacccaattttggagtggtgtgtaagtgcgcattgtcatgaaaaaatggcacgcaatttcatcactttttaaagtgatgtgtaagtgcgcattgtcatgaaaaaatggcacgcaatttcatcactttttaaagtgatgtgtaagtgcgcattgttcgaaaaatcgaactatgatttatttttgctgttgtatttatttttcatcttttgggttttggttttgttttttgttgctatcgtagtttatttgctttttaagggctctatttttcgttctacggcatttttataatttgccaatataatttat

SEQ ID NO: 53 - DNA, 2439 bp, synthetic construct
  source 1..2439: mol_type = other DNA; note = pAP1
aacaagggttgttcgcggggacaaaactagccccaagctcgcgtttccgcgaacaatccgcgttagtaccttgacgcggctttacccagcgcgcctacgcgccgagatttcgcagttcctgcatactttaaccagacagttttaacactacgagacaagaaagcgcccccggcagtccggcaagactccgagggcaactggaagattggtccttcctctaatgaatagattatctgaaagaacggcgttaagccttccggctcgccagattcaaaaagttatccccgccgcaggcggaaggtcgctgaagtccttcgaagggatgacggcgacgtggtcggcgcgaggaggagcctccagcgacgaacgcagcagagacaagcgaagccaaatcccttcgaaccggagggaaggacgctcagcgacccatccccttggcaatacggtattaacttttccagtatcaaacgagagcaagaaaacagctaaatctcgccgttctgagagatacgaactcagagacggattagccgaaatctcgaccattgagtccgtccggaagtgtggccgcgtgcccgtggcacctctcgtctcgttgcgagcaaaatctgacggtaaaggcgccggatatggtggtttgcacacttgtggaagcgtctgggcgtgcccagtctgtagcgcgaaaatcgccgctcgccgaaaaaccgacctccaacaggtcgttgaccacgccgtaaaacacggaatgaccgtctcaatgcttacgctcacccagcgtcaccacaagggacaagggctaaaacacctctgggacgccttgtcgacggcatggaatcgcgttacctctggtcgtcgttggattgagttcaaggagcaatttggtttagtcggttatgttcgagccaatgaaattactcatggaaagcacggctggcatgtgcattcccatgttctgattatttccgagaaagacccgctgaccagcacgtttgtctatcaacgcaaacaaggacgccgccgccttccctaccccccagagatttatatgtcatccgatttcattgctgaacggtgggaagctggccttgcgaagcacggcgttgattttctccgcgattccggaggcttggactggaccgttgcgaaagacgcgcgagccatcggcaactatgtcagcaagatgcagacgtccacagacgcgattagctcggaagtcacgttgggcggcttcaaaaaagcccgaaacgggaacaggacgcccttccagatactcgcggatatcctttcgctcggcgatgtcgacgacctcaagctctggaaagaatatgagaaagcttcgttcggacgccgtgcacttacatggtcgaaagggctcagagattgggcaaatctcggcgttgaacagtccgacgaagagattgcctctgaggaaatcggggacgaagcaatagcgctatttacgcatgacgcttggcgtcaggtgcgacgttttggagccgctgaactactcgatgtgaccgaatccggaggtcgtgcggccgcttaccgctggttggattttagggaaattgattggtcattgcctccgaaaatcgagtgaagtcgtcaaaccatactttaagtagaggtcgagaagtccgtggaaaagtcgcggcgcctctactgcgaaagtaggtatttatcgatgtttttcatcggaaaatatagaactaaattccagcccatcgcgccatgcaaaccctccccgatttttgacggcaacggcaaacgcacaggtgaattttcttcggagaagatgcggactcaagcaatcaccgaagacggccgcctcgtcgagctcacctcgattcctccccagttcgtccagttgattgatgacgctgtgaagagtcgggaacttcttgaattcgagaaggtcaatcttgccgtattgccgcgcaacggaggcggaatttccacctacctgacactaggggaagccgtccacgtccccgctccggtcgtcgtggaggtctcggaatgatgagcgaaccctacggaatcagttcgaacgctgaactgcaactcgtttttagccgtccagctaaacgagaagcccgtctgtatatgtctcggattctcaagaaagaactggaatcagggaagcactcgacgccgacggaggccttggaatcatgcgaaaagctctactggagcgtttttgaaccgcgacttgttgatgttgttttgcacgaggtcggagaatgccggtgcgcgggtacaaattccgcggttgcccgctcgctctcctctcgcgcattcattcctgcaaggttcgttcaatctcttgaaaatgggggacgcaacccagcccagctcctgacccaagaccaagttgcaggtatgcggaaaactttaggaacaaggtgaccacagcgtcacctgaccacccttttcgtt

SEQ ID NO: 54 - DNA, 2697 bp, synthetic construct
  source 1..2697: mol_type = other DNA; note = pWKS1
gaattcatggtgttccaggatgatgacgagttgcgtttcgggcgcaccatcggcggcagggtccagaccgccccaggccgacgctttgcaatcctgcgctgcgccaagatcaagaccctcggcaacatgggcgccagccttcaacacaccttccgggaacgcgaaaccccgaacgccgatcctgcccgccggaccgacaacacggttctgatcggcggaacagacagcgctgcggtcctcgatgcatggcgcgcccgtgcgccggaaaaaatccgcgccaatgccgtgcatgggctggaatacttcgttggcggatcacccgaggccctgaaggccatgagccgggatcagcaggacgcctatttccgcgatgccctgaactggctcaagacccggcatggagccgaaaacatcctctcagccgtcatccaccgcgacgagaccaccccgcacatgacggttatgaccatcccgctggaccaacagggcaagctcaatgcccgcgctttggtcggcagccgtcagcagctctcggctatgcagaccgacttcgcaaaggttgtgggacaggcgcatggccttcagcgcggtctggaaggctccagagccacccacgagcgggtgaagcgggtctatgcccatatcagcgacccggaagcctctgtgagcctcccagagcgccgcagaggcggtttcatgggtcggggtggggaaacggaggcagaatggcgggaaagggccacagaagccgtcacagaggcgctggcgggggtccagcacgccttgcggcgggaacgccgcgacagggctgcagagaccgaggcactgcgtcagcgccttcagggcagtccagatcagcagcaggtgaaccagagactggaacggcaggttgcccggctgaaggccgaaacggcccgcctgcacgacaggctggccaaggtcaaagacgaagccgatgcatatcacctcaatgcgctcaagctggacgcggcccgcgaggttatcctgacccatgcgattgccttcgtccgcgatcacggcctagacgaggccgacatgctggcgcggatggaggccggtctgaacgaagctctggcggagtttaagccggtgcagcaggagcaggtcgggacagaacacgatgccgtgcaaaaaacccgccagcgcgatgaggggctggatcacggagactaagccgatccgccgccagttcaggccgtccggccccggattctgaccataatttcatcgaaaaaaggggcgcagcccttcttgttctaatagttctataagttcaggcgaaaatcgtgcagcaattacaaaaggttgcgcgtctataagtggggaatccagccgcaaaagtggggaatccagccgcaaaagtggggaatccagccgaaatcgcggattgacgagtgggatttcccgcccaataaatccaccatgggaaagacactcgacgttgcccgcgaccgggcctttgaccagaccgcgaccgtgctgcccgccgaaatggcgcggggggtctatatgcgcaacgcccccagcctcgcggccctgaagctgatgcatctgatgatcgccacggcgggcgggcgcatggccgatgacgtgcgccacgaaatgcggctggccgacatccgcaagatcgacggtatggataaccacacccgggccagcctgaccccgctctttgcggaactggcggcggcggtgctgacccacgacgacccggaaaagcgggtcgtgaccatcggtggcctgctggacgaagcccggatcgattaccggcacgaggtcagcggcgatcttctggtgtcgtggaccttccgcagcatgttccgccgcatggcggcggaatcgaaccattgggcgattctcgaccggcagaccgtgttccacctcggcagcaagtattccgtgctgttgttccagcacatcgccagcttcaaggaatacgaccacattaccggcaagacctttaccgtgccggagttgcgggctgtgtttggtatccccgagggcaaaatcaagcgtttcgcagacctcaacagagacgtgctgacgcccgccattgccgaaatcaaccagctttcccgcctgactctgaccgccacgccgaacaagatcgggcgcaccgtggccagcgtgacgattgcttgggaagaaaagcccctcgaaggcaagcgctcgaccaaggccgaactggaccgcccgaaggtgggccggaaggcccggcgcgacggcaccgccgagacggtggcacgggccttcccggcatcgggcgggatcgagttcgaccagcattggcgcgacctcaagcgggcggcgggctgcaacatggacaacaccatgatcgccgacaaattccgggcatggtgcgccgggaagggcctcgctctcgatgcccggaacatcgaacaggcgttcagcagcttctgcaccaaggtgggccgggtctgagacccgccgcgccggtcgctcgatacctgtggtctcgctccctctgcggctaccgtcagcgcctcgcctgcatcgccgccctttccgatcctcatcccgccccagccttatggggggatgaggatcgggccgggactgaaacccgaagggtaatgaatgtgtctttccctgcttggcagggcgaacgacattcggcagaatgtctagtgagtacacattcattacccttcaggt

SEQ ID NO: 55 - DNA, 1952 bp, synthetic construct
  source 1..1952: mol_type = other DNA; note = pLME108
gcagctcaagcgcctggtcgatccgttctcgcacaacgatgaagcgctcgaaaacatcttcttcttcccccggttctgacatcgctcccccagtcatcgtgccgtgctagtgctaccggttcgggtgtggcgggcgggctgcgccaccccaaggggtcccgcgcctccccgtccgccgcacccgaacccatcagcccgagcatcgccgcaacgtccgcaggaagatcatcaggagtcgggagaccgcaacagcacccacccgacaaacagacctccacagccacgagacggcccccagggcggccagctgcccgacgatcagccctgttcatccgagaccccctcagaagaccgcagacgcgccgcaacgtccgcaccccagacgtccacgtcctgcccgaacacccgccgaaactcgttgacctggcggtacatcgtgctctcagccatgaccccggcccccagcaggccggagcgtccgccgtagatgtgccacatcagccagaacccaagcagccgttgcaccgtgctccgagcaacaccgagggagagcgccgacacaccggggatcatgccgctgatcagcgccagcacgtcccacggacccacattcttttccgacttcttcttcgccattttagtccacctccaccaattcgtgttcgattccgtgctcttgcagccaccgcgccaagccagcttgaccgcccaattcgcaacttcgcagacactcgtagagcttctgctgcccgacgaggcgacgccatccgtcacccgtgatcaaagcgacagtgtcagcgaccgagccgacctcctcagccgcgatcacgtcgtcagattcctcaaccatgaggccgaggcggtccctcaggcccgcagaccagccaatctgtctgcgtccccggctacccttctcccactcgaaccacaggccaacctctttcgccaagccgttggccgcgtcgcccagcacctcccaagtcgatcgagtcgagagcgcagaacgcgccgtcttgctctgggagttcgtcaactcgtggccgatcttaccttgaaattgtgctttgctcaggtagcgcgcgaggtggtcgaggccagttgctgcgctcatctgctgaacgtcttgcgcgcgagcaaggggagtcccgaggcccgccgcgagcacgccgcgttcccaacggccgaacatggaccggtgcagcgccagagcgtcgccgaagtcgcccacgaggaacacgagcacatgcagatgcacatgccacccattgcgcccgtgcgtaacctcgaccacacgcacgaagccctcgaccccgtgacggagctggtccgaggtccagcccttgcccgaagtgactcgccgccaccccgaagcgacaccatcccaaacagccgtcaaggaatccttacgagagtgccgaaccgtgaacgtcatgaacgccacacgaccaccgtgcttagtccacgtttcgaccgccgcgccgagttcaaggccacgccgagccatgatcttcgcgttacacaccgggcaggcccagaccgatccgcagctctgcaacccagcgaaaccggcccggccgtcgctgcaccgcacaccaaccgaagcgaccgccgaggcggcaacacgaccgcagaacgcaacgcgcttgagcgacgtatgacgccacaaccaataacggaccgaaaaccggtgtttgcgcttgtcggcggccacttcaccagcggcgggcacggccgaaggtgaaacattgttcgcatgattatctagggcgccccctagcggggccaccgccgggctgccgcccccacacccccggacgccacgacttcgggcgcgggcatcgaccaccattggtcgcgtactctgggacaaggaagaccccctgctagttgatgctgattcgacaccagacacgctagcaggggttctctcagttttgggagttctcagttct

SEQ ID NO: 56 - DNA, 4408 bp, synthetic construct
  source 1..4408: mol_type = other DNA; note = pLS1
ctgcagaagtagtcgctgattggctaattcagcgtatcaaagacaaaggcgaccaaaaatagcttgagttcttttagaacaaaaaagaaagacagtagttgcacctactgtctttcttttgcgttgtgcttttagttcctcgaacttttagcgtcaagcatattatatcatggggcgagaaattctgtcaaaataatgctataatgcttttgaggcacctcagcgatacggtcggtggtgtgaatctcatttacgtagggcgactggaaacggatagctcaaagggcgcgtttgagtgtcgggtgtgggactgccttcagcttcgggctgtaaagacccctgatacttttgaatgagatgaccctttggggtcttttttgtttttttagggagatgttgtgggggattttttctccgaaaaaatctaaaatatgggggggctactacgaccccccctatagtgccgagtgccaaaatcaaaaaaaaaacgcctttagccttagagctgcaagggtttgaggctcgtcaaatctcggcgacttttcggcgacttttcggcgactttttagagattttttgggaaaaatacgaaaaagattgcattgagtgcacggttatgctactatagttttataaaattttgagaggtgacgcatgaaaaaaagattgacgataacattaagtgaatcggtacttgaaaatcttgaaaaaatggcaagagagatggggttatcaaaatctgcaatgatttctgttgccttggaaaattacaagaaaggtcaagaaaaataaaaaaagccgtgctggcaggcactggctaaagtcaaacatttcttgggtatattatactttatggctaaagaaaaagcaagatacttcacttttttactttatcctgaatcaattccaagcgactgggagctgaaacttgaaacgcttggagtgccgatggcaattagtccattgcatgataaggataagagtagtatcaaaggacaaaaatataagaaagctcattatcatgtgctttatatagctaaaaatccagttactgcagatagtgtacgtaaaaagattaaattattgcttggtgaaaaaagtcttgcaatggtgcaggttgttctcaatgtcgaaaatatgtatttgtatttaacgcacgagagcaaggacgctattgctaagaagaaacatgtttatgataaggctgatataaagctaatcaataattttgatattgaccgttatgtgacgttagatgtcgaggaaaagaccgaacttttcaatgtggttgtatcgcttattcgtgcgtacactctccaaaatatttttgatttgtatgatttcattgacgaaaatggagaaacttatgggttgactataaatttggttaacgaagttattgcagggaaaactggttttatgaaattgttgtttgacggagcttatcaacgtagtaagcgtggaacaaagaacgaagagagataaaaagttgatctttgtgaaaactacagaaagtaaagaatgaaaagagtaatgctaacatagcattacggattttatgaccgatgatgaagaaaagaatttgaaacttagtttatatgtggtaaaatgttttaatcaagtttaggaggaattaattatgaagtgtaattaatgtaacagggttcaattaaaagagggaagcgtatcattaaccctataaactacgtctgccctcattattggagggtgaaatgtgaatacatcctattcacaatcgaatttacgacacaaccaaattttaatttggctttgcattttatctttttttagcgtattaaatgaaatggttttgaacgtctcattacctgatattgcaaatgattttaataaaccacctgcgagtacaaactgggtgaacacagcctttatgttaaccttttccattggaacagctgtatatggaaagctatctgatcaattaggcatcaaaaggttactcctatttggaattataataaattgtttcgggtcggtaattgggtttgttggccattctttcttttccttacttattatggctcgttttattcaaggggctggtgcagctgcatttccagcactcgtaatggttgtagttgcgcgctatattccaaaggaaaataggggtaaagcatttggtcttattggatcgatagtagccatgggagaaggagtcggtccagcgattggtggaatgatagcccattatattcattggtcctatcttctactcattcctatgataacaattatcactgttccgtttcttatgaaattattaaagaaagaagtaaggataaaaggtcattttgatatcaaaggaattatactaatgtctgtaggcattgtattttttatgttgtttacaacatcatatagcatttcttttcttatcgttagcgtgctgtcattcctgatatttgtaaaacatatcaggaaagtaacagatccttttgttgatcccggattagggaaaaatataccttttatgattggagttctttgtgggggaattatatttggaacagtagcagggtttgtctctatggttccttatatgatgaaagatgttcaccagctaagtactgccgaaatcggaagtgtaattattttccctggaacaatgagtgtcattattttcggctacattggtgggatacttgttgatagaagaggtcctttatacgtgttaaacatcggagttacatttctttctgttagctttttaactgcttcctttcttttagaaacaacatcatggttcatgacaattataatcgtatttgttttaggtgggctttcgttcaccaaaacagttatatcaacaattgtttcaagtagcttgaaacagcaggaagctggtgctggaatgagtttgcttaactttaccagctttttatcagagggaacaggtattgcaattgtaggtggtttattatccatacccttacttgatcaaaggttgttacctatggaagttgatcagtcaacttatctgtatagtaatttgttattacttttttcaggaatcattgtcattagttggctggttaccttgaatgtatataaacattctcaaagggatttctaaatcgttaagggatcaactttgggagagagttcaaaattgatcctttttttataacaggaattcaagagggcaatggctgatatggaactcaaagaggaacttcttgaaaaatatcatgcaccgctttttgttgatgagagaacaggcgagttgaacaatgacacggaagctttttggcatgaaaaagagtttgctgatatgtttgaagttcaatctccgatacgtgaaacaactaaccaagaaaaaatggactggttaagaaaacagtaccaagaagagctgaaaaaactagaatcgtctaaaaagcccctagaagacgatttaagccatttagaagagttgcttgataaaaagaccaaggaatatattaaaatcgattctgaggcctctgagagggcctcagagctatctaaagccgagggatatataaataccctagaaaatcattcgaagagcttagaagcgaaaatagagtgtttagagagtgataatctacaattggaaaaacaaaaggcgacaaaactcgaagcgaaagcgttgaacgagagtgagttgcgagaactaaagcctaagaagaattttctaggaaaagagcattatgagttaagtcctgaacaatttgaagggttgaaggcagaagtttatcgtagtagaactctattgcaccacaaagatattgaactggaggaagcaaaacgtcaagtatctctgagagcctctaaaaactattttacagctagtttagagcgagctaaggaaaaagctaaaggtgagagtatagaccgtcttaaaagcgaaataaagcgactaaaaaacgaaaattcaattttacgtcagcaaaatgacaagatgctagggaaattaagagagttaatgcctgataaagcctttaagaatttgttatcagaacttaaggcgattaagccaatcgtgaatataattaaaaaggctattgaaaagagcttgttctgagcgatttatgccgtgaaagctatttgacaataagcagtgacagagtacgctaggacgtgccgagccgaaaggctttagcgtttcggacggacacggacaaaggacggcagtcactggttacttgttgtcaaatagaccatggaataaaaagcgtcaaaagtcttgagtggatgataccctatggtactctattcgccttttgacttttttgctataatttaagtgtcgccagttcttccgtcaggtaatgcgaacttagactggaggtgagcgttgtgaagacattcctcgagcttgtctttgtcccttttgtggttggcgttg

SEQ ID NO: 57 - DNA, 5804 bp, synthetic construct
  source 1..5804: mol_type = other DNA; note = pUB6060
actgcgatgtacgatagatgctgtattaagcaagtacacacagcgtcccctctgcgaggtgccgtctgtgactggttcagggggctcgccgccccccgaaacccctagcatccactgcgaaaattcgcacgttgggtgcgaaactttctcagcggattctcatggaaaagcgcaccaaagagatcaaaatcagactcaccgaagcggagcatcagcggttacttgaacgctgtgaccgaacgcatttggccgagtggttacgtgccgttggtttaggcgaatcgcggacagctcgtcgtcgtccgctacctaccgtagacccgatcttgttacgtcaggtcagcgggatcggtaataacctcaatcaaatagcccgttacttgaatcagcatggcttaccgccgcaagaacgggtgtcgttgttagatgtgctcaatagcattgaccaacatcttgccgaactgctggagcaacatcatgatcgttaagattcatggtcgtggtgccggtggcgggagtggccctgtcgattaccttctgggccctgatcgtcagcgtgaacaagcgacggtgttacggggtaaccctgagcacgtcaaagagctgattgatggctgcgaatttgcccgaacctatacctctggcgtgctctcttttcaggagagcgacttacccgcaggcgaaaaaacagcgtttgatggagaatgggagcagacattgatgaccggtctagataaagaccagtatgcctgcctctgggttcaacatcaggacaaagggcgtcttgaattgaattttgttatcccgaacatcgaattgcagagcggaaaacggctgcaaccttactttgatcgggctgaccggcctcgcgttaacgcatggcaaaccctcaccaatgaccggcttggattacgcgacccgaatgaccccgccaatcgccgagcattaaccccctcgaatgaccttcctcgcaacaaacagcaggcagcggaagccattaccaaagggctaatcagcttgattgagcagggagaaattacggatcgtaaaggggtgatttcccaccttaccgatgccggattgtcggtcgtacgggaaaccaaatccagtatcagtattgctgatccggcaggtggcccgaatattcgcttaaaaggagtgctgtatgagcgagattttaaatttagcgcgggagttcgagagcaaatcgaagcagcaagccaagactaccgcaacgagcgtcgcgagcgcattcgagaagcacgagaaacgtatcaccgaggccttgaaattaagctcagggaacatacagaccgctatccaagaagagaacgacaaccagctaaaacagatacaccgcttagtcggaatgacatggctgtacagcctggcattaagcgcgatcctgtttgcgacattgattggagtagcttggtatctcgggactatcgtggtcgaacgccagaacgaaatcagcgagcagagccagatcctgcaggacttaaagagccagaccggagccggcgtatcgataattcacgattccaagaacaagagcgtgtattacctgatccttccgcagggggcgaagcagatcgacgagtacaagaacgctcaacatcgtcaggtcatcaagtacagcgccaaataacctcatcagacgccacagaatcgattctgggcggttttatctatcagggtgaagagattcatgccgaaatggcagcagcagcttctgagcgcattagagagcttacagaggcactacgaacaacagcagcaagcgtggcaggacagctacgccaacttacagcgcatgttcgaggttacctcgcaggagttggcgaaaaacgacagggtttgtcaggccttgagcatgcaagtcaccggcttggcgcagcaagtcgagagcttaaacagaacagtgcgccgcttgagcaattagccaagcggcacgaacagcggcattctcggcggtcacggcatgagtttataagcgtttatcggcagcatcataagcggcagaacgctcgcgcttaccgaccgccaccacgaataccgtaatggtttgatcgcgaacctgatagaccaaacgataaccggatgctcgtagcttgattttgtagcaatccggcagctctcgcaggcgatttttatcgatccgcgggtgctgtagaacctgctcgagttttttcttgaactgcagacggacatcatccccgagcttgcgccattccttcagggctcggggatcaaactcaaggttatagctcatccagtgacacctttacgcccgcctgtgggttttccagacgatcccgaacgatagccatcaaatcggcatcatcctcggtcagcaacacctgctggaacggcaagcgtccgctttgggccacatactccagtgtttggcgcagaacctcggacggcgttacgcccagcttttccagtgcggcataagagcggcttttcagctcgtcatcgatccgaatattaatcgtggccatcatctcacctcttgatgtagtgacaagtgtatctacaagaagtagtatgagcgtaaagccgtgcgagaacaagcaggaataacggattgtcggggatgacaaaaaccgttgttgaggtgtaacttagcggcagaaaaaacaaagccccgaattcatgtgctcaacttggcggaagactcatgaaattcagggctaggtcgaaacctagaaaggatattagcacatgcagcgtgcaaaacaacagccccgccataaggctgggagccatggctaatcaggctttaacgctttttaacgaccggttaccccacaagccgtacttctccgatgatttacagtttggtgtccgcattgccggtaaagagcgtgctctcctcgcaaaatacatccagtttaaccagccccacgccatgtactggctttgctttgacgtggacagggccggagccgcgattgattgggccgatctgggtgcacctgcgccgacactcaccatcaaaaaccccgataacggacatgctcacctgttgtatgccttgaacattgcggtacgcaccgcgccggatggtcgaggccgcctcctcaaatatgccgccgccattgagaatgcgctgcgtaaaaaattgggcgccgatgcggggtattcagggctaatttgcaagaatccgaaccacctgcactggcagatcaccgtctggcagcctgagctctacaccctcgactggctagccgactatctcgaccttggcgctgccaatgaccgagaaatcctgcccgactacggtttaggccgtaactgcaccctattcgataaaacccgcaagtgggcttaccgcgctatccgccaaggctggccggagtatagccaatggctacaagcctgcattgaacgcgctaaagcctacaacctgcagttctccgcacctttagacgagaacgaagtcatgggaattgctaaaagcatttccaagtggacaatggtcacttatcgcagtctggggtttgatgagtatgtgaagttaactcattcacccgaggtacaagcatatcgcggtcggcgaagtaaaggcggtggtagacctagtattggggaaccatggttagctttaggtattagtcgtcgaagttattttagatggaaaaagctaggtaaattatgaaaataattagttttatcaatatgaaaggtggtgttggtaagtctacggttgctattaatgttgctcattgcttagcggagcgaaatcaaaaaaaggtattgataattgatattgatcctcagtttaatgcaactcagtgcgttatgaaggcagaggattacatagagcatatgcgtacgggtaaggatactatttgttctttgtttaactctgaccgagttgcagctaaaagcgttagtggaccatcttttgaaaaatgcaaagatatcagtagcatatctcctgttgaaatgtctgagtatttgcatattttacctggtgaccttggtttgcatcgaattgaggttacagctgggagcgggcaggagttcaagttaaaacgatacttggattctatcagtgacaagtatgattatgtgattgtggatactccgccaacaccatcaatatggatgtctagcgcattgatagcttctgactattatataataccggttaaaccagatccgttatcaaggacggggattgattgcttgatagtataatagcagataaaaaaggaaactttgatttaaaaataaaatgtgctggagtggtgtttaatatggttgaagaaaactgtgtttagagagactaagagtttttttaataacagtgatacttggcgcaattacatttttagatctttcctgcctaagaaagttgcgatagctaaaaggcagacatcaggagaacatatattaaagacaaaagattcctctttgcacatgaaacttgtcagagtggtcgatgaaatcgaagagagaatacagtaataggataacgtatggataagttaacaactaaagaaataaagacgttattgaatttcattgaagagttctcttggatttcaaataaatataaaaatttggataccaataagttatatgaggctcttaataattgcgagtctcgtcgtcaaaatgaatataatgattatatttcgtactcaaaaagtgtaggtaagaataatcatgtgtcatataggaatagccttaaagataagacatttctaattggtaagctcccatctcttttgatggataaagaattgttctctaaaaataaggagctatctgactttgctcgactacttggtgttgaggtcagattccctgagaaacgttctagagatgagataatcggcactattatttgctcattacaagaggaaagtagtgttaaaagaattcatgagattggtgagtttatctatgctttaactagcgatgaaaaactaatgaataacattaaggttgagaagaaaatatataatgatgagtatgattggaataatgtaattagacttttatttatgggtaaatgatgtctagattgatagggaaagactttgatttatttttaggcttctttttaaattacagccttaaagatttggcatctaatggtgattttaaaaagaagctgcgtgaggcacacaaaaaatactatccgttactcactcttagcgctgagcttgatctcatgtttaggggggatgtgggagaggattgtgctgatagggttaaagaaacttgctctgatataggttcgtctatttttttattagcccatggaatgtataagcaatccaacatgtcattgcgtagttctattgagaactttttaaaatcaataggttgcaatcattgccctgacatattaacagataagagtgttttttctgtttttgaaaaggctgggcaattagagttatttttagatcctgtgttcaaatgcaagtttgatgagttgcaatctatttactcatcgttgtgcttatatactcataccgcaagtgctgaacacatggctaaaattagtgctatgggcagtattccaaaacatgataaagcgaaaagcgctattcttgttaatgacctcactaggcttgttcgaatttatcttttcatttacacgaagttgtttaggtgtgaattcttcaaatttaaccatgacaaccgagatgtcattctcagcgcattaaccaagtcgcaacgacgttcgttaatggaaccatcctgatgtggcactaaacaacctatatcagataacagccctgcttttgcggggctttttgtttgtgcgtgatgtg

SEQ ID NO: 58 - DNA, 1711 bp, synthetic construct
  source 1..1711: mol_type = other DNA; note = p545
ggtgccatgagggttctcactgatacctgaatgagtttgacggtggggccgcatgcacgactgccataccgtcaatccgagcgccgcagactgggcatgtccccctgagtaacacttgcccaccgggttcaacgtccgttgctgatgccccccattggctgtagccacaatggccgcaggataagaacgtggcgcgcactgtgcaaccgcacgcgaacgtagtgccaagcggtgcacgatcaagaaaatgctcatcgtgcggcgttacggtcatgcgtcagttcgattcttgttcagcgcgtagtgcacggtgcccacggaaaccccgacctcggcagcaatcgcacgcatgctctgaccctccgagcggagctcacgaatccgcgcgtggcgtgcggcaacgcgggccacgaactcctcgcgtggctcggaagtccaccgcttgacggtggactcggagacaccgagctttttcgcagcagcggcgatgctgtagccgttgcgggggagacgttcacgtgtggtcatgagagaccctccaagaactgttgacgggtatgggcgcgccgtgatgcgccactggcaatgccgcctttgtggccgccttctttgccgcctttggatgctccacggaggctgatcgctttctggcgtgcgcggaaggtttcgggggtgaagttgcgccagacccatcgggaaatggatcgagataagtgcttaagttcgttcaagccgagggggcctgtggcgaattcgtcggcgatgatcgtctcgttcagtaggtggatgtgctcgaatacggtgtgctcccattcggcgaccgggccgccccaggagtgccggacggcccggtatgcccacatgcgggtggtgtcgaacagggtgacgttgcggccgaccgttgatcgggtgacgttgcgacgcgggttccctgcctccggcagtgcgtggatctcgtcgagggtgtgtgcgagggcgcgcagctcgtagagcgcgtctgcggggccccagagggtcgcatgggcggtgctgagcgggttctttgtgatccggtgcccgtaggatgcatcgccgccgagaacgtcgcataggccctgctcgacgcgggcgagcaggttgataggccgtcgccgcgcggcatcggtcagacacacagggttcttcaaggcatagacgatgtgtccggtggtcgtgacacggttcatggacacgtaggacggtgaaggcagcccagcgaggtctgcggcccagtcagcatccgaagcatctcgatcggtgatgaccaaggactgcatgaccaacgggttcgcttcgatgtaaggcagctccagcgccctctgccgagtcacgtgccggtacgccccagacttctcggctgacgccagcggcttgcgtggcagccagctctcagggaacaacgtctcgaacgaatccatacatgcagtgaagcatgcgagtcacgttcagcgtggtccattcctcggcgtgttcaaagtgggcgacgaagaccccatatcagttagttacccggttgagccatgtgagcaaagcgaactctctttccacatcccctgccaaacatcccccgactcccctgacgctgccacctgctccaagggatggctggggcgtgttcgggcattgcggctagttcctcgcgcagctgtgcgatcttcgcctgaactgctcgccggctgtcagggtcgacggc

SEQ ID NO: 59 - DNA, 7426 bp, synthetic construct
  source 1..7426: mol_type = other DNA; note = pJD4
ctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccctatctataaactcttggcttggttctaatccctctaaacgattattatcaatagccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcataaatccgttttatagccgcctgaatttgcttataggcgttatcttggcttatttctggaaactcacggacaaaatcagccaccgtaaaatcaaaaatcttttgattagatttcggatccatagtcccaatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcttataacaggaaatttgttgtcgtataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgaagccgttaggcgtaggcggtagcctataaaagccatttaattttatctttaaatttccgtttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaataaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttaaacgattattatcaatagccgctctaaccgctttttctcggcttaatttttctgtctctgttataaaattgctattcatcttgttcttctctcacactttaactaattcacagttcacaatcttataccctcgattttgcaactcgtttaaaaaatctgaccgcttaccaagttttgatctaaaactagcattgctagtcaaaaaaacccaataatgccgtaaatcttctgttgtaacatcggcaagatcagaataaaaatcttcaaggatttttaggcaatctctcgcatagtttccatattcagcattactcattttgggattttgagtatccaatttcacaaacttcccgtacttgtttttatgcggaaatgcagggcgtttctgttcgatttttaccgcacttttcttactcttgatcgtgaattttaatgctacgattgttcgcccacgcttgataggttcaacatcaacaagcagatcggatttagcattaatttcatttatggatggagttaatactcgctttttaaaatccttaaacagtgggtacttatcagagatacttaaccaacttttaatatcttctacgcttgtttgtcgccaacctgtatcacgatattgagaacacaattcataaaggcgaatagcgtgcgtactacccaaagccccaatattgatcaatttatattttgtgtagttatcgtgtaattcagaaatgtaaggaattagctcatcgtggaactcgatataaaatcgcccttcttttttaaaataggaacgcttatgaattaaagctacttctgttaattcgtgttcgttatcaaccagtgtaacccaacgctttgagatttttaaaacggcatttctaacttgtgtgtaagctatatcaggatttacatcggggaagcttttacaaaaatctgccaccgtgaaatcaaatccacgcttagacggatttttaggattaaaaacccccaaagttaaagccagaatccgcatttcatcaagtgtcattgaatagctggcttgtacaaaattgttagctttatggactgttaaatcatttgtcatatcatcaaggtggacataaaataaagattgtcccattataaccatacagttaaatggtggtcaataaaaaacaaagaccactataacaataaatttgtccacctataacaataaatttgtccacctataacaataaatttgtccacctataaatctcgcaagccttgtgtaacaaggggagccagagcctacaaacaagaatacaaacaagaatacaaaaaaatagagcctaaaggctctttttggggctttcagccctaattttttctttttttcaggatttaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttatttattatttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcaaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgataaaccgtatatttaccgataaggcatccggcagttcaacagaccgggaagggctggatttgctgaggatgaaggtggaggaaggtgatgtcattctggttaagaagctcgaccgtcttggccgcgacactgccgatatgatccaactgataaaggaatttgacgctcagggcgtggcagtccggttcattgatgacgggatcagtaccgacggtgatatggggcaaatggtggtcaccatcctgtcggctgtggcacaggctgaacgccggaggatcctagaacgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgatacgcttatttttataggttaatgtcatgataataatggtttcttagacgtcaggtggcacttttcggggaaatgtgcgcggaacccctatttgtttatttttctaaatacattcaaatatgtatccgctcatgagacaataaccctggtaaatgcttcaataatattgaaaaaggaagagtatgagtattcaacattttcgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggtgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgctgccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgc

SEQ ID NO: 60 - DNA, 8830 bp, synthetic construct
  source 1..8830: mol_type = other DNA; note = pIJ101
ggatcctcgttgccgtccttgccttcggcggcccgggtcgcctcgaggtcgagggcgcggcgggtgaccgcgtgccatccgtcctcggtcacggcgaccccggcccgcagctccccgccgtcggcgtcggccgccaggagcagatcgaggtcgtcggcctcggtgtcgccgccgtcgagcccgagcatctgccgcaggtagcgggtccattcgatggcccggcgtccccgggttgcccgctcgtactcgtgccagcgcgagaggttccactccagcgagccgaccccggcggcgtcgtcctcggtcatgccgccggtcaggtccccgatccgtccgaggagttcgaacggggcgacgttcccgccggtcgccgtcttgaggtcggcgcgggcgagttcgagggcgggcgccttcccgtcctgggtcttggcgatgtactcggcgaggtcgttggcgtcgcgctcggtctccagccgcttgaagtcgacgccgtgccggtcgtcgggcgtgaaggcggggttgaccttgcgcagggcggcggtccacacggaccgccagtgcccctgccactcgtcgagcgcggcgccggtcggctcgaaggtggcgacgatctgcttcgcggaccgctccccctcggtccggccgccgaccaggacgatcgcgtggatgtgcgggtgccagccgttgatctgccccacggtgacttcggtcgcgcggatcatgccgacgtacccgatccggtctcggatgccctcgcggtcggcggcccggtgcccgtccttggcccggcgtccggcccacgtgccgcccgtgatcagtcgctggtaggcgcccggccgccgggggctgtccggcgtcttccgggtgccctggagggcgtccatgaggtccgcgagccggtccgtgtgcccatggcgggccgtgaaggtgaccaggtaggcggtccccccgcgcttgatccactcgaccacggcggcggtgatctcctcggcccgcttgtgccggatcgtggcggcgcagaccgggcagagccagatccgcccgcaccgcatcaggcccaggaccacggacgttccggccgccgtctgggcgacgatcacgccggaggcagggtccatcagggcgcggccgcagcccttgcacgcggcgtccccgctgatccgccacagcgtccggcggcggctgtaccgggcggctttccgcagtcgggcagcctcggtccgcgacgtgcttcctacttcccagaggctgtcgcctctcgggctctccccatccaccccgtccggagaaaccgcaggtcggaggggtgcgggaaactctgttgtttctttcccaaggtgttcgcttttgcctcgggcggcatctcgcgtcacacgcgcgatcgcccgcttcgctgccatccggcagcggtctgagcagtagatacgcggccgtttgcccggtgtgtgggcaattgcggtcccgcagtggcagcggggcccggcgggccgatctggcaatgcctcggcatcgctccgtactctgggcacgagcaacgttcctgtctcgcccggctaaggggcgcgagtctgggagcggacgggtcggaggtgcgaagtccggcccgttgctctttggtctggtgggaatcctggcaccaatcgggccagaggttccctccgccactcccgacgccccttggggctggtgtgacttggagggccgaagagagccccgccgggtatccggcggggctttgacgtgcggtcagtgcgtgtgtcggcgagcgatggccacgaggccctggaagccgagcggtccggcgaagtcggcccagtcgcaaccgggctcagcgcagtgggcggaccagccaccgccgttggggtcctggaccaggttcacggtcccctcggtcaggcgtccgtcgaagtcggtcatggtcggtctcctggtgggtgggggcggggcgccagcacgaagtgccggcgccccgcgggggttggtcgggtcaggcgccgaaccggcgggcggcggcggcgaccaggccgtcggcggcggccatggcgcggtcgcggtcggtggtgagggcggtgcggtcggcggcggcccgcaggtcgtaggccgcttgggcggcggcggtcgctgcgggggcgagggcgggggcgagcaccgacacggtggtgaggggcgcggtgatcgcggagcgggtggcgtgggactcggtgcgggccgcctcgtacgcctcgggggaggcgccggtcaggcgcaggtcctcgcgcacccacatggcgcggcggtggtcggcgagggcggcggcgagggcggcgacggcctggacggccgcgtcgcggcgggagtcgtcgcgggtggtgcgggcggtgcggtgctggaggagtccggcgaggccggtcccggcgagcgtgccgatcacggcaatgagcgtggtcaccatgtgagccccctggcgtcgtgtccgtctgcctacgtgtatcagtctgacacgcacgtgtcaggttgcgcaatgggtaggccccgccggtttccggcggggccatccgtcatgcggcggtgctgagtcgggcggcgagctggtgggtggcggcgaggatgacgcggatctccgcgccgccgtcgaccgccgcgagggcgggggccggggcgggtgccggcggattggtcggggtcgggcggcggcggagggcgacggccaggaccatggcggcgatcgccacggtggcgaggtaggcgagggcgatcacgcggccacctccctcgtgacgcggcgccaggacgcctgcagccgtgcctccccggcggagtagcccatgtcccggaagcgtccggcggcgacccggtacgacccggagtcgaccaggccggacatgatcccgtcgatctcctcgttgctgagcgggaccacgtcggccgacccctcgatggcggccgggacgggctccgtgggtccccagacgggcaggcgccaggagggcggagacgtgggcgtgacacgagaggtgacgcaggtcagagcgcgtgccgtgccctcgccctcgcgggcctcctccagcgtcgaccaggccgacgccagctccccggcggtctcggcctggacccgggcgacccgggccccggcgccggtcacggcctccagccgggtgacctccgtaccggcctcggcccggagctgagcgcgggccacggccgccctgtcgcgggcgtcctgctggatgtcgcggatctggtccagggcggtcggggtgagcgcggtccgctcccagagcccgtgcacgagccagagcgccttggcggcgagggggagccaggcgaccgcgagccaggccccggccgactcctcacccagcgcgtgcgcgaccaggacgccggtcgccacggcgccgaatccccacccgacgccggtgatcgcgcggctgtggtcgccccgcgcggcgaggcgccgctcgtacgcgagggtggccagccatccgccgtcgagcccgaggccgacgaccaaggcaacggcccacggcatggcctccccgagccacatcacgatcacgaccacggtcaggaccatggacaccgccgtcatggcgacggccggggcggcggtcatcgaacgcttcttctccatgatcacttccccttccgggccttgagcgagatggacaggccgaccccggcgggggcggcggcggcgagcgcggtcgccgtggtggcggcggtctggagcaggaggcagagcgtcatgacgacgccgaccgagccgccagcggcgaccagcgccaggaggatcgggccggtccagtcgcgggcctcggcctggtggtggacgacgacgaccgtgcgggggtcggtgccgtccgggatcaggtgggccggaatgtgaccggccctcatctgcgcgtcgggggtgcgcatcaggccgacacctccccgacctgcgtcacgacggacagggagccgtcctcggaccgcaggacctcgcccgcgtcgagcagctgcttgaccgccttcgacacggagcccttgttgatgccggtcacggtggcgacgtcggcgacggtcgtagcgcccgtgccgatcgcggctgcgaccttctcccggttggtcggcgccttggtcggctgggccgggacctcagccgcgggggcggtctccttcaccagccggagcggggccggggcggaggcgccggcacttcgtgcaggcgactcctggcgacgccagaccggccggtcggggagcgcgatcacgtcggccggggagaacgcgcgggtgttgatcgggtgaggctgcaccttcgggccggaccggagcatcgcaaccccgggcatgggcagctcgtgcgcgtgccagcccttctccgtcgcgtcctcgccgaacaccacgcgggactccccggaggtgctgagcgcgagggcggcccggtaggtgatctgcgcgctgatctgcgggtcgatgccgcccttggcgtccatggtcggcttctgcgtggcccagatcaggatgatctcggccgcccgtgccatccgggccagcgtgctcaggttctccatgatccgggaccagtccgggtcacccggctcttccttcgaccccttcgcccgggtcttcttggccatggcgatgacctcggcgccctcgtcgatgaacaccgtgatccggggccgctccgggctgatctggatcacgtcctggccgcgcgggatcagttcaagccgctcgtgcatctcctcgaccagctcgtcggtcacgtccaggacgtcttcgatcgagatggccgtgcgagcccggtgctgccagttgatcgcctcgacccgcttggggtcgacgacgaccaggcggtgatcggcgtactccgagccttccgccagcagggcgcgggtggaccaggacttgcccgagccggacgtaccggcgatcagcatccggcgcccgagcggcacctgcaccggctcgccggtcaccgtgtcgactccccacggggcgccgggcgtccagccggtcaggtcgatcccgtcggccgcgctgcgggtccgcagcgtgatcacggcgcggtcgccgtgcgatccggccttgatctccatgcggaggtcggtccgggctccgagcagggcgcggatctcctcgtgcttggccttgaaggcggacggcttccaccggccgtccaggcgcaccgtggtgaccagcccggccggggtgacctgaaccggcgtcgtcaccgtgcctacgaggccgcgctcgtcggcgtgctgagcccagtacgacgggtcgagccgctcgaccaggcgccgctcctccgctgacagttcgtccgcgaccgcgaccgcgaccttgcggcggccgatcaccagcgcggcgacgttggccgcgatgagtgcgagcgagcccgcgaccggccaggccccggcctggtcccagccgagagcggagatgccggcgcccggcaccgcgtgcatggcgtgcgcctgaacggctcgcgcccggagggtcgccgggtagtccttgcgcgcggccttgagcttggtcttcgcctggtcgcggtgcgtgcgggccgccttgtccgccgtgcgagccgcccgacgcacggtcgacagcgggttcttggacgccgcccgcgcggccgtccgctggctcttggccgtggccgccgtcgaccgcgcggagttgtagcccttctgggcgtccatcagcgccttcaggtgctccggggtccggagcgccatgcggcggtcggcctcggcgtcccagcgggccgcgaacggcgcgagggtcggggccatggcgtcgagcgcgttcgtgatcgctccggcggcgttcttggagccggttgcgatcttcgtagagactgccttcgggtccacggtttgtcctttcgcgagggacgtggatctagggccggagaccgttcacgcggtctccggtccgccccgtttccggggctgtgtgtggcgtcgaacaaggtccatactgtggtcgcacagttgctgtgtcaaggcatacactgtgctagacagctacacaccgcgcaccacactcgaaggagtcgtcatgtccctggagcgcacgcccccgtacctccaagtcgtcgccgcgctgaaggcaaagatcgtcagcggggagctgaagcacggggacacgctgccgtccgtgcgggacctcgcggcgcagtacgagatctcgaccgccacggcccagaaggtccaccggacgctgaaggcggaagggctggcggaggcgaagcagggcagcgcgaccacggtcagcacgcgacggaccctgcaccggaccgcagccgaccggctggagtcggcgctcagcacgggccggatctacgcggacggggagtacgcggtcatcaccagcgccgcccttgccgagccgcccgagtgggtggccgatctcctcggcaccgagagcggccaggccgtgcgacgcgagcgcgtcacccactcagccgacgaccagccggtgagcgccagcgtgagctggttctccgcagacctcgcggagaccgtgcccgccctcctggtccgcgaccggatcatcggcggcaccccgtccgcgatcgaggcagccaccggccgccgggccgtcgccaccgaggaagccaccacggcggccgccgcgaccgaggaccaggccgcgcttctgggcgtagccgcaggcgccccagtcctgctctcacgcaacgtctacgtggacgctcagggcgacacgatcgaggtcggggagtccgtcgctccggcgggccgctggcgcgtccaccgggactgatcactcgcccattgagaagccccgtcaggcaccgcccgacggggcttcttcacgtccagacgacgtggtttccgggggttgcccacccggttgagccgttgcaccccggttgaccccccgcaaccgggtctgacctgcgaagttgcaaggttgcgacggtttccaggagggggccctacgcgtgcgcgcgcgaggaagccatttttgatctagcttccggagcccttctcggcctccgccttggcccacgcccgggccgtgttcggagcgaccgccgtcacctcgttgatgcggcggtaggggacctccatccggaccgcctccgcgatcagcgggcgcagctccttctcaatctcgtccagctccgcgaggagcttgatccgctgctgccccaacggcttcagcgccgcctctgcctcggcccggatctcgcccggtgtcttttgcgtcatgaagtcatcctgaccgactgtgtcagtctgcgcaactagttcaggctgcgttttttgcggtacaactttccctacgtcatcaaggcggcccgcgagcgggccgcgcggcccggcccacggcccggccgacgctcctgtcttcgccccgctccggcccggccgccgaccggcccgcgcacacgacgggggcggcatcggtgggcggtttacgtggcgcctgctccgccgactgcgggcatcgccgttgtgctcgccgacagggcagcggggaggggtgggggactcgcggccctacgcggccgtctgagcgcctgtcagcctcccggagcgccgtacccccgccgtcgcggtgctgagccgcgtgaggcgaccctgagccccgtcgtgggttgctggggagcacctgctgccgcgatgaggtggcggccgtcgagctggtcagccgtgcggctccgtcgtggccggtcatccggctgcccgatcgtggtgggcaagatgccggcggaaccggcggacctcgaccgcgacggctatccggcgggccgcgggcgggcctccgtagagggcgagggcgggcgccatgccgaccgccacggcgggccacaggcccaggagctcccgcacgatcagcgtcccgccgaccaggagcagtgcggccagcactgccgctaacgcctggtcctggtcccggtcctggtggtgcatcagtcctccccgtgatcacttcggcacccaccgtagtgatcacccccgacagcggatcaaggggtttgcgggtcccggtcggcgccgggcgggggaggcaggagccgccgacgctgcctctgggacgggccggacggcagggggaccggcggccgggcgagctgcagccgggggtccggcagggccggagcgggcggaaccgtgctctgacctgcggcccgagtttcgtcacgtgacggaatggaaggctgctgcatttcgtcacgtgacgtatctcggcgagcgactgccgacgccacggcggacacgatcgcctcgcgctggcgccgggcctcgtacgcccgctggcggcaggagcggcggcagtagtcccggctccggccgacgccggattgcttgatctccgagccgcaccaggcgcagagcttcgcgccgtcggcgtccctgggggtggtggtgctcatggccgacgaccgtacgcggcacgtctcgtagcgaggcgagtcgggcgcgaggtaccgcctgcacgaagtgccggcggggccgaccccgggcgagtaatcccaggattactcccgcggcttcgaccccggccgccgtcgccgcgtacgtcaccgacccccgccgtacgtcaccgggatgacgtacggcgggggggagcgagttagtgcgaagtgggcccacttgcgagccgggcgatgtgccgggcggcccgctcctggcggtcgtcggcgtcgtcgtcctggtcgtcgtcctgctctcgccgtcggcgtgcagttgcttcctcgcggcgctgggcgagggcggcgagcatgtcggcgtacgcctcggccacctcccccgccgtgagcaccaccactgtgtcggccgcgtcggccagcgccaggacctcccgcacccgttcgcccacggccgcc

SEQ ID NO: 61 - DNA, 11046 bp, synthetic construct
  source 1..11046: mol_type = other DNA; note = pSN22
ctgcaggccggtgactccagagaagggttgaagcggagttgcggggctagccccccgagcctccatcgttccgaccgcccggcctgtccgggacgggaggacttttcgagcacctggaggagcacgtgaccaaccagcagcaggaccaggagcagccgcagcgcccggcggaccgtggccgccgcgagttcgcgaagaagggagccgtgaccgtgctcgccgctctcgtctccggagctgctcgggcggtggtagcccacctcctcacgggaggtggcgagtgacgcggctcggcagccgggccggccgccggacggaggcaggagcggcccggccggaagccgggccgccgggcccgctcgcgggccgccttgatggagtagggaaagttctaccgcgcccactcgccacgccacgagacgtgccgcgtacggtcgtcggtcatgagcacagagacacccacgaacggcgacggcgagacgctgtgcgcctggtgcggccgtggccccgtgccacccagccggggaaccaagccgcgggcctactgctcgcgcagctgcgtccagcgagcccacgagtcgcgaaagctccgcaagaagctgctcggcgcgtacatgaagggccgggccgaggaggctgagctgcgcggaggaaagtcacgtgacgatgcaggaaagtcacgtgactttcccggtcggcaggccccagcaaagtcacgtgactttccaaaaccccaggtcaaccctggggttccgcgtcccgcggtcccggtgacgtccgcgccccggtcgaaaggccggcgcccgctgctgccccccgcgccgggcgtgacgcgggagacgctcccgctgttcggcgacgacgacacgcagcccggccccctcgatggccgcgccgacacggagtgaccacgacggccgtcaccccatcgcggcagcagcccgctccccatcgacccgagacgggcggtgcggtcgcctcacgcagcccagccccgcgacggcgggggtacggggggtccggcgactcctggcccgccagacggccgcacagagccgccgaccccccacccctccccgccagccgtcggcgacgcgcacaacgacgatgcccggcggccgggtgacggcccgccatgtaaaccgctcagggatgccgctcgtgggcagcagaaagcccccgccgggtctcggacgggggctcaatggggaggtagggcgggggcctggggtcactcgcccatgggcacccgggcgccgggggcgtagacgtcttcccagtagccgagcgcctgttcgtcgtcgtggaacgtgacggtggtgaccatgacggcgatggctgagtgggccgggggcgtcgatctcagagcgttgagttcgtcctgggacgcctgccgtgcgtgggcggcccgctgtcccttcaccacttcgcgaccggtgcgctcgctgtagagcttgtcgaactgcttcaccatccgtacgtcctcccccagctcggggacggcggccaccgtgtggggcgggtagacggagacgccgaccgacgtcggcttgtcgtcctggcggaagacgcgaatgcggatcacggcttcgtcgccgggctccagggtgagagccgtgcagatctcgggatcgtggaccgaccgggtcatcacgcggtggccggaggaggtctcgccgggcgcgtagcgcatcccgttcttctccatgcgcttcaagcggtccgcgccagtgatgacgatggggttcttctccacgaccgtacccagcgccccgcgcgagctgaccaggccctcgctcttcagcacggccaaggcccggctcacggtcttggccgccacgccgaactgggcacggatgtcagcgacggacggcagcgtgtcgcccggtgcgagttccccgctcttgatcagcgtgcggaagtgggtggccacgtcggcatagcccttcccctccggtgccttgtatggcatggcttctccctgtttgtcggagcgcggtacacccccgaaggtacatcaatgacccccccaaggtacatccttgcttcgaatcggtgcttgatgtaccttcgtggcgtcggcaaggtacgttaagtacctcccgctgactgatgcgtaccttcggcaagagagggtctgtcgtggccacgaggaacgttccgccccccggggcgaacaacagcaggaacaacaagttcgccgacatgggggcggcggccggcggtttcgtcggtgcgatgggcggctcgttcgtcccgcccgtgaacgtcacggtcaaccgcacgaccaacaagggcggtggcggacagcagtccggcggccgccagtcgcatttcatcctcggggagccggagttcaactcggctgaggacgtgcgcaactactgcaaccacgtccgcgccctgatgctccaggccgcgatcgagctggccatggccgccaagatcctggaggcccgcctcgcccaggcgcagacgctgcccggtgacaatccgatccagggccggatgcgggcgcggaaggtcggccggagcctcaagaaggccgccgacggcgccacgtccgccgcgaagggcgcggtcaccacctacggcgccttcacccgcgagtacgccgacctgatgcgcccgcgcccccagcgtcaggcgcccaccaaccccttcaagttctgagaggcggtaccgagatgggcaaggacgttcagcagcagcaggaagaccgcctcaactccggcggcacgggaatgggtgcctggctgtggcaccgggccaagccgtacaccccgccgtggatcgtcacgggcgcggtcggcgcggcgggcgccggcgcccacgagctgtggggcaactcgccctgggccggagtcggcctcaccctcgcgggggtcggcctgacggccgcgacctggtgggcgggcaagtccaccgggcagcagcgccgcctccactccgccatcaccgtggcggccggggcgacctggttcaccgcctccgccctctccggcccgctcaccggcccgctgcccgacctgtacctgatgggcggcacgagcctcgccctgacctggaacatccgccaggtcatgcgctcgtcgacgcccgagggcgccggatccgactcggacaagggactcctggagaaggtcgggctcgcccggaccaagctcaaggacgtcaaggtcgagcccaaccgcgtcacggtcccctacgagctgcctgccggggagctgaccaacgacgacatcaacaaggccatcccgcgcatcgcgtcggctctcgacgtgccgaccacggccatccgtgtccagcacgaccccgactccgcgaggaagggccagttcgtgatcgtgcccgaggacatgctgaagcagcccacgatctggcccggcccgttcgcgcccggcgagtccgtggccgtgcgctgcggatcgcgtctacgacgacgcagcgacctggttctcccgctcctcgacgcgatccacctgctcgtcatggggatgaccggctcgggcaagaccgagggcgccgtggacctcctgctggagatcctgacccgcaacgacgtgaccgtgtggctcgccgacgcggccaaggccgggcaggacttccagcccctcgtgcccgccctcgactgggcagccctggacacggcgtcggccggagcgatggtcgacgcggtccaggccgtcatccccgcccgcaccgcctggctgcgggaccacagctaccgggcctgggagcccgcggccgccaagacgcagaccaaccccgcgcactcctgcgcgtcggccggcgcctgcggctgccccgggatgccgtacctgctcacctggttcgaggaggcggccaagctcctgcgcgagctgggcgacgacgtgttcaccggcatcgcccaggaggcccggtcggcgggcgtctccctggtcgtctccatgcagcgcgcctccggctaccagctctcgacggacacgagggcctcgctcccggccgccatgtgcttcggcgtccggggcgacgacgccgggttcgccctccccgaggaggtcctggacgcaggtgccaacccggccgcgtggggcaacaagcgcaagggctacgtgtacctggtgtccgccggggtcgaggaggacctgtacgccaaccccgcccggacgttctggacgggccccccggccgagggcagctacgagcggatggcccgctacgtcgtcgagcacttcgcctcggttcgtgccgagctggacccggtgaccggcgccgccgccgagcaggctgccggaccgctgttcaccaaccgccgtgcccgcgcgggcgccgcctccgccccggcccgcccggtccaggagcagatgctcctcgacgacgacggccaggaggacggcgacctcgtggagatggagcacgacggcatcgacctgagcgccgacctcccgcccgtggagaacgacgcggaactcccgccggccaagccgtcgaccgaggaggcccgcgagctcctcgacgaaatggtcgccacgctcgcctcggtcggccccggcacggtcgctgtccgcgacctcaagccgtacctggagcagatcggccgtgaccgctcctgggtctcccgcgagatgaagcggatggccgaggagggccgcctggccgccacgggcgaggagggcgtctaccgcctcatccccacgctcgccggggtctgagacggcccgcacagccgcacagccgcacagcgcgaatccccacgtcacacggcgtgtgaagagggccgcacaccgcctcgcacaccgtgcgcacagccggaccgcacaccccccgcacaccgaacgacgacggaccgccccgcagcaaccggggcggcccgcccgatgaccacggaggtagagcccgtgaccaccgacccgaagcatctcaccgactccgaggcttccgccgaagctgcccgcctgatccgcgaggcgtaccagccgaccccggagccgcgccccatgaccttccgcgacaccacccggtcacagcgttcggcccgacccgcccgtgccccagcccgagacccggatcgtccccgagtgggccgccggggtcgccgtcgcctccatcggcatcggcgccggcgtcaccggcctcggctgcggagcctggctcatcttccagggcctgtcctccgtgaccctgctcggagtcatcgctatcgccgccccgttcgtcggcgtcgccacggtggccacggccatcggcgccgccatctccaaggccaagcgctcgtcgaccacgaacgtctaccaggggaccgtgatcaagcggaccgacatcacgtcgaccgcccgcggcatcggcgcccgctcccggatcgagggctgagcgccatgcagatgaacactcaggagcaggtcgagcaggcggagaaggtgctccggctgagctggatcatcgtcttcggcgtgatcctgttctccgtcttcacggtgacgcccctggtagagcggtccactccggagggctgggagtggtcggcgccgatcctgccgctcgtggtcgacgtcgccgtcgtcatctcgatccgggtcgacgcgatcgtgtcccggctcggagggtcgaccaccgggtggccgctcgccctgcgggtgctcaccggcggcgcctccgtggcgctcaacgtcgggcactccgtactccagggcgacctggtgggcgcgctcgtgcacacggccgccccggcggtgctcatcgtcgtcgccgaagcgtcgctcaagtggcgcaaggagatcgccgccgccacggcccggatcgaggctgagcaccgtgagcgcgaggacgcccgccgccgtgagcagcgtgagcgcgaggagaaggcgcgggccgaccgcgagcgtgagcaggaggcccggcgcctggagcgtgagcggcaggaggccgccgaccgtgagcgccgccgcgaggaactcgccgaccgtgagcgggagcgtgagcacgccgcccggctcgcccaacaggagcgtgagacgaggcccggctggaggccgagcgcgaggaccgggccgacgcccgccgccgcgaggagcaggaccgccaggagcgtgaacgcgagaaggagcgggagcgcaggagcaggagcgccgtgagcaggaggctgcccgcaaggccaaggaggccgtgcagaaggccgaacgggaccggaaggcagccgaggcccgcaagcccgccctcgcgcccgtgagcgctgctgtgagcaccccccgcccggccgtgagcgccgccgtgagcactcccgctcacgagactgctcacgacgccaagcccgtccagaagatgagcgaggccgacgcccgccaggccgtcgccgacgcggtccgtgagggccgctcacagcgtcaggtggccacgctcaccggctggtcgaccggctgggtcgccgcccgcttcaaggagcttgagggggccgccgcatgagccgcgccctgatgtacgcgctcatcctgccgctgttcgcggcggagtgctgggcccagttcgtggtccatgaccagcgctggacgacgctcttcgccctcctcgccggggccgtgctcgccgtccgctacgccctcggtccgcgcacggacgacgaggaatgcctgcccgactgcccgaagtgccgcgaatccaggggggacctgtgagcaccaccgaccagcacctgaccgcacagcacgccgaagtgaaggccgagatcacccgcaccgacacgaagaccgcgctcttgctcgccttcgtcggcgcggtcttggccggcgcctggtccctcgcccgagacctccacctcaaccccgtcgcgtacctggtcggcgtcctcggactcgccgccctcctcgccgcggccggcctcctgctccggtcggtccgcccgaacctcaacggcgggcacggcttcccgctgtgggccaccctcaccccgcagcagctcaccgccgccgccgagacccgcgacctggccgccgacgtcgtcgcctgtcccgcctcgccgttgccaagttcacctgcctgcgcctggccgtcgacctgacctgcacagggacggcgtcctcctcgtcctggccgccgtgatcgccctcggaggtgccgcatgacccgcaagcccgccatccacgacgccgaggcccacgtcgtcacctcccacggcagccgacttcttcaggcgaggaccgccacccgctcaaccgggtcgcctccctcgccgggtacgccgagggctgcctgccgtacgccgaacagccgcccgctggtcctgctgctgaccaaccccggcgacggcgggaccatgacgctgctcaggccggagagatggccacgctcctgcggaagctcgcccgccaccggttcgtcaagaccagcgccgccgcccacgcccgcgcactgggcgacgccgccgcccgcgccgccgccgacgcgagccctgggaatggcggatcgaagccgctgcctgaacaccgaagccccgccggcctttcggctggcggggcttccttcggcccgtcaaatcacatctgccccacgggccgtgtcgcgtgccggggggaacctccggcacaaaaagtgccaggatcacccccagcaaagcgaaacggccagggattagggcccctgaccgcttctgacgtccgcccggataccaaccaagggactcgtctgttgaacagggtaagggacgctgaggcgtccgcaagagcactcccggctcgcgccgtccgtccgcgctgccactgcggcactgcgatcgagcacacgcccggcaaacggccgcgcgtgtactgctcgaacgcctgcaagcagcgggcgaagcgcgctcttgccaagatcgcccgggaagccgccgacgcgcgtccgcgacccaaaacgtgtcgcgccttgggaaagaaacaacagagtttcccgcacccctccgacctgcggaaacgtcggcgggggcaaaaccggtcgcggacagccgggacgacgccgcccgcgcccggaaggctcgccggtacgcgaaccgccggacgctgtggcggatcaccggggacgccgcgtgcaagggctgcggccgggccctgatggaccccgcctccggcgtgatcgtcgcccagacggcggccggaacgtccgtggtccttgggctgatgcggtgcgggcggatctggctctgcccggtctgcgccgccacgatccggcacaagcgggccgaggagatcaccgccgccgtggtcgagtggatcaagcgcggggggaccgcctacctggtcaccttcaccgcccggcacgggcacacggaccggctcgcggacctcatggacgccttgcagggcacgcggaagacggccgacgctccccggcggccgggtgcctaccaacggctgatcacgggcggcacatgggccggacgccgggccaaggacgggcaccgggccgctgaccgcgaaggcatccgcgaccggatcggctacgtcggcatgatccgcgcgaccgaagtcaccgtgggccagatcaacggctggcacccgcacatccacgcgatcgtcctggtcggcggccggaccgagggcgagaggtccgcgaagcagatcgtcggcaccttcgagccgtccgaggccgcgctcgacgagtggcaaggccagtggcgagccgtgtggaccgctgccctgcgcaaggtcaacccgcagttcacgcccgacgaccggcacggcgttgacttcaagcggctggagaccgaacgcgacgccaacgacctcgccgagtacatcgccaagacccaggacgggaaagcgccggcactcgaactcgcccgcgccgacctcaagacggcgaacggcgggaacgtcgccccgttcgaactcctcggacggatcggggacctgaccggcggcatgaccgaggacgacgccgccggggtcggctcgctggaatggaacctggcccgctggcacgagtacgagcgggcgaccaaggggcgccgggccatcgaatggacccgctacctgcggcagatgctcgggctcgacggcggcgacaccgaggccgacgacctcgacctgctcctggcggccgacgccgacggcggcgaactccgcgccggggtcgccgtgaccgaggacggatggcacgcggtcacccgtcgcgccctcgaccttgccgccacgcaggccgccgagggaaccgacggcaacaccgatccggccgccatgggcgagagggtgcgcgaggtcctggcgcacgccgacgccgccgacgccgtggtggtgctcacctccggcgaggtcgccgaggcgtacgccgacatgctcgccgccctcgccctgcgccgcgaggaagcagctgcacgccgccgccgggagcaggacgacgaccaggacgacgacgccgacgaccgccaggagcgggccgcccggcacatcgcccgactgcggaactgatatcgatccgcactaactcgctgcccgcccctactcccgcgccgacctctccgtgacccgcacggagaggtgtcggcggcggtcggaggcttgcccacgaggcgcgacctgcgaggcagccgcaggcttgcccacggggcctcccaccctcggtcccaccctcggtcccaccttcggtcccacggtggacgcgacggtgggagcaacggccgagccccctgctgaagcaaccccgcccggcgggcgtcactgatatcagtgacccacaactcgctctgcctgtggttactgcctccgaggcaccgccatcgggtccgccagcccaccgccatacgcccgcccacgaccgccatccgaccggaatgcatggcggtcccatggcggtcggatcggaccccatggcggacccttggcggtgccatggcggacccagcggagcgagcaagttatcgcgagagcaatgctctcgcgggcgctcgttggggcgagcaagttatccgcttggagactccagcggtgccccgaccgagggcggtcggggttccccggggaggggaaccccctttgtcctcaccccggttttgatcacgtcggcctacgccgacggacccgcgcggcgcgagccgtgcggaacggaaaacccggctgccgatcccctcgcccgccgcccgcgttcccgccccacctccctctcctcctggtgctcgtggcggtcgtgggtggcgtagaggggatgtctgcccaagcggaagcccccgaccatgcgcggtgacgtgggacgccgcgaagcccggaaccggatccccgcaacacccagcgcaacccatggcgcaacccatggcgcaacacccagcgcaaccccgaccaaggacggccgggaacccgctacgacaccccctcgacgggcagcgcgtcgactcccggtccgagcgtccgccggcctttccgggcggccacgtcctggtgctcgatgtcccccaggagtgcgtccggctccactcgctcgacgggcagcagccgcggactcgcgtccgccgtcggcgtgctcctggtggtgctcgggccggtgccgaggtgacggcgcggggtgctcatgacgggagtctcccgtgccgttcccggagctcccgcagcggccctgatcgagccgtgcggcttgtgcgttcgtgaatgcaagaggtgtgaccgttcttcgcgtgcacgcgtgtgctcgtgcacctgtgcacgcggtacggcttcgccgccgacttcgtcgtgaccggagcggtcacacctcaggcattacgaatgacctcggctggtcgcgccctgcgttgtgccctgggtcgcgtcctggattccggcctgagtcacgtcctgggtcgcaccggacgggtccccggccgcgcccggttcggcgccgtgacctggtggacaagggggcggtcggctgccgttccgcgcgccgacttcttgctgggagcggtgtcggcgggcgccttcggatcggaatgcaagggtgtgcggcttgctgctgccgtacgtcatctcgaagacgtacgcgggccgtcggtgacgtactcgaggaacgaggccgggctcggctcggcttggtcgaccccaggggctttttcgtctgcggtcctgt

SEQ ID NO: 62 - DNA, 3539 bp, synthetic construct
  source 1..3539: mol_type = other DNA; note = pGP01
ccacggtgttggatgacagtcctttgttgccactgtcgcagtttttcttttcgcgaagtgcttgagatccttgatgggtggacagtacggtatcctcaaccgtggattctacaacggaagccacaacgaaatcataggaggatatcccatgaacggacgagtgattgtttggcctgatcctgcgctcttgtctgccatgcctttaagttccggattttccccggtcatgtcgtggaaccgcgagctgaagtgtttcaccgatgtgcagaccacgactgacgacggactgcctgtctggggggcagatgcgtggttgaggatgggatggcaaccgcacgctgacacagtccaactgcgcatcgctgcacctcgaaagcctgcggtccagcctgatcctgcgagaatcgctgatttttttgcgcctcgacgtagtgaggctgagcactgatggcaggcgaacgaacgactgttctgaccggcgtctactggtggctccgtatcttgccggtcttattactcggcgttgccttcgcgtgctggtgtttctcggtgcatctggtctacgtgatcggcttcgctgttgcggccttggcggtggaggcttttccgccgtctgtatggcaggtgcccaagaacatgcaggtgccagcacgatacgtctatcgctggtggtggtccttggcgaaagcgttcaagccgattgaatcctatggcggcgacagaatttactatcgacccggcttgcagtggatccgctctgaccgacatgtactgcatctggtgcttcgcgttcctgcaggtcttgccgactcggcggcatatctggagaaaggtgcagcagagatccaacgacagctacgtggcaagaagtcttggaggacctgcatcgtcaaacctgccgagcatggcctggacatcattcctcgcgacgcgacagctggtgatgagttacttcctgctccgtccacgtcgtcgtggaacgtgccagtcggggttaaacctgacgggtccgaggtcgtcttggatctctcccatccatcccacatcctcgtctccggaaagactcgttccggcaagtcatcgttcgtctacggcctgctcgatcagatgcgtcatcttcctgtcactgtggctggtgtcgacccgaccggaatcctctttaatgagctgggcgacggctggggcggtgatgctctgcgttccaagcgcatcacgaatgacgctgatgctgcagcagttgtccaggtcctctccatgatcaccgatgaaatggatcggcgtatctatcttcttaactgtgagcatcgcgacaagtggagccgcaacgatttcgagtccgacccgggacgtcgactcatcatcgtcatcctcgaggaatatccgggcttgattgagcggctgcagaacttcgattccgcccgcggcgctcgctccagtgatcgttttgcctcgaaggcagctggcctcgttggccgcatcgcgtacgaaggcgccaaggtcggggttgtcctcctccttgtcacacagcgacctgacgccaaaattatcggcggtccactgcgagcccagctcactacgcgggtgacgttcgcccaagactcagacggattgcgtatgtcgcatcctgagctctcatctgagcaggtcaaacagaattcatgggcctcaggtgtcgggttcatcgaagcagatggcgtgattccgctcactcggttccggtcctatcgagcggaactcaccgacctgcatcggcccggggcgtcggtcggccagatcgatctgatccagtgaggagctgctgccgaccgatgccgcggaactctgagatcgacacgaccacagatgccgtagtcacggagacgctcgaggaagagataggattcagtaccagataaaaaatgcctccccacagcgccaactgcggggaggcgagtaagacctttttctcccgggtcatgaccgccaaggaggcgatcggtgttaaacatagtaacactatccaaggcgcagccacatgatcggttgtgctctgagcatgagccgtgtgcggctcgccgcagcgagcgtcagcgagcggcgggcgaccgccttggtaccacgcgagcaacttttccggtatcagattccccctgtagaaagccgaatgagggccgtcgccatcgctatgagatgagggatggtctacgaaatccgcaggtcatgccgctggagcgcgttcgcaagtgcggggcagtgccggtttcgcaacggatcgcgttgatggcgggtcatggtggtgccggttatgccggtttggcgacgtgcggaagtgtgtgggcttgccctgtctgtgcggcaaagatttccgcgcaccgtcgtgatgagctggcccgtgttgtccaggttgcggttggactcggcttcaaggtgtcgatgctgacgcttactcaacgtcatcatgctggtcaggatctcgccgagctgtgggcgtcgctccagtcgggttggaatgctgtcacgagtggtcgacggtggcaggaattttgcgctcagctcggcgtccagggatgggtcaaggcagttgaagtcacccatgggtcgcatgggtggcacgttcacgtgcacgtgctcgtcatctctaagcaggatccgactagcgttgacactaagattcggcatcgccgcaaacaaggtcggcgccggaccccgtatccagaagaggtacagaggcccgaagacttcatcgctgaacggtggtcgcgaggtttgaggaagcgcggcgtcgacttcatcgccggtagtggtggcctcgattggcagactgctgattctggagacgaggaagctctcggtcggtacgtcgcgaagatgaactcgtccgtcgatggcctagcgaacgaggccacgttgggcgggttcaagaaggctcgtagaggtaatcggacgccgttccagatcctcgaagatttcctggatacgggctcggagactgacctgagactctggcgtacctatgtttctgcaagtcatggccgtaaggcattgacgtggtccaagggtttgcgtgactgggctggcatggaatctgagatgagcgatgagcaggtcgccgcccaagaccagtgcggggaagcggtcgccctttttgaccatgacgcgtggcggcagatccgcactgccggtgccgctttcctcctcgacgagctggagctccacggatccgagggcgtctacgcctggctgaagaagcgaagaatccattatgagatacctctagttccttggagtacgagtacctaggagccagtcggggtctgtcaattttttagctcctccatttcatcacactctttctatgatgaagtcatcacaattcggtattctttgactcccctgagaagccgataatcaggccagtagagctatcttatgtgcctaggtggatactatttattcttcacctatcagggactctggtcgatcacagcctccgtcgacgatgacacatctgactaggtactatgatgacttcatcatagacagaggtggagcacagacga

SEQ ID NO: 63 - DNA, 8136 bp, synthetic construct
  source 1..8136: mol_type = other DNA; note = pIP501
cctgctcgggaccccccctagaatttcgttattcaccaaaaaaatacgcatggtataaagcaccaagcgattataaaaaacgtagtcgaaaaaattatgatatggacacaataaggggaatgtatggaagccaaaaaattaaagaagaaaaagcagaaaaaaaatacttacaagcccgatagggtttgtaagtttatcagaaaggaagttttaaagtggatattaaaataaaaaaaataaattttgaaggtaatattttaaaagttataaaagcgacagtaacagaaatgagaggaataaataatcatcaaaaatatgattttgatttatatcaaatagaagcacgttcgccaatgtcaacaagagaaataactttaacagttgactttatagaaaaaaagtatcaggtgatattatcgcttttggtgattggtacgatttggatatagaatcagtaaatgaaatattaaagcaattaaaaaaggaagaacaaacattaagaacaatcaattttatttaaaaaagtatcaaaaaaaggaaaattatttttcctttttttgatactttttaggtttatttgctttaatatattcagaaaaaatttttaaaaattcttccgtttcttctacagttaaatagtcaatttcaaaatacttttctaacaaagcacctttttcaattagacgctttgttcttctttttctgttaatattttgctcgctattcgctcgcaaaatatttttttgaatttttagtttttctattcttttgttgatgtcatttaaatcattatttgccataactatttaagtaattcagaaaaattttgactattcgcatttaataaagtttctaattcgtcatagaataattctttgttttcttcaattgattcaatagaaatattattttttgaaagggtagaaagaacaaagtcaccaatatctgctttagactttagaattaattcttcttttttcttttgtaattctttaatttgaagttcaatatcagaaacagttttcttctttcttttttttggcttcgttttagaattaaaattaccattaattgcattaatattttgatcttgattttccatgaaatacgcctcctaatatattgtgtaactctatcatattatattatttttctaaaatcaataaatataaacggaaatgtcaggttaaacatatttacttttataatgataagtggtaaaattaatttattaaggattcccttagattatttactaagggcgcacttatacgcagtaacttcgttacttcgtatttatgctataaaacttaactgttagttagttttatcgtcaagcgtgtttgttaaaattcgctacgctcatgtttgaaaagaaagagaggtgatacaattggcaatcttccatttatcaatgacaatagcaaaaagagaaaacggaaaaagaagtttaatcgcaatggcttcttatcgaagtggtgaaaaattgtatagtgaactatatgaaaaaactaatctatacaaccatagaactgttaaaccagaagcttttattttaaaacctgattatgtacctaatgagtttttagatagacagacattatggaataaaatggaattagcagaaaaaagtccaaacgctcaactttgtcgagaggtaaatgtagcattgccaattgaattaaataattcagaccaaagaatgttgattgaagattttgttaaagataattttgtcaatgaaggaatgattgcagacgtagccattcatagagatgatgaaaacaatcctcatgctcacattatgctaacaatgagagaagtagatagtgaaggcaatatcttaaacaaaagtcatagaatacctaaactagatgaaaatggcaatcagatttttaatgaaaaggggcaaagagtaaccgtttcaattaaaacaaatgattggggtagaaaatctcttgtttctgaaattcgtaaagattgggcagacaaagttaatcaatatttaaaagatagaaatatcgatcaacaaataacagaaaaatcgcatgcggaacttggaaaaaaagaactaccaacaattcatgaaggtttttactcaaaaaaattagaagacaaaggagttataagcgagttaaaaagaaaaaatttagaaattcaaagttacaatgatattctagccgaacttgataaacttgaaaatcaagaaaaagtattaaaacaagaccaaaactttactttaaaatttgaaaaaactttctcacctttagaaaaaggagaactgaaaaatctttcaaaagaattgaaattatttattaatgatgaaaacattgataaacgattaggtgaattaaaacgatgggaaaattcacttatctttaataataaaatggaaattcaaaaacaacgtttgatgttaagtaaaattagtagtgaacgagatatgcttacaaaggcaaatgaaattttagacaaacaagcagaaagattcttcaaaaaatcttatccaagtttgaatattgacaaattttcaaatcacgaagttagagcaatggttaatgaaaccatatttagaaaacagttattgaataaagaccagttagcagaggtcatttacaatgaaagagtagtagaaaaagaagaaagtaaaaagatttttaaagaaaaaccatttcaaactagccgttatcttgattcaaaaattaaacaaattgaagatagtataacaaaagaaaataaccctgaaagaaaagaaattttatcaattaaaaaagaaaaactaataggaataaaacaaggattgatagaatatgttcaatcagaagttgaaagaaaatttgataaaaatgtttcaatagattcagtcatagaaggtgaaatgttacttgcaaaagctgactattacaaaacaactgatttttctaaagtcgaaggagttgctagattcagcagtgaggaaattaattccatgttggaacaatcaaaaggcttcttaactaacattcagacggtgaaaattcctaatgattgtcaaggtgtattttttgttcaagatagcatgaaacatattgatgaactaagcccattagcaaaacaaaatctgaaaaaggttgttaatcgcaatgcttatttacctgattctgataagatagaattaagtaaagaaattgaaaataccaataaagatcaatcccaagaattggataaagacgtaccagaaaaaaatgaagtgactgtaaaaatgttccaatttgcgaagtcaattaatcgtttgttgagtggtaaccaactacagaaaaaacgaaacctagacaaattgattaagcaaacaaaagcaaaaaaaaatcaatcattacaaaggaatattcctttgcgataaaataaaaacaagaggtgtataaaatgaaaaaatttatcaaagatacaaagtttaagcttggaagtgcggttgttgcgttgggtacattgtttattactgatccagtgtttgcagccactgatccacaagcgaaattagttcaagcgggtaacactataaaaggtgttttaacagccttaattgttgtagttggtgggattgcttgtgcgaagattgttattaaatacttgccgtctattgatgacccacaagaaaaaaataccatgtataaagccttgggaacagccttgcttgttacggcattaggtggggcgttggtttggttagtaccttgggcgtatggcttacttgcttaatagagaagggagttagttatgaatagcgatcaagtgaaacaagccctattagatttgttaaatgcagacactgaaaaagggcggacttggttttttccgtctaatgtatctgatcggtacacagtcattttagggctagatttaaaacaatcagcaaaagcgatcggtacggcattaataagcgtgttattgacaattcttattttccgtagcacagccgtttttcctttaattatctatgtcattgttggtttggtgtcatttggtggtgtatgggcgttttatacgattaaaccaattacagaccgacctaacatttctatatctgattttatgaagcaaagaaaagacttttctaaaagaccaaaagtctattacaaaaagccaaaagaacgagtgtaaaagagaggtgatttaattgtttgattttctaaaaaaaagttccaaaagtaacgataataaaaaaagcgatacgatgaaagaaatgatttattgggaagatagttcccgatttcaaggcgtttttaaagacttttttgtcgtttatcagccagaaaaaaagcaattcagccttgtgagtatgctaaaagttgacggcttaaacgttgataccttgccagtatcagagcaagaagggttaaacgaagattttggtgtctttctatctcaaaacgttctatatgaaccgcagatcacttctaagaatgtaccagtagaaattgacgattttgtagaagcctgggggattacagtagaaaattatcgcaaaatgccagggcataacgaagctttattacaattaaaggctagttactattatcattatagaaatttagcaagtaacatggaaacttcaaagaaacaacattttgtaattaattctgaaccaatttcaaaggaaacatatgatagtttagaattgtcctatcaagtgttacgtgataagacaaggacaatcaggacggctttaattgcttttttaagcaagtatgattgccaagttgaaatgtgtacgattggcgagatgaaaaaggttttgaatagttaggagcgttaaaatggagaagataccaaaagagaaaattgtcttgatacccgaagttgatacggacgttgtatctgatttagcaccatttaactttacagtagaacgtgacaaattattgattgatgattcatacgcagttccctatgtcattacaaaatacaacaataagccacgtgggaattggtttaatcgtattcgtaaaatgagtggagatataaccatatctcattactacactaaagcaaacggtaactcattgaatgattattacaacagaaccattaagaacaagcaagcagagatcgatcgttcgcatgatccgttgacgattatccgtttagaacgtgaaatgaaaattgctcaaacgcagttagaacaagccgttgacgaaaacacttcttatctttacttgtacacctatgttttgattaaaagtaagtcagaagataaattaaaaaaattgtgtgaagattttgaaacacgttgtatcgcaagtggagtaaaagcgttaattccatatactatgattgataaggcgtattggagttcattaccgttacaatctaatgaagtacctgaatacacctatacaatcgctaattcaatcagtgcaagcagtatttttccttttgatgataatgaattaagtgtatttactaaaaatatgattattgagggaattaataaagatactgaaaatatcgttagtattgattacaccaacagaaaattagtagtcaatcgtaataaattcgtttttggtttatctggtgggggaaagaccacttacttaacgtcagactatttaaaaaaatatgctttttctgataactcaacagaattaaggcacagaattgttttatttgatcccgaagatgaacaaacagagcgtgtacgttctctagggggcgaaataatcaatctatcgtctatgtcagatgttcgtatcaatccatttcaaatttactcacgcaatacgctagatgttgatttaaaagaatcattatccgattttgaagaggacgagcttgtagaaaatattgaaataaagcataaagattatgaaatgactgacaatgatattgataaagaaatcagtaaacgaatgaatattttaacgccttatttcctaatggtggatcattctttgactgatagtcaattatccattattaaaatagaagctaaaaaatgctataccactttatacgagaagaaaaacttgtcaaaaatggaaaacaccgattttccaacattttcagacttagaaaatcgattgaaagccttagaagaaactgatccaaaaagatacaaacgaattgaagattttatttattcattagaagattttacaatcggaagtcgtaccatttttaacggtcatacaaatatagacttaaacaatccgttaatttgcttttctttgcgagatttacagaccgaagaagggatcagagatttagcatacctcaacagctttagttatctatttgaagaaataaccaacaatccgcaaattgtaacgtctgtttatgcagatgaatttcactttttattgaagaataaaattagtgctgactttttcttccaagcatataaacgctttagaaaatacaatgctgattgtaccgtatcaacccaacagattgatgatgtattaaaagcacctgataatatcggtaaagcaattattgggaatagctttacaaaagtattcttcggacttgatgaaacggaagcacaaggtatttcaaatgagttgaaacttaaactcacaaaaaaagaattatcgttcattacctcaaaacgtcaaggggaagctttgctttttcatggtacaaagcgagcaaagataaaagtagatttaacacaggaagaaatgcgtttgcttaacccaggcgaatatgaagatatttacggcgttagtccgaagaaagagatcaactggttgttaagatcgaaaattcaatagaagggagaaaaaaatgaaatacaaaatcttgaaaaatttacaattctactatcaagagaatgtcattgtcgtccaaataaacgaaaaatatttgacgaatcgagaacatatttttgatgtagaagaaagtgaacaatattttgttgatgtcgaggagattttgaccaaagacggaaagctagaaattgtttataaccgacctaatggctatacaccactactagatttaaaagaatatgctgatttttataaattggatatagtgaatcgattacttgaaatgaatgtactagaaaaaacaaacacctatctagcaatgcaaaatatcctactcaaagatacacgtgacttgctttttatttataaagcagatcactttgataatttgccttactcaactaaagaagaattagagcagtggaaaaattttatttgtagtttttttggtaaattcacacttgagaagtatgagaagaatcgtattgaggttctaacaaaagaaaaaaattcatttttaaatgatgtagaagcagttgaaagcttggaatcattaagagatttaataaaaaatcgactaaccgaagaacaaaagaatttcttttctgctgaattacaggacaagaaagcagacgtccgaaaaattcgcagaaataaaagcttaaaaattgcgttagttgtaggtgttattgcgttatatggcggtacggttttacttatgaaagtaaatgagaagaaacaagttacggctacacagcaaagcgcagaaacagagatcactattttaaataagattattgataatgatagtgagaatatcgaagaagatatgcaaaagctcaattatcctaagaaaaaacaagttgatatttacgtgaaacttggtgattataccaaggcttatgaacttgataaaaagtcagataaaaaaattattcaaagtctgtacaaacaaggagaaaccgaaaaaatagaagcccttgatttaccaggaagcgactatttagcagacttcaaaaagattttagcgtatgacaattcaacagatattgagtatctggttcaaactagtaccgatacaacaattgttgaagctttaatcgataaatcagtaaaagaaaaagacattccaacagtgaaaaatattcgtcaagtatcaattacacaaaagaaattagcaatcgatcctaaacgtcaaatcagtatgattgacttattgattgaaaataacagtgaagaattagaaaatatgtataaggataattctttaaatgaggacttgaagaaaaaacaaaccaatgacttgttagaagaaaacaacacgttgcttagtgaaaagattgaattaacaaacgctgaaaaagattaggaaggttggtgattt

SEQ ID NO: 64 - DNA, 2053 bp, synthetic construct
  source 1..2053: mol_type = other DNA; note = pCU1
ctgtttccattctgtttctgaaattctgtttttctgagccatctgtgggcctccgtagttttggttacagaaaggatatactcagaataaataggggtcaatacaagtacgatttttataaactttattttatttgagggtgaggcccggtgcggcacgagcgcggcgttgatggtgccgcgaaaggtgcctggcgccatgcttggattaaaacatgaaccgtgaagaactgcgaaacttgttttcgcggttctgaggggttgaccgagccgcgaagtggtaagcgatgatatgcacatatccacaggcatatttttaaaaggtattttatagattttttatctttttaaagtcttttagagctatataactcattgatttaaaatcataaataagtgttatctctgggaatccgcccaccttgttatgggaattggcccacctatctatgggaaacaccccaccttactatgggaattagcccaccttgttatgggaattggcccaccttagacgaaactgtaaaaaatgtatttacttgtttgaactttgtggtagtgtggagagtaatttttaacccacaaaggcaaggctcatggataagttgctgaacaaaaagataaaagttaagcagtctaacgagcttaccgaagctgcttactacctctcgctaaaagcaaagcgcgttctctggttatgtcttatgcagacgtatttcacagcttcagtaagcgaagatgatgatgagatggctgtactcggtgactctactttcaaagtaaaggtggctgactatcagcaaatttttcaggtaagccgtaaccaggctatcaaggatgttaaagaaggcgtgtttgagttaagccgttctgcggtaatcttttacccgaaagaagggagttttgactgcgtcgcgcgcccctggctaacagaggctggcagccgatcagctcgtggtatctgggaaatcgaatttaaccataaactcctgcggtacatttacggcctgacgaaccagttcaccacctactcgctccgcgattgtggcagtcttcgaaatccacggacgatccgcctttatgaaagtcttgctcaattcaaatcttcaggcttatgggttactactcatgcttggttaaatgaccgtttccttttgccggaatcccaacagaagaacttggcagagttgaaacgatctttccttgatcctgctctcaagcagataaatgagaaaacacctttacttgctaagtatagtattgatgattcaggaaaatttctgttctcaataattgataagcaaaatcccgtctgacataaatcagcacacatgagcctgtcatttgacaaatttttgtcatgaagatgggcgaatttccacacagcaccggcgcccggcaaggtgggcggattcccacacggcaccgacgcccggcaaggtgggcggattcccacacggcaccggcgcccggcaacggtgggcggatttccacacagcaccggcgcccggcaaggtgggcggattcccacacggcaccggcgcccggcaaggtgggcggattcccacacagcaccggcgcccggcaaggtgggcggatttccacacagcaccggcgcccggcaaggtgggcggattcccacacggcaccggcgcccggcaaggtgggcggattcccacacggcaccggcgcccggcaaggtgggcggattcccacacggcaccggcgcccggcaaggtgggcggatttccacacagcaccggcgcccggcaaggtgggcggatttccacacagcaccggcgcccggcaaggtgggcggatttccacacggcaccggcgcccggcaaggtgggcggatttccataactttaattatacctttgtgttatttgtggattgtgcagctcagtggggcgctggccgtgacggtgcggtgtcccccgtaaccggccgcgcggccgctaactcgcagtacggcgccgcgacccgcagcgggccgccgtacccgcgccgcacggcgcccactgcgcacccccgtggaggacgtgcggcag

SEQ ID NO: 65 - DNA, 1540 bp, synthetic construct
  source 1..1540: mol_type = other DNA; note = pBAV1K-T5
tccgccgccctagacctagtgtcattttatttcccccgtttcagcatcaagaacctttgcataacttgctctatatccacactgataattgccctcaaaccataatctaaaggcgctagagtttgttgaaacaatatcttttacatcattcgtatttaaaattccaaactccgctcccctaaggcgaataaaagccattaaatcttttgtatttaccaaattatagtcatccactatatctaagagtaaattcttcaattctcttttttggctttcatcaagtgttatatagcggtcaatatcaaaatcattaatgttcaaaatatcttttttgtcgtatatatgtttattcttagcaatagcgtcctttgattcatgagtcaaatattcatatgaacctttgatataatcaagtatctcaacatgagcaactgaactattccccaattttcgcttaatcttgttcctaacgctttctattgttacaggatttcgtgcaatatatataacgtgatagtgtggttttttatagtgctttccatttcgtataacatcactactattccatgtatctttatcttttttttcgtccatatcgtgtaaaggactgacagccatagatacgcccaaactctctaatttttccttccaatcattaggaattgagtcaggatataataaaaatccaaaatttctagctttagtatttttaatagccatgatataattaccttatcaaaaacaagtagcgaaaactcgtatccttctaaaaacgcgagctttcgcttattttttttgttctgattcctttcttgcatattcttctatagctaacgccgcaaccgcagattttgaaaaacctttttgtttcgccatatctgttaattttttatcttgctcttttgtcagagaaatcataactctttttttcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgcctagtgc

SEQ ID NO: 66 - DNA, 383 bp, synthetic construct
  source 1..383: mol_type = other DNA; note = Cos PAC7 383
gaccacatcacacccgtcagccggggaggactcaacaccctcgacaacgggcaaatcatctgcagaacatgcaacagaagcaaaggcaacagaacacaaccaaacatcaaattccaacaacaaaccacaaaaacattgattccatggtgaaaaacccgccaacccccaccgggcacaccccctgcacacccgtgcaagacctcgtacggcttagtgaaatacctcccttttgttgttttatcgttttgtcgactttttgtttggtggtgtgtgtggtgcagcctgagcttcctgatagtcgtgattggtgtggggagacgcgtcggtggtggtgtgtgtggggcgaggatccgcgtgccgggtttgtgtctgatgaggagtgg

SEQ ID NO: 67 - DNA, 511 bp, synthetic construct
  source 1..511: mol_type = other DNA; note = phage PAC7 origin of replication
cgacatcagtcttaaagtcttaaacactttaagtaactttaaagcttcaaggcttagcccttaaggatctaagttactataaaagctttaaacacttaaagtaactataaagctttaagagcttaacatttaaggatataaataaacattaaagctttaaagtcttaaagtaaatatataaccttaacacttaagttaagtataaaaccttaaaggcttagcacttaaggatataaacttaacatcagtgtttaagacttaaagagttaaagtaactattaagacttaaaggcttataagctttaatactttaagtagctataagactttaaaaacctgaagtacttaaagttaaccatcagtcttaaactttaatattataagtattaaagcttataagttataaaagtttttagaagagttaaagggttaacttctttacttctcttctctctttggttctttctctcttctcttcttttcttcatcaggggagaagaggaacctttaa

SEQ ID NO: 68 - DNA, 29768 bp, synthetic construct
  source 1..29768: mol_type = other DNA; note = PAC7
tgcaggtgctgacggtgtgaacggcgttgacggcgctgatggtcgggatggttctgccggtgagcgcggcccgcaaggcccttcaggtcctgccggcccgcaaggtgcacagggtgaacggggtgagcgtggtcccgccggtgcgaatggatcggatggccatgatggtaaggatgggcgctcggtggtgtctgtgtactgttccgggggccgcctggttgtgaaatatagtgacggtgtggcttccacgatatcgggttcggcggcctgccagggtgtgaaaccgtcgcctctagtgactatatcatcccacaaatagaaaggagtggctgtgatggtggtgtttggtggtggtgtgttgtgagatatattcctgcggcgcatcattctgccggctcgaatagtccggtgaatagggttgtgattcatgcgacgtgcccggatgtggggtttccgtccgcctcgcgtaaaggacgggctgtgtccacggcaaactatttcgcttccccatcgtctggtggttcggcgcattatgtgtgtgatattggggagacggtgcaatgcttgtcggagtctacgattgggtggcatgccccgccgaatccgcatagtttgggtatagagatttgcgcggatgggggttcgcacgcctcgttccgggtgccggggcatgcttacactcgtgagcagtggctggatcctcgcgtgtggcctgcggttgagcgtgccgccatcctgtgtagacgtttgtgtgacaagcatggtgttccgaaaaggaaactgtctgtggccgatttgaaggccggtaaacggggtgtttgcgggcatgtggatgttacggatgcgtggcatcagtcggatcatgacgatccggggccgtggtttccgtgggacaaatttatggctgtggttaatggccacggcggcggttcaagtagtgaggagttgagtatggctgatgtacaagcgttacataatcagattaaacagttgtcggcacaggtggcccagtcggtgaataagctgcatcacgatgttggtgtggttcaggttcagaatggtgatttgggtaaacgtgttgatgccttgtcgtgggtgaagaatcctgtgacggggaagctgtggcgcactaaggatgctttgtggagtgtctggtattacgtgttggagtgtcgtagccgtcttgacaggctcgagtctgctgtcaacgatttgaaaaagtgatggtggtttgttgtgggtaaacagttttggttaggtttgctggagcgtgccctgaaaacttttgttcaaacgtttgttgccgtgttgggggttactgcgggtgtcacctatactgcggagtcgtttcgtggtttgccgtgggaatccgcgctgatcacggcaacggttgctgctgtcctgtcggttgctacctcgtttggtagcccgtcgtttgtggccggcaagcccggcaagcagccccaggtggatgcgggtttggttccaccggatgatgggggcttggttgagccgcatatggtggatgtgtcggatcctggcatgatcgagccgacggatgatgcggatcttgccggctatgagcctcggcgtgcagccgagtcggaggttggcacggtagagtctactgttgcataattgaatatagatgtgtgccccagcggtgctgccacgattgtgtggtggcggctgctggggcactatttttgtatatgcggtgtggctatgattcgttgctgtcgatggtgtcttcgagcatctgatacaggtggaggcaggtagagatagtttcgctggcctgatcgagaacgttccggccgataacgtttttgtggttgtcgcggtggcggatgatagcccacatgatctcgtcggctgccgcctgtaatagtttggcctggtatgcgattccggcgagccagtctagtgcttcctggcttgtataggggctctggtcctcgctgttgccgcgggtgttgctgttgtttgtggggtgtcctgcactgtcgcatagccacaggatttcgctgcactcgtctagcgtgtcttggtcgatagcgagatcgtcgaggctgacattgttgacggtaaggttcacgttgtcgagggagatgggtacaccgtactggttttcgacactgtcaacaatgttttccagctgttgcatgttggtgggctgttgttggacgatacggtgtatcgctgtgttgagggtggtgtaggtgatgttgtgtgtgttgtccatggtttttatgccattccttcgttatcgtctggcatgtagtatgtgctgtttgcgtactcggttaacgtcatcagtgtttggtctgcccactgtttcacggtttgccgggtgactccgagtcgttgggcggctgtggcgtaggtttgatcatacccgtatacttcccggaatgctgccaacctagctaggtgtttcctctgtttggatggttcacaggtgagggtgtagtcgtcgatggctagctgtagatcgatcatggagacgatgttgttgccgtggtgttgtggcgcggttggtgggggtggcattcctggctccacggagggtttccaggggccgccgttccagatccattgggcagcttggatgatgtcggcggtggtgtaggttcggttcactggtcaccccctgaacaggtcgttggtgttgttggtgtcgaatcgtccgacgcagtggcagtagtcgtacatgagtttaataatgtgttggtggtctcccaaataggtgtttccgctgatgctgtatgtggctgtgccgtctttcgcgatggtgtatttggcggtgatggtttcggggttttcggtgtcggtgatgattgctgtggtggtggcgcctactgtttggagtatggtggtttgggttccgtcgtcgatggtggttttaaccatggtgtgtgttttcccttttgttagttgcttgtttggttgtcggctagatgaataatatcgggtaaaggtttcggctggtctaggtgttgtatggttttgttggctagccgtttggctaccctgtaacacattttggtgtagtgtttgttgtctaggttgtggtattgttcccgcaccgcaatatatagcagggagtcttggtacaggtcgtctgcactgattgcggggtagtgtgcggctgttttggtgcatgcccggttgagtgtgcgaagatgatggtctgtggcccacacccacgatgcggtggtggccaggtcggcttttgttggtcgtctgctcatggcactatttcatctcgctatctgatagttgtttggtgttttgttgtggatagtgtagcacactagtcctgggtggccggtggtgcctgtgcggtgacggaaccatgtggattcgccttccatggatgggcattggatgaaggtgcgttgtccttgctcggagatttctaggtggtgccggtgcccggccatgagaatattagatacggtgccgttgtggaattcttggccgcgccaccaatcatagtgtttaccggtgcgccattggtgcccgtgggcgtgcagtatccgtgtgcctgccacatcaacggtggtggtcatttcgtctcggctggggaagtggaagtgtaggttggggtattggttattgagctggtaggcttctgcgatggcccggcagcagtccacgtcgaatgagtcatcgtaggtggtgactcctttaccgaagcgcacggcttcaccatggttgccggggatggatgtgatggtcacatttttgcagtggtcgaattggtggatgagttgcatcatggccatgcgggtgagcctgatttgttcggtgaggggtgtttgtgttcgccaggcgttgttgcctccttgtgacacgtatccttcgatcatgtcgccgaggaaggcgatgtggactcgttcgggtttgcctgcttgttgccagcagtgttttgcgactatgagggagtgtaggtagttgtcggcgaagtgtgctgtttctccgccggggatgcctttgccgatttggaagtctcctgccccgatgacgaaggctgcggtgctgtagtcggtgtgggtgtcttgttcgggttttgggggtgtccattcggctagtttatcgacgagttcgtctaccgggtaggggtttgttgcgggttggtggtcgatgattttttgtatggatcggcctgtttctccgttggggagtgtccattcggagatgcgtgtgcggcgcacggtgccgttggctagattgtcgtcgatggtgtcgatggcgttgtcgtggttggctagctgtgtgagtagccggtcaatattgtctatcactgggtatcctcctcttgcggggtggtgctggcttgtttgcggcgatagtctttaataacggtggcggagatggggtatcctgcctgggtgagctgttttgctagccatgaggcggggatagacctgtcggcgagcacgtcggcggctttgttgccgtagcgttgaataagggtttcagttttggttgccatgatgtcctatcggttgtgtggtgggctgccatcctgtgcggcagtcgccgtcgtgtcctggtttgcgtgtgcaccacgatacggttccgtctgtgtggttgagtgttttaccgcacatgacgtttcggagatgctccggcagctggtcatcctggttgctggtttgtgtgtcgaagagtgttttctggttggtgaaatgttctgacacggtgccgttatgcacgggtagtatccatgttttccattgttgttgtagcctggtgttccagtggaattgtttggcggcgttttcggcctgttttaaggttttgtggtagccgactagtatgcgttgatgctgctggtctggagggtttgggcctcgccagtattgtgccgccacggcgtagcggttgctgtctgtgaaggcgtcccagcagtattcgataatgtgttgcaacatactgtctggcaggctgtcagggttgatgttgatgttttgggtgataatgtcacggatggcttgccggtttttggtggtgggtttgaacgagatgctcacgatagtaccggctggtcgtcttgcatgaactggttgaaggtgttgttcccggcgtgttgggcttgtgttatttgttggtcggtccagtctgggtgttgctgtttcagatagtgccagtggcacgcattgtaggtttcgtcttgtagccgtgtgagatggttttcggtgatgatttgtttccacatggcccatgacacgtcgagccggtcgaggatttcgagggctgggatgttgaattggttcaggaagaggatttcgtgggtgtagtagtttttctcgtaggcgtcccatccgcttcggtgcctgttgggctggtttttggggtaggcttcccggcatactttgtgtaaacgcttggccatgtcgtcgggtagtttaatgtcggggttggcgcggatcatggatcgcatcccatcataggtggtgccccaggtgtgcatgatgtaggtggggtcttctccgtcggcccatttttctgcacagatggcgaggcggatacgcctcctggcagcttggctggtgttgcgccggttggggattgggcacgtgtcgaggggatccatgatgttttagtgtacctttctggtttcgtgttgttgacaggttttactgtagcacagtgtctagtgcgtgtgtcaaccctgtttttccggcttgaaggtaggtgtctgtgacatcccctagggtgaggggcacgtgcacagcttgggggagtgccgcctggagggtttgggccatctggtcgcctgcggggtctgggtctgaccagatgtagatgtggtcgtagccttcaaaaaatttggtccaaaaaatttgccacgaggttgcgccgggtagggcgacggccgaccatccgcattgttcgaggatcatggagtcgaattcgccttcgcaaatgtgcatttcggctgccgggttggccatggcggccatgttgtagatggagcctgtgtctcctgccggggttaggtatttggggtggttgtgggttttgcagtcgtgcgggagtgagcagcggaaacgcatttttcttatttcggctgggccgccccaaacggggtacatgtatgggatggtgatgcactggttgtagttttcgtggcctgggatggggtcattgtcgatgtatccaaggtggtggtagcgggctgtttcttcgctgatgcctcttgctgagagcaggtcgagtatgttttcgaggtgggtttcgtagcgggctgaggctttctggattcggcggcgttccgcaatgttgtatgggcgtatgctgtcgtacatttgggttttcttcttctaatcgttgttgtagcttggcgagtccgcctccgacaccgcatgtgtggcagtaccagacgcccttgtcgaggttgatgctcatggagggctggtggtcgtcgtggaacgggcagagtatgtgttgctcgttcctggacggattgtaccgtatctgataatggtcgaggaggcggcaggtgtcagaggtgtgggaggagctcgttgagggttgataccacataggcttcactccatggcttgttgcgctgtttcatcactacgagtccgatggtggaattgttttgtttgtttcggtgtgtttcgtagttgcgtgcctcccggctggcttgtttcacgaattgggctaggtgtggttgcccggctttcgcctcgataatgtaggttttatggccggttgtgaggatgaggtcgccttcgtcttcgcggccgttgaggtggaggcgttcgatattgtgtccggtgtcgcgtagctggtggaggagtcttgtttcccattcggctccggcccgccggttgcgtgcctgctgtgtggccatagttttttagagtcctttgtgtgttgtggtcatgttccagggctgtttttcggcgagtggcccgaagaatgtgtattcggggtatgctctgagtcgttcgtatcgggtgccgtcggggctggatttgcctgtgcgctgtttgagtacggcgatgcgtgcctctgccggtatcgatagcccgttgccgttatcctcgccaccatacaatgagactccgaggatgagttgtggtttttcggagaggccgtttttgatttctcgccgtgctggcgggtgttcgatgtcggttccggttttgtcggttgcgtggtgtgtgacaataatggtggagccagtatccctgcccaatgctgtgatccattgcatggcttcttgctgtgcctggtagtcggattcgcagtcttgaatgtccatcaggttgtcgataacaatgagtggtgggaaggtgttccacatttccatgtaggcttgtaactccatggtgatgtctgtccatgtgatgggtgactggaatgagaatgtgatgtgttggccgtggtggatgctgtctcgatagtattctggcccgtagtcgtcgatgttttgttgtatttgttgggtggtgtgttgtgtgttgagggagatgattcgtgtggaggcctcccagggtgtcatgtcccctgatatgtagagggcgggctggttgagcatcgctgtgatgaacatggctagccctgatttttggctgccggaccgccccgcgatcatcaccaagtcgcccttatggatgtgcaaatcttggttatcatatagtggtgcgagttgtggtatgcggggtagttcggctgcggtttgggaggctctctcgaaggatcgttgtagagagagcatcgggaccttaatctatctgtctgttggttgtgtggctggtcagatggagtcgatatcgatatcagcatcagcagaggctgaagtgtcatctagctgaccattatcgcgcttgtctacgtattcggcaaccttatcgtagatggcgtcgtccaatgttttgagcacgaccgcgttgaaaccgtttttggtgcgcacggtggctagtttgaaggcctgctcctcgccaaggtatgcctctagttcgcggatcatggagtgtgggcggtcgttattgccgcgggctttctcaataatagcgttggggatggtttctggggtgccgttgttgagatcgtctagggtgtggaagatggtgacatcagcgtagatgcggtctgcgacctgtccaccgtagccttcagtgttgtgctggacgtcgtgcactttgaaggcgatggccgtggcgtcctggtttcgggaggggttgaagaaggtgctgttgctgttgttgcggtagtttgcgagtcccataactattgtttccttttactgttgtgtctgtttttgttggcttatattggtttatcgggtgaggctgtttcgcttagtgcggaaagcgtcggaaacatcactgttactggtgatgatcttcttgtactgttttagaaggtctgctagctgtgccttgcttgttgcattgttgattttgttgatgacgatggtgttttctttggatgcgattttgttgacgtagtctttggctgcctggttgtatcggtcttggaggatgattgatgcgctcgctacgagtgttgctagatcccagtctttggacacgtcatcgtttttgagtccgcctagcaggtcgatgatggcctgttttgtctgctctgctgtgtctcctcggatgaccgcccatggtgcagcatagtctccaccatatttgagtgtgatcgtgagtcgatcattgtcgatcttgtctttatctgtcatttggtgtccttttctttattgtctgtttctggtggctgtacggtggattctaccgggtatctgtacgagtttttgccgttgacggcccagcaggcgtctcgtacggggcatcctttacagagtgttgtgacgtgggggacgaagatgccttcgctgattcctttcattgcttgactgtacatggatgatacatgccggtaggtgttgttgtcaaggtcgtagagttcggtggatgtgccttgtgtcggggacttgtcgtcgttgcggctggtggctggcgtccaaaacatgcctttcgtgacatggatgtcgtgttggttgagcatgtaccggtatgtgtgcagctgcatactgtcggcgggtaggcgtccggttttgaggtcgaggatgaaggtttcgccggtgtcggtgtcggtgaaaacacggtcgatgtagccgactatttttgtgtcatcgtcgaggatggtttctaccgggtattcgatgcctggtttaccgtccaggattgcggtgatgtattctgggtggttgcgcctccatgttttccagcggtccacaaaggtggggccgtaaaccatccaccagtcgtagtctttcttgtgtggtccgcctgactcgcacatgtttttgcatattctgccggagggtttgatttctgtgccttcggattcggcgagggctacctgggtgtcgaaaatgtttttgaaggatgagagtttgtctggcagtgcagggtattcggcgggattgtacaggtgtaggtcgtattgttcggtgatgtggtgtatggcgcttccggcgatggtggcgtaccaggtgtggtgttgggcgtgatagccgtgggataggcgccatttttctccgcattcggcccactgggtgagtgaactgtaggagatgtgtcctgggtggctgatggttttcgggtattgtgctagaggcattacttgtcgcttgtgttccatgtgttgcgggtgtcttggccggcgtggtgttgctggtaggcgaggagtgcgaggcagtgccaggctgcgtgtgctagatggggtagcccggattcgtggtcgaggttgttgccttgctgccatgatagtagatgcctgtagagggcgtcgacactgtggctccacgggtatcctccggtccagttgttgtcgccatatttggtggcaccgtatccggctacttcgcctagggcgtgaagggatgctgggtcgatgagggagagcctgcagagtttcaattcttttcgggcaccgctgttggggtcggtgtacatgcgggtgggctcatccatggggtgtgtgctccttaagggtgggttactggttgttgttgtgggctagggcggcggcgagaataatgatggcgagggtttcggctatcagtatgggtgttgtgatcatttggtgtctcggggattgttggtgagtgttgaggcacccaggagggtggcgagggcgcatgcggcaataatggcgagggctgccttgtgtggggtgccggttgcgtacatccatgtgatgatggcaccttggatccaggctaggctggtgaagaaggtttcgtagctgtgcagctcaatgttgttgttgggtgtgttcatgcttgctcctgaagaatggtgttgatggttttataaatgttgtacaggtcggtttcgatagataacagttggttgatttggtggtcgagatcaatgtctgggttgagtgtgttgatgcgggaggcaatatcggtggctgtgcgtagtgtgccgccggtgtggtgaataatgtgtgccgtgtcggcgagtccggtggtgacggcgtagtgggataggagaggcatagcggggatgctccttggcgggttactgttgcgggttgatgttgaggtcggtgacgtgcggtgagttttctgttccggtgacgaggcagtggacggtgacgggtagtttggatgctcccggctggcggacggtggcgccgtagacgatgctgaatgtgtctttaccgatggttttgtggagttggaggtcgatgtcggggttgccgttccagttgacaccttgcgctgcggcctgttgttcggctttgtggttgcaggtgtgtgctgccgtgatcatggtgagtccggtggcggtttcttcaccccttgcttgggcttgcttgtgggctttggcctgctcggcttgtagggatcgggtggcggctgcctgccgtgccgctttctcggctttgcgctgttgggtagtcttgggggtccatgtggtgttggctgtggttgcctgtggggctggctgtgaggtgagtggcgggttgtcgtctggtgctggcatgaatgaggcggcggcaatgatggcggctgtgatgcctgcgatggtgtagccgtttttcttgttcatgttttgtgtcccctttccggggtgttgttcgttgctgacatggttaatactttcagcggctgggcccactgtcaaggctgcgctcagtttgtgtgagcgtttggtgtgtggctaggggttttgtcatgtaagcgtgacatgtcactaccttgcgtccagtatccatggcggttgcgagccatccctttggcgagcatctcgtccacagtgaggcacctgcggcgattggggccttccttgaccccgtgatcgcctatgcggtgcatgtccccggcataagtgccattaaatgtttcgtggcagactgtgcagtgttctggtcggtatccgatgattgtgctatcgcacttgtggcatgtccattgcatgattggtccttctttcgtgttttaagcttgtgctctgaggattagagcgactttcagcccttgggggtaggattatataggtcaggtatttctaggcgattctaggctcattgtgtgtggttggggttttatcgggcgcatagggttagcaggtggcccacattggtgcggctcacattccagtagagttgcgtggcttccttactggtgagcggcttccactcgtcatggctgaacacggtgccatcggatgcgatgaacgtgttggggcgtagcttgtgaagctcggcttccacatgctgccggtaggcttcggcgaggctctcaaaatccatgtggtcgcaggagaggttttcgaggcgtgtcaggtcgaaaggctccgggcagtcgtagctggctggagtgtagagctgggtgaagtggtcggcgatcttctgcatggcgggttcctttctggtgtgtggatggtttttatcgtgtggatgcgacaaggatggcgtctacgtcgatcatgtcgatcatgtcgttgagttcctcggcctcattctcggagaggtggcgccagtcgggtggcccgtatacggcgccgtcgagggtgacagtccacaggggccggatgagtcgtatggcttcttcgactttggcgtggtacatgcggcgcaccatatccagatcgatgtcgtctgaatggtttccggtgaggctgtggaggctgagcgggtcgatttctgtctgcctgtagaggctggtgaatgatggtgtgatgagtgtgccatccatgagtgtgctcctttctaggggttgttgtggtttctagagtgtgtgggctgtgaccccacagtcaaggctacgctcatttggattgagcgtttcatatgggtgtggcatggaatctacaccctcatactgtgtgagatgtatcacatccccctggcttggtgtgcacccctcaagactactctgccgacctggcgtggagggtgtagcccagaaatgccgtttaaagcttcaggggtacgcctaggagcgccttacagggtgggggctaggtatttatacccccagcatattctgatcgattctagacgactcccagagcccgatacacgatcaaccatctcgacatagaccatcagcccctatcctggttagctaagcctcaactatgtggacagtgtgggacactgtgggggaagaaggacacggtacaagaaagaggggggagcatcagccttaaagccttaagatcttagcgcttagcaccgatggtcttagcagttagcaccgagcccttgagggggctcggcatcagcctcatcgggctcagctcatcaggcacagccctgaaaagggtacacgccatcagggaaggcttgagagtacgaggagccctagcgacgagtactcgaaagcctgagggaacaccctcagtactgatgagcctagcgtattcggaaaggacgcaagagtaaagtgtgacagctatccgggagtgaaacccgttccgactaggggtttcagccttaaccaccctcaaaggttacaagactctaagaaaatttaagaaacttcttaggaagaaagttgtgttcatatccccctaaaaacacccaaaatagtcctcaaacccgcctatagagccaaacagtcaagtttgactcgtctagacggcgtatgataggctggacaggtagccagctggacgcaaggccagaaagtgctgacgcacttcccgacctcgcttaccatcagtctaccaaacactttaaagcttcaaggcttagcgctaagcccttaagatcttaacgcttagcaccgagcccccctcaagggctcgacatcagtcttaaagtcttaaacactttaagtaactttaaagcttcaaggcttagcccttaaggatctaagttactataaaagctttaaacacttaaagtaactataaagctttaagagcttaacatttaaggatataaataaacattaaagctttaaagtcttaaagtaaatatataaccttaacacttaagttaagtataaaaccttaaaggcttagcacttaaggatataaacttaacatcagtgtttaagacttaaagagttaaagtaactattaagacttaaaggcttataagctttaatactttaagtagctataagactttaaaaacctgaagtacttaaagttaaccatcagtcttaaactttaatattataagtattaaagcttataagttataaaagtttttagaagagttaaagggttaacttctttacttctcttctctctttggttctttctctcttctcttcttttcttcatcaggggagaagaggaacctttaaccgtcaacgctgatggacttttcaccgtgtgactcgtgtgcttctggtcgcaagctcccatcgcacactccccacactctttcacccgtgcccctttacggcttagcgtgttcgtcggaaggcgtacggcgtgtcacgcttaaacccttaacaccaggtaagacttaaagtgcatattataagtagaagactttaaaacctataaggtgttcccgcttagcccgtgttcctttaacgctaggcgctcagcgctaagatgtgaaacgtgaacacccatccacccccatttttcttccgtgtccttctccttttgacaccgctggggggcgatgtgatatttctcacatgccagggggtagtggagaaaacaaccaccccggaacgtttaagacaccccctcaaacgaacaaaacagggcctagaatcgatcagcagggcaccggtagggtattcctacccccagacgattcaaggccattacaggagcaatgagaggctcacaggggccatgggagattggggggcgtgatggcacacaccaaccgcacagccagccaagcccaccggcgctggcgggcaaggctcatcacccaagcccgacaacaaggccaaaccgaatgcccactctgcggagtcaccatcacctggaacacccacgacctgccaaccagccccgaagccgaccacatcacacccgtcagccggggaggactcaacaccctcgacaacgggcaaatcatctgcagaacatgcaacagaagcaaaggcaacagaacacaaccaaacatcaaattccaacaacaaaccacaaaaacattgattccatggtgaaaaacccgccaacccccaccgggcacaccccctgcacacccgtgcaagacctcgtacggcttagtgaaatacctcccttttgttgttttatcgttttgtcgactttttgtttggtggtgtgtgtggtgcagcctgagcttcctgatagtcgtgattggtgtggggagacgcgtcggtggtggtgtgtgtggggcgaggatccgcgtgccgggtttgtgtctgatgaggagtggttgtttctcatggatgctgcggtgattcatgatgtggtgtggcgtgagggtcgcgcggatttggtggcttcgttgcgtgctcatgtgaaggcttttatgggtatgttggataggtattcggttgatgtggcgtctggtggccgtggtgggggttctgcggtagcgatgattgaccggtataggaagcgtaggggggcttgagtaggtgtctggtgttgttgggtctcaggttcctcgtcaccgggtggctgtggcgtattcggtgtctgctggcggggatgctggggagcttggtagggcttatgggttgacgcctgatccgtggcagcagcaggtgttggatgattggcttgctgtgggtggtaatggcaggcttgcttcgggtgtgtgtggggtgtttgttccgcggcagaatggcaagaatgctattttggagattgtggagttgtttaaggcgactattcagggtcgccgtattttgcatacggctcacgagttgaagtcggctcgtaaggcgtttatgcggttgcggtcgttttttgagaatgagcggcagtttcctgacttgtatcgtatggtgaagtcgattcgtgcgacgaatggccaggaggctattgtgttgcatcatccggattgtgccacgtttgagaagaagtgtggttgtccgggttggggttcggttgagtttgtggctcgtagccggggttctgctcgcgggtttacggttgatgatttggtgtgtgatgaggctcaggagttgtcggatgagcagttggaggctttgcttcctaccgtgagcgctgccccgtctggtgatcctcagcagatttttttgggtacgccgccggggccgttggctgacgggtctgtggtgttgcgtcttcgcgggcaggctttgtcgggtggtaaacggtttgcgtggacggagttttcgattcctgacgagtctgatccggatgatgtgtcgcggcagtggcggaagttggcgggtgacactaatccggcgttggggcgccgcctgaatttcgggacagtctcggatgagcatgagtcgatgtctgctgccgggtttgctcgggagcggcttggctggtgggatcgtggccagtctgcttcgtctgtgattccggcggataagtgggttcagtcggctgtggttgaggcggctctggttggcgggaaggtttttggtgtctcgttttctcgctcgggggatcgtgtcgcgttggctggtgctggtaaaacggattctggtgtgcatgttgaggttattgatggcctgtctgggacgattgttgatggtgtgggccagctggctgattggttggcgttgcgttggggtgacactgaaaaggttatggttgcagggtctggtgcggtgttgttgcagaaggctttgacggatcgtggtgttccgggtcgtggcgtgattgtggctgatactggggtgtatgtggaggcgtgtcaagccttcctggagggtgtcaggtctgggagcgtgtctcatcctcgtgccgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaagcgaagcggcgtgaacggtctggtaggaagcgggtgtctgtggtatgaactcggatgagttggctctgattgagggcatgtacgatcgtattcaagggttgtcttcgtggcattgccgtattgagggctactatgagggctctaatcgggtgcgtgatttgggggttgctattccttcggagttgcagcgggtgcagacggtggtgtcatggcctgggattgcggtggatgctttggaggagcgtctggattggcttggctggactaatggtgacggctacggtttggatggtgtgtatgctgcgaatcggcttgctacggcgtcgtgtgatgttcaccttgatgcactgatttttgggttgtcgtttgtggcgatcattccccaagaggatgggtcggtgttggttcgtcctcagtcgccgaagaattgtactggccggttttctgccgatgggtcttgtttggatgctggccttgtggtgcagcagacgtgtgatcctgaggttgttgaggcggagttgttgcttcctgatgtgattgttcaggtggagcggcggggttcgcgtgagtgggttgagacgggccgtatcgagaatgtgttgggtgcggttccgttggtgcctgttgtgaatcgtcgccgtacttctaggattgatggccgttcggagattacgaggtctattagggcttacacggatgaggctgttcgcacactgttggggcagtctgtgaatcgtgatttttatgcgtatcctcagcgttgggtgactggcgtgagcgcggatgagttttcgcagccgggttgggttctgtcgatggcttctgtgtgggctgtggataaggatgatgatggtgacactccgaatgtggggtcgtttcctgtgaattctcctacaccgtattctgatcagatgcgtttgttggcgcagttgactgcgggtgaggcggctgttccggaacgctatttcgggtttatcacttctaacccgccttctggggaggctttggctgcggaggagtctcggcttgtgaagcgtgctgaacgcaggcagacgtcgtttggtcagggctggctgtcggttggtttcctggctgcccgggcgttggattcgagtgttgatgaggccgcgttttttggtgatgttggtttgcgttggcgtgatgcgtcgacgccgactcgggcggctacggctgatgctgtgacgaagcttgtgggtgctggtattttgcctgctgattctcggacggtgttggagatgttgggtttggatgatgtgcaggttgaggctgtgatgcgtcatcgtgccgagtcttcggatccgttggcggcactggctggggctatttcccgtcaaactaacgaggtttgataggcgatggcttcgggtgctgtgtcgaggcttgctgcgactgagtatcagcgtgaggctgtcaggtttgctgggaagtatgcgggctattatgccgagttgggtcgtttgtggcgtgccggcaggatgagtgacacgcagtatgtgcgtttgtgtgtggagttggagcgtgccggccatgacggttcagcagctatggcgggcaaattcgtttcagattttcgccggttgaatggtgtcgatcctggtttgatcgtgtatgacgagtttgatgctgcggcggctttggctaggtcgttttcgactatgaagattatgaatagtgacccggatagggcgaatgatacgattgatgcgatggctgcgggtgttaatcgggctgttatgaatgctggtcgtgacacggttgagtggtcggcgggtgcgcagggtaggtcgtggcgtcgggtgactgatggtgatccgtgtgctttttgtgccatgttggctacgaggtcggattatacgactaaagagcgggcgcttactactggtcatacgcggcgtcataagcgtgccggtaggcgtccgtttggttcgaagtatcatgatcattgtggttgtacggtggttgaggttgttggtccttgggaaccgaatagggctgatgccgagtatcagaggacgtatgagaaggctcgtgagtgggttgatgatcatgggttgcagcagtcgtctggcaatattttgaaggctatgcgtactgttggtggcatgagataatttgatgtggtttccggttgtgtgccgccggttatcggtgcacagggttgtctcccgcacgggggtcaacaatgttgtgttgttttccgcaaggagtgtagggttaggctatggccgatcagagtattgaggaacagaatgttgacaatgatgttgtggagtccggaaaggataacggcattgttgatacagtaaaagacgatggcgggcaggaggtagccgacaatcagttgaagaatgaaggcgagggtaaatcgccggggactgattggaaggcggaggcccgtaagtgggagtctcgtgctaaaagtaatttcgccgagttggagaagcttcgtacatcgagtgacgattctggatctactattgatgagcttcgccgcaagaatgaggaactcgaagaccggattaacgggtttgttcttgagggtgtgaagcgcgaggtggctgccgagtgtggcctgtcgggtgatgcgatcgcttttcttcacggtagcgataaggagtcgcttgccgagtctgctaaggctttgaagggtttgatcgaccatagtagtggtggtggcgcgggtgtgcgccgtcttgcggggagtgcccccgttgatgatgttaaacgacgtgagggtgtcgcgtttgtggatgctcttgtcaataattctaggagatgatttatcatggctgacgattttctttctgcagggaagcttgagcttcctggttctatgattggtgcggttcgtgaccgtgctatcgattctggtgttcttgctaaactgtcaccggagcagccgactattttcgggcctgttaagggcgccgtttttagtggtgttccgcgcgctaagattgttggcgagggcgatgttaagccttccgctagcgttgatgtttctgcgtttactgcgcagcctatcaaggttgtgactcagcagcgtgtctcggacgagtttatgtgggctgacgccgattaccgtctgggtgtgcttcaggatctgatttccccggccctgggtgcttctattggtcgcgccgttgatcttattgctttccatggtattgatcctgctacgggtaagcctgctgcggctgtcaaggtgtcgctggataagacgaataagacggttgatgccaccgattccgctacggctgatcttgttaaggctgttggtctgattgctggtgctggtttgcaggttcctaacggtgttgctttggatccggcgttctcgtttgctctgtcaactgaggtgtatccgaagggttcgccgcttgccggtcagccaatgtatcctgccgccgggttcgccggcctggataattggcgcggcctaaatgttggttcttcttcgactgtttctggtgccccggagatgtcgcctgcttctggtgttaaggctattgttggtgatttctctcgtgtccattgggggttccagcgtaacttcccgattgagctgatcgagtatggtgacccggatcagacggggcgtgacttgaagggccataatgaggttatggttcgtgccgaggctgtgctgtatgttgcgattgagtcgcttgattcgtttgctgtcgtgaaggagaaggctgccccgaagcctaatccgccggccggtaactgattcatttgttgcgataatgtttatgctgtgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatatagggatgccgctaaggctattctgcgtagggctttgttgcgctggaatgatactggcgtgtcgggtcaggtgcagtatgagtctgcgggcccgtttgctcagactacacggtcgaatactcctacgaatttgttgtggccttctgagattgccgcgttgaagaagttgtgtgagggtgatagtggggctggtaaggcgttcactattacaccgaccatgaggagtagtgtgaatcattctgaggtgtgttccacggtgtggggtgagggttgctcgtgcgggtcgaatattaacggctatgctggcccgttgtgggagatatgatatgaccggttttccttacggtgaaacggttgtgatgcttcagccgactgttcgtgtcgatgatcttggtgacaaggtggaggattggtctaagcctgtcgagactgtgtaccataacgtggccatctatgcttccgtttcgcaggaggatgaggccgcggggcgtgactcggattatgagcattggacactgctgttcaagcagcctgtcaaggctgctggttatcggtgtcgttggcgtattcggggtgttgtgtgggaggctgacgggtctcctatggtgtggcatcatccgatgtctggctgggatgctggtacgcaggttaatgtgaagcgtaagaagggctgatgggttgtggcacgtgatgttgatgtgaagctgaacttgccgggtattcgtgaggtgttgaagtcttctggggtgcagggcatgttggctgagcgtggtgagcgtgtcaagcgtgcggcctcggcgaatgtgggcggtaacgcttacgatagggcccagtatcgtgccgggttgtcgtctgaggtgcaggttcaccgtgttgaggctgtggcgcgtattggcaccacctataagggtggtaaaaggattgaggctaagcatggcacgttggcgaggtcgattggggctgcgtcgtgatcgtttacggtgatcctcgaatatgggctaaacgtgtgttggcggatgatggttggctgtctgatgtaccgtgcacgggtactgtgccggatacatttgagggtgatctgatttggttggcgttggatggtggcccggagttgcatgttcgtgagcgtgtttttttgcgtgtgaatgtgttttcggatacgccggatcgtgctatgtctttggctcgccgggttgaggctgtgctggctgatggtgtggatggtgatccggtggtgttttgcaggcgttcgactgggcctgatttgctggtggatggtgcacgttttgatgtgtattcgctttttgagctgatatgtaggcctgcggagtctgaataagcttattgtttttgttttaatgtaattgtttgatatttaatgggggttgtgatggctgctacacgtaaagcgtctaatgttcgttcagcggttactggcgacgtttatattggtgacgcgcacgcgggtgattctattaagggtgtggaggcggttccttccgggcttacagctttggggtatctgtctgatgacgggtttaagattaagcctgagcgtaaaacggatgatttgaaggcttggcagaatgcggatgttgttcgcactgtggctacggagtcgtctatcgagatttctttccagctgattgagtcgaagaaggaggttatcgaactgttttggcagtcgaaggttactgccggatctgattcgggttcgttcgatatttctcctggtgccacaacaggtgttcacgccctgttgatggatattgttgatggcgatcaggttattcgctactatttccctgaggttgagctcattgatcgtgacgagattaagggcaagaatggcgaagtgtacgggtatggtgtgacgttgaaggcgtatcctgcccagattaataagactggtaatgcggtgtcgggtcgggggtggatgacggctttaaaagctgatactcctccgactcctccgccggccccggttcctccgaagcctcagccggatccgaatccgccgtccggtaactgatacacgattttaggggattgttaatagatgagtgacactggtttcacgttgaagattggtgatcgtagctgggtgttggcggatgcggaggagacggctcaggctgttcctgcccgcgttttccgtcgtgccgccaggattgcccagtcgggggagtctgcggatttcgcccaggttgaggtgatgttttctatgttggaggctgccgccccagctgacgcggtggaggccctggaggggcttcctatggttcgtgtggcggaggttttccgtgagtggatggaatacaagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccaccttaatggttggcagcgcccgtttgagtggtgcgagtgggctgtgttggacatgttggatcattacaggtctgctaatagtgaggggcagccggagcctgtggcgaggccgactgatgagcgtcgggcaaggtttacgtctgggcaggtggacgatattttggcgcgtgttcgtgccggtggcggggtgtctcgcgagattgatattatggggtgaatagtgtatgtctggtgagattgcttccgcatatgtgtcgttgtatacgaagatgcctggccttaaaagtgatgttggtaaacagttgtcgggtgttatgcctgctgaggggcagcgttcgggtagcctgtttgctaaaggcatgaagttggcgcttggtggtgcggcgatgatgggtgccatcaatgttgctaagaagggcctcaagtctatctatgatgtgactattggtggcggtattgctcgcgctatggctattgatgaggctcaggctaaactgactggtttgggtcacacgtcttctgatacgtcttcgattatgaattcggctattgaggctgtgactggtacgtcgtatgcgttgggggatgcggcgtctacggcggcggcgttgtctgcttcgggtgtgaagtctggcggtcagatgacggatgtgttgaagactgtcgcggatgtgtcttatatttcgggtaagtcgtttcaggatacgggcgctatttttacgtctgtgatggctcgcggtaagttgcagggcgatgacatgttgcagcttacgatggctggtgttcctgtgctgtctttgcttgccaggcagacgggtaaaacctcggctgaggtttcgcagatggtgtcgaaggggcagattgattttgccacgtttgcggctgcgatgaagcttggcatgggtggtgctgcgcaggcgtctggtaagacgtttgagggcgctatgaagaatgttaagggcgctttgggctatttgggtgctacggctatggcgccgtttcttaacggcctgcggcagatttttgttgcgttgaatccggttattaagtctatcacggattctgtgaagccgatgtttgctgccgtcgatgctggtatccagcggatgatgccgtctattttggcgtggattaaccgtatgccggctatgatcacgagaatgaatgcacagatgcgcgccaaggtggagcagttgaagggcatttttgcgagaatgcatttgcctgttcctaaagtgaatttgggtgccatgtttgctggcggcaccgcagtgtttggtattgttgctgcgggtgtggggaagcttgttgcagggtttgctccgttggcggttgcgttgaagaatctgttgccgtcgtttggtgctttgaggggtgccgccggggggcttggtggcgtgtttcgcgccctgggtggccctgtcgggattgtgatcggcttgtttgcggcaatgtttgccacgaacgcccagttccgtgccgctgttatgcagctggtggctgtggttggtcaggcgttgggccagattatggcagctgtgcagccgctgtttggtttggttgctggcgtggttgccaggttggcgccggtgttcggccagattatcggtatggttgctggtttggctgcccggctggtgcctgttattggtatgcttattgcccggctggttcctgttatcacccagattattggtatggtaacccaggttgctgccatgttgttgcctatgctgatgccggttattcaggctgttgttgctgtgatacggcaggttattggtgtcattatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgctgtgctacaggtggtggcccgtattattccggttgttatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatattaaatcgtttacgtctgcgtttattaacggttttaagtcgatcatttctggcggcgtgaacgcggttgtggggttttttacgcggcttggtttgtcggttgcttcccatgtgaggtccggttttaacgctgcgaggggtgctgtttcttccgccatgaatgctattcggagtgttgtgtcttcggtggcgtctgctgttggcgggtttttcagttcgatggcgtctcgtgttcggaatggtgctgtgcgcgggtttaatggtgcccggagtgcggcttcttctgctatgcatgctatggggtccgctgtgtctagtggtgtgcatggtgtgctgggttttttccggaatttgcctgacaatattcggcgtgcgcttggtaatatggggtccctgttggtgtcggctggccgtgatgtggtgtccggtttaggtaatggtatcaagaatgctttgagtggcctgttggatacggtgcgtaatatgggttctcaggttgctaatgcggcgaagtcggtgttgggtattcattccccgtctcgggtgtttcgtgacgaggttggccggcaggttgttgccggtttggctgagggtattactggtaatgctggtttggcgttggatgcgatgtcgggtgtggctgggaggctgcctgatgcggttgatgcccggtttggtgtgcgatcgtctgtgggttcgtttaccccgtatggcaggtatcagcgcatgaatgataagagtgttgtggtgaatgtgaatgggcctacttatggggatcctgccgagtttgcgaagcggattgagcggcagcagcgtgacgctttgaacgcgttggcttacgtgtgattttgggggtgtggtgcatgtttattcctgacccgtctgatcgttctggtttgactgtgacttggtctatgttgccgttgattggtaatgatccggagcgtgtgcttcatttgacggattatacggggtcgtctccgataatgttgttgaatgattcgttgcgcggtttgggtgttcctgaggtggagcatttttctcaaactcatgttggggtgcatggctcggagtggcgcgggtttaatgtgaagcctcgcgaggtgacgctaccggtgttggtgtcgggtgttggcccggatccggtgggcggttttcgtgacggttttttgaaggcgtatgacgagttgtggtctgcttttcctcctggcgaggtgggggagttgtctgtgaagactcctgccggtcgtgagcgtgtgttgaagtgccggtttgattcggtggatgacacgtttacggtggatccggtgaacaggggttatgcgcgttatctgttgcatttgacggcttatgacccgttttggtatggggatgagcagaagtttcgtttcagtaacgctaagttgcaggattggttgggtggcggccctgtcgacggtaagggtaccgcgtttccggtggtgttgacgcctggtgttggttcgggttgggataatctgtctaataagggtgatgtgcctgcgtggcctgtgattcgtgttgaggggccgttgtcgtcgtggtctgtgcagattgatggtttgcgtgtgtcctcggattggccggtggaggagtatgattggatcactattgatacggatcctcgtaagcagtctgcgttgttggacgggtttgaggatgtgatggatcgtttgaaggagtgggagtttgcgcctatcccgcctggcggttctcggagtgtgaatattgagatggttggtttgggtgccattgttgtgtcggtgcagtacaggtttttgagggcttggtgaatagttgatggctggttttgttccgcatgtaacattgtttacaccggattatcgccgtgtggcgcctatcaatttttttgagtcgttgaagttgtcgttgaagtggaatggtttgtccactttggagttggtggtgtctggtgatcattctaggcttgacgggttgactaggccgggtgcgcggcttgtggttgattatggtggtggccagattttttctgggcctgtgcgtcgggtgcatggtgtgggtccgtggcgttcttcgcgtgtgactatcacgtgtgaggatgatattcgtctgttgtggcgtatgttgatgtggcctgtgaattatcgtcctggtatggttggtatggagtggcgtgcggatcgggattatgcccattattcgggtgcggcggagtcggtggctaagcgggtgttgggggataatgcttggcgttttccgtctggtttgtttatgaacgatgatgagagtcgtggccgctatattaaggattttcaggtgcggtttcacgtgtttgccgataagttgttgccggtgttgtcgtgggctcggatgactgtcacggtgaaccagtttgagaatgcgaagtttgatcagcgtggtttggtgtttgattgtgtgcctgctgtgacccggaaacatgtgttgactgccgagtcgggttcgattgtgtcgtgggagtatgtgcgtgacgccccgaaggcgacatctgtggtggttggtggccgtggcgagggtaaggatcggctgttttgtgaggatgttgattcggcggccgaggatgattggtttgatcgtgtcgaggtgtttaaggatgcccgtaacacggattccgagaaggtgtctctcttcgatgaggctgagcgggtgttgtccgagtcgggggctacgtcggggtttaagattgagttggctgagtcggatgtgttgcggtttggtcccggcaatctgatgcctggggatttgatctatgtggatgtgggttctgggcctattgcggagattgtgcggcagattgatgtggagtgtgtatcgcctggtgatggttggacgaaggtgactccggttgcgggggattatgaggataatccgtcggccctgttggctcgccgtgtggctggtttggctgcgggtgtgcgggatttgcaaaagttttagtaagtgattggggtttgttgtgggtattgtgtgtaaagggtttgatggtgtgttgaccgagtatgattgggctcaaatgtctggtctgatgggtaatatgccgtctgtgaaggggcctgacgattttcgtgtcggcacgacgattcagggttctacggtgttgtgtgagatcctgccggggcaggcttgggctcacggggtgatgtgcacgtcgaatagtgttgagacggtgacgggtcagcttccgggcccgggtgagactcgatacgactatgtggtgttgtctcgggattggcaggagaatacggccaagttggagattgttcccggtgggcgtgcggagcgtgccagggatgtgttgagggctgagcctggcgtgtttcatcagcagctactggcgactttggtgttgtcgtctaacgggttgcagcagcagttggataggcgtgctgtggcggctagggttgcgtttggggagtctgctgcgtgtgatcctacccctgtggagggtgaccgtgtgatggttccttcgggggctgtgtgggctaaccatgccggcgagtggatgttgttgtctcccaggattgagacgggttcgaagtcgatcatgtttggtggttctgctgtgtatgcttacacgatcccgtttgagcgccagttcagtagtccgcctgttgtggtggcgtctatggctacggcggctgggggcacggcacagattgatgtgaaagcctacaatgtgactgcccaaaattttagtttggcgtttattacgaatgatggttcgaagccgaatggtgtgcctgcggtggcgaattggattgctgtcggcgtgtgactgcacgggtgttgtggcggatggtgtgatgttggggggctgtggtgtcgtggtttactcctgcactggtggcctctatttgtaccgcgttggccacggttttgggttctgttcaggctgtcacatcccggtctaggaagcgtttacgcaggctgtcggctcaggtggatgcgatggaagagtatacgtggggtgtgcggcgcgaggtgcgaaggtttaacgccgggcttcctgatgatgtggagccgatgcatcttcctgatttgcccgagtttttgaaagatactgttgatggtggaggtgagtagggttgagggagttggaggaggagaagcggcagcgccgcaattttgagaaggcttcactggtgttgttgtttttgtcgcttgtgttgttggcggtggttgctgcgggtgctttgcgtttcggggctgtatcctctgagcgggattcggagcaggcgagggcccagtcgaatggtacggctgccaggggtttggctgcccgtgtgaagcaggcgtgtgcttcgggtggggtggagtctgtgcgtcttcaccgttctggtttgtgtgtggatgctgtgcgtgttgagcagcgtgttcagggtgtgccgggtcctgccggtgagcgcggcccgcaaggcccttcaggtcctgccggccgggatggtgttaatggttcggctgggctggttggccctgttggtccgcaaggttctccgggtttgaatggtgtgaaaggtcctgacggcttgcctggcgctaacggttcggatggccgtgatggtgttccaggtcg

SEQ ID NO: 69 - DNA, 29238 bp, synthetic construct
  source 1..29238: mol_type = other DNA; note = PAC1
tagcgtacagggtgtgccaggtcccgccggtgagcgcggcccgcaagggccggctggtgctgatggtcgggatggtgttaatggttcgactgggctggttggccctgtgggtccgcagggttctcctggcttgaatggtgtggctggcccggacgggttgcctggtgcgaacggatcggatggccatgatggtgttccaggtcgtgcaggtgctgacggtgtgaacggcgctgatggtcgggatggttcggccggtgagcgcggtgatgtgggcccttcaggtcctgccggcccgcaaggtgcacagggtgaacggggtcctattgggcctcagggtccgcagggttctgccggtgctgacggcacgaatggtaaagacggtaaagatgggcgctcggttgtgtctgtgtactgttccgagggccgcctggttgtgaaatatagtgacggtgtggcttctacaatatcgagctcggtggcctgccagggtgtgaaaccgtcgcctatagtgactatatcatcccacaagtaaaaaagaaaagggaagggtgttactagtgttgattgtggtgttaggtggtgtgtggtgagatacattcctgcggcgcatcattctgccggctcgaatagtccggtgaatagggttgtgattcatgcgacgtgcccggatgtggggtttccgtctgcctcgcgtaaagggcgggcggtgtctacagcaaactatttcgcgtccccatcgtcgggtggttcggcgcattatgtttgcgatattagtgagacggtgcagtgcttgtcggagtctacgattgggtggcatgccccgccgaatccgcatagtttgggtatcgagatttgcgcggatgggggttcgcacgcctcgttccgtgtgccggggcatgcttacactcgggagcagtggcttgatcctagggtgtggcctgcggtggagaaggctgccatcctgtgtagacgtttgtgtgacaaatataatgttccgaagaggaagcttagtgcagccgatttgaaggctggtaggcggggcatctgcgggcatactgatgtgacggatgcgtggcaccagtcggatcatgacgatcctgggccgtggtttccgtgggacaggtttatggccgtcgtcaacggcaaagatgagagtggggagttaacggtggctgatgtgaaagccttgcatgatcagattaaacaattgtctgcccagcttactggttcggtgaataagctgcaccatgatgttggtgtggttcaggttcagaatggtgatttgggtaagcgtgttgacgccttgtcgtgggtgaagaatccggtgacggggaagctgtggcgcacaaaggatgctttgtggagtgtctggtattacgtgctggagtgtcgcagccgcatcagtaggctggagtctactgtcaacgatttaaagaagtgatctatggtgggtaaacagttttggttgggcttgtttgagcgtgccctgaaaacttttattcaaacgtttgttgctgtgttgggtgtgactgcgggtgtcacttatactgcggagtcgtttcgcggtttgccgtgggagtctgctctgattacggccggggttgctgcaatactgtcggttgctacctcgtttggtagcccgtcgtttgtggctggcaaacctaaaactacggttgtggatgcgggtttggttccaccggatgatgggggcttggttgagccgcacatggtggatgtgtcggatcctggcatgatcgagcctgcagatgatgcggatcttggtgtaggctatgtgccgaaacacgctgccgagtcggaggttggcacggtagagtctactgttgcataattgaatatgtgtgtgccccagcggtgctgccacgatcgtgtggtggttgccgctggggcactatttttgtatattgcggtgtggctatgattcgttgctgtcgatggtgtcttcgagcatctgatacaggtggaggcaggtagagatagtttcgctggcctggtctagaacgttccggccgataacatttttgtgattgtcgcggtggcggatgatagcccacatgatctcgtcggctgccgcttgcaatagttttgactggtatgcgattccggcgagccagtctatggcttccgggcttgccggtgtgtcgtctggaatgccacaggtgttgctgttgtttgtggggtatcctgcactgtcgcaaaaccacaggatttcgctgcactcgtctagcgtgtcctggtcgatagcaagatcgtcgaggctgacttcgttgacggtaaggttcacgttgtcgagggagatgggtacaccgtactggttttcgacactgtcaacaatgttttccagctgttgcatgttggtgggctgttgttggacgatacggtgtatcgctgtgttgagggtggtgtaggtgatattgtgtgtgttgttcatggttttattccatctctgtgctgtcgtcttggtcgtatcgactgtttgcgtagcctgtgagggtgatgagtgtttggtctgcccattgtttcactgtttgccgggtgacacccaatcgttgggcggctgtggcgtaggtttggtcgtatccgtatacttctcggaatgctgccagccgtgctaaatgttttcgctgtttggatggctggcaggtgagggtgtagtcgtcgatggctagctgtagatcgatcatggcgacaatgttgttgccgtggtgttgtggcgcggttggtgggggtggcattcccggctccacggagggtttccatgggccgccgttccaaatccattgggcggcctgaataatatctgcggtggtgtaggtcctgttcatgtgtcatcccctgaacaggttgtcgaagtcgtctgtgttgctggtgttggtggtatcgaatcgtccgacgcagtggcagtagtcgtacatgagtttgataatgtgttggtggtctcccaaataggtgttgccgctgatgctgtaggtggctgtgccgtctttgctgatggtgtatttggcggtgatggtttcggggttttctgtgtttgtgatgatggctgtggtggtggcgcctacggtttgtagcctggtggtttgggttccgtcgtcgagggtggtagtaaccatagttggggttctccttaaatactggtttggttgtcggctagatgaataatatcggataaaggtttcggctggtctaggtgttgtatggttttgttggcgagccgtttggctaccctgtagcacattttgatgtagtgtttgttgtctaggttgtggtattgttcccgtaccgcaatatatagtagggagtcttggtacaggtcgtctgcgttgattgcggggtagtgtgtggctatttttgtgcatgcccggttgagtgtgcgtagatgatggtctgtggcccatccccacgatgctgtggtggccaggtctgatttggtgggtcgtctgctcatggcgctatttcatctcgctatctgatagttgtttggtgttttgttgttgatagtgtagcacactagtccgggatggccggtggtgcctgtgcggtgccggaaccatgtggattctccttccatggatgggcattggatgaaggtgcgttgtccttgctcggagatttctaggtggtgccggtgcccggccatgagaatattagatgtggtgccgttgtggaattcttggccgcgccaccattcgtattgtttgccggttttccattggtgcccgtgtgcgtgcaggatttgtgtgccggctacttcgacggtggtggtcatttcgtcccgtgcggggaagtggaagtgaaggttgggatattggttgtcgagctggtaggcttctgcgatggcgcggcagcagtccacgtcgaaggagtcatcgtaggtggtgacgcctttaccgaaacgcacggcttcaccgtggttgccggggatggatgtgatggtcacatttttgcagtggtcgaacatgtggacgagttgcatcatggccatgcgagtcaaccggatttgttccgtcaagggtgtttgtgtgcgccaggcgttgttgcctccttgtgacacgtatccttcgatcatgtcgccgaggaatgcgatgtggactcgttgcggctgtccggcttgctgccagtagtgttttgcgactatgagggagtgcaaatagtcgtctgcgaagtgtgatgtttccccgccggggatgcctttgccgatttgaaagtctcccgcccctaccacgaacgcaacattgctgtagtcggtgtgtgtgtcttggttgggtttggggggtgtccattcggctagtttatcgacgagttcgtcgaccggataggggtcggttgcgggttggtggtcgatgattttttgtatggatcggccggtttctccgttggggagtgtccattcggagatgcgtgtgcgccgtacggtgccgtttgcgagatcatcgcggatggtgtctgcttcgttgtcgtggttggctagctgtgtgaggagccggtctatattgtctatcatcgggtatcctcctcttgtggggtggtgttggcttgtttgcggcgatagtcttttataacggtggcggagatggggtatcctgcctgggtgagctgttttgctagccatgaggcggggatagacctgtcggcgaggacgtcggcggctttagccccgtagcgttgaataagggtttcagttttggttgccatgatgtcctaggggttgtgtggtgggctgccatcctgtgcggcagtcgccgtcgtgtcctggtttgcgggtgcaccacgatacggttccgtctgtgtggttgagtgttttaccgcacatgacgtcacgtaggtgctcgggaaactcatcgttgttgttgtccccgtgcatgtcgatcaagtgttgggttttagtaaccatcatgcctcctatgtgtgaaagagtgtgcaaatactatgcaggtgtcatggatgtttatgcgggtatggttttcatcaccttgctgaacgttacttggttactgtacatcatctgagtgatttcctgatcagtcttatcggggtgctgctttcgcaggttcgcccactggcaggcgttgtcggtctcctgctgtaaacgtgtcaggtgctgctcgttgatgatgtgtttccacattgtccatgacacgtcgagcctgcggagcatgttcatggctggcacgttgaaggagttgaggaagagtatttcttcggtgtagtactgtttttcgtattggtcccatccgcttcggtgcctgttgggctggtttttggggtaggcttcccggcagattttgtgtaaccgtttggccatgtcgtcgggtagtttaatgtcggggttggcgcggatcatggatcgcatcccgtcataggtggtgccccaggtgtgcatgatgtggagtgggtcttcaccatcggcccatttttcggcgatgatggcgaggcggatgcgcctcctggcggctttactggtgttgcgccggtgggggatggggcatgtgtcgaggggatccatgatattttagtgtacctttccgtgttgtggttgtttgtctggttttattgtagcactgtgttgagggcttgtgtcaaccctgttttgccggttttcaggtatgtgtctgtgacatcccccagggtgaggggcacgtgggtggcttgggggagtgctgcctggagggtttgggccatctggtggcctgcctggtctgggtcggaccagatgtagatgtggtcgtagccttcgaagaatttggtccagaaggtttgccacgaggttgcgccgggtagggcgacggctggccatccgcattgttcgaggatcatggagtcgaattcgccttcgcaaatgtgcatttcggctgccggggtggccatggcggccatgttgtagatggagcctgtgtctcctgccggggtgaggtatttggggtggttgtgggttttgcagtcgtgtgggagtgagcagcggaaacgcatttttcgtatttcggctggccgcccccaaactgggtacatgtatgggatggtgatgcactggttgtagttttcgtggcctggtatggggtcattgttgatgtatccaaggtggtggtagcgagctgtttcttcgctgatgcctcttgccgagaggaggtcgagtatgttttcgaggtgggtttcgtagcgggctgaggctttctggattcggcggcgttccgcaatgttgtatgggcgtatgctgtcgtacattcgggttttctttctctaattgttgttgtagtttggcgagtccgcctccgataccgcatgtgtggcagtaccagacgcccttgtcgaggttgatgctcatggagggctggtggtcgtcgtggaacgggcagaggatgtgttgctcgtttttggacgggttgtaccgtatctggtaggtgtcgaggaggcggcaggtgtcagaggtgtgggaggagctcgttgagggttgataccacataggcttcgctccatggcttgttgcgctgtttcatcactacgagtccgatggtggactgactttcgcggtttcggtgggtttcgtagttgcgtgcctcccggctggcttgtttcacgaattcggctaggtggggctggccggctttcgcctctatcacataggttttgtggccggttgtgaggataaggtcgccttcgtcttcacggccgttgaggtggaggcgttctatatcatggccggtgtcgcgtagttggtggaggagtcgtgtttcccattctgcgccggccctgcggtttcttgattgttgtgtcgacatgatagtcctttgtgtgttgtggtcatattccagggctgtttttcggcgaggggcccgaagaaggtgtattcggggtaggctcgtagccgctcgtatcgggtgccgtcggggctggatttgcctgtgcgctgtttgaggacggcgatgcgtgcctctgccgggatcgatagcccgttgccgttatcctcgccaccatacaatgagactccgaggatgagttgtggtttttcggagaggccgtttttgatttctcgccgggcgggcgggtgttcgatgtcggagccggttttgtcggttgcgtggtgtgtgacaataatggtggagccagtatcgcggccgagggctgtgatccattgcatggcttcttgctgggcctgatagtcactctcgcagtcttggatgtccatcaggttgtcgataacgatgatgagtgggaaggtgttccacatttccatgtaggcttgcagttccatggtgatatcggtccaggtgatgggtgactggaatgagaatgtgatgtgttggccgtggtggatgctgtctcgatagtattctggcccgtaatcgtcgatgttttgttgtatctgggcggtggtgtgttgggtgttgagtgagatgattcgtgtggaggcctcccagggtgtcatgtcccctgatatgtagagggctggctggttgagcatcgctgtgatgaacatggctagccctgatttttggctgccggaccgccccgcgatcatgacgagatcccctttgtggatgtgcatatcctggttgcggtagaggggttctagttgtggtatgcggggcagctcggctgcggtttgggaggccctctcgaaggatcgttggagagagagcatcgggaccttatctatctatcggttacgatttgtatgaatattggcggttagatggagtcgatgtctacatcatcactaccagtggtgttgggctgactgtctcgctggtcaacgtaggctgctacaaggtcgtagatggcgtcgtccaatggtttgagcacgaccgcgttgaagccgtttttggtgcgcacggtggcgagtttgaaggcttgctcttcgccaaggtaggtttcgaggtcgcggatcatggagtgtgggcggtcgttgctgccgcgtactttttcgatgatggcgttggggatggtttctggggtgctgttgttgaggtcgtctagggtgtggaagatggtgacatcagcgtagatgcgatcggcggtctgtccaccgtagccttcagtgttgtgctcgacgtcgtggactttgaaggcgatggcggtggcgtcctggtttcgggaggggttgaagaaggtgctgttgctgttgtttcggtagtttgcgagtcccattgttgtttcctttactatttttgttggtttgtgtcggtttttatcgggtgaggctgtttcgtttgctgcggaacgcctcggatacgtcagtgttgctggtgatgatcttcttgtactgtttcagaaggtcggctagctgtgctttgcttgttgcattgttgattttgtcaatgatggtgttgtttccttcactggcaatgttgtctacgtagtctttggcggcctggttgtatcggtcttggaggatgatggatgcggaggcgatcagtgttgccaggtcccagttccttgccgccgaactgtttttgagtccgcctagcaagtcgatgatagtcttctttacttcgtcggcggtgtctccacggatgactgtccatggggcggcgtagtctccgccgtatttgagtgtgatggtgatgcgatcatcagtgctgttggtgttatcgttcactggtgctccttgctttcttctgttggggctgtgatggtggtttctgtagggtacctgtaggcgtctttcccgttgacggcccagcaggcgtccttgacggggcatcctttgcagagtgctgtgacgtggggtacgaagatgccttgactgattcctttcattgcttgactgtacatggatgatacatgccggtaggtgttgttgtcaaggtcgtacagttcggtggatgtgccttgtgtcggggacttgtcgtcgttgcggctggtggcgggtgtccaaaacatgcctttcgtcacatgaatgtcgtgttgggcgagcatgtaccggtatgtgtgcagctgcatactgtcggcgggtaggcggccggttttgaggtcgaggatgaaggtttcgccggtgtcggtatctgtgaaaacacggtcgatgtagccgacaatctgggtgccgtcggggagggtggtttctaccgggtattcgatgcctggctggccgtcaataacagcggtgatgtattctgggtggttgcgcctccatgttttccagcggtctacaaaggtggggccgtaaaccatccaccagtcgtagtcttttttgtgtggtccgcccgactcgcacatgtttttgcatattctgccggagggtttgatttctgtgccttcggattcggcgagggctacttgggtgtcgaaaatgtttttgaaggatgcgagtttgtctggcagtgcagggtattcggcgggattgtacaggtgtaggtcgtattgttcggtgatgtgatgtatggcgcttccggcgatggtggcgtaccaggtgtggtgttgggcgtggtagccgtgggataggcgccatttttctccgcattcggcccactgggtgagtgaactgtaggagatgtggcctggatggttgatggttttcggatattgtgctaggggcattactggtcgcctttgtgtgtgttccatgggttgcgggtgtcttggccggcgtggtgttgctggtaggcgaggagtgcgaggcagtgccaggcagcatgggctagatggggtagcccggattcataatcgaggttgttgccttgctgccatgatagtaggtgcctgtagagggcgtcgacgctgtggctccacgggtagccgccggtccagttgttgtcgccgtatttggtggcaccgtagcctgccacggagccgagggcgtgcaaggctgtagggtcgatgagggatagcctgcaaagtttcaattcttttttggcaccgctgttggggtcggtgtacatgcgggttggcttatccatgggggtgtgctccttaggggtgggttactggttggggttgtgggcgagtgctacggcgagaataatgatggcgagggtttcagcaataagtatgggtgttgtgatcatttgctgtctcggggattgttggtgagtgtggaggcgcctaggagggtggcgagggcgcatgcggcaataatggcgagggctgccttgtgtggggtgccggttgcgtacatccatgtgatgatggcgccttggatccaggcgaggctggtgaagaacgtttcgtagctgtgtagctcaatgttgttgggtgtgttcatgcttgctcctgaagaatggtgttgatggttttataaatgttgtacaggtcggtttcgatagataacagttggtggatttcgtggtcgagatcaatgtctgggttgagggtgttgatgcgggaggcaatatcggtggctgtgcgtagtgtgccgccggtgtggtgaataatgtgtgccgtgtcggcgagtccggtggtgacagcgtagtgggagaggagaggcatagctgggggtgctccttggcgggttactgttgcgggttgatgttgaggtcggtgacgtgcggtgagctttctgttccggtgacgaggcagtggacggtgacgggtagtttggatgctcccggctggcgtacggtggcgccgtaggcgatggagaaggtgtctttgccaataattttgtggagttggaggtcgatgtcggggttgccgttccatttgacaccgtgtgcggcggcctgttgttcggctttgcggttgcaggtgtgtgctgccgtgatcatggtgagtccggtggcggtttcttccccccttgcttgggcttgccggtggtttttggcctgctcggctcgcagtgactgttctgcggctgcctggcgggctttcttttcggctttgcgctgttggacggttttgggggtccacgcggtgttggctgtggtggcctgtggggctggctgtgaggcaagtggcggattgtcgtctggggctggcatgaatgaggcggcggcaatgatggcgactgtggcgccggcgatggtgtagccttttttcttgttcatgactgttgtcccctttccggggtgttgttcgttgctgacatgatcaatacttccagcgaatgaacctcgtgtcaaggctgcgctcaacgattgtgagcgattcgtgtgtggctaggggttttatcggctgtacagggtgaggaggtggcctacgttgatgcgggtcacattccagtagagttgcgtggcttcacccccggtgagtggcttccactcgttgtggctgaacacggtgccgtcggtggcgatgaatgtgttggggcgtagcttgtggagttcggcttccacgctctgccggtaggcttcggcgaggccctcaaaatccatgtggtcgcaggagaggttttcgaggcgtgtcaggtcgaagggtgtgggacagtcgtagctggcggggctgtagagctgggtgaagtggttggcgatcttctgcatcatgattccttttctggtgatggtgtgttgatggttttatcgtgtggcttcggcgatgatggcgtccacatcgattgtgtcgatcatgtcgtggagttcctcagcctcatccgcggtgagtggctgccagtcctggggtccgtatatggcaccgtcgagggtgacagtccacaggggccggatgagtcgtacggcttcttcgactttggcacggtgcaggcggcagatgatagacgtgtgggtgttgcctatgtcacatcctgccaggtgtgtggggtggagtgggttgatttctgtctgcccgtagaggttggtgaaggatggtgtgatgagtgtgccatccatgagggtgtgctcctttcggtggtgtatgggttgttgtggtttctagagtgtgtaggttgcgatcccatagtcaaggctgcactcattcggattgagcgtttcatgggatgtggcaggggatgtggcgtatctcacttaagcctttatggcctctctcagtgcctcaaatcctctgagggtaggattatgcagggttgaccctgctgatcgattctaggggccttctagggcgtctcaggggtatgtctgggttatggcgggtgtggcagatgatctagcgagtcaaggtgccgagctgagacataagatctatcatctaggtgtgtgagatgcatcacatcctcctggcgtggtgtacacccttaaggctactcggtcgatctggcgtggagggtgtagtaaagaaatgccgtttaaagccttcgcacggcgcctaggagcgccttacggggtgggggctaggtatttatacccccagcacattctgatcgattctagacgcctccaggatcctgatacacgatcagctatccagacgcagatcaccagtccctatcctggttagctaagcctcaactatgtggacagtgtgggatactgtgggggaagaaggacacggtaaaaagaagaggggggagcatcagccttcacaccttcaagccttaaggttttagcgcttagcaccgatggtcttagcagttagcaccgagccccctcacgggctcggcatcagcccgaacaggcacagccctgaaaggagtacacgccatcagggaaggcttgagagtacgaggagccctagcgacgagtactcgaaagcctgagggaacaccctcagcactgatgggcctagcgtgttcggaaaggacacaagagtgaagtgtgacagctatccgggagtgaaacccgttctgactaggggtttcagccttaaccacctgtaaaggttacaagactctaagaaaatttaaggaaaagtttaggtttaatttttggacctttactaccaaaaacacccgtttacacccctcaaacccgcctatagagccaaatccaccagtttgactcatcccaggtggggtatgataggctggacaggtagccagctggacgcaaggccgaaatccgctgacgcggctttcacccttacatccatcagtctaccaaagacttaaagacctaagggcttagcgctaaggtgctgatagcttagcaccgagcccctcaagggctcggcatcagtcttaaagctttaaacactttaagtaaacttaagagcttagcacttaaagttaattaataaccttaaaggcttacacacttagcactgagccctttaaggctcagcatcagtataaagatcttaacacctaagttaagtataaaaccttaaaggcttagcacttaaggatataaacttaacatcagtgtttaagactttaaaacttaaaataactattaagacttaaagacttataagctttaaacacttaaagtaactataagactttaaagaccttaagtatttaaagttaaccatcagtcttaaactttaatattataacctataagtcttaaagcttataggtataataatataatataagttataaaagttttagaagagctaaggggttaacttctttacttctctactctctttggtactttctctcttctcttcttttcttcatcaggggagaagaggaacctttaaccgtcaacgctgatggacttttcaccgtgtgactcgtgtgcttctggtcgcacgctcccatcgcacactccccacactctgacacccgtgcccctttcaggcttgacgtgttcggctgaaggcgtacggcgtgtcacgcttaaacccttaacaccaggtaagacttaaagtgtatattataagtagaagactttaaaaccttaaggtgttcccgcttagcctgtgtcctttagcgctaggcgccaagcgctaagctgtgaaacgcgaacacccatccacccccatttttcttccgtgtccttcttcttttgacaccgctggggggcgatgtgatatttctcacatgccagggggtagtggagaaaacaaacaccccggcacaaacagaacaccccctcaaacgaacaaaacaccccccagaatcgatcagcagggcaagggcaaggtattcatacccccaacacctttcaggccgttacaggagcaatgagaggctcacaggggcaaggggagatcaggggacgcgatggcacacaccaaccgcaccgcatcatcagcccaccggcgttggcggcaacgactcatcacccaagcccaacaacaaggccaaaccgaatgcccactctgcggagcccagatagcctgggacacccatgacctaccaaccagccccgaagccgaccacatcacacccgtcagcagaggaggactcaacaccctcgacaacgggcaaatcatctgcagaacatgcaacagaagcaaaggcaatcgcagcgaaccaaacatcaaattccaacaacaaaccacaaaaacattgattccatggtgaaaaacctgtcaacccccaccggggacaccccctgcacaggcgtgcaagacctcgtacggcttagtgaaatacctcccttttgtggatttgtctgtttgtcgactttttgtgttggtggtgagtgttgtgcagcctgagcttcctgatggtcgtgattggtgtggggagacgcgtcgttggtggcgtgtgtggggtgaggattcgcgtgccgggttggtgtctgatgaggagtggctgtttctcatggatgctgcggtgattcatgatgtggtgtggcgtgagggtcgcgcggatttggtggcttcgcttcgtgctcatgtgaaggcgtttatgggcatgctggaggctcattctggggatgctggcactactgtgggtggtgggggttctgcggtggcgatgattgaccggtataggaagcgcaagggggcctgattaggtgtctggtgttgtgggttctcaggttcctcgtcaccgtgtggctgcggcgtatcaggtgactgccggcaatgatgctggtgctcttggggctgcgtatgggttgactccggatccgtggcagcagcaggtgttggatgattggctggctgtcggtggtaatggcaggcttgctgcgggtgtgtgtggggtgtttgtgcctcgccagaacggcaaaaacgcgatccttgaggttgttgagctttttaagatggtggttcagggtcggcgtattttgcatacggctcacgagttgaagtcggctcgtaaggcgtttatgcggttgagatcgttttttgagaatgagcgccgctatccggatttggctcgtatggtgaaggcgattcgggcgacgaatggtcaggagtcgatcattttgcatcatcctgattgcagtgtgggtggtaagaagtgtggctgccctggttggggttcggttgagtttgtggctcgtagccggggttcggctcgcgggtttacggttgatgatttggtgtgtgatgaggctcaggagttgtcggatgagcagttggaggctttgcttcctacggtgtctgcggctccttcgggtgatccgcagcagattttccttggtaccccgccggggcctttggctgatggttctgtggtgttgcgtttgcgtgggcaggctttgtcgggtggtaaaaggtttgcgtggacggagttttcgattcctgacgagtctgatccggatgatgtgtcgcggcagtggcggaagttggcgggtgacactaatccagccttgggtaggcgtctgaattttgggactgtgtcggatgagcatgaatcgatgtctgctgccgggtttgctcgggagcggcttggctggtgggatcgtggccagtctgctacgtctgttgttccggcggataagtgggctcagtctgctgtggatgaggcggctctggttggcggcaaggtgtttggtgtctcgttttctcgttctggggatcgggttgctttggcgggtgccggccggactgatgctggggttcatgttgaggttattgatgggctgtcggggacgattgttgatggtgtgggccggttggctgactggttggcggttcgttggggtgatactgaccggatcatggttgccgggtctggtgcggtgttgttgcagaaggcgttgacggatcgtggtgttccgggccgtggcgtggtggttgctgatactggcacctatgtggaggcgtgtcaggcgtttttggagggtgtgaggtctgggaatgtttctcatcctcgtgctgattctcgccgtgacatgttggatattgctgtgaggtcggcggttcagaagaagaagggttctgcgtggggttggggttcctcgtttaaggatggttctgaggttcctttggaggctgtgtctttggcgtatcttggtgtgaagatggcgaaggctaggcggcgtgagaggtctggtaggaagcgggtgtctgtggtatgaactcggatgagttggctttgattgagggcatgtacgatcgtatccaaaggttgtcttcgtggcattgtcgtattgagggctactatgagggttctagccgggtgcgtgatttgggggtggctattcctccggagttgcagcgtgtgcagacggtggtgtcgtggcctggtatagctgtggatgctttggaggagcgtctggattggcttggctggactaatggtgacggctacggcctggatggtgtgtatgctgcgaatcggcttgctacggcgtcgtgtgatgtgcatttggatgcgctgatttttggtttgtcgtttgtggctgttatccctcagggggatgggtcggtgttggttcgtccgcagtcaccaaagaattgtactggccggttttcggctgacgggtctcgtttggatgctggccttgtggtgcagcagacgtgtgatcctgaggttgttgaggcggagttgttgctgcctgatgtgattgttcaggtggagcggcgtgggtctcgtgagtgggttgagacgggccgtatcgtgaatagtcttggtgcggttccgttggtgccgattgtgaatcgtcgccgtacgtctaggattgatggccgttcggagatcactcggtcgattagggcttacacggatgaggctgtgcgcacactgttggggcagtctgtgaatcgtgacttctatgcctaccctcagcgttgggtgactggcgtgtcggctgacgagttttcgcagcctggctgggtcctgtcgatggcttctgtgtgggctgttgataaggatgatgacggtgacaccccgaatgtggggtcgtttcctgtgaattcgcctacaccgtattcagatcagatgcggctgttggcgcagttgactgcgggtgaggctgcggttcctgaacgctatttcgggtttatcacgtctaacccacctagtggggaggctttggctgcggaggagtctcggcttgtgaagcgtgctgaacgcaggcagacgtcgtttggtcagggctggttgtcggttggtttcctggctgccagggcgcttgattcgagtgttgatgaggccgcgtttttcggtgatgtgggtttgaggtggcgtgatgcttcgacgccgactcgggcggctacggcggatgctgtgacgaagcttgttggtgccggtattttgcccgcggattctcggacggtgttggagatgttggggcttgatgatgtgcaggttgaggctgtgatgcggcatcgtgccgagtcgtcggatccgttggctgcgcttgctggggctatatcgcgtcaaactaacgaggtttgataggcgatggcttcgggggttgcgtcgaggttggctgctgccgggtatcagcggcaggcgattcgttttgccgggaagtatgcgggctattatgccgagttggggcgtttgtggcattccgggaagatgacagatgcgcagtatgtgcgtttgtgtgtggagttggagcgtgccggccatgacggttccgcggcgctggcgggcaagttcgtgtcggattttcggaagcttaacggtgtggatcctggtttgatcgtgtatgacgagtttgatgctgccgccgcgttggctaggtcgttttcgactatgaagattatgaatagtgacccggatagggcgaatgatacgattgatgctatggcggcgggtgttaatcgggctgtcatgaatgctggccgtgacacggttgagtggtctgctggcgcgcagggtaggtcgtggcgcagggtgactgatggtgatccgtgcgcgttttgtgccatgttggctacgaggtcggattatacgaccaaagagcgggcgcttactactggtcatacgcggcgtcataagcgtgccggtaggcgtccgtttggttcgaagtatcatgatcattgtgggtgtacggtggttgaggttgttgggcgttgggagccaaatagggctgatgccgagtatcagaggacgtatgagaaggcccgtgagtgggttgatgatcatggtttgcagcagtcgcctggcaatattttgaaggctatgcgtactgttggcggcatgagataatttgatgtggtttccggttgtgcgccgccggttatcggtgcacagggttgtctcccgcacgggggtcaacaatgttgtgttgttttccgcaaggagtgtaaggttaggctatggccgatcagagtgttgaggaacagaatgtcgacaatgatgctgttgagcccggaaagggtggagacattgttgatgttgtgaaggatgggcgggctgccggcgatgatcatgccggtgatgtttccgtgaagggtgaggcttctgggtcttcgggcacggattggaaggctgaggctcgtaagtgggagtctcgtgctaaaagtaatttcgccgagttggagaagcttcgtacatcgagtgacgattctggatctactattgatgagcttcgccgcaagaatgaggaactcgaagacaggatcaacgggtttgttcttgagggtgtgaagcgtgaggtggctgccgagtgtggcctgtcgggtgatgcggtcgctttcttgcacggtagcgatcgtgaagcactggtggagtctgctaaggctttgaagggtttgattgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctttcgccggagcagccgactatttttggccctgttaagggtgccgtgtttagtggtgttcctcgcgccaagattgttggtgagggtgaggttaagccttctgcgtctgttgatgtttcggcgtttactgcgcagcctatcaaggttgtgactcagcagcgtgtctcggacgagtttatgtgggctgatgctgattaccgtctgggtgttttgcaggatctgatttcgcctgctcttggcgcttcgattggtcgcgctgttgatctgattgctttccacggtattgatccggctacgggtaagcctgctgcggctgtcaagtcttcgctggataagacgaagaatattgttgatgcaaccgatagtgctacggctgatctgattaaggcggttgggctgattgctggtgccggtttgcaggttcctaacggggttgctttggatccggcgttctcgtttgccctgtctactgaggtgtatccgaaggggtctccgcttgccggccagcctatgtatcctgccgccgggttcgccggtttggataattggcgtggcttgaatgttggtgcttcttcgactgtttctggcgccccggagatgtcgcctgcctctggtgttaaggctattgttggtgatttctctcgtgttcattggggtttccagcgtaacttcccgatcgagcttatcgagtatggtgacccggatcagactgggcgtgacctgaagggccataatgaggttatggttcgcgccgaggcggtgctgtatgtggctatcgagtcgcttgattcgtttgctgttgtgaaggagaaggctgccccgaagcctaatcctccggccgagaactgatttattgttgcggtgatgtgtcaatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgcagggctttgttgcgctggaatgataccggggtttctggtcaggtgcagtatgagtctgcgggtcctttcgctcagactacacggtctaatactcccacgaatttgttgtggccttctgagattgccgcgttgaagaagctgtgtgagggtgatggtggggctggtaaagcgttcactatcactccaactattaattgtcgatatgcacattctgaggtgtgttccacggtgtggggtgagggttgctcgtgcgggtcgaatattaacggctacgctggccctttgtgggagatatgatatgaccagttttccttatggtgaaacgattgtgatgcttcagccgactgttcgtgtcgatgatcttggtgacaaggtggaagactggtctaagcctgtcgagactgtgttccataacgtggccatctatgcttcgttgtcgcaggaggatgaggccgcggggcgtgactcggattatgagcattggtcgatgcttttcaagcagcctattgtgggtgctgattatcgttgcaggtggcgtatccggggtgttgtgtgggaggctgacgggtctcctatcgtgtggcatcatccgatgtctggctgggatgcgggcacgcaggttaatgtgaagcgcaagaagggctgataggttgtggctcaggatgtgaatgtgaagctgaacttgccgggtattcgtgaggtgttgaagtcttctggggtgcagtctatgttggctgagcgtggcgaaagggttaggcgtgcggcctcggcgaatgttggcggtaacgctttcgatagggcccaatacagtaatggtttgtcgtcggaggtgcaggttcaccgggttgaggctgtggcgaggattggtaccacctataagggtggtaaaaggattgaggcgaagcatggcacgttggcgaggtcgattggggctgcgtcgtgatcgtttacggtgatccgcgcgtgtgggctaaacgcgtactcaaggatgatggctggctgtctgatataccatgtaccgggacagtgccggatagctttgagggtgaccttatttggttggctcttgatggtggcccacagttgcatgtgcgtgagcaggtttttttgcgcgtgaatgtgttttcggatacgccggatcgtgctatgtcgttggcgcgtcgtgttgaggctgtgctggctgatggtgtggacggtgaccctgtggtgtactgtaaacggtctactggccctgatttgctggttgacggtgcacgttttgatgtgtattcgctttttgagctgatatgtaggcctgcggagtctgaataagcttattgtttttgttttaatgtaattgtttgatatttaatgggggttgtgatggctgcaacacgtaaagcgtctaatgttcgctctgctgttacgggtgacgtttatattggtgccgctcatgctggtgacgctattgatggtgtgaagacggttcctgacggtcttaccgctttagggtacctgtctgatgacgggtttaagattaagcctgagcgtaaaacggatgatttgaaggcttggcagaatgcggatgttgttcgcacggttgctactgagtcgtctatcgagatttcttttcagctgatcgagtctaagaaggaggttatcgaactgttttggcagtcgaaggttactgccggatccgattcgggttcgtttgatatttctccgggtgccacgacgggtgttcacgccctgttgatggatattgtggatggcgatcaggttatccgttactatttccctgaggttgagcttgtcgatcgtgacgagattaagggtaagaatggcgaggtgtacgggtatggtgtgacgttgaaggcgtatcctgcccagattaataagaagggtgatgcggtgtctggtcgggggtggatgacggctttaaaagctgatactcctccggttccgccttctccgaagcctcagccggatccgaatccgccgtccgataattgatacacgagtttgagggattgttgatagatgagtgacacaggttacacgttgaagatcggtgaccgtagttgggtgttggcggatgcggaggagacggctcaggctgttcctgcccgcgtgtttcgtcgtgcagctaagattgcccagtcgggggagtctgcggatttcgcccaggttgaggtgatgttttctatgttggaggctgccgccccggctgacgcggtggaggctctggaggggcttcctatggttcgtgttgccgagattttccgccagtggatggagtggaagcctgaaggtaagggtgcctctttgggggaatagtttggctccacggcctgattgatgagtatcgtggggccatcgaatatgattggcgcacaaggtttggtgtgtgcatatacgatataggtggtcctgcaatggggtggggtgaggctgtccggctggctggcgtgttgtgtaccgatacgtctagccagttggcggcccacctgaatggttggcagcgcccgtttgagtggtgcgagtgggctgtgttggacatgctggatcattacaggtctgctaatagtgaggggcagccggagcctgtggcgaggcctacggatgagcgtagggcccggtttacgtctgggcaggtggacgatattttggcgcgtgttcgtgccggtggcggggtgtctcgcgagattaatattttggggtgaatagtgtatgtctggtgagattgcttccgcatatgtgtcgttgtatacgaagatgccgggtttgaaatcagatgttggtaaacagctttctggggtgatgccggctgagggtcagcgttcgggtagcttgtttgctaaaggcatgaagctggctttgggtggcgccgcaatggtgggcgccattaatgttgctaagaagggcctcaagtcgatttatgatgtgactattggtggcggtattgctcgcgctatggctattgatgaggctcaggctaagttgactggtttgggtcatacgtcttctgatacgtcttcgattatgaattcggctattgaggctgtgactggtacgtcgtatgcgttgggggatgcggcttctactgcggcggcgttgtctgcttcgggtgtgaagtctggcgggcagatgacggatgtgttgaagactgtcgccgatgtgtcttatatttcgggtaagtcgtttcaggatacgggcgctatttttacgtctgtgatggctcgcggtaagttgcagggcgatgacatgttgcagcttactatggcgggtgttcctgtcctgtctttgcttgccaggcagacgggtaaaacgtctgctgaggtgtcgcagatggtgtcgaaggggcagattgattttgccacgtttgcggctgcgatgaagcttggcatgggtggtgctgcgcaggcgtctggtaagacgtttgagggcgctatgaagaatgttaagggtgccctgggctatcttggtgctacggctatggcgccgtttcttaacgggttgcggcagatttttgttgcgttgaatccggttatcaagtcggtgacggattctgtgaagccgatgtttgctgccgtcgatgctggtattcagcgtatgatgccgtctattttggcgtggattaaccgtatgccgggcatgatcactcgaatgaatgcacagatgcgcgccaaggtggagcagttgaagagtatttttgcaaggttgcatttgcctgttcctaaagtgaatttgggtgccatgtttgcgggtggcacagccgtgttcggtattgtggctgccggtgtggggaagcttgttgcagggtttgccccgttggcggtgtcgttgaagaatctgttgccgtcgtttggtgctttgaagggtgccgctggtgggcttggcggcgtgtttcgcgccctgggtggccctgttggtattgtgatcggcttgtttgcggccatgtttgctacgaacgcccagttccgtgccgctgttatgcagcttgtggctgtggttggccaggcgttgggccagattatggccgctgtgcagccgctgttgggtttggttgctgggctggtggcacggttggctcccgtttttggccagattgttggtttggtggctggtttggctgcgcagcttgttcctttgattagtatgctggttgcccggctagttcctgtgatcacccagattattggtgcagtgacgcaggtggcggccatgttgttgccggcgttgatgccggtgcttcaggcgattgttgctgtgatacggcaggttgttggtgttgtgatgcaactggtgcctgttttgatgcctgtgattcagcagattttgggtgctgtcatgtctgtgctgccgcctatcatcggcctgatccggtcgttgataccagtcatcatgtcggttatgcgtgtggtggttcaggttgttgcggttgtgatacaggtggtggcccgtattcttgctgttgtggctccgatggtggctgctgtggtgggttttgttgcccgtattgttggtgctgtcgtgtcggctgtggcccgtgtgattgcggctgtggcccgtgtgatcggatgggttgtggcccattttgtgtctggtttggcacgcatgggttcggttattcaggctggctggaatcatattagagcgtttacgtcggcgtttatgagcggtttcaagtcgatcatttctggcggcgtgaacgctgttgtggggttttttacgcggcttggttcttcggttgcttcccatgtgaggtctggttttaacgcggctcgtggcgctgtttcttctgcgatgaatgctatccggagtgttgtgtcttcggtggcgtctgctgttggcgggtttttcagttcgatggcgtctagggttcgtagtggtgctgtgcgcgggtttaatggtgcccggagtgcggcttcttctgctatgcatgctatggggtccgctgtatctagcggggtgcatggtgtgctgggttttttccggaatttgcctggtaatattcggcgtgcgcttggtaatatggggtccttgttggtgtctgcgggccgtgatgtggtgtctggtttgggtaatggtatccggaatgctatgagtggcttgttggatacggtgcgtaatatgggttctcaggttgcgaatgcggcgaagtcggtgttgggtattcattcaccgtctagggtgtttcgtgaccaggttggccggcaggttgttgccggtttggctgaggggatcacagggaatgctggtttggcgttggatgcgatgtcgggtgtggctggaaggctgccggatgctgttgatgcccggtttggtgtgcgatcgtctgtgggctcgtttaccccgtacgaccggtatcggcgtgcgagcgagaagagtgttgtggtgaatgtgaatgggcctacttatggggatccgaacgagtttgcgaagcggattgagcggcagcagcgtgacgctttgaacgcgttggcttacatgtgatcgagggggtgttgtgcatgtttattcctgacccgtctgatcgttctggtttgactgttacctggtctatgttgccgttgattggtaatgatccggagcgtgtgcttcatttgacggattatacgggtgcgtcgcctgtcatgttgttgaatgattcgttgcgcggtttgggtgttcctgaggttgagcatttttctcaaactcatgttggggtgcacggctcggagtggcgcgggtttaatgtgaagcctcgcgaggtgacattacctgtcctggtgtcgggtgtggatccggatccggtgggcgggtttcgtgacggtttcatgaaagcctatgacgagttgtggtcggcgtttcccccgggcggggtgggggagttgtctgtgaagactcctgctggtcgtgagcgtgtgttgaagtgccggtttgattcggtggatgatacgtttacggttgatccggtgaatcgtggctatgctcgctatctgttgcatttgacagcttatgacccgttttggtatggggatgagcagaggtttcgttttagtaacgcgaagttgcaggattggttgggtggcggccctgtcggtaaggatggcacggcgtttcctgtggtgttgacgcctggtgttggttcgggttgggataatctgtcgaataagggtgatgtgcctgcgtggcctgtgattcgtgttgaggggcctttggagtcgtggtctgtgcagattgatggtttgcgtgtgtcttcggattatcctgttgaggagtatgattggatcactattgatacggatcctcgtaagcagtctgcgttgttggatgggtttgaggatgtgatggatcgtttgacagagtgggagtttgcgcctatcccgcctggcggttctcggagtgtgaatattgagatggttggtttgggtgccattgttgtgtcggtgcagtacaggtttttgagggcttggtgaatagttgatggctggtcttgtcccgcatgtaacgttgtttacgccggattatcgtcgtgtggcgcctatcaatttttttgagtcgttgaagttgtcgttgaagtggaatggtttgtctacgctggagttggtggtgtctggggatcattctaggcttgacgggttgactaggccgggtgcgcggctggttgttgattatggtggtggccagattttttctgggcctgtgcgtaaggttcatggtgtgggtccgtggcgttcttcgcgggtgactatcacgtgtgaggatgatatccgcctgttgtggcgtatgctaatgtggcctgtgaattatcgtcccggcatggttggttcggagtggcgtgccgacagggattatgctcactattcgggtgcggcggagtcggtggctaagcaggtgttgggggataatgcttggcgttttccgcctggtttgtttatgaacgatgatgagagtcgtggccgctatattaaggattttcaggcccggttccatgtgtttgccgataagttgttgccggtgttgtcgtgggctcggatgactgtttcggtgaaccagtttgagaatgcgcagtttgatcagcggggtttgctgtttgattgtgtgcctgctgtgacccggaagcatgtgttgactgccgagtctggttcgattgtgtcgtgggagtatgtgcgtgacgccccgaaggctacgtctgtggtggttggtggccgcggcgagggcaaagatcggctgttttgtgaggatgttgattcgatggccgagggggattggtttgatcgtgtcgaggtgtttaaggatgcccgtaacacggattctgaacatgtgcatctcatcgatgaggctgagcaggtgctgtccgagttaggggccacgtcggggtttaagatcgagttggctgagtcggatgtgttgcggtttgggccaggcaatctgatgccgggtgatcttatctatgtggatgtgggttctggccctattgcggagattgtgcggcagattgatgtggagtgtgattcgcctggtgatggttggacgaaggtgactcctgttgcgggggattatgaggataatccgtcggcgttgttggctcgccgtgtggctggtttggctgcgggtgtgcgggatttgcaaaagttttagtaagtgattggggtttgttgtgggtattgtgtgtaaagggtttgatggtgtgttgaccgagtatgattgggctcaaatgtctggtctgatgggtaatatgccgtctgtgaagggcccggatgattttcgtgtcggtacgacggttcagggtgccacagtgttgtgtagtgttttgccggggcaggcgtgggctcacggggtgatgtgcacgtcgaatagtgttgagacggtgacggggcagcttccgggccctggcgagactagatacgactatgttgtcctgtctcgggattgggagcagaacacggccaagttggagattgttcctggggggcgtgcggagcgtgccagggatgtgttgcgcgccgagcctggcgtgtttcatcagcaactgttggcgactttggtggtgtcgtctaacgggttgcagcagcagttggataggcgtgctatagcggctagggtggcgtttggcgagtctgctgcgtgtgatcctacccctgtggagggtgaccgggtgatggttccttcgggggctgtgtgggctaatcatgctaacgagtggatgctactgtctccgaggattgagacgggttctaagtcgatcatgtttggcgggtctgctgtgtatgcttacacgatcccgtttgcccgcccgtttagtagtccgcctgttgtggtggcgtctatggctacggcggctgggggcacgcagcagattgatgtgaaagcctacaatgtgactgccaaggattttggtttagcgtttattacgaatgacgggtctaagccttctggtgtgcctgcggtagctaactggattgctgtcggcgtgtaatgcgctgcttgtgtgtgcgggatatgttgtggtggttgtagtggtagggggctgtagtgtcatggtttacacccacacttgtagcctctatttgtaccgctatcgctactgtccttggttcgattcaggcggttacttacaggtcgaagaagaggcttaggcagttgtctgcacaggttgatgcgatggaagaatacacatggaatattcgccatattgttcatcgctataacgcgaatttgcctgagaatgttgagcctgtaaaaatgcctgatttgcccgagtttttgaaggatactgttgatagtggtggggggtgaattgtgagggagttggaggaagagaaacggcagcgccgcaattttgagaaggcttcactggtgttgttgtttttgtcgcttgtgttgttggctgtggttgctgtgggtgctttgcgtttcggggctgtatcctctgagcgggattcggagcaggctagggcccagtcgaatggtacggccgctaaggggttggctgcgagtgtaaggcgggcgtgcgtctctggtgggcaggagtcggtgcgtcttcaccagtctggcttgtgtgtggatgctcagcgtgttgagcg

SEQ ID NO: 70 - DNA, 29699 bp, synthetic construct
  source 1..29699: mol_type = other DNA; note = PAC9
tgatggtcgggatggttcggccggtgagcgcggtgatgtgggcccttcaggtcctgccggcccgcaaggtgcacagggtgaacggggtgagcgcggccccgccggtgcgaacggatccgatggtaaagacggtaaggatggtgctgatggccgtgatgggcgttcggtgatatcggtgtactgttccgggggccgcctggttgtgaaatatagtgacggtacggcctctaccgtgtcgggttctgcggcctgcgagagtgtgaaaccatcacctgtggttactgtatcatcccataggtgaacaagaagagggaagggtgttactagtgttgattgtggtgtttgggggtggtgtgtggtgagatacattccagcggcgcatcactctgccggttcgaatagtccggtgaacagggttgtgattcatgcaacatgcccggatgtggggtttccgtccgcttcgcgtaagggtcgggctgtgtctacagcgaactatttcgcttccccatcatcggggggttcggcgcattatgtttgtgatattggggagacggtgcagtgcctgtcagaggggactatagggtggcatgccccgccgaatccgcattctttgggtatagagatttgcgcggatgggggttcgcatgcctcgttccgtgtaccggggcatgcttacacgagggagcagtggcttgatccgcaggtgtggcccgcagtggagagggccgctatcctgtgtcggcagttgtgtgacaagcatggtgttccgaaaaggaaactgtctgtggccgatttgaaggccggtaaacggggtgtgtgcgggcatgtggatgttacggatgcgtggcatcagtcggatcatgacgatccggggccgtggtttccgtgggacaaatttatggctgtggtgaatggccacggcggcggttcaagtagtgaggagttgagtatggctgatgtacaagcgttacataatcagattaaacagttgtcggcacaggtggcccagtcggtgaataagctgcatcacgatgttggtgtggttcaggttcagaatggtgatttgggtaaacgtgttgatgccctgtcgtgggtgaagaatccggtgaccgggaagctgtggcgcaccaaagacgccctgtggagcatctggtattacgtgttggagtgtcgcagccgcatagacaggcttgagtctgctgttaatggtttgaaaaagtgatggtggtttgttgtgggtaaacagttttggttgggcttgtttgagcgtgccctgaaaacttttattcaaacgtttgttgctgtgcttggggtgacggcgggtgttacttatactgcggagtcgtttcgcggtttgccgtgggagtctgccctgataacagcaacggttgctgcggtgctgtctgttgctacatcgtttggtagcccgtcatttgtggccggcaaacctaaaaccacggttgtggatgctgggcttgttccacccgacgatgggggcatggttgagccgcactcggtggatgtgtcggatcctggcatgatcgagccgacagatgatgtggatggttttgctggctatgtgccgaagcgtgcagccgagtcggaggttagcacggtggagtctactgttgcataattgaacatagatgtgtgccccagcggtgctgccacgatcgtgtggtggttgccgctggggcactctttttgtgtctataggagttttacaggttgtcgtctagtgtgtcttcgagcatctggtccaggtagaggcaggcggagatagtatcgttggcctggtctagaacgttctggccgataacatttttatgattgtcgcggtggctgatgatagaccgcatgatatcgtcggccgccgcctgcaatagtttggcctggtatgcgattcctgcgagccagtctagtgcttcctggcttgccagtgtgtcgtctggaatgccacgggtgttgctgttgtttgtggggtgtcctgcactgtcgcagcaccacaagatttcgctgcactcgtctagcgtgtcctggtcgatagcaagatcgtcgaggctgacttctttgacggtaaggttcacattgtcgagggagatgggtacaccgtattggttttcgacactgtcaacaatgttttccaactgttgcatgttggtgggctgttgttggatgatacggtgtactactgttttgatggcggtgtaggggatattgtgtgtgttgttcatggtttttatcccacccctgtgttgtcgtcgttattgtctggatagtatctactgtttgcgtagcctgtgagggtgatgagtgtttggtctgcccactgtttcactgtctgccgggtgacacccaatcgttgggcggctgtggcgtaggtttgatcatacccgtatacttcacggaatgcggctagcctggctaggtgttttcgctgtttggagggttcacatgatagggtgtagtcgtcgatggcgagctgtagatcgatcatggtggcaatgttgttgccgtgatgctggggggcggttggtgggggtggcattcctggctccacactgggtttccatgggccgccgttccagatccattgggcggcttggatgatgtcggcggtggtgtaggttcggttcatgtgtcaccccctgaacaggtcgttgctggtgctggtgttggtggtgtcgaatcgtccgacgcagtggcagtagtcgtacatgagtttgataatgtgttggtggtctcccaaataggtgtttccgctgatactgtaggtggctgtgccgtctttactgatggtgtatttggcggtgatggtttcggggttttcggtgtcggtgatgattgctgtggtggtggtgcctactgtttgtagcacggtggtttgggttccgtcgtcgatagtggttttaaccatggtgtgtgttctccctttttagatgctggtttggttgtcggctagatgaatgatgtcgggtaagggtttcggctggtctaggtgttgtatggttttgttggctagccgtttggctaccctgtaacacattttggtgtagtgtttgttgtctaggttgtggtattgttcccgcaccgcaatatatagtagagagtcttggtacaggtcgtctgcactgattgcggggtagtgtgcggctgttttggtgcatgcccggttgagtgtgcgtagatgatggtctgtggcccacacccacgatgcggtggtggctaggtcggcttttgttggtcgtctgctcatggcatttctttcatcgggctatctggtagttgtttggtgttttgttgttgatagtgtagcacacgagtccggggtttccggtggtgccagtcttgtgccggtaccatgtggattcgccttccatggatgggcattggatgaaggtgcgttgtccttgttcggagatttctaggtggtgccggtgcccggccatgaggatgtgggatgtggtgccgttgtggaattcttggccgcgccaccaatcatagtgtttgccggtgcgccattggtgtccgtgggcgtgcaggatttgtgtgccggccacgtcgacggtggtggtcatttcgtcccgttgggggaagtggaagtgaaggttggggtattggttgttgagctggtaagcttctgcgatggcgcggcagcagtccacgtcaaaggagtcgtcgtaggtggtgactcctttgccgaagcgcacggcttcgccgtggttgccggggatggatgtgatggtcacatttttgcagtggtcgaacatgtggacgagttgcatcatggccatgcgggtgagcctgatttgttcggtgaggggtgtttgtgtgcgccaggcgttgttgcctccttgtgacacgtatccttcgatcatgtcgccgaggaatgcgatgtggactcgttcgggtttgcctgcctgttgccagtagtgttttgcgactatgagggagtgcaaatagtcgtctgcgaatcggctggtttctccgccggggatgcctttgccgatttggaagtcgcctgccccgataacgaaggctgtctcgtcactgctttgggtgtcttgttcgggtttgggtggctgccattcggctagtttgttgacgagttcgtcgacggggtaggggtcggttgcgggttggtggtcgatgattttttgtatggatcggcctgtttctccgttggggagtgtccattcggagatgcgtgtgcggcgtacagtaccgttggctagattgtcgtcgatggtgtcgatggcgttgtcgtggttggctagctgtgtgagtagccggtctatattgtctatcactggttttcctcctctggcggggtggtgttggcttgtttgcggcggtagtcttttataacggtggcggagatggggtatcctgcctgggtgagctgttttgctagccacgaggcgggtatagacctgtcggcgaggacgtctgcagccttgttgccgtagcgttgaataagggtttcagttttggttgccatgatgtcctatcggttgtgtggcgggctgccatcctgtgcggcagtcgccgtcgtggcctggtttgcgtgtgcaccacgatacggttccgtctgtgtggttgagtgttttgccgcacatgacgttttgtagatgctcgggcagggcgccgtcaccctggttgctggtttgtgtgtcgaagagtgttttctggttggtgaaatgctctgacacggtgccgttgtgtacgggtagtatccatgttttccattgttgttgtagccgggtgttccagtggaattgtttggccgcgttcgtggcttgtttgatggttttgtagtagccgacgaggatgcgctggtgttcactgtcgggtgggttttggcctcgccagtattgtgccgcgacggcatacctgttgttgtctgtgaaggcgtcccagcagtattcgataatgtgttgtagtacactatcgggaatgtctcgtacttggttttcgtcgagccacgcgtcgacaatgatgttgcgtatggcgtgtttgtctttggtggtgggtttgaacgagatactcaccatgctggcctgtcgtcttgcatgaaatcgttaaaggatgattcgcttgtgcggcgtgcctgggtgatttgctggtcagtccagtcggggtgttgctgtttcagatagtaccagcggcaggcatcatatgtttcgttctgcaagcgggtgagatggttttcggtgatgatttgtttccacattgtccacgaaacgtcgagcctgcggagcatgtccatggccggcacattaaacgagtcaaggaagagtatttcgtgggtgtagtagtttttctcgtaggcgtaccatccgcttcggtgcctgtggggctggtttttggggtaggcttcccggcatactttgtgtaaacgtttggccatgtcgtcgggtagttcaatgtcggggttggcgcggatcatggatcgcatcccgtcgtaggtggtgccccaggtgtgcatgatgtgtagtgggttgtctccatcggcccatttttctgcacagatggcgaggcggatgcgcctcctggctgtttggctggtgttgcgccggttggggattgggcacgtgtcgaggggatccattatgttttagtgtacctttctggtttcgtgttgttgacgtgttttactgtagcacagtgtctagtgcttgtgtcaaccctgtttttccggcctgcaggtaggtgtctgtgacatctccgaccgtgaggggcacatgggtggcttgggggagtgctgcctggatggtttgtgccatctggtcgcctgcggggtctgggtctgaccagatgtagatgtggtcgtagccttcgaagaatttggtccagaagttttgccacgaggttgcgccgggtagggctacggccggccatccgcattgttcgaggatcatggagtcgaattcgccttcgcaaatgtgcatttcggctgccgggttggccatggcggccatgttgtagatggagcctgtgtccccggctggggtcaagtatttggggtggttgtgggttttgcagtcgtgtgggagtgagcagcggaaacgcatttttcgtatttcggctggccgctcccaaacggggtacatgtatgggatggtgatgcactggttgtagttttcgtggcctggtatggggtcattgtcgatgtatccaaggtggtggtagcgggctgtttcttcgctgatgcctcttgctgagagcaggtcgagtatgttttcgaggtgggtttcgtagcgggctgaggctttctggattcggcggcgttccgcaatgttgtagggttgtatgctgtcgtacattcgggttttcttcttctagtcgttgttgtagtttgtggagtcctcctccgacaccgcatgtgtggcagtaccagacgcccttgtcgaggttgatgctcatggagggctggtggtcgtcgtggagcgggcagagtatgtgttgctcgtttttggacgggttgtagcgtatctggtagatgtcgaggatgcggcgggtgtcagaggtgtgggaggagctcgttgagggttgataccacataggcttcgctccagggtttgttgcgttgtttcatcactacgagtccgatggtggaattgttttcgcggtttcggtgtgtttcgtagttgcgtgcctcccggctggcttgtttcacgaattcggctaggtggggctggccggctttcgcctcgataatgtaggttttgttgctggttgtgaggatgaggtcgccttcgtcttcgcggccgttgaggtggaggcgttcgatatcgtgtccggtgtcgcgtagctggtgcaataatcgtgtttcccattcggctccggcccgccggttgcgtgcctgctgtgtggccatagtttttagagtcctttgtgtgttgtggtcatgttccagggctgtttttcggcgaggggcccgaagaatgtgtattcggggtaggctcgtagtcgttcatatcgggtgccgtcggggctggatttgccggtgcgctgtttcaatactgcgatgcgtgcctcggccggtatcgtgagaccgttgccgttatcctcgccaccatacaatgagactcccaatatgagttgtggtttttcggagaggccgtttttgatttctcgccgtgccggggggtgttcgatgtcggttccggttttgtcggtggcgtggtgtgtgacaataatggtggatccggtgtcgcggcctaatgctgtgatccattgcatggcttcttgctgtgcctgatagtcactctcgcagtcttggatgtccatcaggttgtcgataacaatgagtggcgggaaggtgttccacatttccatgtaggcttgcagctccatggtgatgtctgtccatgtgatgggtgactggaatgagaatgtgatgtgttggccgtggtggatgctgtctcgatagtattctggtccgtagtcgtcgatgttttgttgtatctgtgtggtggtgtgttgggtgttgagtgagatgattcgtgtggaggcctcccagggtgtcatgtcccctgatatgtagagggcgggctggttgagcatggcggtgatgaacatggctagcccggatttttggctgcctgagcgccccgcaatcatgacgagatcccctttgtggatgtgcatgtcctggttgcggtagaggggttctagttgtggtatgcggggcagctcggctgcggtttgggaggctctctcgaaggatcgttggagagagagcatcgggaccttatctatctatcggttgggtgtgttttggtggtcagatggagtcgatgtcgatgtcagcatcggcgggggctgtggtgtcgtctagctggccgttatcgcgcttgtctacgtattcggcaaccttatcgtagatggcgtcatcgagggggtggtgtcgtctagctggccgttatcgcgcttgtctacgtattcggcaaccttatcgtagatggcgtcatcgaggggtttgagcacgaccgcattgaacccgtttttggtgcgcacggtggcgagtttgaaggcctgctcctcgccaaggtaggcttcgaggtcgcggatcatggaatgtgggcggtcgttgttgccgcgcgctttctcaataatagcgttgggaatgatttctggggtgccgttgttgagatcgtctagggtgtggaagattgtgacatcagcgtagatgcgatcggctgtctgtccaccgtagccttcggtgttgtgttctacgtcgcggattttgaaggcgatggcggtggcgtcctggtttcgggaggggttgaagaaggtgctgttgctgttgttgcggtagttggcgagtcccatggttgtttcctttactgtttgtgttggtttgtgtcggttttatcgggtgaggctgtttcgtttgctgcggaaagcctcggacacgtcactgttactggtgatgattttcttgtactgtttcagaaggtcggctagctgtgccttgcttgttgcattgttgattttgtcgatgataatctcgttttcgtttgatgcgatgttgtctacgtagtctttggctgcctggttgtagcggtcttggaggatgatggatgcgcttgctacgagtgttgctagatcccagtctttggacacgtcaccgtttttgaggccgcctagcagatcaataatggattgtttgatgtcttctgcggtgtctccgcggatgactgtccatggggctgcgtagtctccaccgtatttgagtgtgatagttagctttccgctgtctgtggtgtgctcgtcggtcacgtgttttccttttcgttgttttcggcttctggtggctgtacggtggtttctaccgggtatctgtacgagtttttcccgttgacggcccagcaggcgtccttgacggggcatcctttgcagagtgctgtgacgtggggtacgaagatgccttggctgattcctttcattgcttgactgtacatggatgatacatgccggtaggtgttgttgtcaagatcaatgagttcggtggatgtgccctgctcaaccgattgctcgtctcccttggtggtagcgggtgtccaaaacattcctttcgtcacatggatgccgtgttggttgagcatgtaacggtaggtgtgcagctgcatactgtcggcgggtaggcgtccggttttgaggtccaaaatgaaggtttcacccgtattcgtatctgtgaatacccggtcgatgtagccaacgatctgggtgccgtcggggagggtggtttctaccgggtattcgatgcccggctcgccgtcaataacagcggtagcatattctgggtggttgcgcctccatgttttccaccggtccacaaaggtggggccgtaaatcatccaccaattgtagtctttcttgtgtgtcccgcccgactcgcacatgtttttgcatattctgccggagggtttgatttctgtgccttcggattcggcgagggcgacttgggtgtcgaaaatgtttttgaaggatgagagtttgtctggcagtgcagggtattcggcgggattgtacaggtgtaggtcgtattgttcggtgatgtggtgtatggcgcttccggcgatggtggcataccaggtgtggtgttgggcgtggtagccgtgggataggcgccatttttcaccgcattcggcccactgtgacagtgatgagtaggagatgtggcctggatggtcaatggtggacggtttttgtgctaggggcattacttgtcgcttttgtgggtgttccatgggtttcgggtgtcttggccggcattgtgttgctggtatgcgaggagtgcgaggcagtgccaggcagcatgggccagatggggtagcccggattcatcatcgaggttgttgccttgctgccatgataacaggtgccggtagagggcgtcaacactgtggctccacggatagccgccggtccagttgttgtcgccgtatttggtggcgccgtatccggccacagagccgagggcgtgtaaggctgtagggtcgatgagggatagcctgcaaagtttcaattctttcttggcgccagtatcagggtcggtgtacatgctggtgggctcatccatggtgtgtgtgctccttaagtatggggttactggttggggttgtgggcgagtgctacggcgagaataatgatggcgagggtttcagcgatcagtatgggtgttgtgatcatttgtggtcgcggggattgttggtgagggttgaggcgcccaggaggatagtgagggcgcatgcggcgatgatggcgagggctgccttgtgtggggtgccggtggcgtacatccatgtgatgatgccgccttggatccaggcgaggctggtgaagaacgtttcgtagctgtgtagctcaatgttgttgttgggtgtgttcatgcttgctcctgaagaatggtgttgatggttgtgtaaatgttgtacaggtcggtttcgatagataacagttggtggatttggtggtcgagatcaatgtcggggttgagggtgttgatgcgggaggcgatgtcggtggctgtgcgtagtgtgccgccggtgtggtgaatgatgtgtgccgtgtcggcgagtccggtggtgacagtgtagtgggagaggagaggcatagctgggggtgctccttgacggggttactgttgcgggttgatgttgaggtcggtgacgttggggtggtcttctgttccggtgacgaggcagtggacggtgactgggagtttggatgcgccgggctgtttcgcggttgcgccgtagacgatggagaaggtgtctttgccaataattttgtggagttggaggtcgatgtcggggttgccgttccatttgacgccttgtgtggcggcctgttgttcggctttgcggttgcaggtgtgtgctgcggtgatcatggtgagtccggtggcggtttcttcaccccttgcttgggcttgcttgtgggttttctgctgttcggctcgcagtgactgttctgctgctgcctgccgtgctttcttttcggctttgcgctgttgggtagtcttgggggtccattcggtgttggctgtggtggcttgcggtgcgggttgtgatgcgagtggcggattgtcgtctggggctggcatgaaggatgctgcggcgatgatggcggctgtgattccggcgatggtgtagccgtttttcttgttcatgattttgtgttcccctttccggggtgttgttcgttgctgacatgattaatactttcagcggctgggcccactgtcaaggctgcgctcaacgattgtgagcgatacttgtgtggctaggggttttgtccttgaggtgggagatgtctttcccttgcgtccagtatccatggcggttgcgagtcatccctttggcgagcatctcgtccacggtgagacacctgcgacgatctggaccctccttgactccctgatcgcctgtgcggtgcatgtcaccggcacaagtaccattaaatgtctcgtggcagattgtgcaatgctctggtcggtatccgatgattgtgctatcgcacttgtggcatgtccattgcatgattggtccttctttcgtgttttaagcttgtactctgaggattagagcgactttcagcccttggggggtatgattatataggtcaggtatttctaggcgattctaggctcattgtgtgtggctgggggttatcgggcacacagggtgaggagttggccaacattgatgcgggtcacattccagtagagttgcgtggcttccccaccggtgagtggcttccactcgtcatggctgaacacggtgccgtcggttgcgatgaatgtgttggggcgtagcttgtgaagctcagtctctacacgctgccggtaggcttcggcgaggccctcgaaatccatgtggtcgcaggggaggttttcgaggcgtgtcaggtcgaagggtgtggggcagtcgtagctggcggggctgtagagctgggtgaaatggttggcgatcttctgcatgacgggttccttttctcgtatggtgagttgatagttttatcgggtggatgcgacaaggatggcgtctacatcgatcatgtcgatgagatcgtggagttcctcggcctcattctcggagaggtggcgccagccatagtcgccgtatacggcgccgtcgagggtgacagtccacaggggccggatgagtcgtatggcttcttgtactttagcgtggtacatgcggcgcaccatatccagatcgatgtcgtctgaatggtttccagtgaggctgtagaggctgagcgggtcgatttctgtctgcctgtagagggatgtgaatgatggtgtgatgagtgtgccatccatgagagtgtgctcctttcggtggtggaggggttgttgtggtttctagagtgtgtaggctgcgacccatagtcaaggctgcgctcattcggattgagcgtttcatatgggtgtggcatggaatctacacccccatactgtgtgagataggccacatcctcctggcttggtgtgaaccctcgagactactctgcctatctggcgtggagggtgtagcccagaaataccgtttaaagccttcatacggcgcctaggagcgccttacagggtgggggctaggtatttatacccccaagcaattctgatcgattctagacgcctcccaggagcccgatacacgatccgctatccagacacagatcatcagcccctatcctggttagctaagcctcaactatgtggacagtgttgattactgtggggtaagaaggacacggtaaaagaaagaggggggagcatcggctttcaagccttaaggtcttagcagttagcaccgagcccctcaagggctcgtcgtcagcccatcaggcacggccctgaacggggtacacgccatcagggaaggcttgagagtacgaggagccttagcgacgagtactcgaaagcctgagggaacaccctcagcactgatgggtctagcgtgttcggaaaggacacaggagtaaagcgtgacagctgtccgggagtgaaacccgttctgactaggggtttcagccttaaccaccctcaaaggttacaagactctaagaaaatttaaggaaaagtttaggtttaatttttggacctttactaccaaaaacacccgtttacacccctcaaacccgcctatagagccaaatccaccagtttgactcatcccaggtggcatatgataggctggacaggtagccagctggacgcaaggccgaaatccgctgacgcggctttcacccttacatccatcagtctaccaaagacttaaagacctaagggcttagcgctaaggtgctgatagcttagcaccgagcccttgaggggctcggcatcagccctaaagccttaaacacttaaagtacatataaaactttaaaagcttaacacttaaggttataaataaacattaaagctttaaagtcttaaagtacatatataaccttaacacctaagttaagtataaaaccttaaaggcttagcactgaaggatataaacttcacatcagtttttaagactttaaaacttaaaataactattaagacttaaagacttataagttttaaacacttaaagtaactataagactttaaagaccttaagtacttaaagttaaccatcagtcttaaactttaatattataacctataagtcttaaagcttataagttataaaagttttagaagagctaagaggttaacttctttacttctcttctctctttggttctttctctcttctcttcttttcttcatcaggggagaagaggaaccttttaccatcagcgccgatggactgtcaccgtgtgactcgtgtaccaccggtcgcacgctcccggtttcacactccccacactctgacacccgtgtccctttcaggcttagcgtgttcggctgaaggcgtacggcgtgtcgcgccaacacccttaacaccaggtaagacttaaagtgtatattatatgtagaagactttaaaacctataaggtgttcccgcttagcctgtgtcctacaccgctaggcgccaagcgttaagtcttgaaacgcgaacacacacccacccccatttttctttcgtgtccttctcttttgacaccgctggggggcgatgtgatctttctcactacccatgggggtagtggagaacacacccaccccaccatcaacagaacaccccctcaaacgaacaaaacagggcctagaatcgatcggcagggcaagggcaaggtattcatacccccaacacattccaggccgtcagagaggcaaataagacccgtacagggctagtcgaggatcggagacgtgatggcacacaccaatcgcaccgcatccgccgcacaccgacactggcggcaacgactcatcacccaagcccgacagcaaggccaaaccgaatgcccactctgcggagcaaccatcacctgggacacctaccagctgccaactagccccgaagccgaccacatcacacccgtcagcaggggaggactcaacaccctcgacaacgggcaaatcatctgcagaacatgcaacagaagcaaaggcaacagaacacaaccaaacatcaaattccaacaacaaaccacaaaaaaccttgttccatggtgacaaaacccgccaacccccaccggggacaccccctgcacacccgtgcaagacctcgtacggcttagtgaaatacctcccttttgtggatttgtctgtttgtcgactttttgtgttggtggtgagtgttgtgcagcctgagcttcctgagggacacgagtggtgtggggagacgcgtcgttggtggcgtgtgtggggtgaggatagccgcgcgcagtacgtgtctgatgaggagtggctgtttcttatggatgctgcggtgattcatgattgtgtgtggcgtgagggtcgcgcggatttggtggcttcgcttcgtgctcatgtgaaggcttttatgggtatgttggatcgttattcggttgatgtggcgtctggtggccgtggtgggggttctgcggtggcgatgattgaccggtataggaagcgtaggggggcctgattaggtgtctggtgttgttgggtctcaggttcctcgtcatcgtgtggctgcggcgtattcggtgtctgctggcggtgatgctggggagttgggtcgtgcgtatgggttgacgcctgatccgtggcagcagcaggtgttggatgattggctagctgtgggtggtaatggcaggcttgcttcgggtgtgtgtggggtgtttgtgcctcgccagaatggcaagaatgctattttggaggttgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgtgcgacgaatggccaggaggctattgtgttgcatcatccggattgtgccacgtttgagcgtaagtgtggttgtccgggttggggttcggttgagtttgtggcccgttctcgtggttctgctcgcgggtttacggttgatgatttggtgtgtgatgaggctcaggagttgtcggatgagcagttggaggcgttgcttcctacggtgtctgcggctccttcgggtgatcctcagcagattttcttgggtacgccgcctgggccgttggctgacgggtctgtggtgttgcgtttgcgcgggcaggctttgtcgggtggtaaaaggtttgcgtggacggagttttctatcccggatgagtctgatccggatgatgtgtcgcggcagtggcggaagcttgctggtgagacgaatcctgcgctgggtaggcgtctgaatttcgggacggtgagcgatgagcatgagtcgatgtctgctgccgggtttgctcgggagcggcttggctggtgggatcgtggccagtctgcttcttcggtgattccggcggataagtgggttcagtcggctgtggatgaggcggctctggttggcgggaaagtgtttggtgtctcgttttctcgttcgggggatcgtgtcgctttggctggtgctggccggactgatgctggtgttcatgttgaggtgattgatgggctgtcggggacgattgttgatggtgtgggccggttggctgactggttggcggttcgttggggtgatactgaccggatcatggttgccgggtctggtgcggtgttgttgcagaaggcgttgacggatcgtggtgttccgggccgtggcgtgattgtggctgatactggggtgtatgtggaggcgtgtcaggcgtttttggagggtgtcaggtcgggtgtggtttctcatcctcgtgccgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaggctaggcggcgtgaacggtctggtaggaagcgggtgtctgtggtatgaattcggatgagttggctctgattgagggcatgtacgatcgtatccgaaggttgtcttcgtggcattgccgtattgagggctactatgagggctctagccgggtgcgtgatttgggggttgctattcctccggagttgcagcgtgtgcagacggtggtgtcgtggcctggtattgcggtggatgctttggaggagcgtctggattggcttggctggactaatggtgacggctacggtctggatggtgtgtatgctgcgaatcggcttgctacggcgtcgtgtgatgtgcatttggatgcgctgatttttgggttgtcgtttgtggctgttattccccagggtgatgggtcggtgttggttcgtccgcagtcgccgaagaattgcacgggccggttttcggctgacgggtctcgtctggatgctggccttgtggtgcagcagacgtgtgatcctgaggttgttgaggctgagcttttgttgcctgatgtgattgttcaggtggagcggcgaggtagccgtgagtgggttgagacgggccgtataccgaatgtgcttggggctgttccgttggtgcctgttgtgaatcgtcgccgtacgtctaggattgatgggcgttcggagatcactcggtcgattagggcttacacggatgaggctgttcgcacactgttggggcagtctgtgaatcgtgacttttatgcctatcctcagcgttgggtgacgggtgtgtcggctgacgagttttcgcagcctggctgggtcctgtcgatggcttctgtgtgggctgtggataaggatgacgacggtgacactccgaatgtggggtcgtttcctgtgaattctcctacaccgtattcggatcagatgcgtttgttggctcagctgacggcgggtgaggctgcggttccggagcgctatttcgggtttatcacgtctaacccgccttctggggaggctttggctgcggaggagtcgaggcttgtgaagcgtgccgagcggcgtcagacgtcgtttggtcagggctggctgtcggttggtttcctggctgccagggcgcttgattcgagtgttgatgaggccgcgtttttcggcgatgtgggtttgcgttggcgtgacgcttcaaccccgactcgggcggctacggctgatgctgtgacgaagcttgtgggtgccggtattcttccggcggattctcgtacggtgttggagatgctggggcttgatgatgtgcaggttgaggctgtgatgcgtcatcgtgccgagtcttcggatccgttggcggcactggctggggctatatcgcgtcaaactagcgaggtttgataggcgatggcttcgggtgttgcgtcaaggttggctgctgccgggtatcagcgtgaggcggtcaggtttgccgggaagtatgcgggctattatgccgagcttggtcgtttgtggcattccgggaagatgacagatgcgcagtatgtgcgtttgtgtgtggagttggagcgtgccggccatgacggttcagcggcgttggcgggtaagttcgtgtcggattttcggaagcttaacggtgtggatcctggtttgatcgtgtatgacgagtttgatgctgccgccgcgttggctaggtcgttttcgactattaagatgatgaatagtgacccggatagggctaaggatacggttgatgcgatggcggcgggtgttaatcgggctgtcatgaatgctggccgtgacacggttgagtggtctgcgggtgcgcagggtaggtcgtggcgccgggtgacggatggtgatccgtgcgcgttttgtgccatgttggctacgaggtcggattatacgaccaaagagcgggcgcttactactggtcatactcggcgtcataagcgtggcggtaggcgtccgtttggttcgaagtatcatgatcattgtggttgtacggtggttgaggttgttggcccttgggagccaaatagggctgatgccgcatatcagaggacgtatgagaaggctcgtgagtgggttgatgatcatgggttgcagcagtcgcctggcaatattttgaaggctatgcgtactgttggtggcatgagataatttgatgtggtttccggttgtgtgccgccggttatcggtgcacagggttgtctcccgcacgggggtcaacaatgttgtgttgttttccgcaaggagtatagggttaggctatggccgatcaaaaagttgaagaacagaatgttgacaatgatgctgttgagcccggaaagggtggagacgttgttgatgttgtgaaggatgggcaggctgccggcgatgatcatgccggtgatgtttccgtgaaggaggagtcttcttctggcacggattggaaggctgaggctcgtaagtgggagtctcgtgctaaaagtaatttcgccgagttggagaagcttcgcgcctcggatggtgatgcggggtctgtgattgatgagcttcgccgcaagaatgaggaactcgaagaccggattaatgggtttgttcttgagggtgtgaagcgcgaggtggctgccgagtgtggcctgtcgggtgatgctgtcgcttttttgcacggtggcgatcgtgaagcactggtggagtctgctaaggctttgaagggtttgatcgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctttcgccggagcagccgactatttttggccctgttaagggtgccgtgtttagtggtgttcctcgcgctaagattgttggtgagggcgaggttaagccttccgcgtctgttgatgtttcggcgtttactgcgcagcctatcaaggttgtgactcagcagcgtgtctcggacgagtttatgtgggctgatgctgattaccgtctgggtgttttgcaggatctgatttccccggctcttggtgcttcgattggtcgcgccgtggatctgattgctttccatggtattgatcctgccactggtaaagcggctgccgctgtgcatacttcgctggataagacgacgcatattgttgatgccacggattctgctacggctgatcttgttaaggctgtcggcctgattgctggtgctggtttgcaggttcctaacggggttgctttggatcccgcgttctcgtttgccctgtctactgaggtgtatccgaaggggtctccgcttgccggccagcctatgtatcctgccgccgggtttgccggtttggataattggcgcggcctgaatgttggtgcttcttcgactgtttctggcgccccggagatgtcgcctgactcgggtgttaaggctattgtgggtgatttctctcgtgttcattggggtttccagcgtaacttcccgatcgagcttatcgagtatggcgatccggatcagactggccgcgatttgaagggccataatgaggttatggttcgtgccgaggctgtgctgtatgtggctatcgagtcgcttgattcgtttgctgttgtgaaggagaaggctgccccgaagcctaatccgccggccgagaactgatttattgttgcggtgatgtgtcaatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgcagggctttgttgcgctggaatgatactggcgtgtcgggtcaggtgcagtacgagtctgcgggtcctttcgctcagactacacggtctagtactcccacgaatttgttgtggccttctgagattgtcgcgttgaagaagctgtgtgagggtgatggtggggctggtaaagcgttcactattacaccgaccatgaggagtagtgtgaatcattctgaggtgtgttccacggtgtggggtgagggttgctcgtgcgggtcgaatattaacggctacgctggccccttgtgggagatatgatatgaccagttttccttatggtgaaacggttgtgatgcttcaaccgactgttcgtgtcgatgatcttggcgacaaggtggaagactggtctaagcctgtcgagactgtgtaccataacgtggccatatatgcttccgtttcgcaggaggatgaggctgcggggcgtgactcggattatgagcattggtcgatgctgttcaagcagcctgttgtgggcgctgattatcgttgtaggtggcgtattcggggtgttgtgtgggaggctgacgggtctcctatggtgtggcatcaccccatgtccggttgggatgctggtacgcaggttaatgtgaagcgtaagaagggctgatgggtagtggctcaggatgtgaatgtgaagctgaacttgccgggtattcgtgaggtgttgaagtcttctggagtgcatggcatgttggctgagcgtggcgagcgtgtcaagcgtgccgcagcggcgaatgtgggtggtaacgcgtttgatagggcccaataccgtaatggtttgtcgtcggaggtgcaggttcaccgtgttgaggctgtggcgaggattggcaccacctataagggtgggaagcgtattgaggcgaagcatggcacgttggcgaggtcgattggggctgcgtcgtgatcgtttacggtgatccgcgtgtgtgggctaaacgcgtgctcaaggatgatggctggctgtctgggataccgtgtacggggacggtgcctgaggatttcagcggtgacctgatctggttggcgttggatggtggcccacagttgcatgttcgtgagcgtgtttttttgcgcgtgaacgtgttttcggatacgccggatcgtgctatgtcgttggcgcgtcgtgtcgaggctgtgctggctgatagtgtggacggtgaccctgtggtgtactgtaaacggtctactggccctgatttgctggttgatggtgcacgttttgatgtgtattcgctttttgagctgatatgtaggcctgcggagtctgaataagcttattgtttttgttttaatgtaattgtttgatatttaatgggggttatgatggctgcaacacgtaaagcgtctaatgttcgctcagcggttactggcgacgtttatattggtgacgcgcacgcgggtgatactattaagggtgtggaggcggttccttccgggcttaccgctttagggtatctgtctgatgacgggtttaagattaagcctgagcgtaaaacggatgatttgaaggcttggcagaatgcggatgttgttcgcactgtggctacggagtcttctatcgagatttctttccagctgatcgaatccaaaaaagaggttatcgaactgttttggcagtcgaaggttactgccggatccgattcgggttcttttgatatttctcctggtgccacgacgggtgttcacgctctgttgatggatattgttgatggtgatcaggttattcgctactatttccctgaggttgagctcattgatcgtgacgagatcaagggtaagaatggtgaagtgtacgggtatggtgtgacgttgaaggcgtatcctgcccagattggtaagactggtaatgcggtgtctggtcgggggtggatgacggctttaaaagctgatactcctccttctccgaagcctcagccggatccgaatccgccggccgagaactgatacacgattttaggggattgttgatagatgagtgacactggtttcacgttgaagattggtgatcgtagctgggtgttggcggatgctgaggagacggcgcaggctgttcctgcccgcgttttccgtcgtgccgccaggattgcccagtcgggggagtctgcggatttcgcccaggttgaggtgatgttttctatgttggaggctgccgccccggctgacgctgtggaggccctggaggggcttcctatggttcgtgtggcggaggttttccgtgagtggatggaatataagcctgacggtaagggtgcctcgctgggggaatagtttggctccacggcctgattgatgattatcgtggggccatcgaatacgatttccgcactaaatttggtgtttctgtttatagtgttggtggcccgcagatgtgttggggtgaggctgtccggctggctggcgtgttgtgtactgatacgtctagccagttggcggcccacctgaatggttggcagcgcccgtttgagtggtgtgagtgggctgtgttggacatgttggatcattacaggtctgctaatagtgaggggcagccggagcctgtggcgaggccgacggatgagcgtagggcccggtttacgtctgggcaggtggacgatattttggcgcgtgttcgtgccggtggcggggtgtctcgcgagattaatattatggggtgaatagtgtatgtctggtgagattgcttccgcgtatgtgtcgttgtatacgaagatgcctggccttaaaagtgatgttggtaaacagctttctggggtgatgcctgcggagggtcagcgttcgggtagcttgtttgctagcgggatgaagttggcgcttggtggtgcggcgatgatgggtgccatcaatgttgctaagaagggcctcaagtctatctatgatgtgactattggtggcggtattgctagggcgatggctattgatgaggctcaggctaaactgactggtttgggtcatacgtcgtctgacacgtcttcgattatgaattcggctattgaggctgttactggtacgtcgtacgcgttgggggatgcggcgtctacggctgcggcgttgtctgcttcgggtgtgaagtctggcgggcagatgacggatgtgttgaagactgtcgccgatgtgtcttatatttcgggtaagtcgtttcaggatacgggcgctatttttacgtccgtgatggctcgcggtaagttgcagggcgatgacatgttgcagcttactatggcgggtgttcctgtgctgtctttgcttgccaggcagacgggtaaaacgtctgctgaggtgtcgcagatggtgtcgaaggggcagattgattttgccacgtttgcggctgcgatgaagcttggcatgggtggtgctgcgcaggcgtctggtaagacgtttgagggcgctatgaagaatgttaagggtgccctgggttatttgggtgctacggctatggcgccgtttcttaacgggttgcggcagatttttgttgcgttgaatccggttattaagtctatcacggattctgtgaagcctatgtttgcgtcggtggatcaggggattcagcgggtgatgccgtctattttggcgtggattaaccgtatgccgggcatgattacgagaatgaatgcacagatgcgcgccaaggttgagcagttgaagggcgtttttgcgaggctgcatttgcctgttcctaaggtgaattttggtgccatgtttgctggcggcaccgcagtgttcggtattgttgctgcgggtgttgggaagcttgttgcggggtttgccccgttggcggtgtctttgaagaatctgttgccgtcgtttggtgctttgaggggtgccgctggggggcttggtggcgtgtttcgcgccctgggtggccctgttggtattgtgatcgggctgtttgctgccatgtttgctacgaacgcccagttccgtgccgctgttatgcagcttgtgggggttgttggccgggctttggggcagattatggtcgctgtgcagccactgttcgggattgttgctggcgtggttgccaggttggcgccagtgttcggccagattatcggtatggttgctggtttggctgcccggctggtgcctgttattggtatgcttattgcccggctggttcctgttatcacccagattattggtatggtaacccaggttgctgccatgttgttgcctatgctgatgccggttattcaggctgttgttgctgtgatacggcaggttattggtgtgatcatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgccgtgttgcaggtggtggcccgtattattccggttgttatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatatcaaatcgtttacgtcggcgtttattaacggtttcaagtcgatcatttctgccggtgttgccgcggttgtggggttttttacgcggcttggtttgtcggttgcttctcatgttcggtctgggtttaacgcggcccgtggcgctgtttcggctgcgatgaatgctattcggagtgttgtgtcttcggtggcgtctgctgttggcgggtttttcgggtcgatggcgtctagggttcgtagtggtgctgtgcgcgggtttaatggtgcccggagtgcggcttcttctgctatgcatgctatgggctcggctgtgtctagtggtgtgcatggtgtgctaggatttttccggaatttgcctggcaatattcggcatgctctcggcaatatggggttcttgttggtgtcggctggccgtgatgtggtgtctggtttgggtaacggtattaagaatgctatgagtggcctgttggatacggtgcgtaacatgggttctcaggttgctaatgcggctaagtctgtgttgggtattcattccccgtctcgagtgtttcgtgaccaggttggccggcaggttgttgccggtttggccgaggggatcaccgggaatgcgggtttggcgttggatgcgatgtcgggtgtggctggacggctgcctgatgcggttgatgcccggtttggtgtgcgatcatcggtgggctcgtttaccccgtatgacaggtatcggcggatgggcgagaagagtgttgtggtgaatgtgaatgggcctacttatggtgatcctaacgagtttgcgaagcggattgagcggcagcagcgtgacgctttgaacgcgttggcttacgtgtgattgggggtgttgtgcatgtttattcctgacccgtctgatcgtgccggtttgactgttacctggtctatgttgccgttgattggtaatgatccggagcgtgtgcttcatttgacggattatacgggtgcgtctcctgtcatgttgttgaatgattcgttgcgcggtttgggtgttcctgaggtggagcatttttctcaaactcatgttggggtgcacggctcggagtggcgcgggtttaatgtgaagcctcgcgaggtgacattacctgtcctggtgtcgggtgttggtgtggatccggttggcgggtttcgtgacggttttttgaaggcgtatgacgagttgtggtctgcttttcctccgggcgaggagggggagttgtctgtgaagaccccgtctggccgtgagcgtgtgctaaaatgccggtttgattcggtggatgacacgtttactgtggatccggtgaacaggggttatgcgcgctatctgttgcatttgacagcttatgacccgttttggtatggggatgagcagaagtttcgttttagtaatgcgaagttgcaggattggttaggtggcggccctgtcggcaagaagggtaccgcttttccggtggtgttgacgcctggtgttggttcgggttgggataatctgtctaataggggtgatgtgcctgcgtggcctgtgattcgtgtggagggcccgttggagtcgtggtctgtgcagattgatggtttgcgtgtgtcttcggattacccggtggaggagtttgattggatcactattgatacggatcctcgcaaacagtctgcattgttgaacgggtttgaggatgtgatggatcgtttgacagagtgggagtttgcccctatcccgcctggcggttctaagagtgtgaatattgagatggttggtttgggtgccattgttgtgtcggtgcagtacaggtttttgagggcttggtgaatagttgatggctggtcttgttccgcatgtaacattgtttacacctgattatcgccgtgtggcgcctatcaatttttttgagtcgttgaagttgtcgttaaagtggaatggtttgtccactttggagttggtggtgtctggtgatcattctaggcttgacgggttgactaggccgggtgcacggctggttgttgattatggtggtggccagattttttctgggcctgtgcgtcgggttcatggtgtgggtccgtggcgttcttcccatgtgactatcacgtgtgaggatgatattcgtctgttgtggcgtatgttgatgtggcctgtggattatcgtcctggtttggttggtatggagtggcgtgctgaccgggattatgcccactattcgggtgcggctgagtcggtggctaagcaggtgttgggggataatgcttggcgttttccgcctggtttgtttatgaacgatgatgagagtcgtggacggttcattaaggattttcaggtgcggtttcacgtgtttgccgataagttgttgccggtgttgtcgtgggctcggatgactgtcacggtgaaccagtttgagaatgcgaagtttgatcagcgtggtttggtgtttgattgtgtgcctgctgtgacgcgtaagcatgtgttgactgccgagtctggttcgattgtgtcgtgggagtatgtgcgtgacgccccgaaggcgacatcggtggtggttggtggccgcggcgagggcaaagatcggctgttttgtgaggatgttgattcgatggccgaggatgactggtttgatcgtgtcgaggtgtttaaggatgcccgtaacacggattctgagcatgtgcatctcattgatgaggctgagcaggtgttgtccgagttgggggccacgtcggggtttaagatcgagttggctgagtcggatgtgttgcggtttgggcccggcaatctgatgcccggggatttgatctatgtggatgtgggttctggccctatcgcagagattgtgcggcagattgatgtggagtgtgagtcgccgggtgacgggtggacgaaggtgactcctgttgcaggggattatgagaataatccgtcggccctgttggcgcggcgtgttgctggtttggctgcgggtgtgcgggatttgcaaaaattctagaaaagattaggggtttgttgtgggtattgtgtgtaaagggtttgatggtgtgttgaccgagtatgattgggctcaaatgtctggtctgatgggtaatatgccgtccgtgaaagggccggacgattttcgtgtcggcactactgttcagggtgccacagtgttgtgtgaggtcctgccggggcaggcttgggcccacggggtgatgtgcacgtcgaatagtgttgagacggtgaccggccagcttccgggcccgggtgagacccgatacgactatgtggtgttgtctcgggattgggaggcgaatacggccaagttggagattgttcctggggggcgtgcggagcgtgcccgtgacgtgttgagggccgagcctggcgtgtaccatcagcagttgttggctactttggtggtgtcgtctaacgggttgcagcagcagctggataggcgtgctatagcggctagggtggcgtttggcgagtctgctgcgtgtgatcctaccccagtggagggtgaccgtgtgatggttccctctggggctgtgtgggctaatcatgccggcgagtggatgctgttgtcccccaggattgagacgggttctaagtcgatcatgtttggcgggtctgctgtgtatgcttacacgattccgtttgagcggccgtttagtagtgcgcctgttgtggtggcgtctatggctacggcggctgggggcacgcagcagatcaatgtgaaagcctacaatgtgactgtccaaaattttagtttggcgtttattacgaatgatggttcgaagccgaatggtgtgcctgcggcggctaattggattgctgtcggcgtgtgactgtacaggtgttgtggcggatggtgtgatgttggggggctgtggtgtcgtggtttactcctgcactggtggcctctatttgtaccgcgttggccacggttttgggttctgttcaggctgtcacatcccggtctaggcggcgtttacgcaggctgtctgcgcaggtggatgcgatggaagagtatacgtggggtgtgcggcgcgaggtgcgaaggtttaacgccgggcttcctgatgatgtggagccgatgcatcttcctgatgtgcccgagtttttgaaggatactgttgatggtggaggtgagtagggttgagggagttggaggaggagaagcggcagcgccgcaattttgagaaggcttccctgatactgttatttttgtcgcttgtgttgttggcggtggttgccgggggtgctttgcggtacgggtctgtggcttctcaaagggattcggagcaggcgagggcccagtcgaatggtacagccgctaaagggttggctgcccgtgtgaagcaggcgtgtacccagggtggcgtggagtctgtgaagctgcacaggtctggtttgtgtgtggatgctgtgcgtgttgagcagcgtgttcagggtgtgcagggtcctgccggtgagcgtggcccgcaagggcccgctggtgttgatggccgggatggtagcaatggttctgctgggctggttggccctgttgggccgcagggttcccctggtttgaatggtgttccaggtcgtgcaggtgtcgatggtgtgaacggcgc

SEQ ID NO: 71 - DNA, 29596 bp, synthetic construct
  source 1..29596: mol_type = other DNA; note = PAC2
ctggtttgaatggtgtgaaaggtcctgacgggttgcctggcgctaacggttcggatggccgtgatggtgttccaggtcgtgcaggtgtcgatggtgtgaacggcgctgatggtcgggatggttcggccggtgagcgcggtgatgtgggcccttcaggtcctgccggcccgcaaggtgcacagggtgaacggggtgagcgcggccccgccggtgcgaacggatccgatggtaaagacggtaaggatggtgctgatggccgtgatgggcgttcggtgatatcggtgtactgttccgggggccgcctggttgtgaaatatagtgacggtacggcctctaccgtgtcgggttctgcggcctgcgagagtgtgaaaccatcacctgtggttactgtatcatcccataggtgaacaagaagagggaagggtgttactagtgttgattgtggtgtttgggggtggtgtgtggtgagatacattccagcggcgcatcactctgccggttcgaatagtccggtgaacagggttgtgattcatgcaacatgcccggatgtggggtttccgtccgcttcgcgtaagggtcgggctgtgtctacagcgaactatttcgcttccccatcatcggggggttcggcgcattatgtttgtgatattggggagacggtgcagtgcctgtcagaggggactatagggtggcatgccccgccgaatccgcattctttgggtatagagatttgcgcggatgggggttcgcatgcctcgttccgtgtaccggggcatgcttacacgagggagcagtggcttgatccgcaggtgtggcccgcagtggagagggccgctatcctgtgtcggcagttgtgtgacaagcatggtgttccgaaaaggaaactgtctgtggccgatttgaaggccggtaaacggggtgtgtgcgggcatgtggatgttacggatgcgtggcatcagtcggatcatgacgatccggggccgtggtttccgtgggacaaatttatggctgtggtgaatggccacggcggcggttcaagtagtgaggagttgagtatggctgatgtacaagcgttacataatcagattaaacagttgtcggcacaggtggcccagtcggtgaataagctgcatcacgatgttggtgtggttcaggttcagaatggtgatttgggtaaacgtgttgatgccctgtcgtgggtgaagaatccggtgaccgggaagctgtggcgcaccaaagacgccctgtggagcatctggtattacgtgttggagtgtcgcagccgcatagacaggcttgagtctgctgttaatggtttgaaaaagtgatggtggtttgttgtgggtaaacagttttggttgggcttgtttgagcgtgccctgaaaacttttattcaaacgtttgttgctgtgcttggggtgacggcgggtgttacttatactgcggagtcgtttcgcggtttgccgtgggagtctgccctgataacagcaacggttgctgcggtgctgtctgttgctacatcgtttggtagcccgtcatttgtggccggcaaacctaaaaccacggttgtggatgctgggcttgttccacccgacgatgggggcatggttgagccgcactcggtggatgtgtcggatcctggcatgatcgagccgacagatgatgtggatggttttgctggctatgtgccgaagcgtgcagccgagtcggaggttagcacggtggagtctactgttgcataattgaacatagatgtgtgccccagcggtgctgccacgatcgtgtggtggttgccgctggggcacactttttgtgtctataggagttttacaggttgtcgtctagtgtgtcttcgagcatctggtccaggtagaggcaggcggagatagtatcgttggcctggtctagaacgttctggccgataacatttttatgattgtcgcggtggctgatgatagaccgcatgatatcgtcggccgccgcctgcaatagtttggcctggtatgcgattcctgcgagccagtctagtgcttcctggcttgccagtgtgtcgtctggaatgccacgggtgttgctgttgtttgtggggtgtcctgcactgtcgcagcaccacaagatttcgctgcactcgtctagcgtgtcctggtcgatagcaagatcgtcgaggctgacttctttgacggtaaggttcacattgtcgagggagatgggtacaccgtattggttttcgacactgtcaacaatgttttccaactgttgcatgttggtgggctgttgttggatgatacggtgtactactgttttgatggcggtgtaggggatattgtgtgtgttgttcatggtttttatcccacccctgtgttgtcgtcgttattgtctggatagtatctactgtttgcgtagcctgtgagggtgatgagtgtttggtctgcccactgtttcactgtctgccgggtgacacccaatcgttgggcggctgtggcgtaggtttgatcatacccgtatacttcacggaatgcggctagcctggctaggtgttttcgctgtttggagggttcacatgatagggtgtagtcgtcgatggcgagctgtagatcgatcatggtggcaatgttgttgccgtgatgctggggggcggttggtgggggtggcattcctggctccacactgggtttccatgggccgccgttccagatccattgggcggcttggatgatgtcggcggtggtgtaggttcggttcatgtgtcaccccctgaacaggtcgttgctggtgctggtgttggtggtgtcgaatcgtccgacgcagtggcagtagtcgtacatgagtttgataatgtgttggtggtctcccaaataggtgtttccgctgatactgtaggtggctgtgccgtctttactgatggtgtatttggcggtgatggtttcggggttttcggtgtcggtgatgattgctgtggtggtggtgcctactgtttgtagcacggtggtttgggttccgtcgtcgatagtggttttaaccatggtgtgtgttctccctttttagatgctggtttggttgtcggctagatgaatgatgtcgggtaagggtttcggctggtctaggtgttgtatggttttgttggctagccgtttggctaccctgtaacacattttggtgtagtgtttgttgtctaggttgtggtattgttcccgcaccgcaatatatagtagagagtcttggtacaggtcgtctgcactgattgcggggtagtgtgcggctgttttggtgcatgcccggttgagtgtgcgtagatgatggtctgtggcccacacccacgatgcggtggtggctaggtcggcttttgttggtcgtctgctcatggcatttctttcatcgggctatctggtagttgtttggtgttttgttgttgatagtgtagcacacgagtccggggtttccggtggtgccagtcttgtgccggtaccatgtggattcgccttccatggatgggcattggatgaaggtgcgttgtccttgttcggagatttctaggtggtgccggtgcccggccatgaggatgtgggatgtggtgccgttgtggaattcttggccgcgccaccaatcatagtgtttgccggtgcgccattggtgtccgtgggcgtgcaggatttgtgtgccggccacgtcgacggtggtggtcatttcgtcccgttgggggaagtggaagtgaaggttggggtattggttgttgagctggtaagcttctgcgatggcgcggcagcagtccacgtcgaaggagtcgtcgtaggtggtgactcctttgccgaagcgcacggcttcgccgtggttgccggggatggatgtgatggtcacatttttgcagtggtcgaacatgtggacgagttgcatcatggccatgcgggtgagcctgatttgttcggtgaggggtgtttgtgtgcgccaggcgttgttgcctccttgtgacacgtatccttcgatcatgtcgccgaggaatgcgatgtggactcgttcgggtttgcctgcctgttgccagtagtgttttgcgactatgagggagtgcaaatagtcgtctgcgaatcggctggtttctccgccggggatgcctttgccgatttggaagtcgcctgccccgataacgaaggctgtctcgtcactgctttgggtgtcttgttcgggtttgggtggctgccattcggctagtttgttgacgagttcgtcgacggggtaggggtcggttgcgggttggtggtcgatgattttttgtatggatcggcctgtttctccgttggggagtgtccattcggagatgcgtgtgcggcgtacagtaccgttggctagattgtcgtcgatggtgtcgatggcgttgtcgtggttggctagctgtgtgagtagccggtctatattgtctatcactggttttcctcctctggcggggtggtgttggcttgtttgcggcggtagtcttttataacggtggcggagatggggtatcctgcctgggtgagctgttttgctagccacgaggcgggtatagacctgtcggcgaggacgtctgcagccttgttgccgtagcgttgaataagggtttcagttttggttgccatgatgtcctatcggttgtgtggcgggctgccatcctgtgcggcagtcgccgtcgtggcctggtttgcgtgtgcaccacgatacggttccgtctgtgtggttgagtgttttgccgcacatgacgttttgtagatgctcgggcagggcgccgtcaccctggttgctggtttgtgtgtcgaagagtgttttctggttggtgaaatgctctgacacggtgccgttgtgtacgggtagtatccatgttttccattgttgttgtagccgggtgttccagtggaattgtttggccgcgttcgtggcttgtttgatggttttgtagtagccgacgaggatgcgctggtgttcactgtcgggtgggttttggcctcgccagtattgtgccgcgacggcatacctgttgttgtctgtgaaggcgtcccagcagtattcgataatgtgttgtagtacactatcgggaatgtctcgtacttggttttcgtcgagccacgcgtcgacaatgatgttgcgtatggcgtgtttgtctttggtggtgggtttgaacgagatactcaccatgctggcctgtcgtcttgcatgaaatcgttaaaggatgattcgcttgtgcggcgtgcctgggtgatttgctggtcagtccagtcggggtgttgctgtttcagatagtaccagcggcaggcatcatatgtttcgttctgcaagcgggtgagatggttttcggtgatgatttgtttccacattgtccacgagacgtcgagcctgcggagcatgtccatggccggcacattaaacgagtcaaggaagagtatttcgtgggtgtagtagtttttctcgtaggcgtaccatccgcttcggtgcctgtggggctggtttttggggtaggcttcccggcatactttgtgtaaacgtttggccatgtcgtcgggtagttcaatgtcggggttggcgcggatcatggatcgcatcccgtcgtaggtggtgccccaggtgtgcatgatgtgtagtgggttgtctccatcggcccatttttctgcacagatggcgaggcggatgcgcctcctggctgtttggctggtgttgcgccggttggggattgggcacgtgtcgaggggatccattatgttttagtgtacctttctggtttcgtgttgttgacgtgttttactgtagcacagtgtctagtgcttgtgtcaaccctgtttttccggcctgcaggtaggtgtctgtgacatctccgaccgtgaggggcacatgggtggcttgggggagtgctgcctggatggtttgtgccatctggtcgcctgcggggtctgggtctgaccagatgtagatgtggtcgtagccttcgaagaatttggtccagaagttttgccacgaggttgcgccgggtagggctacggccggccatccgcattgttcgaggatcatggagtcgaattcgccttcgcaaatgtgcatttcggctgccgggttggccatggcggccatgttgtagatggagcctgtgtccccggctggggtcaagtatttggggtggttgtgggttttgcagtcgtgtgggagtgagcagcggaaacgcatttttcgtatttcggctggccgctcccaaacggggtacatgtatgggatggtgatgcactggttgtagttttcgtggcctggtatggggtcattgtcgatgtatccaaggtggtggtagcgggctgtttcttcgctgatgcctcttgctgagagcaggtcgagtatgttttcgaggtgggtttcgtagcgggctgaggctttctggattcggcggcgttccgcaatgttgtagggttgtatgctgtcgtacattcgggttttcttcttctagtcgttgttgtagtttgtggagtcctcctccgacaccgcatgtgtggcagtaccagacgcccttgtcgaggttgatgctcatggagggctggtggtcgtcgtggagcgggcagagtatgtgttgctcgtttttggacgggttgtagcgtatctggtagatgtcgaggatgcggcgggtgtcagaggtgtgggaggagctcgttgagggttgataccacataggcttcgctccagggtttgttgcgttgtttcatcactacgagtccgatggtggaattgttttcgcggtttcggtgtgtttcgtagttgcgtgcctcccggctggcttgtttcacgaattcggctaggtggggctggccggctttcgcctcgataatgtaggttttgttgctggttgtgaggatgaggtcgccttcgtcttcgcggccgttgaggtggaggcgttcgatatcgtgtccggtgtcgcgtagctggtgcaataatcgtgtttcccattcggctccggcccgccggttgcgtgcctgctgtgtggccatagtttttagagtcctttgtgtgttgtggtcatgttccagggctgtttttcggcgaggggcccgaagaatgtgtattcggggtaggctcgtagtcgttcatatcgggtgccgtcggggctggatttgccggtgcgctgtttcaatactgcgatgcgtgcctcggccggtatcgtgagaccgttgccgttatcctcgccaccatacaatgagactcccaatatgagttgtggtttttcggagaggccgtttttgatttctcgccgtgccggggggtgttcgatgtcggttccggttttgtcggtggcgtggtgtgtgacaataatggtggatccggtgtcgcggcctaatgctgtgatccattgcatggcttcttgctgtgcctgatagtcactctcgcagtcttggatgtccatcaggttgtcgataacaatgagtggcgggaaggtgttccacatttccatgtaggcttgcagctccatggtgatgtctgtccatgtgatgggtgactggaatgagaatgtgatgtgttggccgtggtggatgctgtctcgatagtattctggtccgtagtcgtcgatgttttgttgtatctgtgtggtggtgtgttgggtgttgagtgagatgattcgtgtggaggcctcccagggtgtcatgtcccctgatatgtagagggcgggctggttgagcatggcggtgatgaacatggctagcccggatttttggctgcctgagcgccccgcaatcatgacgagatcccctttgtggatgtgcatgtcctggttgcggtagaggggttctagttgtggtatgcggggcagctcggctgcggtttgggaggctctctcgaaggatcgttggagagagagcatcgggaccttatctatctatcggttgggtgtgttttggtggtcagatggagtcgatgtcgatgtcagcatcggcgggggttgagcacgaccgcattgaacccgtttttggtgcgcacggtggcgagtttgaaggcctgctcctcgccaaggtaggcttcgaggtcgcggatcatggaatgtgggcggtcgttgttgccgcgcgctttctcaataatagcgttgggaatgatttctggggtgccgttgttgagatcgtctagggtgtggaagattgtgacatcagcgtagatgcgatcggctgtctgtccaccgtagccttcggtgttgtgttctacgtcgcggattttgaaggcgatggcggtggcgtcctggtttcgggaggggttgaagaaggtgctgttgctgttgttgcggtagttggcgagtcccatggttgtttcctttactgtttgtgttggtttgtgtcggttttatcgggtgaggctgtttcgtttgctgcggaaagcctcggacacgtcactgttactggtgatgattttcttgtactgtttcagaaggtcggctagctgtgccttgcttgttgcattgttgattttgtcgatgataatctcgttttcgtttgatgcgatgttgtctacgtagtctttggctgcctggttgtagcggtcttggaggatgatggatgcgcttgctacgagtgttgctagatcccagtctttggacacgtcaccgtttttgaggccgcctagcagatcaataatggattgtttgatgtcttctgcggtgtctccgcggatgactgtccatggggctgcgtagtctccaccgtatttgagtgtgatagttagctttccgctgtctgtggtgtgctcgtcggtcacgtgttttccttttcgttgttttcggcttctggtggctgtacggtggtttctaccgggtatctgtacgagtttttcccgttgacggcccagcaggcgtccttgacggggcatcctttgcagagtgctgtgacgtggggtacgaagatgccttggctgattcctttcattgcttgactgtacatggatgatacatgccggtaggtgttgttgtcaagatcaatgagttcggtggatgtgccctgctcaaccgattgctcgtctcccttggtggtagcgggtgtccaaaacattcctttcgtcacatggatgccgtgttggttgagcatgtaacggtaggtgtgcagctgcatactgtcggcgggtaggcgtccggttttgaggtccaaaatgaaggtttcacccgtattcgtatctgtgaatacccggtcgatgtagccaacgatctgggtgccgtcggggagggtggtttctaccgggtattcgatgcccggctcgccgtcaataacagcggtagcatattctgggtggttgcgcctccatgttttccaccggtccacaaaggtggggccgtaaatcatccaccaattgtagtctttcttgtgtgtcccgcccgactcgcacatgtttttgcatattctgccggagggtttgatttctgtgccttcggattcggcgagggcgacttgggtgtcgaaaatgtttttgaaggatgagagtttgtctggcagtgcagggtattcggcgggattgtacaggtgtaggtcgtattgttcggtgatgtggtgtatggcgcttccggcgatggtggcataccaggtgtggtgttgggcgtggtagccgtgggataggcgccatttttcaccgcattcggcccactgtgacagtgatgagtaggagatgtggcctggatggtcaatggtggacggtttttgtgctaggggcattacttgtcgcttttgtgggtgttccatgggtttcgggtgtcttggccggcattgtgttgctggtatgcgaggagtgcgaggcagtgccaggcagcatgggccagatggggtagcccggattcatcatcgaggttgttgccttgctgccatgataacaggtgccggtagagggcgtcaacactgtggctccacggatagccgccggtccagttgttgtcgccgtatttggtggcgccgtatccggccacagagccgagggcgtgtaaggctgtagggtcgatgagggatagcctgcaaagtttcaattctttcttggcgccagtatcagggtcggtgtacatgctggtgggctcatccatggtgtgtgtgctccttaagtatggggttactggttggggttgtgggcgagtgctacggcaagaataatgatggcgagggtttcagcgatcagtatgggtgttgtgatcatttgtggtcgcggggattgttggtgagggttgaggcgcccaggaggatagtgagggcgcatgcggcgatgatggcgagggctgccttgtgtggggtgccggtggcgtacatccatgtgatgatgccgccttggatccaggcgaggctggtgaagaacgtttcgtagctgtgtagctcaatgttgttgttgggtgtgttcatgcttgctcctgaagaatggtgttgatggttgtgtaaatgttgtacaggtcggtttcgatagataacagttggtggatttggtggtcgagatcaatgtcggggttgagggtgttgatgcgggaggcgatgtcggtggctgtgcgtagtgtgccgccggtgtggtgaatgatgtgtgccgtgtcggcgagtccggtggtgacagtgtagtgggagaggagaggcatagctgggggtgctccttgacggggttactgttgcgggttgatgttgaggtcggtgacgttggggtggtcttctgttccggtgacaaggcagtggacggtgactgggagtttggatgcgccgggctgtttcgcggttgcgccgtagacgatggagaaggtgtctttgccaataattttgtggagttggaggtcgatgtcggggttgccgttccatttgacgccttgtgtggcggcctgttgttcggctttgcggttgcaggtgtgtgctgcggtgatcatggtgagtccggtggcggtttcttcaccccttgcttgggcttgcttgtgggttttctgctgttcggctcgcagtgactgttctgctgctgcctgccgtgctttcttttcggctttgcgctgttgggtagtcttgggggtccattcggtgttggctgtggtggcttgcggtgcgggttgtgatgcgagtggcggattgtcgtctggggctggcatgaaggatgctgcggcgatgatggcggctgtgattccggcgatggtgtagccgtttttcttgttcatgattttgtgttcccctttccggggtgttgttcgttgctgacatgattaatactttcagcggctgggcccactgtcaaggctgcgctcaacgattgtgagcgatacttgtgtggctaggggttttgtccttgaggtgggagatgtctttcccttgcgtccagtatccatggcggttgcgagtcatccctttggcgagcatctcgtccacggtgagacacctgcgacgatctggaccctccttgactccctgatcgcctgtgcggtgcatgtcaccggcacaagtaccattaaatgtctcgtggcggatggtgtgatgctctggtcggtatccgatgattgtgctatcgcacttgtggcatgtccattgcatgattggtccttctttcgtgttttaagcttgtactctgaggattagagcgactttcagcccttggggggtatgattatataggtcaggtatttctaggcgattctaggctcattgtgtgtggctgggggttatcgggcacacagggtgaggagttggccaacattgatgcgggtcacattccagtagagttgcgtggcttccccaccggtgagtggcttccactcgtcatggctgaacacggtgccgtcggttgcgatgaatgtgttggggcgtagcttgtgaagctcagtctctacacgctgccggtaggcttcggcgaggccctcgaaatccatgtggtcgcaggggaggttttcgaggcgtgtcaggtcgaagggtgtggggcagtcgtagctggcggggctgtagagctgggtgaaatggttggcgatcttctgcatgacgggttccttttctcgtatggtgagttgatagttttatcgggtggatgcgacaaggatggcgtctacatcgatcatgtcgatgagatcgtggagttcctcggcctcattctcggagaggtggcgccagccatagtcgccgtatacggcgccgtcgagggtgacagtccacaggggccggatgagtcgtatggcttcttgtactttagcgtggtacatgcggcgcaccatatccagatcgatgtcgtctgaatggtttccggtgaggctgtagaggctgagcgggtcgatttctgtctgcctgtagagggatgtgaatgatggtgtgatgagtgtgccatccatgagagtgtgctcctttcggtggtggaggggttgttgtggtttctagagtgtgtaggctgcgacccatagtcaaggctgcgctcattcggattgagcgtttcatatgggtgtggcatggaatctacacccccatactgtgtgagataggccacatcctcctggcttggtgtgaaccctcgagactactctgcctatctggcgtggagggtgtagcccagaaataccgtttaaagccttcatacggcgcctaggagcgccttacagggtgggggctaggtatttatacccccaagcaattctgatcgattctagacgcctcccaggagcccgatacacgatccgctatccagacacagatcatcagcccctatcctggttagctaagcctcaactatgtggacagtgttgattactgtggggtaagaaggacacggtaaaagaaagaggggggagcatcggccttcaagccttaaggtcttagcagttagcaccgagcccctcaagggctcgtcgtcagcccatcaggcacggccctgaacggggtacacgccatcagggaaggcttgagagtacgaggagccttagcgacgagtactcgaaagcctgagggaacaccctcagcactgatgggtctagcgtgttcggaaaggacacaggagtaaagcgtgacagctgtccgggagtgaaacccgttctgactaggggtttcagccttaaccaccctcaaaggttacaagactctaagaaaatttaaggaaaagtttaggtttaatttttggacctttactaccaaaaacacccgtttacacccctcaaacccgcctatagagccaaatccaccagtttgactcatcccaggtggcatatgataggctggacaggtagccagctggacgcaaggccgaaatccgctgacgcggctttcacccttacatccatcagtctaccaaagacttaaagacctaagggcttagcgctaaggtgctgatagcttagcaccgagcccttgaggggctcggcatcagccctaaagccttaaacacttaaagtacatataaaactttaaaagcttaacacttaaggttataaataaacattaaagctttaaagtcttaaagtacatatataaccttaacacctaagttaagtataaaaccttaaaggcttagcactgaaggatataaacttcacatcagtttttaagactttaaaacttaaaataactattaagacttaaagacttataagttttaaacacttaaagtaactataagactttaaagaccttaagtacttaaagttaaccatcagtcttaaactttaatattataacctataagtcttaaagcttataagttataaaagttttagaagagctaagaggttaacttctttacttctcttctctctttggttctttctctcttctcttcttttcttcatcaggggagaagaggaaccttttaccatcagcgccgatggactgtcaccgtgtgactcgtgtaccaccggtcgcacgctcccggtttcacactccccacactctgacacccgtgtccctttcaggcttagcgtgttcggctgaaggcgtacggcgtgtcgcgccaacacccttaacaccaggtaagacttaaagtgtatattatatgtagaagactttaaaacctataaggtgttcccgcttagcctgtgtcctacaccgctaggcgccaagcgttaagtcttgaaacgcgaacacacacccacccccatttttctttcgtgtccttctcttttgacaccgctggggggcgatgtgatctttctcactacccccatgggtagtggagaacacacccaccccaccatcaacagaacaccccctcaaacgaacaaaacagggcctagaatcgatcggcagggcaagggcaaggtattcatacccccaacacattccaggccgtcagagaggcaaataagacccgtacagggctagtcgaggatcggagacgtgatggcacacaccaatcgcaccgcatccgccgcacaccgacactggcggcaacgactcatcacccaagcccgacagcaaggccaaaccgaatgcccactctgcggagcaaccatcacctgggacacctaccagctgccaactagccccgaagccgaccacatcacacccgtcagcaggggaggactcaacaccctcgacaacgggcaaatcatctgcagaacatgcaacagaagcaaaggcaacagaacacaaccaaacatcaaattccaacaacaaaccacaaaaaaccttgttccatggtgacaaaacccgccaacccccaccggggacaccccctgcacacccgtgcaagacctcgtacggcttagtgaaatacctcccttttgtggatttgtctgtttgtcgactttttgtgttggtggtgagtgttgtgcagcctgagcttcctgagggacacgagtggtgtggggagacgcgtcgttggtggcgtgtgtggggtgaggatagccgcgcgcagtacgtgtctgatgaggagtggctgtttcttatggatgctgcggtgattcatgattgtgtgtggcgtgagggtcgcgcggatttggtggcttcgcttcgtgctcatgtgaaggcttttatgggtatgttggatcgttattcggttgatgtggcgtctggtggccgtggtgggggttctgcggtggcgatgattgaccggtataggaagcgtaggggggcctgattaggtgtctggtgttgttgggtctcaggttcctcgtcatcgtgtggctgcggcgtattcggtgtctgctggcggtgatgctggggagttgggtcgtgcgtatgggttgacgcctgatccgtggcagcagcaggtgttggatgattggctagctgtgggtggtaatggcaggcttgcttcgggtgtgtgtggggtgtttgtgcctcgccagaatggcaagaatgctattttggaggttgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgtgcgacgaatggccaggaggctattgtgttgcatcatccggattgtgccacgtttgagcgtaagtgtggttgtccgggttggggttcggttgagtttgtggcccgttctcgtggttctgctcgcgggtttacggttgatgatttggtgtgtgatgaggctcaggagttgtcggatgagcagttggaggcgttgcttcctacggtgtctgcggctccttcgggtgatcctcagcagattttcttgggtacgccgcctgggccgttggctgacgggtctgtggtgttgcgtttgcgcgggcaggctttgtcgggtggtaaaaggtttgcgtggacggagttttctatcccggatgagtctgatccggatgatgtgtcgcggcagtggcggaagcttgctggtgagacgaatcctgcgctgggtaggcgtctgaatttcgggacggtgagcgatgagcatgagtcgatgtctgctgccgggtttgctcgggagcggcttggctggtgggatcgtggccagtctgcttcttcggtgattccggcggataagtgggttcagtcggctgtggatgaggcggctctggttggcgggaaagtgtttggtgtctcgttttctcgttcgggggatcgtgtcgctttggctggtgctggccggactgatgctggtgttcatgttgaggtgattgatgggctgtcggggacgattgttgatggtgtgggccggttggctgactggttggcggttcgttggggtgatactgaccggatcatggttgccgggtctggtgcggtgttgttgcagaaggcgttgacggatcgtggtgttccgggccgtggcgtgattgtggctgatactggggtgtatgtggaggcgtgtcaggcgtttttggagggtgtcaggtcgggtgtggtttctcatcctcgtgccgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaggctaggcggcgtgaacggtctggtaggaagcgggtgtctgtggtatgaattcggatgagttggctctgattgagggcatgtacgatcgtatccgaaggttgtcttcgtggcattgccgtattgagggctactatgagggctctagccgggtgcgtgatttgggggttgctattcctccggagttgcagcgtgtgcagacggtggtgtcgtggcctggtattgcggtggatgctttggaggagcgtctggattggcttggctggactaatggtgacggctacggtctggatggtgtgtatgctgcgaatcggcttgctacggcgtcgtgtgatgtgcatttggatgcgctgatttttgggttgtcgtttgtggctgttattccccagggtgatgggtcggtgttggttcgtccgcagtcgccgaagaattgcacgggccggttttcggctgacgggtctcgtctggatgctggccttgtggtgcagcagacgtgtgatcctgaggttgttgaggctgagcttttgttgcctgatgtgattgttcaggtggagcggcgaggtagccgtgagtgggttgagacgggccgtataccgaatgtgcttggggctgttccgttggtgcctgttgtgaatcgtcgccgtacgtctaggattgatgggcgttcggagatcactcggtcgattagggcttacacggatgaggctgttcgcacactgttggggcagtctgtgaatcgtgacttttatgcctatcctcagcgttgggtgacgggtgtgtcggctgacgagttttcgcagcctggctgggtcctgtcgatggcttctgtgtgggctgtggataaggatgacgacggtgacactccgaatgtggggtcgtttcctgtgaattctcctacaccgtattcggatcagatgcgtttgttggctcagctgacggcgggtgaggctgcggttccggagcgctatttcgggtttatcacgtctaacccgccttctggggaggctttggctgcggaggagtcgaggcttgtgaagcgtgccgagcggcgtcagacgtcgtttggtcagggctggctgtcggttggtttcctggctgccagggcgcttgattcgagtgttgatgaggccgcgtttttcggcgatgtgggtttgcgttggcgtgacgcttcaaccccgactcgggcggctacggctgatgctgtgacgaagcttgtgggtgccggtattcttccggcggattctcgtacggtgttggagatgctggggcttgatgatgtgcaggttgaggctgtgatgcgtcatcgtgccgagtcttcggatccgttggcggcactggctggggctatatcgcgtcaaactagcgaggtttgataggcgatggcttcgggtgttgcgtcaaggttggctgctgccgggtatcagcgtgaggcggtcaggtttgccgggaagtatgcgggctattatgccgagcttggtcgtttgtggcattccgggaagatgacagatgcgcagtatgtgcgtttgtgtgtggagttggagcgtgccggccatgacggttcagcggcgttggcgggtaagttcgtgtcggattttcggaagcttaacggtgtggatcctggtttgatcgtgtatgacgagtttgatgctgccgccgcgttggctaggtcgttttcgactattaagatgatgaatagtgacccggatagggctaaggatacggttgatgcgatggcggcgggtgttaatcgggctgtcatgaatgctggccgtgacacggttgagtggtctgcgggtgcgcagggtaggtcgtggcgccgggtgacggatggtgatccgtgcgcgttttgtgccatgttggctacgaggtcggattatacgaccaaagagcgggcgcttactactggtcatactcggcgtcataagcgtggcggtaggcgtccgtttggttcgaagtatcatgatcattgtggttgtacggtggttgaggttgttggcccttgggagccaaatagggctgatgccgcatatcagaggacgtatgagaaggctcgtgagtgggttgatgatcatgggttgcagcagtcgcctggcaatattttgaaggctatgcgtactgttggtggcatgagataatttgatgtggtttccggttgtgtgccgccggttatcggtgcacagggttgtctcccgcacgggggtcaacaatgttgtgttgttttccgcaaggagtatagggttaggctatggccgatcaaaaagttgaagaacagaatgttgacaatgatgctgttgagcccggaaagggtggagacgttgttgatgttgtgaaggatgggcaggctgccggcgatgatcatgccggtgatgtttccgtgaaggaggagtcttcttctggcacggattggaaggctgaggctcgtaagtgggagtctcgtgctaaaagtaatttcgccgagttggagaagcttcgcgcctcggatggtgatgcggggtctgtgattgatgagcttcgccgcaagaatgaggaactcgaagaccggattaatgggtttgttcttgagggtgtgaagcgcgaggtggctgccgagtgtggcctgtcgggtgatgctgtcgcttttttgcacggtggcgatcgtgaagcactggtggagtctgctaaggctttgaagggtttgatcgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctttcgccggagcagccgactatttttggccctgttaagggtgccgtgtttagtggtgttcctcgcgctaagattgttggtgagggcgaggttaagccttccgcgtctgttgatgtttcggcgtttactgcgcagcctatcaaggttgtgactcagcagcgtgtctcggacgagtttatgtgggctgatgctgattaccgtctgggtgttttgcaggatctgatttccccggctcttggtgcttcgattggtcgcgccgtggatctgattgctttccatggtattgatcctgccactggtaaagcggctgccgctgtgcatacttcgctggataagacgacgcatattgttgatgccacggattctgctacggctgatcttgttaaggctgtcggcctgattgctggtgctggtttgcaggttcctaacggggttgctttggatcccgcgttctcgtttgccctgtctactgaggtgtatccgaaggggtctccgcttgccggccagcctatgtatcctgccgccgggtttgccggtttggataattggcgcggcctgaatgttggtgcttcttcgactgtttctggcgccccggagatgtcgcctgactcgggtgttaaggctattgtgggtgatttctctcgtgttcattggggtttccagcgtaacttcccgatcgagcttatcgagtatggcgatccggatcagactggccgcgatttgaagggccataatgaggttatggttcgtgccgaggctgtgctgtatgtggctatcgagtcgcttgattcgtttgctgttgtgaaggagaaggctgccccgaagcctaatccgccggccgagaactgatttattgttgcggtgatgtgtcaatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgcagggctttgttgcgctggaatgatactggcgtgtcgggtcaggtgcagtacgagtctgcgggtcctttcgctcagactacacggtctagtactcccacgaatttgttgtggccttctgagattgtcgcgttgaagaagctgtgtgagggtgatggtggggctggtaaagcgttcactattacaccgaccatgaggagtagtgtgaatcattctgaggtgtgttccacggtgtggggtgagggttgctcgtgcgggtcgaatattaacggctacgctggccccttgtgggagatatgatatgaccagttttccttatggtgaaacggttgtgatgcttcaaccgactgttcgtgtcgatgatcttggcgacaaggtggaagactggtctaagcctgtcgagactgtgtaccataacgtggccatatatgcttccgtttcgcaggaggatgaggctgcggggcgtgactcggattatgagcattggtcgatgctgttcaagcagcctgttgtgggcgctgattatcgttgtaggtggcgtattcggggtgttgtgtgggaggctgacgggtctcctatggtgtggcatcaccccatgtccggttgggatgctggtacgcaggttaatgtgaagcgtaagaagggctgatgggtagtggctcaggatgtgaatgtgaagctgaacttgccgggtattcgtgaggtgttgaagtcttctggagtgcatggcatgttggctgagcgtggcgagcgtgtcaagcgtgccgcagcggcgaatgtgggtggtaacgcgtttgatagggcccaataccgtaatggtttgtcgtcggaggtgcaggttcaccgtgttgaggctgtggcgaggattggcaccacctataagggtgggaagcgtattgaggcgaagcatggcacgttggcgaggtcgattggggctgcgtcgtgatcgtttacggtgatccgcgtgtgtgggctaaacgcgtgctcaaggatgatggctggctgtctgggataccgtgtacggggacggtgcctgaggatttcagcggtgacctgatctggttggcgttggatggtggcccacagttgcatgttcgtgagcgtgtttttttgcgcgtgaacgtgttttcggatacgccggatcgtgctatgtcgttggcgcgtcgtgtcgaggctgtgctggctgatagtgtggacggtgaccctgtggtgtactgtaaacggtctactggccctgatttgctggttgatggtgcacgttttgatgtgtattcgctttttgagctgatatgtaggcctgcggagtctgaataagcttattgtttttgttttaatgtaattgtttgatatttaatgggggttatgatggctgcaacacgtaaagcgtctaatgttcgctcagcggttactggcgacgtttatattggtgacgcgcacgcgggtgatactattaagggtgtggaggcggttccttccgggcttaccgctttagggtatctgtctgatgacgggtttaagattaagcctgagcgtaaaacggatgatttgaaggcttggcagaatgcggatgttgttcgcactgtggctacggagtcttctatcgagatttctttccagctgatcgaatccaaaaaagaggttatcgaactgttttggcagtcgaaggttactgccggatccgattcgggttcttttgatatttctcctggtgccacgacgggtgttcacgctctgttgatggatattgttgatggtgatcaggttattcgctactatttccctgaggttgagctcattgatcgtgacgagatcaagggtaagaatggtgaagtgtacgggtatggtgtgacgttgaaggcgtatcctgcccagattggtaagactggtaatgcggtgtctggtcgggggtggatgacggctttaaaagctgatactcctccttctccgaagcctcagccggatccgaatccgccggccgagaactgatacacgattttaggggattgttgatagatgagtgacactggtttcacgttgaagattggtgatcgtagctgggtgttggcggatgctgaggagacggcgcaggctgttcctgcccgcgttttccgtcgtgccgccaggattgcccagtcgggggagtctgcggatttcgcccaggttgaggtgatgttttctatgttggaggctgccgccccggctgacgctgtggaggccctggaggggcttcctatggttcgtgtggcggaggttttccgtgagtggatggaatataagcctgacggtaagggtgcctcgctgggggaatagtttggctccacggcctgattgatgattatcgtggggccatcgaatacgatttccgcactaaatttggtgtttctgtttatagtgttggtggcccgcagatgtgttggggtgaggctgtccggctggctggcgtgttgtgtactgatacgtctagccagttggcggcccacctgaatggttggcagcgcccgtttgagtggtgtgagtgggctgtgttggacatgttggatcattacaggtctgctaatagtgaggggcagccggagcctgtggcgaggccgacggatgagcgtagggcccggtttacgtctgggcaggtggacgatattttggcgcgtgttcgtgccggtggcggggtgtctcgcgagattaatattatggggtgaatagtgtatgtctggtgagattgcttccgcgtatgtgtcgttgtatacgaagatgcctggccttaaaagtgatgttggtaaacagctttctggggtgatgcctgcggagggtcagcgttcgggtagcttgtttgctagcgggatgaagttggcgcttggtggtgcggcgatgatgggtgccatcaatgttgctaagaagggcctcaagtctatctatgatgtgactattggtggcggtattgctagggcgatggctattgatgaggctcaggctaaactgactggtttgggtcatacgtcgtctgacacgtcttcgattatgaattcggctattgaggctgttactggtacgtcgtacgcgttgggggatgcggcgtctacggctgcggcgttgtctgcttcgggtgtgaagtctggcgggcagatgacggatgtgttgaagactgtcgccgatgtgtcttatatttcgggtaagtcgtttcaggatacgggcgctatttttacgtccgtgatggctcgcggtaagttgcagggcgatgacatgttgcagcttactatggcgggtgttcctgtgctgtctttgcttgccaggcagacgggtaaaacgtctgctgaggtgtcgcagatggtgtcgaaggggcagattgattttgccacgtttgcggctgcgatgaagcttggcatgggtggtgctgcgcaggcgtctggtaagacgtttgagggcgctatgaagaatgttaagggtgccctgggttatttgggtgctacggctatggcgccgtttcttaacgggttgcggcagatttttgttgcgttgaatccggttattaagtctatcacggattctgtgaagcctatgtttgcgtcggtggatcaggggattcagcgggtgatgccgtctattttggcgtggattaaccgtatgccgggcatgattacgagaatgaatgcacagatgcgcgccaaggttgagcagttgaagggcgtttttgcgaggctgcatttgcctgttcctaaggtgaattttggtgccatgtttgctggcggcaccgcagtgttcggtattgttgctgcgggtgttgggaagcttgttgcggggtttgccccgttggcggtgtctttgaagaatctgttgccgtcgtttggtgctttgaggggtgccgctggggggcttggtggcgtgtttcgcgccctgggtggccctgttggtattgtgatcgggctgtttgctgccatgtttgctacgaacgcccagttccgtgccgctgttatgcagcttgtgggggttgttggccgggctttggggcagattatggtcgctgtgcagccactgttcgggattgttgctggcgtggttgccaggttggcgccagtgttcggccagattatcggtatggttgctggtttggctgcccggctggtgcctgttattggtatgcttattgcccggctggttcctgttatcacccagattattggtatggtaacccaggttgctgccatgttgttgcctatgctgatgccggttattcaggctgttgttgctgtgatacggcaggttattggtgtgatcatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgccgtgttgcaggtggtggcccgtattattccggttgttatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatatcaaatcgtttacgtcggcgtttattaacggtttcaagtcgatcatttctgccggtgttgccgcggttgtggggttttttacgcggcttggtttgtcggttgcttctcatgttcggtctgggtttaacgcggcccgtggcgctgtttcggctgcgatgaatgctattcggagtgttgtgtcttcggtggcgtctgctgttggcgggtttttcgggtcgatggcgtctagggttcgtagtggtgctgtgcgcgggtttaatggtgcccggagtgcggcttcttctgctatgcatgctatgggctcggctgtgtctagtggtgtgcatggtgtgctagggtttttccggaatttgcctggcaatattcggcatgctctcggcaatatggggttcttgttggtgtcggctggccgtgatgtggtgtctggtttgggtaacggtattaagaatgctatgagtggcctgttggatacggtgcgtaacatgggttctcaggttgctaatgcggctaagtctgtgttgggtattcattccccgtctcgagtgtttcgtgaccaggttggccggcaggttgttgccggtttggccgaggggatcaccgggaatgcgggtttggcgttggatgcgatgtcgggtgtggctggacggctgcctgatgcggttgatgcccggtttggtgtgcgatcatcggtgggctcgtttaccccgtatgacaggtatcggcggatgggcgagaagagtgttgtggtgaatgtgaatgggcctacttatggtgatcctaacgagtttgcgaagcggattgagcggcagcagcgtgacgctttgaacgcgttggcttacgtgtgattgggggtgttgtgcatgtttattcctgacccgtctgatcgtgccggtttgactgttacctggtctatgttgccgttgattggtaatgatccggagcgtgtgcttcatttgacggattatacgggtgcgtctcctgtcatgttgttgaatgattcgttgcgcggtttgggtgttcctgaggtggagcatttttctcaaactcatgttggggtgcacggctcggagtggcgcgggtttaatgtgaagcctcgcgaggtgacattacctgtcctggtgtcgggtgttggtgtggatccggttggcgggtttcgtgacggttttttgaaggcgtatgacgagttgtggtctgcttttcctccgggcgaggagggggagttgtctgtgaagaccccgtctggccgtgagcgtgtgctaaaatgccggtttgattcggtggatgacacgtttactgtggatccggtgaacaggggttatgcgcgctatctgttgcatttgacagcttatgacccgttttggtatggggatgagcagaagtttcgttttagtaatgcgaagttgcaggattggttaggtggcggccctgtcggcaagaagggtaccgcttttccggtggtgttgacgcctggtgttggttcgggttgggataatctgtctaataggggtgatgtgcctgcgtggcctgtgattcgtgtggagggcccgttggagtcgtggtctgtgcagattgatggtttgcgtgtgtcttcggattacccggtggaggagtttgattggatcactattgatacggatcctcgcaaacagtctgcattgttgaacgggtttgaggatgtgatggatcgtttgacagagtgggagtttgcccctatcccgcctggcggttctaagagtgtgaatattgagatggttggtttgggtgccattgttgtgtcggtgcagtacaggtttttgagggcttggtgaatagttgatggctggtcttgttccgcatgtaacattgtttacacctgattatcgccgtgtggcgcctatcaatttttttgagtcgttgaagttgtcgttaaagtggaatggtttgtccactttggagttggtggtgtctggtgatcattctaggcttgacgggttgactaggccgggtgcacggctggttgttgattatggtggtggccagattttttctgggcctgtgcgtcgggttcatggtgtgggtccgtggcgttcttcccatgtgactatcacgtgtgaggatgatattcgtctgttgtggcgtatgttgatgtggcctgtggattatcgtcctggtttggttggtatggagtggcgtgctgaccgggattatgcccactattcgggtgcggctgagtcggtggctaagcaggtgttgggggataatgcttggcgttttccgcctggtttgtttatgaacgatgatgagagtcgtggacggttcattaaggattttcaggtgcggtttcacgtgtttgccgataagttgttgccggtgttgtcgtgggctcggatgactgtcacggtgaaccagtttgagaatgcgaagtttgatcagcgtggtttggtgtttgattgtgtgcctgctgtgacgcgtaagcatgtgttgactgccgagtctggttcgattgtgtcgtgggagtatgtgcgtgacgccccgaaggcgacatcggtggtggttggtggccgcggcgagggcaaagatcggctgttttgtgaggatgttgattcgatggccgaggatgactggtttgatcgtgtcgaggtgtttaaggatgcccgtaacacggattctgagcatgtgcatctcattgatgaggctgagcaggtgttgtccgagttgggggccacgtcggggtttaagatcgagttggctgagtcggatgtgttgcggtttgggcccggcaatctgatgcccggggatttgatctatgtggatgtgggttctggccctatcgcagagattgtgcggcagattgatgtggagtgtgagtcgccgggtgacgggtggacgaaggtgactcctgttgcaggggattatgagaataatccgtcggccctgttggcgcggcgtgttgctggtttggctgcgggtgtgcgggatttgcaaaaattctagaaaagattaggggtttgttgtgggtattgtgtgtaaagggtttgatggtgtgttgaccgagtatgattgggctcaaatgtctggtctgatgggtaatatgccgtccgtgaaagggccggacgattttcgtgtcggcactactgttcagggtgccacagtgttgtgtgaggtcctgccggggcaggcttgggcccacggggtgatgtgcacgtcgaatagtgttgagacggtgaccggccagcttccgggcccgggtgagacccgatacgactatgtggtgttgtctcgggattgggaggcgaatacggccaagttggagattgttcctggggggcgtgcggagcgtgcccgtgacgtgttgagggccgagcctggcgtgtaccatcagcagttgttggctactttggtggtgtcgtctaacgggttgcagcagcagctggataggcgtgctatagcggctagggtggcgtttggcgagtctgctgcgtgtgatcctaccccagtggagggtgaccgtgtgatggttccctctggggctgtgtgggctaatcatgccggcgagtggatgctgttgtcccccaggattgagacgggttctaagtcgatcatgtttggcgggtctgctgtgtatgcttacacgattccgtttgagcggccgtttagtagtgcgcctgttgtggtggcgtctatggctacggcggctgggggcacgcagcagatcaatgtgaaagcctacaatgtgactgtccaaaattttagtttggcgtttattacgaatgatggttcgaagccgaatggtgtgcctgcggcggctaattggattgctgtcggcgtgtgactgtacaggtgttgtggcggatggtgtgatgttggggggctgtggtgtcgtggtttactcctgcactggtggcctctatttgtaccgcgttggccacggttttgggttctgttcaggctgtcacatcccggtctaggcggcgtttacgcaggctgtctgcgcaggtggatgcgatggaagagtatacgtggggtgtgcggcgcgaggtgcgaaggtttaacgccgggcttcctgatgatgtggagccgatgcatcttcctgatgtgcccgagtttttgaaggatactgttgatggtggaggtgagtagggttgagggagttggaggaggagaagcggcagcgccgcaattttgagaaggcttccctgatactgttgtttttgtcgcttgtgttgttggcggtggttgccgggggtgctttgcggtacgggtctgtggcttctcaaagggattcggagcaggcgagggcccagtcgaatggtacagccgctaaagggttggctgcccgtgtgaagcaggcgtgtacccagggtggcgtggagtctgtgaagctgcacaggtctggtttgtgtgtggatgctgtgcgtgttgagcagcgtgttcagggtgtgcagggtcctgccggtgagcgtggcccgcaagggcccgctggtgttgatggccgggatggtagcaatggttctgctgggctggttggccctgttgggccgcagggttccc

SEQ ID NO: 72 - DNA, 29124 bp, synthetic construct
  source 1..29124: mol_type = other DNA; note = PAC10
gggtttagccagccgtgtgcggcaggcgtgtgcttcgggtggggtggagtctgcgcggcttcaccggtctggtttgtgtgtggatgctgtgcgtgttgagcgtagcgtgcagggtgtgccgggtcctgccggtgtacggggcccgcaaggccctgcaggtgctgacggcagggatggtgttaatggttcggctgggctggttggccctgttggtccgcaaggttcccctggcttgaatggtgtgaaaggtcctgacgggttgcctggtgcgaatggatcggatggccatgatggtgttccaggtcgtgcaggtgctgacggtatgaacggcgttgacggcagggatggtgttaatggttcggctggtgagcgcggtgatgtgggcccttcaggtcctgccggcccgcaaggtgcacagggtgaacggggtgagcgcggccccgccggtacgaacggatacgatggtaaggatggtaaggatggccgttctgttgtgtccgtgtactgttccgggggcagcctggttgtgaaatatagtgacggtgtggtttctaccgtatcggactcggcggcctgccagggtgtgaaaccgtcgcctatagtgactatatcatcccacaaatagaaaggagtggctgtgatggtagtgtttggtggtgtgtggtgaggtttattcctgcggcgcatcactcaagcggttcgaatagtccggtgaatagggttgtgattcatgcgacatgcccggatgtggggtttccgtctgcctcgcgtaaggggcgggcggtgtctacagcaaactattttgcttccccgtcttcgggtggttcggcgcattatgtgtgtgatattggggagacggtgcagtgcttgtctgagtctacgattgggtggcatgccccgccgaatccgcatagtttgggtatagagatttgcgcggatgggggttcgcacgcctcattccgggtgccggggcatgcttacactcgtgagcagtggctggatcctagggtgtggcctgcggtggagaaggctgccatcctgtgtagacgtttgtgtgacaaatataatgttccgaaaaggaagcttagtgcagccgatttgaaggctggtaaacgtggtgtttgcgggcatgtggatgttacggatgcgtggcatcagtcggatcatgatgatcctgggccgtggtttccgtgggacaggtttatggccgtcgtcaacggcggcagtggagatagtggggagttaactgtggctgatgtgaaagccttgcatgatcagattaaacaattgtctgctcagcttactggttcggtgaataagctgcaccacgatgtgggtgtggttcaggttcagaatggtgatttgggtaaacgtgttgatgccctgtcgtgggtgaagaatccggtgacggggaagctgtggcgcgccaaggatgctttgtggagtgtctggtattacgtgctggagtgtcgtagccgtattgacaggcttgagtcgactgttaatggtttgaaaaagtgatggtggtgtgttgtgggtaaacagttttggttgggcctgttggagcgtgccctgaaaacttttattcaaacgtttgttgctgtgcttggggtgacggcgggtgtcacgtatactgcggagtcgtttcgcggtttgccgtgggagtctgcactgattacggctacggttgctgctgtgttgtcggtggctacttcgtttggtagcccgtcgtttgtggccggcaagcctaaaaccacggttgtggatgcgggtttggttccaccggatgatgggggcttggttgagccgcatatggttgatgtgtcggatcctggcatgatcgagcctgcagatgatgcggatcttggtgtaggctatgtgccgaaacacgctgccgagtcggaggttgggacggtagagtctactgttgcataattgaacatagatgcgtgccccagcggtgctgccacgatcgtgtggtggttgccgctggggcactatttctgtttatgcggtgtggctatgattcgttgcggtcgatggtgtcttcgagcatctgatacaggtggaggcaggtagagatcgtatcgctggcctggtctagaacgttccggccgataacgtttttgtggttgtcgcggtggcggatgatagcccacatgatctcgtcggcctccgcttgtaatagttttgcctggtatgcgattccggcgagccagtctagtgcttcctggcttgcataggggctctggtcctcgctgttgtcacgggtgttgctgttgtttgtggggtgtcctgcactgtcgcataaccacaggatttcgctgcactcgtctagcgtgtcctggtcgatagcgagatcgtcgaggctgacttcgttgacggtaaggttcacgttgtcgagtgagatgggtacaccgtactggttttcgacactgtcaacaatgttttccagctgttgcatgttggtgggctgttgttggacgatacggtgtatcgctgtgttgagggtggtgtaggtgatattgtgtgtgttgttcatggttttatcccatccctgtgctgtcgtcgttttcgtctggatagtatctactgtttgcgtagcctgttagggtgatgagtgtttggtctgcccactgtttcacggtttgtcttgtcaccccgagtcgttgggctgccaccgaataggtttgatcatacccgtatacttctctgaatgctgccagccgtgccaaatgttttcgctgtttggatggctggcaggtgagggtgtagtcgtcgatggctagctgcaaatcgatcatggtgacaatgttgttgccgtggtgttgtggcgcggttggtggtggtggcattcctggttcgacactcggtttccatgggcctccgttccagatccattgggcggcttggatgatgtcggcggtggtgtaggttcggttcactggtaatccttaaacaagtcgttcatgttgctggtgttgctggtgttgctggtgtcgaatcgtcccacacagtggcagtagtcgtacatgagtttaataatgtgttggtggtctcccaaataggtgttgccgctgatgctgtaggtggctgtgccgtctttactgatggtgtatttggcggtgatggtttcggggttttcggtgtcggtgatgatggctgtggtggtggtgcctacggtttgtagcacggtggtttgggttccgtcgtcgatggtggttttaaccatgaggggttctccttttaaatgcttgtttggttgtcggctagatgaataatatcggataaaggtttcggctggtctaggtgttgtatggttttgttggctagccgtttggctaccctgtagcacattttggtatagtgtttgttgtctaggttgtggtattgttcccgcaccgcaatatatagtagggagtcttgatagaggtcgtctgcactgattgcggggtagtgtgtggctgttttggtgcatgcccggttgagtgtgcgtagatgatggtttgtggcccatccccacgatgcggtggtggctatgtctgcttttgttggtcgtctgctcatggcatctctttcatctggctatctggtagttgtttggtgttttgttgttgatagtgtagcacacgagtccggggtttccggtggcgcctgtgcggtgccggaaccatgtggattcgccttccatggatgggcattggatgaaggtgcgttggccttgctcggagatttctaggtggtgccggtgcccggccatgaggatgtgggatgtggtgccgttgtggaattcttggccgcgccaccattcgtagtgttggttgttgcgccattggtggccgtgggcgtgcaggatttgtgtgccggccaccccaacggtggtggtcatttcgtcccggctggggaagtggaagtgaagattggggtagttgttgttgagctggtaggcttctgcgatggcccggcagcagtccacgtcgaaggagtcgtcgtaggtggtgactcctttgccgaagcgtacggcttctccgtggttgccggggattgaggtgatggtgacgttttggcagtggtcgaacatgtggatgagttgcatcatggccatgcgggtgagcctgatttgttccgtcaagggtgtttgggtgcgccaggcgttgttgcctccttgtgacacgtatccttcgatcatgtcgccgaggaaggcgatgtggactcgttgcggctgtcctgcctgttgccagtagtgttttgctgctgtgagggagtgcaaatagtcgtcggcgaagtgtgctgtttctccgttggggatgcctttgccgatttggaagtctcccgcccctaccacgaacgcaaccttgttgttgctgcgggtgtgggtgtctggttttgggggtgtccattcggctagtttatcaacgagttcgtccacggggtaggggtctgttgcgggttggtggtcgatgattttttgtatggatcggcctgtttctccgttggggagtgtccattcggagatgcgtgtgcggcgtacggtgccgttggctagattgtcgcagatggtgtctgcttcgctatcgtggttggctagctgtgtgaggagccggtctatattgtctatcactggttttcctcctcttgcggggtggtgttggcttgtttgcggcgatagtctttaataacggtggcggagatggggtatcctgcctgggtgagctgttttgctagccatgaggcggggatggttttgtcggcgagcacgtctgcagctttgttgccgtagcgttgaataagggtttcagttttggttgccatgatgtggttttgtcggcgagcacgtctgcagctttgttgccgtagcgttgaataagggtttcagttttggttgccatgatgtcctaggggttgtgtggtgggctgccatcctgtgcggcagtcgccgtcgtgtcctggtttgcgtgtgcaccacgatacgttgccggcattgtggatgatggcacggccgcatatgacgtcacgtagatgctcgggaaacttgtcgttgttgtttccgttcgtgtcgatcaagtgttgggttttagtaaccatcatgtctcctatgtgtgaaagagtgtgcaaatactatgcaggtgtcatggatgtttatgcgggtatggttttcatcaccttgctgaatgtgacttggttactgtacatcatctgggtgatttcctgatcggtcttgtcggggtgctgctttcgcaggttcgcccattggcaggcgttgtcggtctcttgctggagccgggtcaggtgctgctcgttgatgatgtgtttccacattgtccacgacacgtcgagcctgcggagcatgttcatggctggcacgttaaacgagtcgaggaagagtatttcttcggtgtagtactgtttttcgtattggtcccatccgcttcggtgcctgttgggctggtttttggggtaggcttcccggcatactttgtgtaaccgtttggccatgtcgtcgggtagtttaatgtcggggttggcgcggatcatggatcgcatcccatcgtaggtggtgccccagcggtgcatgatgctgagtgggtcttcaccatcggcccatttttctgcacagatggcgaggcgtatgcgcctcctggcggctttgctggtgtcgcggcggccggggatggggcacgtgtcgagaggatccatgatgttttatatgcctttctttgtttggtttgcttgtgtggttttattgtagcactgtgtctagtgcttgtgtcaaccctgtttttccggcctgcaggtaggtgtctgtgacatcgcccagggtgaggggcacgtgtatggcttgggggagtgctgcctggagggtttgtgccatctggtggcctgccttgtctgggtcggaccagatgtagatgtggtcgtagccttcgaagaatttggtccaaaagttttgccacgaggttgcgccgggtagggcgacggccgaccatccgcattgttcgaggatcatggagtcgaattcaccttcgcaaatgtgcatttctgctgccgggttggccatggcggccatgttgtagatggagcctgtgtcaccggccggggttaggtatttggggtggttgtgggttttgcagtcgtgcgggagtgagcagcggaaacgcatttttcttatttcggctggccgcccccaaacggggtacatgtatgggatggtgatgcactggttgtagttttcgtggccgggtatggggtcattgtcgatgtatccaaggtggtggttgcgggctgtttcttcgctgatgcctcttgctgagagcaggtcgagtatgttttcgaggtgggtttcgtagagggccgaggctttctggattcggcggcgttccgcaatgttgtatgggcgtatgctgtcgtacattcgggttttctttctctagttgttgtttcagttgggcgagtccgcctccgataccgcatgtgtggcagtaccagacgcccttgtcgaggttgatgctcatggagggctggtggtcgtcgtggaatgggcagaggatgtgttgctcgttcctggatgggttgtaacggatgcggtaggtgtcgaggaggcggcaggtgtcagaggtgtgggaggagctcgttgagggttgataccacataggcttcgctccagggtttgttgcgctgtttcatcactacgagtccgatggtggactggctttctcggtttcggtgggtttcgtagttgcgtgcctccaggctggcttgtttcacgaattcggctaggtggggctgcccggctttcgcctcgataatgtaggttttatggccggttgtgaggatgaggtcgccttcatcctctttaccgttgaggtggaggcgttctatatcatagccggtgtcgcgtagctggtggaggagtcttgtttcccattcggccccggcccgccggttgcgtgcctgctgtgtaaccatcatagtcctttgtgtgttgtggtcatgttccagggatgtttttcggcgagtggcccgaagaatgtgtattcggggtaggctcgtagccgctcatattttgttccgtctgggctggatttgccggtgcgctgtttcaacactgcgatgcgcgcctcggctggtatcgtgagcccgttgccgttatcctcgccaccataaagtgagactcccaatatgagttgtggtttttcggagaggccgtttttaatttcccgtctagctggcgggtgttcgatgtcggagccggttttgtcggttgcgtggtgtgtgacaataatggtggagccagtatccctgcccaatgctgtgatccattgcatggcttcttgctgtgcctggtagtcggattcgcagtcttgaatgtccatcaggttgtcgataacaatgagtggtgggaaagtgttccacatttccatgtaggcttgtagctccatggtgatgtcggtccaggtgatgggtgactggaatgagaaggtgatgtgttggccgtggtggatgctgtctcgatagtattctggcccgtagtcgtcgatgttgtgttgtatctgtgtggtggtgtgttgggtgttgagtgagatgattcgtgtggaggcctcccagggtgtcatgtcccctgatatgtagagggcgggctggttgagcatggcggtgatgaacatggctagcccggatttttggctgccggagcgccccgcgatcatgaccaaatcccctttgtggatgtgcatgtcctggttgcggtagaggggttctagttggggtatgcggggcagctcggctgcggtttgggaggctctcgcaaaggatctttggagagagagcatcggagcctttatctatcgatcggttggatgtgttgtggtggtcagatggagtcgatgtctacatcatcactatcagtggtgttgggctggctgtctcgccgatcaacgtaggctgctacaaggtcgtagatggcgtcgtccaatggtttgagcacgaccgcgttgaacccgtttttagtgcgcacctgatcgagtttgaaggcctgctcctcgccaagatatgcctctaaatcgcggatcatggagtgtgggcggtcgttgttgcctcgcactttttcgataatggcgttggggatggtttctggggtgccgttgttgaggtcgtctagggtgtggaagatggtgacatcagcgtagatacgatcggcgacctgtccaccgtagccttcagtgttgtgctgaacgtcgtggactttgaaggcgatggcggtggcgtcctggtttcgggaggggttgaagaaggtgctgttgctgttgttgcggtagtttgcgagtcccattattgtttcctttactgttttgttggtttgtgtcggtttttatcgggtgaggctgtttcgtttgctgcggaaagcctcggaaacgtcactgttactagtgatgatctttttgtactgtttcagtagatcggctagctgtgctttgcttgttgcattgttgattttgtcgatgatggtgttgtttccttctgaggcgatgttgtctacgtagtctttggcggcctggttgtatcggtcttggaggatgatggatgctgtggcgatcagtgttgccaggtcccagttccttgccgcggagctgtttttgagtccgcctaacaggtcgatgatggctttctttacctggtcggcggtgtctcctcggatgacggtccatggggcggcgtagtctccgccgtatttgagggtgacggtgaatcggtcgtcgtctgtgttgtcggtcactggtgctccttgtcttcttgtgttggggctgtgatggtggtttctatagggtacctgtaggcgtctttcccgttgacggcccagcaggcgtctctgacggggcatcctttacagagtgctgtgacgtgtgggacgaagatgccttggctgattcctttcattgcttgactgtacatggatgatacatgccggtaggtgttgttgtcaaggtcgtacagttcggtggccgttccctgcttggcggactgtttgtctgttttggttgatgcgggtgtccaaaacatgccttttgtcacatcgttgccgtgttgggcgagcatgtaccggtaggtgtgcagctgcatgctgtctgctggtaggcggccggttttgaggtcgaggatgaaggtttcgccggtgtcggtgtcggtgaagatacggtcgatgtagccaacgatctgggtgccgtcctggagggtggtttctaccgggtattcgatgcctggctggccgtctaggactgctgtgtggtattgcggattgtttgtgcgccagtgtttccaccggtcgacgaaggtttgcccgtaaaccatccaccagtcgtagtcttttttgtgtggcccgcccgactcgcacatgtttttgcacaccctgccggagggtttaatctccataccctctgatcgggtgagggcgacttgggtgtcgaaaatgtttttgaaggatgagagtttgtctggcagtgcagggtattcggcggggttgtacaggtgtaggtcgtattgttcggtgatgtggtgtatggcgcttccggcgatggtggcgtaccaggtgtggtgttgggcgtggtagccgtgggataggcgccatttttctccgcattcggcccactgtgacagtgatgagtaggagatgtggcctggatggtggatggttttcggatattgtgctagaggcattacttgtcgcttttgttccatgggttgcgggtgtctaccccggcattgtgttgctggtatgcgaggagtgctaggcagtgccaggcagcatgtgccaggtggggtagcccggattcataatcgaggttgtttccttgctgccaggatagcacatggcggtagagggcgtcaacgctgtggctccacggatagccgccggtccagttgttgtcgccgtatttggtggcaccgtagcctgcaacctcgccgagggcgtgtaaggctgcggggtcgatgagggagagcctgcaaagtttgagttctttcttggcgccagtatcagggtcggtgtacatgcgggtgggctcatccatggggtgtgtgctccttaagggtgggttactggttggggttgtgggcgagtgctactgcgagaataatgatggcgagggtttctgcgatgaggatgggtgttgtgatcatttgttgtctcggggattgctggtgagtgtggaggcgcctaggagggtggtgagggcgcatgcggcgatgatggcgagggctgccttgtgtggggtgccggtggcgtacatccatgtgatgatggcgccttggatccaggcgaggctggtgaagaacgtttcgtagctgtgtagctcgctgttgttgctggtgatgtcattcatggtagttttctgctttgtgtgcgatggttgtgtacatgtcgttgagtgtggtttcgatggtgatgagagtgttgatttcttggttgaggtcgatgttgtctttgagggtgtcgatgcgggcggcgatgtcggtggcggtgcgtaggcttactgctgcaccgtggatgatgtggcacatgtcggtgaggccgaccttggcgatatagtgtgacatgagaggcatgatgggtgtgtcgtctttctggtcagcgtgacgggttgatggacatgtcttctacctgtggcttgtcttcggtgcctgatacttggcaaaagactttcacgtgcgccttggatgctccgggttgcttggcggtggcaccgtaggcgatagtaaaggcgtctttgtgggcgccgatgactttgtgtaggaagaggtcgatgtcggggtttccgttccatttgacaccgttttctgcggctgcctgggtggctttctggttgcaggcgtgtgctgccgtaatcatggtgagtccggtggcggtttcttcaccccttgcttgggcttgcttgtgggtttttgcttgttcggcttgtagggagcggactgcggctgcctgccgtgctttcttttcggctttgcgctgctgggtagtcttgggggtccattcggtgttggctgtggtggcctgtggggctggctgtgaggcgagtggcggattgtcgtctggggctggcatgaatgaggcggcggcaatgatggcggctgtgattccggcgatggtgtagccgtttttcttgttcatgactgttgtcccctttccggggtgttgttcgttgctgacatgattaatcatggtgtggacggttccccatgtcaaggctgcgctcaacgattgtgagcgtttggtgtgtggctaggggttttatcgggcacacagggtgagtagatggccaacattgatgcggctcacattccagtagagttgtgtggcttcaccgccggtgagcggcttccactcgttgtggctgaacacggtgccatcggatgcgatgaatgtgtcggggcgtagcttgtgaagctcggcttccacgctctgccggtaggtttcggcgaggccctcaaaatccatgtggtcgcaggagaggttttcgaggcgtgtcaggtcgaagggtgtggggcagtcgtagctggcgggggtgtagagctgggtgaagtggttggcgatcttctgcatgatgatgtccttttggttgctgataaccttgttgagggtttatcgggtggatgtgataaggatggcgtccacgtcgatcatgtcgatgagatcgtggagttcctcggcctcgttttcggtgagtggctgccagttgttgtcgccgtacacggcgccgtcgagggtgacagtccacagtggccggatgaggcgtacggcttcttgtactttagcgtggtacatgcggcgcaccatatccagatccatgtcgtctgaatggtttccgatgaggttgtggaggctgagcgggtcgatttctgtctgcctgtagagggatgtgaaggatggggtgatgagtgtgccatccatgggtgatgttcctttctggattgtcttggttggttgttgtggtttctagagtgtgcgggttgcaaccgggagtcaaggctgcgctcattcggattgagtgtttcatgctggagtgtcgggtgtgacagatgtcacttaagcctttattgcctctctcggcgtctcacatcatctgggggtaagattatgcagggttgaccctgctgatcgattctagggcccttctagggcgtctcaggggtacgtctgggtgatagcgggtgtggcagatgatctagcgagtcaaggtaccgagcttagacgtaagatctatcatctaggcgtgtgagatgtatcacatcctcctggctgggtgtgcaccctcaaggctactctgccgatctggcgtggagggtgtagcccagaaatgccgtttaaagccttcacatggcgcctagaagcgccttgcagggtgggggctaggtatttatacccccaacacattctgatcgattctagacgcctataggagcctgatacacgatcaaccatctcggcatagatcatcagcccctatcctagttagctaagcctgaactatgtggacagtgtaggatgctaagagggaagaaggacacggtaaaagaaagaggggggggcatcaaccttcacgcccgaggtacttaagttaaccttagggtcttagcaccgagcccctcaagggctcggcatcagcatcatcgggatcagccgatccggcacagccttagcaagtacacaccatcagggaaggcttgagagtacgaggagccctagcgacgagtactcgaaagcctgagggaacaccctcagcactgatgggcctagcgtgttcggaaagtacacaggggtacagtgtgagagctgttcgggagctaaaccccttccggctagggcaaacaccagtcctagactatcccacaccctcatctgttaaccttccgttcattaaacgttaaggaaacttttaggtttgatttttggaccttaaccaccaaaaacacccatttacacccctcaaacccgccaatagagccaaacgccggtgttgagggtatctctacctagtgtgataggctggacaggtagccagctggacgcaaggccagaaagtgctgacgcacttcccgacctcgcttaccatcagtctaccaaacacttaaaagcttaacagctaagcgctaagcccttaagacctcaacgcttagcaccgagcccttgaggggctcggcatcagtcttaggtacttaaagtaactttaaaccttaaaggcttagcacttaaggatataaacttaacatcagtgtttaagactttaatactttaagtaactataagaccttaaagctttaaacacttaaagttaaccatcagtcttaaactttaatattataacttataagctttaatacttatattatattataacctataagtcttaaagcttataggttataaaagttttagaagagctaagaggttaacttctttacttctctactctctttggttctttctctcttctcttcttttcttcatcaggggagaagaggaatctttaccatcagcgccgatgacctttcaccgtgtggatcgtgtgcttctggtcgcaagctcccatcgcacactccccacactcttacacccgtgtccctttcgggcttggcgtgttcggctaaaggcgtacggcgtgtcacgctaacacccttaacaccgggtaagacttaaagtgtatattatatgtagaagactttaaaacctataaggtgttcccgcttagcccgtgtcctacaccgctaggcgccaagcgctaagccttgaaacgcgaacacacacccacccccttttttcttccgtgtccttctcttttgacaccgctggggggcgatgtgatctttctcacacccatgggggtagtggagaaaacaaacaccccggcacaaacagaacaccccctcaaacgaacaaaacagccccccagaatcgaccagcagggcaagggtagagtatccatacccccaacggtttccaggccgttacagaggcaaataagacccgtacagggctaggcgaggaacagacacatcatggcacgcaccaaccgcacagccgccacggcacaccgacgctggcggcaacgactcatcacccaagcccaacagcaaggccaaaccacctgcccactctgcggagtcaccatcacctgggacacccaccagctaccaaccagccccgaagccgaccacatcacacccgtcagccggggaggactcaacaccctagacaacgggcaaatcatctgcagaacatgcaacagaagcaaaggcaatcgcagcgaaccaaacatcaaattccaacaacaaaccacaaaaaaccttgtttcatggtagaaaacctgccagcccccaccggggacaccccctgcacaggcgtgcaagacctcgtacggcttagtgaaatacctcccttttgtggatttgtctgtttgtcgactttttgtgttggtggtgagtgttgtgcagcctgagcttcctgatagtcgtgattggtgtggggagacgcgtcgttggtggcgtgtgtggggtgaggatagccgtgcatcgtacgtgtctgatgaggagtggttgtttctccttgatgcggctgtgattcatgatgtggtgtggcgtgagggtcgcgcggatttggtggcttcgcttcgtgctcatgtgaaggcttttatgggtatgttggatcggtattcggttgatgtggtgtctggtggccgtgccggtggttctgcggtggcgatgattgatcggtataggaagcgtaaaggggcctaatgtcgagtgttgttggttctcaggttcctcgtcatcgtgtggctgcggcgtattcggtgtctgctggtggtgatgctggggagttgggtcgtgcgtatgggttgacgcctgatccgtggcagcagcaggtgttggatgattggctggctgtcggtagcaatggcaggcttgcttcgggtgtgtgtggggtgtttgtgcctcgccagaatggcaagaatgctattttggaggttgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgggcgacgaatggtcaggaggctattgtgttgcatcacccggattgtccgacttttgagaagaagtgtggctgcagcggttggggttcggttgagtttgtggcccgttctcggggttctgctcgcgggtttacggttgatgatttggtgtgtgatgaggctcaggagttgtcggatgagcagttggaggcgttgcttcctacggtaagtgctgccccgtctggtgatccgcagcagattttccttggtacgccgcctgggccgttggctgatggttctgtggtgttgcgtttgcgtgggcaggcgcttggtggcggtaaaaggtttgcgtggacggagttttcgattcctgacgagtctgatccggatgatgtgtcgcggcagtggcggaagttggcgggggatacgaatccggcgttggggcgtcgcctgaattttgggaccgtaagcgatgagcatgagtcgatgtctgctgccggttttgctcgggagcggcttggctggtgggatcgtggccagtctgctgtgtctgtggttcctgctgataagtgggctcagtctgcggtggatgaggcgagtctggttggcgggaaagtgtttggtgtctcgttttctcgttctggggatcgggttgctttggcgggtgccggcaagactgatgctggggttcatgttgaggttattgatgggctgtcgggaacgattgttgatggtgtgggccggttggcggactggttggcggttcgttggggtgatactgaccggatcatggttgccgggtctggtgcggtgttgttgcagaaggcgttgacggatcgtggtattccgggccgtggcgtggtggttgctgatactggcgtttatgtggaggcttgtcaggcgtttcttgagggtgtcaggtcgggtgtgatcagtcatcctcgtgctgattctcgccgtgacatgttggatattgctgtgaggtcggctgtgcagaagcgtaaggggtctgcgtggggttggggttcctcgtttaaggatggttctgaggttcctttggaggctgtgtctttggcgtttttgggggctaaacgtgttcgtcgtggccgtcgggagcgtagtggtaggaagcgggtgtctgtggtatgaactcggatgagttggctctgattgagggcatgtacgatcgtatccaaaggttgtcttcgtggcattgtcgtattgagggctactatgagggctctaatcgggtgcgtgatttgggggtggctattcctccggagttgcagcgtgtgcagacggtggtgtcgtggcctggtatagctgtggatgctttggaggagcgtctggattggcttggctggatgaatggtgacggctacggcctggatggtgtgtatgctgcgaatcggcttgctacggcgtcgtgtgatgtgcatttggatgcgctgatttttgggttgtcgtttgttgcgataattcctcatggtgatggtacggtgtcggttcgtccgcagtcaccaaagaattgtacgggcaagttttcggctgacgggtctcgtttggatgctggtttggtggtgcagcagacgtgtgatcctgaggttgttgaggctgagcttttgcttcctgatgtgattgttcaggtggagcggcgtggttcgcgtgaatgggttgaggtggatcgtataccgaatgtgttgggtgcggttccgttggtgcctattgtgaatcgtcgccgtacttctaggattgatggccgttcggagattacgaggtctattagggcttacacggatgaggctgtgcgcacactgttggggcagtctgtgaatcgtgatttttatgcgtatcctcagcgttgggtgactggcgtgagcgcggatgagttttcgcagcctggctgggtcctgtcgatggcttctgtgtgggctgtggataaggatgatgacggtgacactccgaatgtggggtcgtttcctgtcaatagtcctacaccgtattcggatcagatgagactgttggcgcagttgactgcgggtgaggcggctgttccggaacgctatttcgggtttatcacgtctaacccacctagtggggaggctttggctgccgaggaatctcggcttgtgaagcgtgctgagcggcgtcaaacgtcgtttggtcagggttggctgtcggttggttttttggctgccaaggcgttggattctcgtgttgatgaggccgatttttttggtgatgttggtttgcgttggcgtgatgcttcgacgcctacccgggcggctacagctgatgctgtgacgaagcttgttggtgccggtattttgcctgctgattctcgtacggtgttggagatgttggggcttgatgatgtgcaggttgaggctgtgatgcgtcatcgtgctgagtcgtctgacccgttggcggtgcttgctggggctatatcgcgtcaaactaacgaggtatgataggcgatggcttcgggggttgaggcgaggcttgcggcgactgagtatcagcgtgaggcggtcaggtttgctgggaagtatgcgggctattattctgagcttggtcgtttgtggcgtgccggcaggatgagtgacacgcagtatgtgcgtttgtgtgtggagttggagcgtgccggccatgatggttcggcatcgttggctgccaggtttgtgtcggattttcgccggttgaatggtgtggatccgggtttgattgtgtatgacgagtttgatgctgcggcggctttggctaggtctatttcgaccacgaagattcttgagagtgacccggatagggcgaatgacacgattgatgcgatggcggcgggttttgatcgggctgttatgaatgctggtcgtgacacggttgagtggtctgcgggtgcgcagggtaggtcgtggcgtcgtgtgacggatggtgatccgtgtgctttttgtgccatgttggctacgaggtcggattatacgacaaaagagagggcacttactactggacatactcggcgtcataagcgtggtggtaagcgtccgtttggttcgaagtatcatgatcattgtggttgtacggtggttgaggttgttggcccttgggaaccaaatagggctgatgccgagtatcagaggacgtatgagaaggcccgtgagtgggttgatgatcatgggttgcagcagtcgcctggcaatattttgaaggctatgcgtactgttggcggcatgagataatttgatgtggtttccggttgtgcgccgccggttattggtgcacagggttgtctcccgcacgggggtcaacaatgttgtgttgttttccgcaaggagtgtagggttaggctatggccgatcagagtgttgaggaacagaatgttgacaatgatgttgtggagtccggaaaggataacggcattgttgatacagtaaaagacgatggcggacaggaggtggccgacaatcagttgaagaatgaaggcgagggtaaatcgccggggactgattggaaggcggaggcccgtaagtgggagtctcgtgctaaaagtaatttcgctgagttggagaagcttcgcgcctcggatggtgatgcggggtctgtgattgatgatcttcgccgcaagaatgaggaactcgaagaccggattaacgggtttgttcttgagggtgtgaagcgcgaggtggcttcagagtgtggcctgtcgggtgatgctgtcgctttcttgcacggtagcgatcgtgaagcgctggtggagtctgcgaaagctttgaagggtttgatcgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctttcgccggagcagccgactatttttggccctgttaagggtgccgtgtttagtggtgttcctcgcgctaagattgttggtgagggcgaggttaagccttccgcgtctgttgatgtttcggcgtttactgcgcagcctatcaaggttgtgactcagcagcgtgtctcggacgagtttatgtgggctgatgctgattaccgtctgggtgttttgcaggatctgatttccccggctcttggtgcttcgattggtcgcgccgtggatctgattgctttccatggtattgatcctgccactggtaaagcggctgccgctgtgcatacttcgctggataagacgaagcatattgttgatgccacggattctgctacgaccgatctggtcaaggctgtcggtcttatcgctggtgctggtttgcaggttcctaacggggttgctttggatccggcgttctcgtttgctttgtctactgaggtgtatcctaagggttcgcctcttgctggccagccgatgtatcctgccgccgggtttgccggtttggataattggcgtggcttgaatgttggttcttcttcgactgtttctggcgccccggagatgtcgcctgcctctggtgttaaggctattgtgggtgatttctctcgtgttcattggggtttccagcgtaacttcccgatcgagcttatcgagtatggtgacccggatcagactgggcgtgacctgaagggccataatgaggttatggttcgtgctgaggctgtgctgtatgtggctatcgagtcgcttgattcgtttgctgttgtgaaggagaaggctgccccgaagcctaatccgccggccgagaactgatttattgttgcggtgatgtgtcaatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgctgctaaggctattctgcgtagggctttgttgcgctggaatgatactggcgtgtcgggtcaggtgcagtatgagtctgcgggtcctttcgctcagactacacggtctagtactcccacgaatttgttgtggccttctgagattgccgcgttgaagaagctgtgtgagggtgatggtggggctggtaaagcgttcactattacaccgaccatgaggagtagggtgaatcattctgaggtgtgttccacggtgtggggtgagggttgctcgtgtgggtcgaatattaacggctacgctggccctttgtgggagatatgatatgaccagttttccttatggtgaaacggttgtgatgcttcaaccgactgttcgtgtcgatgatcttggtgacaaggttgaggattgggggcatcttgtagaaacagtgtaccataacgtggccatctatgcttccgtttcgcaggaggatgaggccgcggggcgtgactctgactatgagcattggtcgatgcttttcaagcagtctgttgttggtgctgattatcgttgcaggtggcgtatccggggtgttgtgtgggaggctgacgggtctcctatggtgtggcatcatccgatgtctggctgggatgcgggcacgcagatcaatgtgaagcgtaagaagggctgatgggtagtggctcaggatgtgaatgtgaagctgaacttgccgggtattcgtgaggtgttgaagtcttctggggtgcaggctatgttggctgagcgtggcgagcgtgtcaagcgtgcggcctcggcgaatgtgggcgggaacgctttcgataaggcccaataccgtaatggtttgtcgtcggaggtgcaggttcaccgtgttgaggctgtcgctcgtataggcaccacatataagggtgggaagcgtattgaggcgaagcatggcacgctggctcgttcgattggggctgcgtcgtgatcgtctacgatgaccccaggaagtgggctaaacgcgtgctcaaggatgatggctggctgtctgatataccctgtgtggggacggtgcccgatgattttatgggtgacctggtttggttggcgttggatggtggcccgcagttgcatgttcgtgagcgtgtttttttgcgcgtgaatgtgttttctgatacgcctgatcgggctatgtctttggcgcgtcgtgttgaggctgtgctggctgacggggttgatggtgatccggtggtgtactgtaaacggtctactggtcctgatttgctggttgatggtgcacgttttgatgtgtattcgctgttcgagctgatatgtaggcctgtcgaatccgagtaaacgtatttgtttttgttttaatgtaattgtttgatatttaatgggggttgtgatggctgcaacacgtaaagcgtctaatgttcgttcagcggttactggcgacgtttatattggtgacgcgcacgcgggtgatactattaagggtgtggaggcggttccttccgggcttacagctttagggtatctgtcggatgacgggtttaagattaagcctgagcgtaaaacggatgatttgaaggcttggcagaatgcggatgttgttcgcacggttgctaccgagtcttctatcgagatttcttttcagctgatcgagtctaagaaggaggttatcgaactgttttggcagtcgaaggttactgccggagccgattcgggttcgttcgatatttctcctggtgccaccactggcgtgcacgctttactgatggatattgttgatggggatcaggttattcgctactatttccctgaggttgagcttatcgatcgtgacgagattaagggtaagaatggcgaagtgtacgggtatggtgtgacgttgaaggcttaccctgctcagattaataagactggtaatgcggtgtcgggtcgagggtggatgacggctttaaaagctgatactcccccttctccgaagcctcagccggatccgaatccgccgtctgagaactgatacacgattttagggattgttgatagatgagtgacacaggttacacgttgaagattggtgaccgtagctgggtgttggcggatgcggaggagacggctcaggctgttcctgctcgcgtttttcgtcgtgcagctaagattgcccagtctggggagtctgcggatttcgctcaggttgaggtgatgttttctatgttggaggctgcagccccggctgacgcggtggaggccctggaggggcttcctatggttcgtgttgccgagattttccgccagtggatggagtggaagcctgaaggtaagggtgcctctttgggggaatagtttggctccacggcctgattgatgagtatcgtggggccatcgaatatgattggcgcacaaggtttggtgtgtgcatatacgatataggtggtcctgcgatggggtggggtgaggctgtccggctggctggcgtgttgtgtggtgatacgtcgagccagttggcggcccacctgaatggttggcagcgcccgtttgagtggtgcgagtgggctgtgttggacatgctggatcattacaggtctgctaatagtgaggggcagccggagcctgtggcgaggcctacggatgagcgtaggggccggtttacgtctgggcaggtggatgatattttggcgcgtgttcgtgctggtggcggggtgtctcgcgagattaatattatggggtgaatagtgtatgtctggtgagattgcttccgcatatgtgtctttgtatacgaagatgcctggtttgaaggcggatgttggtaaacagctttctggtgtgatgcctgctgagggtcagcgttcgggtagtctttttgctaagggcatgaagttggcgcttggtggtgccgcaatggtgggtgccatcaatgttgctaagaagggcctcaagtcgatttatgatgtgactattggtggtggtattgctagggcgatggctattgatgaggctcaggctaaactgactggtttgggtcatacgtcttctgacacgtcttcgattatgaattcggctattgaggctgtgactggtacgtcgtatgcgttgggtgatgcggcttctactgcggcggcgttgtctgcttcgggtgtgaagtctggcgggcagatgacggatgtgttgaagactgtcgccgatgtgtcttatatttcgggtaagtcgtttcaggatacgggcgctatttttacgtctgtgatggcccgcggtaagttgcagggcgatgacatgttgcagcttacgatggcgggtgttcctgtgttgtctttgcttgccaggcagacgggtaaaacgtcggctgaggtgtcgcagatggtgtcgaaggggcagattgattttgccacgtttgcggctgcgatgaagcttggcatgggtggtgctgcgcaggcgtctggtaagacgtttgagggcgctatgaagaatgtgaagggcgccctgggttatttgggtgctacggctatggccccgtttcttaatgggttgcggcagatttttgttgcgttgaatccggttatcaagtcggtgacggattctgtgaagccgatgtttgctgccgtcgatgctggtattcagcgtatgatgccgtctattttggcgtggattaatcgtatgccgggcatgatcactcgaatgaatgcacagatgcgcgccaaggttgagcagttgaagggcatttttgcgagaatgcatttgcctgttcctaaagtgaatttgggtgccatgtttgctggcggcaccgcagtgtttggtgttgttgctgccggtgtagggaagcttgttgcagggtttgccccgttggcggtgtcgttgaagaatctgttgccgtcgtttggtgctttgaagggtgtcgctggcgggcttggtggcgtgtttcgcgccctgggtggccctgttggtattgtgatcggcttgtttgctgccatgtttgctacgaacgcccagttccgtggcgcggtgatgcagcttgtgggggttgttggccaggctttggggcagattatggccgctgtgcagcctgtgtttggtttggttgccggtttggtggcccggttggcgccagtgtttgcccagattatcggtttggttgcaggtttggctgcccagcttatgccggtgattagtatgcttgttgcccggctggttcctgtgatcacccagattattggtgcggtgacacaggttgctgcaatgttgttgcctgcgttgatgccggtgttgcaggctgttgtggctgtgattcggcaggttgttggcgtgatcatgcagttggtgcctgttttgatgcctgtgattcaacagattttgggtgctgtcatgtctgtgctgccacccattattggtcttatccggtcgttgatgcctgtgattgcggcggttatgcgtgtggtggtgcaggttgttgcggttgtgatacaggtggtggcccgtattcttgcggttgtggctccgatggtggcggctgtggtagggtttgtggcccgtattgttggtgctgtcgtgtcggctgttgcccgtgttattgctgctgttgcccgtgtcatcgggtggattgtggcccattttgtgtctggtttggcacgtatgggttcggtggttcaggctggctggaatcggattagggcgtttacgtcagcgtttattaacggtttcaagtcggtgatttctggcggcgtgaacgcggttgtggggttttttgcccggctgggttcttctgttgcttctcatgtgaggtctggttttaacgcggcccgtggcgctgtttcttctgcgatgaatgctatccggagtgtggtgtcttcggtggcgtctgctgttggcgggtttttcagttcgatggcgtctagggttcgtagtggtgctgtgcgcgggtttaatggtgcccggagtgcggcatcttctgctatgcatgctatggggtccgctgtgtctaacggtgtgcatggtgtgctgggttttttccggaatttgcctggcaatattcggcgtgcgcttggtaatatggggtccctgttggtgtcggctggccgtgatgtggtgtctggtttgggtaatggtatccggaatgctatgagtggcctgttggatacggtgcgtaatatgggttctcaggttgctaatgcggcgaagtcggtgttgggtattcattccccgtctcgggtgtttcgtgaccaggttggccgtcaggttgttgctggtttggctgagggtattactggtaatgctggtttggcgttggatgcgatgtcgggtgtggcgggacggctgcctgatgcggttgatgcccggtttggtgtgcgatcgtctgtgggctcgtttaccccgtatggcaggtatcagcgtatgaatgataagagtgttgtggtgaatgtgaatgggcctacttatggtgatcctaacgagtttgcgaagcggattgagcggcagcagcgtgacgcgttgaatgcgttggcttacgtgtgattgggggtgttgtgcatgtttattcctgacccgtctgatcgttcgggtttgactgtgacatggtcgatggatccgctgtttggtggggggcgtgtgcttcatttgacggattatacgggtgcgtctcctgctatgttgttgaatgattcgttgcgcggtttgggtgttcccgaggttgagcatttttctcaaacacatgttggggtgcacggctcggagtggcgcgggtttaatgtgaagcctcgcgaggtgacgctaccggtgttggtgtcgggtgttgactcggatccggatggcgggtttcgtgacggttttttgaaagcctatggcgagttgtggtctgcttttcctcctggcgaggagggggagttgtcggtgaagactcctgcaggtcgtgagcgtgtgttgaagtgtcggtttgattcggtggatgacacgtttacggttgatcctgtgaatcgtggctatgcgcgttatgtgattcatttgacagcttatgacccgttttggtatggggaggagcagaagtttcgttttagtaacgcgaagttgcaggattggttgggtggcggccctgtcggcaaggatggcacggcgtttcctgtggtgttgacgcctggtgttggttctggttgggataatctgtctaataagggtgatgtgcctgtgtggcctgtgattcgtgttgaggggcctttggagtcgtggtctgtgcagattgatggtttgcgtgtgtcttcggattatcctgtcgaggagtttgattggatcactattgatacggatcctcgtaaacagtctgcgttgttgaatgggtttgaggatgtgatggatcgtttgacagagtgggagtttgccccgattccgcctggcggttcgaagagtgtgaatattgagatggttggtttgggtgccattgttgtgtcggtgcagtacaggtttttgagggcttggtgaatagttgatggctggtctggttccgcagataacattgtttacgccggattatcgccgggttgcgcctatcaatttttttgagtcgttgaagttgtcgttgaagtggaatggtttgtcgacgctggagttggtggtgtcgggggatcattctaggcttgacgggttgactaggccgggtgcacggctggtggttgattatggtggtggccagattttttctgggcctgtgcgtcgggtgcatggtgtgggtccgtggcgttcttcgcgggtgactatcacgtgtgaggatgatattcgcctgttgtggcgtatgttgatgtggcctgtgaattatcgtcctggtttggttggtatggagtggcgtgccgacagggattatgctcactattctggtgcggcggagtcggttgctaagcaggtgttgggggataatgcttggcgttttccgcctggtttgtttatgaacgatgatgagagtcgtggccgctatattaaggattttcaggtgcggtttcacgtgtttgccgataagttgttgccggtgttgtcgtgggctcggatgactgtttcggtgaaccagtttgagaatgcgaagtttgatcagcggggtttgctgtttgattgtgtgcctgctgtgacgcgtagtcatgtgttgactgccgagtctgggtctattgtgtcgtgggagtatgtgcgtgacgccccgaaggctacttcggtggtggttggtggccgtggcgagggtaaggatcggctgttttgtgaggatgttgattcgatggccgagggggattggtttgatcgtgtagaggtgtttaaggatgcccgtaacacggattctgaacatgtgcatctcatcgatgaggctgagcaggtgctgtccgagttaggggccacgtcggggtttaagatcgagttggctgagtcggatgtgttgcggtttgggccaggccgcctgatgcccggggatttgatctatgtggatgtgggctcggggcctattgcggagattgttcggcagattgatgtggagtgtgattcgcctggtgatggttggacgaaggtgacaccggttgcgggggattatgaggataatccgtcggcactgttggctcgccgtgtggctggtttggctgccggtgtgcgggatttgcaaaaattttagtaagtgattggggtttgttgtgggtattgtgtgtaaagggtttgatggtgtgttgaccgagtatgattgggctcaaatgtctggtctgatgggtaatatgccgtctgtgaaagggccggacgattttcgtgtcggcacgacgattcagggtgccacagtgttgtgtgaggtcctgccggggcaggcttgggctcacggggtgatgtgcacgtcgaatagtgttgagacggtgacggggcagctgcctggtcctggtgagacccgctacgactatgtggtgttgtcgcgggattgggagcagaacacagccaagttggagattgttcagggtggccgtgcggagcgtgcccgggatgtgttgcgtgccgagcctggcgtgtttcatcagcagctactggcgactttggtgttgtcgtctaacgggttgcagcagcagctggataggcgtgctgttgcggctagggttgcgtttggcgagtctgcggcttgcgatcccacccctgtggagggtgaccgtataatggtgccttcgggggctgtgtgggctaaccatgccggcgagtggatgctgttgtcacccagaattgagacgggttcgaagtcgatcatgtttggtggttctgctgtgtatgcttacacgatcccgtttgagcgccagttcagtagtccgcctattgtggtggcgtctatggctacggcggctgggggcacgcagcagatcgatgtgaaagcctacaatgtgactgcccaaaattttagtttggcgtttattacgaatgatggttcgaagccgaatggtgtgcctgcggttgcgaattggattgctgtcggagtgtgactgcacgggtgttgtggcggatggtgtgatgttggggggctgtagtgtcgtggtttactcctgcactggtggcctctatctgtaccgcgttggccacggttttgggttctgttcaggctgtcacatcccggtctaggaagcgtttacgcaggctgtcggctcaggtggatgcgatggaagagtatacgtggggtgtgcggcgcgaggtgcgaaggtttaacgccgggcttcctgacgatgtggagccgatgcatcttcctgatttgcccgagtttttgaaagatactgttgatggtggaggtgagtagggttgagggagttggaggaagagaaacggcagcgccgcaattttgagaaagcttcactgttgctgttgtttttgtcgcttgtactgttggcggtggttgctgcgggtgctttgcgtttcggggctgtatcctctgagcgggattcggagcaggcgagggcccagtcgaatggtacggctgccag

SEQ ID NO: 73 - DNA, 30016 bp, synthetic construct
  source 1..30016: mol_type = other DNA; note = PAC22
gcgcagggtgagcgtggccccgccggtgtgaacggatccgatggtaaagacggtaaagatggtaaggatggcgctgatgggcgttcggtgatatcggtgtactgttccgggggccgcctggttgtgaaatatagtgacggtacagcctccaccgtgtcgggttctgcggcctgtgagagtgtgaaaccgtcacctgtggttaccgtatcatcccacaaatagaatatgaagagggaagggtgttactagtgttgattgtggtgtttggtggtggtgtgttgtgagatacattccagcggcgcatcactcggccggttcgaatagtccggtgaacagggttgtgattcatgcgacatgcccggatgtggggtttccgtctgcttcgcgtaaggggcgggcggtgtctacggcgaactattttgcttccccatcgtctggtggttcggcgcattatgtgtgtgatgttggggagactgtgcagtgcctgtcggagtctacgattggttggcatgccccgccgaatccgcattctttgggtatagagatttgcgcggatggcggttcgcacgcctcgtttcgtgtgccagggcatgcttacactcgtgagcagtggcttgatccgcaggtgtggcccgcggtggagagggcggctgtcctgtgtcggcagttgtgtgacaagcatggtgttccgaaaagaaaactgtctgtggccgatttgaaggccggtaaacggggcatctgcgggcacactgatgtgacggatgcgtggcatcagtcggatcatgacgatccggggccgtggtttccgtgggacaaatttatggctgtggtgaatggccacggctgcggttcaagtagtgaggagttaacggtggctgatgtgaaagcgttacatgatcagattaaacaattgtctgcccagcttactggttcggtgaataagctgcatcacgatgttggtgtggttcaggttcagaatggtgacctggcgcgccgcgtggaggctttgtcgtgggtgaagaatccggtgacggggaagctgtggcgcagcaaggatgccctgtggagtgtctggtattacgtgttggagtgtcgtagccgtattgacaggcttgagtctgctgtcaacgatttgaaaaagtgatggtggtttgttgtgggtaaacagttttggttgggcctgctggagcgggcgttgaagacttttgtgcaaacgtttgttgctgtgttgggggtgacggcgggtgtcacgtatactgcggagtcgtttcgtggtttgccgtgggagtcggcgctgattacggctacggttgctgcggttttgtcggttgctacctcgtttggtagcccgtcgtttgtggctggtaagccaagcaagcctcaggtggatgcgggtttggttgagcctcacatggtggatgtgtcggatcctggcatgatcgagccgacggatgatgctgatcttggtgtgggctatgtgccgaaacacgctgccgagtcggaggttggcacggtagagtcgactgttgcataagtgaatatagatgtgtgccccagcggtgctgccacggttgtgtggtggttgccgctggggcactatttttgtgtctatagtattttatgattcgttgctgtcgatggtgtcttcgagcatctggtacaggtggaggcaggtgcagatggtttcgctggcctggtctagaacggttcggccgataacgtttttgtggttgtcgcggtggcggatgatagcccacatgatctcgtcggctgccgcctgtaatagttttgcctggtatgcgattccggcgagccagtctagtgcttcctggcttgcgtaggggctctggtcctcgctgttgccgcgggtgttgctgttgtttgtggggtgtccttcactgtcgcatagccataggatttcgctgcactcgtctagcgtgtcttggtcgatagcgagatcgtcgaggctgacattgttgacggtaaggttcacgttgtcgatggagatgggtacaccgtactggttttcaacactgtcaacaatgttttgtagttgttgcatgttggtgggctgttgttggacgatgcggtgtatcgctgtgttgagggtggtgtaagtgatgttgtgtgtgttgttcatggtttttatgccattccttcgttatcgtctggcatgtagtatgtgctgtttgcgtactcggtgagggtgatgagtgtttggtctgcccactgtttcacggtttgccgggtgactccgagtagttgggcggctgtggcgtaggtttggtcgtatccgtatacttcccggaatgctgccaacctagctaaatgttttcgctgtttggagggttcacaggcgagggtgtagtcgtcgatggcgagttgtagatcgatcatggtaacaaggttgttgccgtgatgctggggggcggttggtgggggtggcatgcccggctccacactgggtttccatggtccgccgttccagatccattgtgcggcttggatgatgtcggcggtagtgtaggttcggttcatgtgtcaccccctgaacaggtcgttggtgttgctggtgcgggtggtgtcgaatcgtccgacgcagtggcagtagtcgtacatgagtttgataatgtgttggtggtctcccaaataggtgttgccgctgatgctgtaggtggctgtgccgtctttgctgatggtgtatttggcggtgatggtttcggggttttcggtgtcggtgatgattgctgtggtggtggtgcctactgtttggagcacggtggtttgggttccgtcgtcgatggtggttttaaccatggtgtgtgttctcccctttgtgttagttgctggtttggttgtcggctagatgaatgatgtcgggtaagggtttcggctggtctaggtgttgtgtggttttgttggctaaacgtttggctaccctgtagcacattttggtgtagtgtttgttgtcgaggttgtggtattgttcccgcaccgcaatatatagcagggagtcttggtataggtcgtctgcactgattgcggggtagtgtgcggctgttttggtgcatgcccggttgagtgtgcgtagatgatggtttgtggcccacacccacgatgcggtggtggctaggtcggcttttgttggtcgtcggctcatggcatctctttcatctggctatctggtagttgtttggtgttttgttgttgatagtgtagcacacgagtccggggttgccggtggtgcctgttttgtgccggaaccatgtggattctccttccatggaggggcattggatgaaggtgcgttgtccttgctcggagatttctaggtggtgccggtgcccggccatgagaatgtgggatgtggtgccgttgtggaattcttgtccgcgccaccattcgtagtgtttgccggtgcgccattggtggccgtgggcgtgcagtatccgtgtgcctgccacatcaacggtggtggtcatttcgtcgcgttgggggaagtggaagtgtaggttggggtattggttgttgagctggtaggcttctgcgatggcgcggcagcagtccacgtcgaaggagtcgtcgtaggtggtgactcctttgccgaagcgtacggcttctccgtggttgccggggatggatgtgatggtgacgtttttgcagtggtcgaattggtggatgagttgcatcatggccatgcgtgtcaaccggatttgttccgtcaagggggtttgtgtgcgccaggcgttgttgccgccttgtgacacgtatccttcgatcatgtcgccgaggaatgcgatgtggactcgttgcggttttccggcttgttgccagtagtgtttagctgatgtgagggtgtgtaggtagtcgtcggcgaagtgtgatgtttctcctccggggatgcctttgccgatttggaagtctcctgccccgatgacgaaggccgcggtgctgtagtcggtgcgggtgtcttgttcgggttttgggggtgtccattcggctagcttgtcgacgagttcgtctaccgggtaggggttggttgcgggttggtggtcaataattttttgtatggatcggcctgtttctccgttcggtaaggtccattcggagatgcgtgtgcggcgcacggtgccgttggctagattgtcgtcgatggtgtcgatggcgttgtcgtggttggcgagctgtgtgaggagccggtctatgttgtctatcatcgggtatcctcctcttctgtttgtggtgtggtggcttgtttgcggcggtagtctttgatgacggtggcggagatgggggggtatcctcctcttctgtttgtggtgtggtggcttgtttgcggcggtagtctttgatgacggtggcggagatggggtatccggcttcagtgagcattcgggctagctgtgtggcggggatcgtcttgtcggcgaggacgtctgcagccttatcaccgtagcgttggatgagggtttcagttttggttgccatggtgtcctatcggttgtgtggtgggctgccatcctgtgcggcagtcgccgtcgtgtcctggtttgcgtgtgcaccacgatacggttccgtctgtgtggttgagtgttttgccgcacatgacgttttgtagatgctcgggcagtgcgccgttactctggttgctggtttgtgtgtcgaagagtgttttctggttggtgaaatgctcggatacggtgccgttgtggactgggagtatccatgttttccattgttgttgcatccgggtgttccagtggaattgtttagccgcgttttctgcctgtttggcggttttgtagtagcctacaatgattcgctggtggttgttgtctggctggtgtggccctttccagtattgtgccgccacggcgtagcggttgctggctgtgaagcgctcccagcagtattcaataatgtgttgcagtacactatcgggaatgtcttgtgcttggttttcgttaagccattcttcaacaatgatgtcgcgtatggcgcgtttgtctttagtggtgggtttgaacgagatgctcacgatagcaccggctggtcgtcttgcatgaactggttgaaggtgttgttcccggcgtgttgggcttgtgtgatttgctggtcggtccagtctgggtgttgctgtttcagatagtgccagtggcacgcattgtaggtttcgtcttgtagccgtgtgagatggttttcggtgatgatttgtttccacatagtccacgagacgtcgagcctgttgaggatttcgagggctgggatgttgaattggttgaggaacaggatttcgtgggtgtagtattccttctcgtaggcgtcccatccgcttcggtgcctgttgggctggtttttggggtaggcttcccggcagattttgtgcaaatgtttggccatgtcgtcgggtagtttaatgtcagggttggcgcggatcatggatcgcatcccatcataggtggtgccccaggtgtgcatgatgtaggtggggtcttcaccatcagtccatttttctgcacagatggcgaggcggatacgcctcctggcggcttggctggtgttgcgccggttggggattgggcacgtgtcgaggggatccatgatgctttttatgcctttctttgtttgggttgtttgtctagttttactgtagcacagtgtctagtgcttgtgtcaaccctgtttttccggcctgcaggtaggtgtctgtgacgtcgccgagggtgaggggcacatgggtggcttgggggagtgctgcctggagggtgtgggccatctggtcgcctgctttgtctgggtctgaccatatgtagatgtggtcgtagccttcaaaaaatttggtccaaaagttttgccacgaggtggcgccgggtagggcgacggccgaccatccgcattgttcgaggatcatggagtcgaattcgccttcgcaaatgtgcatttcggctgcctggttggccatggcggccatgttgtagatggagcctgtgtcccctgccggtgtcaagtatttggggtggttgtgggttttgcaatcatgggggagtgagcagcggaaacgcatttttcgtatttcggctggcccttcccagacggggtacatgtaggggatggtgatgcactggttgtagttttcgtggcctgggatggggtcattgtcgatgtatccaaggtggtggtagcgggctgtttcttcgctgattcctcttgccgagagcaggtcgagtatgttttcgaggtgggtttcgtatagggccgaggctttctggattcggcggcgttccgcaatgttgtaggggcgtatgctgtcgtacattcgggttttcttcctctaatcgttgtttcagtttgtggagtccacctccgataccgcatgtgtggcagtaccagacgcccttgtcgaggttgatgctcatggagggctggtggtcgtcgtggaacgggcagaggatgtgttgctcgttcctggacgggttgtagcgtatctggtgggcgtcgaggaggcggcaggtgtcagaggtgtgggaggagctcgttgagggttgataccacataggcttcgctccagggtttgttgcgctgtttcatgatgacgagtccgatggtggactggttttctcggtttcggtgggtttcatagttgcgtgcctcccggctggcttgtttcacgaattcggcgagatgtggttgcccggctttcgcctcgataatgtaggttttgtggccggttgtgaggatgaggtcgccttcgtcttcgcggccgttgaggtggaggcgttcgatatcatgtccgatgtcgcgtagctggtggaggagtcttgtttcccattcggccccagctcgcctattcctggattgctgtgtagccatcatagtcctttgtgtgttggggtcatgttccagggctgtttttctactaggggtccgaagaatgtgtattcggggtaggctcgtagtcgttcgtatcgggtgccgtctgggctggatttgccggttctctgtttgagtacggcgatgcgcgcctcggcggggatggtgagcccgttgccgttgtcttcgccaccgtagagtgagactcccaggattagttgtggtttttcggagaggccgtttttgatttcccgcctagctggggggtgttcgatgtcggtgccggttttgtcggttgcgtggtgggtgacaataatggtggagccagtgtcgcggcctagtgctgtgatccattgcatggcttcttgctgtgcctgatagtcactttcgcagtcttggatgtccatcaggttgtcgatgacgatgatgggtgggaaggtgttccacatttccatgtaggcttggagttccatggtgatgtctgtccatgtgatgggtgactggaatgagaatgtgatgtgtccgccgtggtggatgctgtctcgatagtattctggcccgtagttgtcgatgttgtgttgtatctgttgggtggtgtgttgggtgttgagtgagatgattcgtgtggaggcctcccagggtgtcatgtcccctgatatgtagagggctggctggttgagcatcgcggtgatgaacattgctagccctgatttttggctgccggaccgccccgcgatcatgactaggtcccctttgtggatgtgcatgtcctggttgtcatacaagggtgctagttggggtatgcggggcagttcggcggcggtttgggaggccctctcgaaggatctttggagagagagcatcggagccttaatctatctgtctgttggttgggtgttggtggtcagatggagtcgatgtcgatgtcagcatcggcgggggctgtggtgtcgtctagctggccggtgtcgcgcttgtctacgtattcggcaaccttatcgtagatggcgtcgtccaatggtttgaggacgaccgcgttgaacccgtttttggtgcgaacggtggcgagtttgaaggcctgctcttcgccgagataggcttctaggtcgcggatcatggagtgtgggcggtcgttgttgcctcgcgctttctcgatgatagcgttggggatagtttctggggtgccattgttgagatcctggagtgtgtggaagatggtgacatcggcgtaaatacggtcggcgacctgtccgccgtagccttcggtgttgtgctggacgtcgcggattttgaaggcgatggcggtggcgtcctggtttcgggaggggttgaagaaggtgctgttgctgttgttgcggtagttggcgagtcccattgttgtttcctttactatttgtgttggtttttgttgtcttatattggtttatcgggtgaggctgtttcgtttactgcggaacgcctcagacacgtcactgttactggtgatgatcttcttgtactgtttgaggaggtctgctagttgtgtcttgctggtggctttgttgatccggtcgatgatgatgtcgttttcctggttggcgattttgttgacgtagtctttggcggctttatcgtatcgatcttgaagcaggattgctgcgctagcgatcaaggtggctaaatcccagtctttggatacggtttcgtctttcaatcctcctagcaggtcaatgatggattgtttgatgtcttctgcggtgtctccgcggatgactgtccatggggcggcatagtctccaccgtatttgagtgtgatagttagttttccgttgtctgtggtgtgctcgtcggtcacgtgttttccttttcgttactgtcggtttggggtggctgtacggtggtttctatcgggtatctgtacgagtttttgccgttgacggcccagcaggcgtccttgacggggcatcctttgcagagtgctgtgacgtggggtacgaagatgccttggctgattcctttcattgcttgactgtacatggatgatacatgccggtaggtgttgttgtcaaggtcgtagagttcggttgctgtgccctgctcgactgattgctcgtctcccttggtggtggcgggtgtccaaaacatgcctttcgtcacatggatgccgtgttggttgagcatgtaccggtatgtgtgcagctgcatactgtcggcgggtaggcggccggttttgaggtcgaggatgaaggtttcgccggtgttggtgtcggtgaatacccggtcaatatatccgacaatctgggtgccgtcttggagggtggtttctaccgggtattcgatgcctggctggccgtcaataacagcggtagcgtattctgggtggttgcgcctccatgttttccaccggtccacaaaggtggggccgtatatcatccaccaattgtagtctttcttgttggggcccccgctttcgcacatgtttttgcacactcggccggagggtttgatgtttgtgccttcggattcggcgagggcgatttgggtgtcgaaaatgtttgtgaaggatgcgagtttgtctggtagtgcagggtattcggcgggattgtacaggtgtaagtcgtattgttcggtgatgtggtgtatggcgcttccggcgatggtggcgtaccaggtgtggtgttgggtgtggtagccgtgggataggcgccatttttctccgcattcggcccactgtgacagtgaactgtaggagatgtggcctggatggtggatggttttcgggtattgtgctaggggcattacttgtcgcctttgtgggtgttccatgggttgcgggtgtctaccccggcatcgtgttgctggtaggcgaggagtgccaagcagtgccaggcagcatgtgccagatgcggcaaatgtgattcgtggtcgaggttgtttccttgctgccatgatagcaggtgcctgtagagggcgtcgacactgtggctccacgggtagccgccggtccagttgttgtcgccgtatttggtggcaccgtagcctgccacttcgccgagggcgtgcaaggcggtagggtcgatgagggatagcctgcaaagtttcaattctttcttggcacccgtatcagggtcggtgtacatgctggttggctcatccatggtgtgtgtgctccttacgtgtggggttactggttggggttgtgggcgagtgctacggcgagaataatgatggcgagggtttctgcgatcagtattggtgttgtgatcatttgctgtcgcggggattgttggtgagggtggatgcgcctagcagggtggtgagggcgcatgcggcgatgatggcgagggcggctttgtggctggtgccggtggcgtacatccatgtgatgatggcgccctgtatccatgccagtgtggtgaagaacgtttcgtagctgtgcagctcgatactgttgggtgtgttcatgcttgctcctgaagaatggtgttgatggttgtgtaaatgttgtacaggtcggcttcgatggtttgtagctgtttgatttggtggtcgaggtcaatgtttgggttgagggtgttgatgcgggatgcgatgtcggtggctgtgcgtagtgtgccgccggtgtggtgaatgatgtgtgccgtgtcggcgagtccggtggtgacagcgtagcgggagaggagaggcatgactggggggtgctccttgacggggttactgttgcgggttgatgttgaggtcggtgacgtgcggtgagctttctgttcctgtgacgaggcagtggacggtgacggggagtttggatgctcccggctggcggacggtggcgccgtagacgatgctgaacgtgtctttgccaataattttgtggagttggaggtcgatgtcggggttgccgttccatttgacaccctgtgctgcagctgcctgttcagccttgtcattgcaggcgtgtgccgcggtgatcatggtgagacctgtggaggtttcttcaccccgtgtttgggcttgccggtgggcgcgctgctgttcggcttggagggagcggactgctgcagcctgcttggcggctttctcggctttgcgctgttggacggtttcaggtgtccattcggtgttggctgtggtggcttgtggggctggctgtgaggcgagtggcggattatcatcgggtgccgggaggaaggatgctgcggcgatgatggcgatggtggcgccggcgatggtgtagcctgttttcttgttcatgattttgtgttcccctttccggggtgttgttcgttgctgacatgatcaatactttcagcggctggaccctgtgtcaaggtgtcgctcagtattcttgagcgaatgtggtttgactgggggtgatggcttctttcgcccaataggatgtgccaccgctggtccagtatccgagtttgttgcgctgcatgcccttggcttccatctcatccacggtgaggcacctgcggcgattggggccttccttgaccccgtggtcgcctacccggtgcatgtcgcctgaggtggtactcgtgaatgtttcgtggcagattgtgcagtgctctggtcggtatccgatgattgtgctatcgcacttgtggcatgtccattgcatgattgctcctattttccattataagacttcctgtagtgccattttagcgccttgcgggtcttgggggtacaactatataggtcaggtatttctaggcgattctaggctcattgtgtgcgactggtggttatcgggcgcacagagtgagcaggtggccaacattgatgcgggtcacattccagtagagttgcgtggcttcctcactggtgagcggcttccactcgttgtggctgaacacggtgccatcggatgctatgaacgtgttggggcgtagcttgtggagttcagtctctacatgccgacggtaggtttcggcgaggccctcgaaatcgaggtggtcgcaggagaggttttcgaggcgtgtcaggtcgaaaggctcagggcagtcgtagctggcggggctgtagagctgggtgaagtggttggcaatcttctgcatcatgattccttttctggtgatggtgtgttgatggttttatcgggtggatgctttgaggatggcgtctacatcgatcatgtcgatcatgtcgttgagttcctcggcctcattctcggagaggtggcgccagccgggtggcccgtatagggcgccgtcgagggtgacagtccacaggggccggatgagtcgtatggcttcttcgactttggcgtggtacatgcggcgcaccatatccagatcgatgtcgtctgaatggtttccggtgaggctgtggaggctgagtgggtcgatttctgtctgcccgtagaggctggtgaatgatggtgtgatgagtgtgccatccatgagggtgctcccttctgaactgtttgggttggttgttgtggtttctagagtgtgtaggttgcaaccccatagtcaaggctacgctcattcggattgagcgtttcatgctggagtgtgtcgggtgtgacagatgtcactgaatccttgatggcctctctcagcgcctgaaatatgtccggggtgggattatgcagggttgaccctgctgatcgattctaggccccctacagggcgtctcaggggtatgtctgggtgatagcaggttcggtagatgatctagcgagtcaaggtgccaagctgagacagaagatctaccatctaggtgtgtgagatgtatcacactcgcctggcttagtgtgcaccctcaagaccacctagtcgatctggcgtggagggtgcagcccagaaataccgtttaaagccttcgcgcggagcctaggagcgccttacagggtgggggctaggtattcatacccccaagcaattctgatcgattctagacgcctccaggggcccgatacacgatcagtagtccagacacagatcatcaacccctatcctggttagctaagcctcaactatgtggacagtgtgggatgctaagagggaagaaggacacggtaaaagaaagaggggggagcatcagccttcaagcctgaaggtcttagcgcttagcaccgagccccctcaagggctcggcatcagcccgaacaggctcagctcatcaggcacagccctgaaaagggtacacgccatcagggaaggcttgagagtacgaggagccctagcgacgagtactcgaaagcctgaggaaacaccctcagcactgatgggcctagcgtgttcggaaaggacacaagagtaaagtgtgacagctgtccgggagtgaaacccgttccggctaggggtttcagccttaaccaccctcaaaggttacaagactctaagaaaatttaagaaaactcttaggaagaaagttgtgttcatatccccctaaaaacacccaaaatagccctcaaacccgcctatagagccaaaaccaccagtttgactcatcccaggtggggtatgataggctggacaggtagccagctggacgcgaggccagaaagtgctgacgcacttcccgacctcgcttaccatcagtctaccaaacactttaaagcttcaaggcttagcgctaagcccttaagaccttaacactgaacaccgagccccctcaagggctcggcatcagccttaaagccttaaacactttaagtaactttaaaaccttaacagcttaacacttaaggttataaataaacattaaagctttaaagtcttaaagtaaatatataaccttaacacttaagttaagtataaaaccttaaaagctaagcacttaaagatataaacttaacatcagtgtttaagacttaaagagttaaagtaactattaagacttaaaggcttataagctttaaacacttaaagtaactataagactttaaaaaccttaagtacttaaagttaaccatcagtcttaaactttaatactataacctataagtcttaaagcttataggtataataatataatataagtattaaagcttataagttataaaagttttagaagagttaaagggttaacttctttacttctcttctctctttggttctttctctcttctcttcttttcttcatcaggggagaagaggaacctttaaccgtcaacgctgatggactttcaaccgtgtgactcgtgtaccaccggtcgcacgctcccgatggcacactccccacatgctacctgtgtccctttcaggcttagcgtgttcggctgaaggcgtacggcgtgtcacgcttaaacccttaacaccaggtaagacttaaagtgtatattataagtagaagactttaaaacctttaaggtgttcccgctgagcctgtgttcttcaccgctaggcgctaagcgctaagccttgaaacgcgaacacccatccacccttttcttttaccgtgtccttcttcttttgacaccgctggggggcgatgtgatctttttcacatgccagggggtaggagaagaaaacaaccaccccggcacaaacagaacacccccctaaacgaacaaaacagggcccaggatcgaacagcagggcaccggtagagtattcctacccccagacaattccaggccgttacaggagcaatgagaggctcacaggggccataggagatcaggggacgtgatggcacacaccaaccgcacagccagccaagcccaccggcgctggcggcaacgactcatcacccaagccaaacagcaaggccaaaccgaatgcccactctgcggagccaccatcacctggggcacacatgacctgccaaccagccccgaagccgaccacatcacacccgtcagcaggggaggactcaacaccctcgacaacgggcaaatcatctgcagaacatgcaacagaagcaaaggcaatcgcagcgaaccaaacattcaattccaacaacaaaccacaaaaacgttgatcccatggtgaaaaaacagccaacccccacgggaaccaccccctgcacacccgtgcaagacctcgtacggcttagtgaaatacctcccttttgtggttttgtctgtctgtcgactttttgtgttggtggtgagtgttgtgcagcctgagcttcctgatagtcgtggatggtgtggggagacgcgtcgttggtggcgtgtgtggggtgaggatagtcgcgcgcagtacgtgtctgatgaggagtggctgtttctcatggatgctgcggtgattcatgattgtgtgtggcgtgagggtcgcgcggatttggtggcttcgcttcgtgctcatgtgaaggcttttatgggcatgttggatcggtattcggttgatgtggtgtctggtggccgtggtgggggttctgctgtggcgatgattgaccggtatcggaagcgtaaaggggcctaatgtcgagcgttgttggttctcaggttcctcgtcaccgggtggctgcggcttattcggtgtctgctggcggtgatgcgggtgagcttggtagggcttacgggttgacgcctgatccgtggcagcagcaggtgttggatgattggctggctgtgggtggtaatggcaggcttgcttcgggtgtgtgtggggtgtttgtgcctcgccagaatggcaagaatgctattttggagattgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgggcgacgaatggccaggaggctattgtgttgcatcatccggattgtgccacgtttgagaagaagtgtggctgtccgggttggggttcggttgagtttgtggcccgtagccggggttcggctcgcgggtttactgttgatgatttggtgtgtgatgaggctcaggagttgtcggatgagcagttggaggctttgcttcctacagtgagcgctgccccgtctggtgatccgcagcagattttccttggcacgccgcctgggccgttggctgacgggtctgtggtgttgcgtcttcgcgggcaggcgcttggtggcggtaagcggattgcgtggacggagttttcgattcctgacgagtctgatccggatgatgtgtcgcggcagtggcggaagcttgctggtgacactaatccggcgttgggtcgtcgtctgaattttgggactgtgtcggatgagcatgagtcgatgtctgctgccggttttgctcgggagcggcttggctggtgggatcgtggccagtctgcttcgtcggtgattccggcggataagtgggttcagtctgctgtgggtgaggcgagtcttgttggcggtaaagtgtttggtgtctcgttttctcgctcgggggatcgtgtcgcgttggcgggtgctggccggactgatgctggggttcatgttgaggttattgatggcctgtctggcacgattgttgatggtgtgggccagctggctgactggttggcgttgcgttggggtgacactgaaaagattatggttgccgggtcgggtgcggtgttgttgcagaaggcgttgacggatcgtggtgttccgggccgtggcgtgattgtggccgatactggggtgtatgtggaggcgtgtcaagccttcctggagggtgtcaggtcgggtgtgatcagtcaccctagggctgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaagcgaagcggcgtgaacggtctggtaggaagcgggtgtctgtggtatgaactcggatgagttggctctgattgagggcatgtttgatcgtatccgaaggttgtcttcgtggcattgtcgtattgagggctactatgagggttctgcccgggtgcgtgatttgggggtggctattcctccggagttgcagcgtgtgcagacggtggtgtcgtggcctgggattgcggtggatgctttggaggagcgtctggattggcttggctggacgaatggtgacggctacggcctggatggtgtgtatgctgcgaatcggcttgctacggcgtcgtgtgatgtccaccttgatgcgctgatttttgggttgtcgtttgttgcgatcattccccaagaggatgggtcggtgttggttcgtccgcagtcgccgaagaattgtacgggccggttttctgccgatgggtctcgtttggatgctggccttgtggtgcagcagacgtgtgatcctgaggttgttgaggcggagttgttgcttcctgatgtgattgttcaggtggagcggcgaggtagccgtgagtgggttgagacgggccgtatcgagaatgtgttgggtgcggttccgttggtgcctgttgtgaatcgtcgccgtacttcgaggattgatggccgttcggagatcactcggtcgattcgtgcttacacggatgaggctgttcgcacactgttggggcagtctgtgaatcgtgatttttatgcctatccgcaaaggtgggttacgggtgtgtcggctgacgagttttcgcagcctggctgggttctgtcgatggcttctgtgtgggctgttgataaggatgatgacggcgataccccgaatgtggggtcgtttcctgtgaattctcctacaccgtattcggatcagatgcgtttgttggcgcagttgactgcgggtgaggcggctgttccggaacgctatttcgggtttatcacgtctaacccgccttcgggtgaggctttggctgcggaggagtctcggcttgtgaagcgtgctgagcggaggcagacgtcgtttggtcagggctggttgtcggttggtttcttggctgccagggcgttggattcgagtgttgatgaggccgcgttttttggtgatgtgggtttgcgttggcgtgatgcttcgacgccgactcgggcggctacggcggatgctgtgacgaagcttgtgggtgtcggtattttgccggcggattctcggacggtgttggagatgttggggcttgatgatgtgcaggttgaggctgtgatgcggcatcgtgccgagtcgtcggatccgttggcggcgctggctggggctatttctcgtcaaactaacgaggtttgataggcgatggcttcgggggttgtgtcgaggcttgctgcgactgagtatcagcgtgaggcggtcaggtttgccgggaagtatgcgggctattatgccgagctgggtcgtttgtggcattccgggaagatgacagatgcgcagtatgtgcgtttgtgtgtggagttggagcgtgccggccatgacggttccgcggcgttggcgggcaaattcgtgtccgattttcgccggttgaatggtgtggatccgggtttgatcgtgtatgacgagtttgatgctgcggcggcgttggctaggtcgttttcgactatgaagattcttaagagtgacccggatagggcgaatgatacgattggtgcgatggctgcgggttttgatcgggctgtgatgaatgctggccgtgacacggttgagtggtctgcgggtgtgcagggtaggtcgtggcgcagggtgactgatggtgatccgtgtgctttttgtgccatgttggctacgaggtcggattatacgactaaagagcgggcgcttactacgggtcatactcggcgtcataagcgtgccggtaggcgtccgtttggttcgaagtatcatgatcattgtggttgtacggtggttgaggttgttggcccttgggagccgaatagggctgatgccgcatatcagaggacgtatgagaaggctcgtgagtgggttgatgatcatgggttgcagcagtcgcctggcaatattttgaaggctatgcgtactgttggcgatatgagatgatggtttccggttgtgtgccgccggttattggtgcacagggttgtctcccgcacgggggtcaacaatgttgtgttgttttccgcaaggagtgtaggttaggctatggccgatcagagtgttgaagaacagaatgttgacaatgatgctgttgagcccggaaagggtggagacgttgttgatgttgtgaaggatgggcaggctgccggcgatgatcatgccggtgatgtttccgtgaaggaggagtcttcttctggcacggattggaaggctgaggcccgtaagtgggagtctcgtgctaaaagtaattttgccgagttggagaagcttcgcgcctcggatggtgatgcggggtctgtgattgatgagcttcgccgcaagaatgaggaactcgaagaccggattaatgggtttgttcttgagggtgtgaagcgtgaggtggctgccgagtgtggcctgtcgggtgatgcggtcgctttcttgcacggtggcgatcgtgaagcactggtggagtctgctaaggctttgaagggtttgatcgaccagagtggtggtggcgcgggtgtgcgccgtcttgcggggagtgcccccgttgatgatgttaaacgacgtgagggtgtcgcgtttgtggatgctcttgtcaataattctaggagatgatttataatggctgacgattttctttctgcagggaagcttgagcttcctggttctatgattggtgcggttcgtgaccgtgctatcgattctggtgttttggcgaagctttcgccggagcagccgactattttcggcccggtgaagggtgccgtgtttagtggtgttcctcgcgcgaagattgttggtgagggcgaggttaagccttccgcgtctgttgatgtttcggcgtttactgcgcagcctatcaaggttgtgactcagcagcgtgtgagcgacgagtttatgtgggctgacgcggattaccgtctgggtgttttgcaggatctgatttcgcctgctctgggtgcctcgattggtcgcgccgtggatttgattgctttccacggtattgatcctgccactggtaagcctgccgcggctgtcaaggtgtcgctggataagacgaagcatattgttgatgccacggattccgctacgactgatcttgtgaaggctgtcggcctgatcgctggtgctggtttgcaggttcctagcggggttgctttggatccggcgttctcgtttgctctgtctactgaggtgtatccgaaggggtctccgcttgccggtcagccgatgtatcctgccgccgggtttgccggtttggataattggcgtgggctgaatgttggtgcttcttcgactgtttcgggtgccccggagatgtcgcctgcctctggtgttaaggctattgttggtgatttctctcgtgttcattgggggttccagcgtaacttcccgatcgagctgatcgagtatggtgacccggatcagactgggcgtgatctgaagggccataatgaggttatggttcgtgccgaggctgtgctgtatgtggctatcgagtcgcttgattcgtttgctgttgtgaaggagaaggctgcaccgactcctcctccggctggtaactgatacaagataagcgaatgtgtactatgtgcagggggtggtgttgatgggtatcattttgaagcctgaggatattgagcctttcgctgatattcctagagagaagcttgaggcgatgatcgctgatgtggaggctgtggctgtcagtgtcgccccctgtatcgctaaaccggatttcaaatacaaggatgccgctaaggctattctgcgcagggctttgttgcgctggaatgataccggggtttcgggtcaggtgcagtatgagtctgcgggcccgtttgctcagactacacggtctaatactcccacgaatttgttgtggccttccgagattgctgcgttgaagaagttgtgtgagggtgatggtggggctggtaaagcgttcactatcacacccactattaatagtagatatgcacattctgaggtgtgttccacggtgtggggtgagggttgctcgtgcgggtcgaatattaacggctacgctggccctttgtgggagatatgatatgaccagttttccttatggtgaaacggttgtgatgcttcagccgactgttcgtgtcgatgatcttggcgacaaggtggaagactggtctaagcctgtcgagactgtgtaccataacgtggccatttatgtctctgtttcgcaggaggatgaggctgccggccgtgactctgattatgagcattggtcgatgcttttcaagcagcctgttgtgggtgccggttatcgttgccggtggcgtattcggggtgttgtgtgggaggctgacgggtctcctatcgtgtggcatcaccccatgtccggttgggatgctggtacgcaggttaatgtgaagcgtaagaagggctgatgggttgtggctcaggatgtgaatgtgaagctgaacttgccgggtattcgtgaggtgttgaagtcttctggggtgcagtcgatgttggctgagcgtggcgagcgtgtcaagcgtgcggcctcggcgaatgtgggcggtaatgcttttgataaggcccaataccgtagcggtttgtcgtcggaggtgcaggttcaccgtgttgaggctgtggcccgtattggcaccacctataagggtgggaagcgtattgaggcgaagcatggcacgctggcccggtcgattggggctgcgtcgtgatcgtttatggtgatccgcgtgtgtgggctaaacgtgtgctcaaggatgatggctggctgtccgatataccttgtgtggggacggtgcctgaggattttagcggtgacttgatttggttggctcttgatggtggcccgcagttgcatgttcgtgagcgtgtttttttgcgtgtgaatgtgttttctgatatgccggatcgtgctatgtcgttagctaggcgtgttgaggctgtgctggctgatggtgtggacggtgacccggtggtgttttgtcggcgttctactggccctgatttgctggttgatggtgcacgttttgatgtgtattcgctttttgagctggtgtgtcggcctgtcgaatccgagtaagcgtatcgttgttttttagtttgattgttttgtagtttgattgttttttgggggttatgatggctgaaacacgtaaagcgtctaatgttcgctctgctgttactggcgacgtttatattggtaaagcgcacgcgggtgattctattaagggtgtggaggcggttccttccgggcttacagctttagggtatctgtctgatgacgggtttaagattaagcctgagcgtaaaacggatgatttgaaggcttggcagaatgcggatgttgttcgcactgtagctacggagtcgtctatcgagatttctttccagctgatcgagtctaagaaagaggttatcgaactgttttggcagtcgaaggttactgccggatccgattcgggttcgttcgatatttcaccaggcgccaccactggcgtgcatgctttactgatggatattgttgatggtgatcaggttattcgctactatttccctgaggtcgagttgatcgatcgtgacgagattaagggcaagaatggcgaggtgtatgggtatggtgtgacgttgaaggcgtatcctgcccagattaataagactggtgatgcggtgtctggtcgggggtggatgacggctttaaaagctgatactcctccgactcctcctccagccccggttcctccgaagcctcagccggatccgaatccgccgtccgataactgatacacgattttaggggattgttgatagatgagtgacactggttacacgttgaagattggtgaccgtagctgggtgttggcggatgcggaggagacggctcaggctgttcctgcccgcgtgtttcgccgtgccgccaggattgcccagtcgggtgagtctgcggatttcgcccaggttgaggtgatgttttcgatgttggaggctgccgcaccggctgacgcggtggatgctttggaggggcttcctatggttcgtgttgccgagattttccgcgagtggatggaatataagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccatctgaatggttggcagcgcccgtttgagtggtgtgagtgggctgtgttggacatgttggatcattacaggtctgctaatagtgaggggcagccggagcctgtggtgaggccgacggatgagcgtagggcccggtttacgtctgggcaggtggacgatattttggcgcgtgttcgtgctggtggcggggtgtctcgcgagattaatattatggggtgaatagtgtatgtctggtgagattgcttccgcgtatgtgtcgttgtatacgaagatgcctggccttaaaagtgatgttggtaaacagctttctggggtgatgcctgcggagggtcagcgttcgggtagcttgtttgctggcgggatgaagttggcgcttggtggtgcggcgatgatgggtgccatcaatgttgctaagaagggcctcaagtcgatttatgatgtgactattggtggcggtatagctagggctatggctattgatgaggctcaggctaaactgactggtttgggtcatacgtcgtctgacacgtcttcgattatgaattcggctattgaggctgtgactggtacgtcgtatgcgttgggtgatgcggcgtctacggcggcggcgttgtctgcttcgggtgtgaagtctggcgggcagatgacggatgtgttgaagactgtcgccgatgtgtcttatatttcgggtaagtcgtttcaggatacgggtgctatttttacgtctgtgatggctcgcggtaagttgcagggcgatgacatgttgcagcttacgatggcgggtgttcctgtgctgtctttgcttgccaggcagactggtaaaacgtctgctgaggtgtcgcagatggtgtcgaaggggcagattgattttaacacgtttgcggctgcgatgaagcttggcatgggtggtgctgcgcaggcgtctggtaagacgtttgagggcgctatgaagaatgttaagggtgccctgggttatcttggtgctacggctatggcgccgtttcttaacgggttgcggcagatttttgttgcgttgaatccggttatcaagtcggtgacggattctgtgaagcccctgtttgcatcggtggatcaggggattcagcggatgatgccgtctattttggcgtggattaaccggatgccgggcatgatcactcgaatgaatgcacagatgcgcgccaaggtggagcagttgaagggcgtttttgcgaggctgcatttgcctgtccctaaagtgaatttgggtgccatgtttgctggcggcaccgcagtgtttggtattgttgccgccggtgtggggaagcttgttgcagggtttgccccgttggcggtgtcgttgaagaatttgttgccgtcttttggtgctttgaggggtgccgctggggggcttggtggcgtgtttcgcgccttgggtggccctgttggtattgtgatcggcttgtttgctgccatgtttgctacgaatgcccagttccgtgccgctgttatgcagcttgtgggggttgttggccgggctttggggcagattatggtcgccttgcagccactgttcgggattgttgctggcgtggttgccaggttggctcccgtttttggccagattattggtatggttgctggtttggcggcccggctggtgcctgttattggtatgcttattgccaggctggttcctgttatcacccagattattggtatggtaacccaggttgctgccatgttgttgcctatgctgatgccggttattcaggctgttgttgctgtgatacggcaggttattggtgtggtcatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgccgtgctacaggtggtggcccgtattattccggttattatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatatcaaatcgtttacgtctgcgtttattaacggtttcaagtcgatcatttctgccggtgttgccgcggttgtggggttttttacgcggcttggtttgtcggttgcctcccatgtgaggtctggttttaatgcggcccgtggagctgtttcttccgctatgggtgcgattcggagtgttgtgtcttcggtggcgtctgctgttggcgggtttttcgggtcgatggcttctcgggtccggaatggtgctgtgcgcgggtttaacggggccaggagtgcggcttcttctgctatgcatgctatggggtccgcggtgtctaacggtgtgcatagtgtgctggggtttttccggaatctgcccagcaatattaggggcgccttgggtagtatggggtctttgttggtgtctgctggccgtgatgtggtggccggtttgggtaacggtattaagaatgctttgagtggcctgttggatacggtgcgtaatatgggttctcaggttgcgaacgcggcgaagtctgtgttgggtattcattctccgtctcgggtgtttcgtgacgaggttggccgtcaggttgttgccggtttggctgagggtattactgggaatgctggtttggcgttggatgctatgtcgggtgtggctggtcggctgccggatgttgtggatgcccggtttggtgtgcgatcgtctgtgggctcgtttaccccgtatgaccggtatcggagtgcgagcgagaagagtgttgtggtgaatgtgaatgggcctacttatggtgatcctaatgagtttgcgaagcggattgagcggcagcagcgtgacgctttgaacgcgttggcttacgtgtgattgggggtgttgtgcatgtttattcctgacccttctgatcgtgccggtttgactgttacttggtctatgttgccgttgattggtaatgatcctgagcgtgtgcttcatttgacggattatacgggtgcgtctcctgtcatgttgttgaatgattcgttgcgtggccttggtgttcctgaggttgagcatttttctcaaactcatgttggggtgcatggctcggagtggcgcgggtttaatgtgaagcctcgcgaggtgacgttgccggtgttggtgtcgggtgtcgacgaggatccggtgggcgggtttcgtgacggttttttgaaagcctatgatgcgttgtggtctgcttttcctcccggcgaggagggggaactgtcggtgaagactcctgccggcaaagagcgtgtgttgaagtgccggtttgattcggctgatgacacgtttacggtggatccggtgaacaggggttatgcccgctatctgttgcatttgacagcttatgacccgttttggtatggggatgagcaaaagtttcgtttcagtaacgcgaagttgcaggattggttgggtggcggccctgtcggcaagaagggtaccgcgtttcctgtggtgttaacaccgggtgtgggctctggctgggataacctgtctaacaggggtgatgtgccggcgtggcctgtgattcgtgttgagggccccctggagtcgtggtctgtgcagattgatggtttgcgtgtgtcttcggactatccggtggaggagtatgattggattactattgatacggatcctcgtaagcagtctgcgttgttggacgggtttgaggatgtgatggatcgtttgacggagtgggagtttgctcctattcctccgggtggttcgaagagtgtgaatattgagatggttggtttgggtgccattgttgtgtcggtgcagtacaggtttttgagggcttggtgaatggttgatggctggtcttgttccgcatgtcacattgtttacacctgattatcgccgggtagcccctatcaatttttttgagtcgctaaaactgtcgttgaagtggaatggtttgtccactttggagttggtggtgtcgggggatcattccaggcttgacgggttgacgaagcctggggctcggctggttgttgattatggtggtggccagattttttctgggcctgtgcgtaaagtgcatggtgtgggtccttggcgttcttcccgtgtgactatcacgtgtgaggatgatatccgcctgttgtggcgtatgctgatgtggcctgtgaattatcgtcctggtttggtgggttcggagtggcgtgcggaccgggattatgcccactattcgggtgcggctgagtcggttgctaagcaggtgttgggggataatgcttggcgttttccgcctggtttgtttatgaacgatgatgagagtcgtggccgctatattaaggattttcaggcccggtttcacttgtttgccgataaactgttgccggtgttgtcgtgggctcggatgactgtcacggtgaaccagtttgaggatgcgaagtttgatcagcgtggtttgctgtttgattgtgttccggctgtgactcgtgagcatgtgttgactgccgagtcgggttcgattgtgtcgtgggagtatgtgcgtgacgcaccgaaggctacgtcggtggttgtgggtggccgcggcgagggccgggacaggctgttttgtgaggatgttgattcggcggccgaggaggactggtttgatcgtgtagaggtgtttaaggatgcccgtaacacggattctgagaaggtgtctctcttcgatgaggctgagcaggtgctgcaagagtcgggggccacgtcggggtttaagatcgagttggccgagtcggatgtgttacggtttgggcccggcaatctgatgcccggtgatcttatctatgtggatgtgggctcggggcctattgcggagattgttcggcagattgatgtggagtgcgattcgccgggtgacgggtggacgaaggtgactcctgttgctggggattatgaggataatccgtcggcgctgttggctcgccgtgtggctggtttggctgcgggtgtgcgggatttacaaaaattctaattgttgggggtttgttgtgggtattgtgtgtaaagggtttgatggtgtgttgaccgagtatgattgggctcaaatgtctggtctgatgggtaatatgccgtccgttcatggcccggatgattttcgtgtcggcacgacgattcagggttccacggtgttgtgtgaggtcctgccggggcaggcttgggctcacggggtgatgtgcacgtcgaatgctgttgagacggtgacaggtcagcttccgggcccgggtgagacccgctatgactatgtggtgttgtcgcgggattgggagcagaatacggcgaagttggagattgttcctggtgggcgtgctgagcgtgctagggatgtgttgcgtgcggagcctggcgtgttccatcagcagttgttggctactttggtggtgtcgtctaacgggttgcagcagcagcttgacaggcgtgctatagctgcccgtgtggcgtttggggagtctgctgcgtgtgaccctacccctgtggagggtgaccgtgtgatggttccttcgggggctgtgtgggctaaccatgctaacgagtggatgctcctgtctcctcgggttgagacgggttctaagcagatccagtttggcgggtctgctgtgtatgcttacacgatcccgtttgcccggccgtttagtagcccgcctatcgtggtggcgtctatggctacggcggctgggggcacgacacagattgatgtgaaagcctacaatattactagcaaggattttagtttggcgtttattacgaatgatggttcgaagccgaatggtgtgcctgcggcggctaattggattgctgtcggcgtgtaatgtacggcttgcgtgtgcgggacgtgttgtggtggttgtagtggtagggggctgtagtgtcatggtttacacctacgcttgtggcctctctttgtaccgctatcgctactgttcttggttcgattcaggcggctatgtacaggtcgaagaagaggcttaggcagttgtctgcgcaggttgatgcgatggaagagtacacgtggaatattcgccatattgttcaccgctataacgcgaatttgccggatgatgttgagccggtgaagatgcctgatttgcccgagtttttgaaggatactgttgatggtggtggggggtgaattgtgagggagttagaggaggagaagcggcagcgccgctcgtttgagaaggcttccctgatattgttgttcttgtcgcttgtcctgttggcggtggttgccgggggtgctttacgtttcggggctgtatcctctgagcgggattcggagcaggctaaagcccagtctaatggtacagccgccaggggtttggctgcccgtgtgtggcaggtgtgtgcttctggtggatgggagtctgtgcggcttcaccagtctggtttgtgtgtggatgctgtgcgtgttgagcggagtgtgcagggtgttccgggtccggctggtgtgcgtggcccgcaggggccggctggtgttgatggccgggatggtagcaatggttctgctgggctggttgggcctgttggtccgcagggttcccctggcttgaatggcgtgaagggtcctgacgggctgcccggcagtgacggccaggatggccgtgatggtgttccgggccgtgcaggagtggacggtgtgaacggatccgatggcaaggatggtcgtgatggttcggctggtgagcgcggcgatgtggggccttcgggtcctgccggaccccctggc

SEQ ID NO: 74 - DNA, 29913 bp, synthetic construct
  source 1..29913: mol_type = other DNA; note = PAC13
tgagcgtggccccgccggtgtgaatggatccgatggtaaagatggtaaggatggccgctcggtggtgtctgtgtactgttctgatggtcgcctggttgtgaaatatagtgacggtgtggcttctaccatatcgggttcggtggcctgccagggtgtgaaaccgtcgcctatagtgactatatcatcccaaaaatagaaaggagtggctgtgatggtagtgtttggtggtggtgtgtggtgagatacattcctgcagcgcatcactcggccggttcgaatagtccggtgaacagggttgtgattcatgcgacatgcccggatgtggggtttccgtccgcctcgcgtaagggtcgggcggtgtctacagcaaactattttgcttccccatcgtcgggtggttctgctcattatgtttgcgatattagtgagacagtgcaatgtttgagtgagtctacgattggttggcatgccccgccgaatccgcattctttgggtatagagatttgcggcgatgggggttcgcacgcctcgttccgtgtgccggggcatgcttacacgagggagcagtggctggatcctagggtgtggcctgcggtggagaaggctgccatcctgtgtagacgtttgtgtgacaaatataatgttccgaaaaggaaactgtcggctgccgatttgaaggccggtaaacgtggtgtttgcgggcatgtggatgttacggatgcgtggcatcagtcggatcatgacgatccggggccgtggtttccgtgggacaaatttatggctgtggtgaatggccacggcggcggttcaagtagtgaggagttgagtatggctgatgtacaagcgttacatgatcagattaaacagttgtcggcacaggtggcccagtcggtgaataagctgcatcacgatgttggtgtggttcaggttcagaatggtgatttgggtaagcgtgttgatgccttgtcgtgggtgaagaatcctgtgacggggaagctgtggcgcactaaggatgctttgtggagtgtctggtattacgtgttggagtgtcgtagccgtattgacaggctcgagtctgctgtcaacgatgtgaaaaagtgatggtggtttgtggtgggtaaacagttttggttgggcctgctggagcgggcgttaaagacttttgtgcaaacgtttgtggctgtgttgggggtgacggcgggtgtcacgtatacggcggagtcgtttcgtggtttgccgtgggaatcggccctgatcacagccggggttgctgcggttttgtcggttgctacctcgtttggtagcccgtcgtttgtggccggcaaacctggcaagcagcccctggtggatgagggtttggttccaccggatgatcctggaatagtggagtctcactcggtggatgtgtcggatcctggcatgatcgagccgacggatgatgcggatcttggtgtaggctatgtgccgaaacatgctgccgagtcggaggttggcatgatagagtctactgttgcataagtgaatatagatgtgtgccccagcggtgctgccacggttgtgtggtggttgccgctggggcactctttttatgttctatagtattctatgattcgttgctgtcgatggtgtcttcgagcatctggtacaggtggaggcaggtagagatagtttcgctggcctggtcgagaacggttcggccgataacgtttttgtgattgtcgcggtggcggatgatagcccacatgatctcgtcggccgccgcctgcaatagtttggcctggtatgcgatcccggcgagccagtctagtgcttccgggcttgcatgggggctctggtcctcgctgttgccgcgggtgttgctgttgtttgtggggtgtcctgcactgtcgcataaccacaggatttcgctgcactcgtctagcgtgtcctggtcgatagcgagatcgtcgaggctgacattgttgacggtaaggttcacgttgtcgagggagatgggtacaccgtactggttttcgacactgtcaacaatgttttgtagttgttgcatgttggtgggctgttgttggacgatgcggtgtatcgctgtgttgagggtggtgtaggtgatgttgtgtgtgttgtccatggtttttatgccattccttcgttatcgtctggcatgtagtatgtgctgtttgcgtactcggttaacgtcatcagtgtttggtctgcccactgtttcacagtctgccttgtcactccgagtcgttgggcggcagacgcatatgtttggtcatacccgtatacttccctgaatgctgccaaccgtgccaaatgttttcgctgtttggatggctggcaggtgagggtgtagtcgtcgatggctagctgtagatcgatcatggtaacgatgttgttgccgtggtgttgtggcgcggttggtgggggtggcatgcctggctccacactgggtttccatggtccgccgttccagatccattgggcggcttgaataatgtcggcggtagtataggttcggctcacttggtcaccccctgaacaggtcgttgctggtggtggtgtcgaatcgtccgacgcagtggcagtagtcgtacatgagtttaataatgtgttggtggtctcccaaataggtgtttccgctgatactgtaggtggctgtgccgtctttactgatggtgtatttggcggtgatggtttcggggttttcggtgtcggtgatgatggctgtggtggtggcacctactgtttggagcacggtggtttgggttccgtcgtcgatggtggttttaaccatgaggtgttctccctttgtgttagttgctggtttggttgtcggctagatgaatgatgtcgggtaagggtttcggctggtcgaggtgttgtatggttttgttggctagccgtttggctaccctgtaacacattttggtgtagtgtttgttgtctaggttgtggtattgttcccgcaccgcaatatatagcagggagtcttggtataggtcgtctgcactgattgcggggtagtgtgcggctactttggtacatgcccggttgagtgtgcgtagatgatggtctgtggcccacacccacgatgcggtggtggctaggtcggcttttgttggtcgtctactcatggcatctctttcatctggctatctggtagttgtttggtgttttgttgttgatagtgtagcacacgagcccggggtttccggtggtgcccgtcttgtgccggtaccatgtggattcgccttccatggatgggcattggatgaaggtgcgttgtccttgctcggagatttctaggtggtgccggtgccctgccatgagaatatgggatgtggtgccgttgtggaattcttggccgcgccaccaatcatagtgtttgccggtgcgccattggtggccgtgggcgtgcagtatccgtgtgcctgccacatcaacggtggtggtcatttcgtctcggctggggaaatggaagtgtaggttggggtattggttgttgagctggtaggcttctgcgatggcgcggcagcagtctacgtcgaaggagtcgtcgtaggtggtgacgcctttgccgaagcgtacggcttctccgtggttgccggggatggatgtgatggtgacgtttttgcagtggtcgaattggtggatgagttgcatcatggccatgcgggtgagcctgatttgttctgtcaggggtgtttgggtgcgccaggcgttgttgcctccttgtgacacgtatccttcgatcatgtcgccgaggaaggcgatgtggactcgttgcggtttgcctgcctgttgccagtagtgttttgcgactatgagggagcgtaggtagttgtcggcgaagtgtgctgtttctcctccggggatgcctttgccgatttggaagtctcccgccccgatgacgaaggccgcggtgctgtagtcggtgtgggtgtcttgttcgggttttgggggtgtccattcggctagtttatcaacgagttcgtctaccgggtaggggttggttgctggttggtggtcaataattttttgtatggatcggccggtttctccgttcggtaaggtccattcggagatgcgtgtgcggcgcacggtgccgttggctagattgtcgtcgatggtgtcgatggcgttgtcgtggttggctagctgtgtgagtagccggtctatgttgtctatcactgggtatcctcctcgtgtgtggtggtggcttgtttgcggcggtagtctttgataacggtggcggagatggggtatccggcttgggtgagttgttttgctagccacgaggcggggatggttttgtcggcgagcacgtctgcagccttatcaccgtagcgttggatcaatgtttcagttttggttgccatgatgtcctatcggctgtgtggcgggctgccatcctgtgcggcagtcgccgtcgtgtcctggtttgcgggtgcaccacgatacggttccgtctgtgtggtggagtgttttgccgcacaggacgttttggagatgctccggcagctggtcattctggttgctggtttgtgtgtcgaagagtgttttctggttggtgaaatgctcggacacggtgccattatgcacgggtagtatccatgttttccattgttgttgcatccgggtgttccagtggaattgtttggcagctgtctcggcttgtttggcggttttgtagtagccgactagtatgcgctggtgttcactgtcgggcgggttttggcctcgccagtattgtgccgcaaccgcgtacctgttgttgtcggtgaagcgctgccagcagtattcgatgatgtgttgcagtacactatcgggaattttttgtgtttggttttcgttgagccattcggcttcgatgatgccgtgtatggcgcgtttgtctttggtggtgggtttgaacgagatgctcacgatagtaccggctgatcgtcttgcatgaactggttgaaggtgttgttcccggcgtgttgggcttgtgtgatttgctggtcggtccagtctgggtgttgctgtttcagatagtgccagtggcacgcattgtaggtttcgtcttgtagccgtgtgagatggttttcggtgatgatttgtttccacatagtccatgacacgtcgagcctgttgaggatttctatggctgggatgttgaattggtcgaggaagaggatttcgtgggtgtagtagtttttctcgtaggcgtcccatccgcttcggtgcctgttgggctggtttttggggtaggcttcccggcatactttgtgtaaccgtttggccatgtcgtcgggtagtttaatgtcggggttggcgcggatcatggatcgcatcccatcataggtggtgccccaggtgtgcatgatgtaggtggggtcttcaccatcggcccatttttctgcacagatggcgaggcggatgcgcctcctggctgtttggctgatgttgcgccggttggggatggggcacgtgtcgaggggatccatgatgttttttatgcctttcttggtttcgtgttgttgacgggttttactgtagcacagtgtctagtgcttgtgtcaaccctgtttttccggcctgcaggtaggtgtctgtgacatcccccagggtgaggggcacgtgggtggcttgggggagtgctgcctggagggtttgtgccatctggtcgcctgctttgtctgggtcggaccagatgtagatgtggtcgtagccttcgaagaatttggtccaaaagttttgccacgaggtggcgccgggtagtgctacggccgaccatccgcattgttcgaggatcatggagtcgaattcgccttcgcaaatgtgcatttcggctgccgggttggccatggcggccatgttgtagatggagcctgtgtcccctgccggggtcaaatatttggggtggttgtgggttttgcagtcgtgcgggagtgagcagcggaaacgcatttttcgtatttcggctggctgtccccaaacggggtacatgtatgggatggtgatgcactggttgtagttttcgtggcctgggatggggtcattgtcgatgtatccaaggtggtggtagcgggctgtttcttcgctgatgcctcttgccgagagcaggtcgagtatgttttcgaggtgggtttcgtagcgggctgaggctttctggattcggcggcgttccgcaatgttgtaggggcgtatgctgtcgtacattcgggttttttttctctagtcgttgttgtagtttggcgagtcctcctccgataccgcatgtgtggcagtaccagacgcccttgtcgaggttgatgctcatggagggctggtggtcgtcgtggaacgggcagaggatgtgttgctcattcctagacggattgtaccgtatctggtaggtgtcgaggaggcggcaggtgtcagaggtgtgggaggagctcgttgagggttgataccacataggcttcgctccatggcttgttgcgttgtttcatgacgacgagtccgatggtggactggttttcgcggtttcggtgtgtttcgtagttgcgtgcctcccggctggcttgtttcacgaattgggctaggtggggttgtcctgctttcgcctcgataatgtaggttttatggccggttgtgaggatgaggtcgccttcgtcctctttaccgttgaggtggaggcgttctatatcatggccggtgtcgcgtagctggtgcaataatcgtgtttcccattctgcgcctgccctgcggttgcgtgactgttgtgtcgacatgatagtcctttgtgtgttgtggtcatgttccatggctgtttttcggcgagtggcccgaagaatgtgtattccgggtatgccctgagccgctcatattttgttccgtctgggctggatttgcctgtgcgctgtttcaacactgagatgcgtgcctcggcggggatcgtgagcccgttgccgttatcctcgccaccataaagtgagactcccaatatgagttgtggtttttcggagaggccgtttttgatttcccgcctagccggggggtgttcgatgtcggtgccggttttgtcggtggcgtggtgggtgacaatgatggtggagccagtatctctacctaatgctgtgatccattgcatggcttcctgctgggcctggtagtcgctctcgcagtcttggatgtccatcaggttgtcgataacgatgatgggtgggaaggtgttccacatttccatgtaggcttgcagttccatggtgatgtctgtccatgtgatgggtgactggaatgagaatgtgatgtgttggccgtggtggatgctgtctcgatagtattctggcccgtagtcgtcgatgttttgttgtatctgtgtggtggtgtgttgggtgttgagtgagatgattcgcgtggaggcctcccagggtgtcatgtcccctgatatgtagagggcgggctggttgagcatggcggtgatgaacattgctagcccggatttttggctgccggaccgccccgcgatcatgactaggtcccctttgtggatgtgcatgtccaggttgtcatacaagggtgctagttgtggtatgcggggcagttcggcggctgtttgggaggctctctcgaaggatcgttggagagagagcatcggagccttaatctatctgtctatcggttggatgatgttttggtggtcagatggagtcgatgtcgatgtcagcatcagcaggggctgtggtgtcgtctagctgaccgttatcgcgtttgtctacgtattcggcaaccttatcgtagatggcgtcatcgaggggtttgagcacgaccgcgttgaagccgtttttggtgcgcacggtggcgagtttgaaggcctgctcctcgccaaggtaggcttcgaggtcgcggatcatggagtgtgggcggtcgttgttgccgcgggctttctcaatgatagcgttggggatggtttctggggtgccgttgttgagatcctcgagggtgtggaagatagtcacatcagcgtaaatacgatcggcggtctgtccaccgtagccttcggtgttgtgttccacgtcgcggattttgaaggcgatggcggtggcgtcctggtttcgggaggggttgaagaaggtgctgttgctgttgttgcggtagtttgcgagtcccattgttgtttcctttactgtttgtgttgttttgtttgttggtttgtgtcggtttttatcgggtgaggctgtttcgtttgctgcggaaagcctcagacacgtcactgttactagtgatggtcttcttgtactgtttgaggaggtcggctagctgtgccttgcttgttgcattgttgattttgtcgatgacgatgctgttttctttggatgcgatgttgtccacgtagtctttggcggcctggttgtatcggtcttggaggatgatggatgctgtggcgatcagggttgccaggtcccagttccttgccgcggagctgtttttgagtccgcctagcaggtcgatgatagtcttctttacctggtcggcggtgtctccgcggatgacggtccatggggcggcgtagtcgcctccgtatttgagtgtgacggtgaatcggtcgtcgtctgtgttgtcggtcactggtgctccttgccttcttttgttggggctgtgatggtggtttctatagggtacctgtaggcgtctttcccgttaacagcccagcaggcgtccttgacggggcatcctttacagagtgctgtgacgtggggtacgaagatgccttgactgattcctttcattgcttgactgtacatggatgatacatgccggtaggtgttgttgtcaagatcgtacagttcggtggatgtgccctgttcgaccgattgctcgtcccccttggtggtggcgggtgtccaaaacatgcctttcgtcacatcgttgccgtgttgggcgagcatgtaccggtaggtgtgcagctgcatactgtcggcgggtaggcgtcctgttttgaggtcgagaatgaaggtttcgccagtgtcggtgtcggtgaaaacgcggtcgatgtagccaacgatctgggtgccgtcctggagggtggtttctaccgggtattcgatgcctggctggccgtctaggattgcggtgatgtattctgggtggttgcgcctccatgttttccagcggtccacaaaggtggggccgtacatcatccaccagtcgtagtctttcttgtgtggcccgcccgactcgcacatgtttttgcatattctgccggagggtttgatttctgtgccttcggattcggcgagggcgacttgggtgtcgaaaatgtttttgaaggatgagagtttgtttggtagtgcagggtattcggtggggttgtacaggtgtaggtcgtattgttcggtgatgtggtgtatggcgcttccggcgatggtggcataccaggtgtggtgttgggcatggtagccgtgggataggcgccatttttcaccacattcggcccactgtgacagtgatgagtaggagatgtggcctggatggtggatggttttcgggtattgtgctaggggcattactggtcgcctttgtgggtgttccatgggtttcgggtgtcttggccggcattgtgttgctggtaggcgaggagtgcgaggcagtgccaggcagcatgggctagatggggtagcccggattcgtggtcgaggttgttgccttgctgccatgatagtaggtgcctgtagagggcgtcgacgctgtggctccacgggtatccgccggtccagttgttgtcgccgtatttggtggcaccgtagcctgcgacttcgccgagggcgtgtaaggctgcggggtcgatgagggagagtcggcatagtttgagttcttttttggcgcctgtgtctgggttggtgtacatgcgggtgggcttatccatggggtgtgtgctccttaggggtgggttactggttggggttgtgggcgagtgctacggcgaggatgatgatggcgagggtttctgcgatgatgatgggtgttgtgatcatttggtgtctcggggattgttggtgagggttgaggcgcctaggagggtggtgagggcgcatgcggcgatgatggcgagggctgccttgtgtggggtgccggttgcgtacatccatgtgatgatggcgccttggatccatgccagtgtggtgaagaatgtttcgtagctgtgtagctcgcttttgttgctggtgatgtcattcatggtagttttctgctttgtgtgcgatggttgtgtacatgtcgttgagtgtggtttcgatggtgatgagagtgttgatttcttggttgaggtcgatgttgtctttgagggtgtcgatgcgggcggcgatgtcggtggcggtgcgtaggcttactgctgcaccgtggatgatgtggcatatgtcggtgaggccgactttggcgatgtagtgtgacatgagaggcatagcggggatgctccttggcgggttactgttgcgggttgatgttgaggtcggtgacgttggggtggtcttctgttccggtgacgaggcagtggacggtgactgggagtttggatgcgccgggctgtttcgcggttgcgccgtagacgatggagaaggtgtctttaccaataattttgtggagttggaggtcgatgtcggggttgccgttccagttgacaccgtgtgctgcggcctgctgttcggctttgcggttgcaggtgtgtgctgcggtgatcatggtgagaccctgtgaggtttcttcaccccttgcttgggcttgccggtgggttttctgctgttcggctcgcagtgactgttctgcggcggcctggcgtgctttcttttcggctttgcgctgttggatagtcttgggtgtccattcggtgttggctgtggtggcttgcggtgcgggctgtgatgcgagtggcggattgtcgtcgggtgctggcaggaaggatgctgcggcgatgatggcggctgtgattccggcgatggtgtagcctgttttcttgttcatggctttgtgttcccctttccggggtgttgttcgttgctgacatgattaatactttcagcggctgggcccactgtcaaggctgcgctcagtttgcgtgagcgatacttgtgtggctaggggtgatggcttctttcgcccaataggatgtgccaccgctggtccagtatccgagtttgttgcgctgcatgcccttggcgtccatctcgtcgatcgtgaggcacctgcgacgactggggcctgtcttgactccatggtcgcctacccggtgcatgtcgcctgaggtggtactcgtgaatgtttcgtggcagattgtgcagtgctctggcttgtatccgatgatggtgctatcgcacttgtggcatgtccattgcatgattgctcctattttccattataagacttcctgtagtgccattttagcgccttgcgagtcttgggggtacaactatataggtcgggtatttctaggcgattctaggctcgttgtgtgtggttgggggtttatcgggcgcacagggtgagcaggcttccgatgttgatgcgtatcacattccagtagagttgtgtggcttcaccgtcggtgagtggcttccactcgtcatggctgaacacggtgccatcggatgcgatgaacgtgttggggcgtagcttgtggagttcagtctctacacgctgccggtaggcttcggcgaggccctcaaaatccatgtggtcgcaggagaggttttcgaagcgtgtcaagtcgatgggtgtggggcagtcgtcgttggtgggggtgtagagctgggtgaagtggttggcgatcttctgcatgacgggttccttttctcgtgtgatgggttgatagttttatcgggttgcggcggcaataatggcatccacgtcgatcatgtcgatcatgtcgttgagttcctcggcctcattctcggagaggtggcgccagccatagtcgccgtatacggcgccgtcgagggtgacagtccacaggggccggatgagtcgtacggcttcttcgactttggcgtggtacatgcggcgcaccatatccagatcgatgtcgtctgaatggtttccggtgaggctgtggaagctgagcgggtcgatttctgtctgcctgtcgaggctggtgaatgatggtgtgatgagtgtgccatccatagggtgtgtgctcctttcggtggtggaggggttgttgtggtttctagagtgtgtaggttgcgaccccacagtcaaggtggcgctcattcggattgagcgtttcatggaaggtgacggatgtcactgaagccttgatggcctctctcatcgcctgaaatcttctagaggtaggattatgcagggtttaccctgctgatcgattctaggggccttctagggcgtctcaggggtgtatctgggtgatagcaggtccggtagatctatcttggctttcatgacgggggtcgaggtgccagatctggtcatggaatccacaccctcatactgtgtgagatgtatcacatcctcctggcttggtgtgccctctcgaggctactctgccgatctggcgtgaagggtgtagcccagaaatgccgtttaaagcctccctatggcgcctaggagcgccttacagagtgggggctaggtattcatacccccaagcaattctgatcgattctagacgcctcccagagcctgatacacgatcaaccatctcggcatagaccagcagcccctatcctggttagctaagcctcaactatgtggacagtgtgggatactaagggggaagaaggacacggtaaaagaaagagggggagcatcagccttagggtcttagcacttagcgcttagcaccgagcccctcaagggctcggcatcagcccgacagcccgagcaggctcagccgatcaggcacagccctgaaaggggtacacgccatcagggaaggcttgagagtacgaggagccctagcgacgagtactcgaaagcctgagggaacacccatcagcactgatgggcctagcgtgttcggaaaggacacaagagtacagtgtgacagctgttcgggagtgaaacctgttctgactaggggtttcagccttaaccaccctcaaaggttacaagactctaagaaaatttaaggaaaagtttaggtttaatttttggacctttactaccaaaaacacccgtttacacccctcaaacccgcctatagagccaaaaccaccagtttgactcatcccaggtggggtatgataggctggacaggtagccagctggacgcaaggccgaaatccgctgacgcggctttcacccttacatccatcagtctaccaaagacttaaaagcttaacagctaagcgctaagcccttaagaccttaacacttagcaccgagcccctcaagggctcggcatcagtcttaaagccttaaacacttaaagttataaataaacattaaagctttaaagtcttaaagtaaatatataaccttaacagttaaacgtttaaagctttaaaccttaacacctaagttaagtataaaaccttaaaggcttagcacttaaggatataaacttaacatcagtgtttaagactttaagactttaaaacttaaaataactattaatacttaaaagcttataagtattaaacacttaaagtaactataagactttaaaaaccttaagtacttaaagttaaccatcagtcttaaactttaatattataacctataagtcttaaagcttataggtgtaataatataatataagtattaaagcttataagttataaaagttttagaagagttaaagggttaacttctttacttctctactctctttggtactttctctcttctcttcttttcttcatcaggggagaagaggaacctttaccatcagcgccgatggacttttcgccgtgtgtctcgtgtaccaccggtcgcacgctcccggtttgtacactccccacactctgacacctgtgtccctttacggcttggcgtgttcggctgaaggcgtacggcgtgtcacgctcacacccttaacaccaggtaagacttaaagtgtatattataagtagaagactttaaaacctgtaaggtgttcccgcttagcccgtgtccttccacgctaggcgccaagcgctaagctgtgaaacgcgaacacacacccacccccttttttctttcgtgtccttctctttttgacacagctggggggcgatgtgatctttttcacatgccagggggtagtggagaaaacaagcaccccggaatgttcaagacaccccctcaaacgaacaaaacgccccccataatcgatgagcagggcaagggcaaggtattcatacccccaacggttcccaggctgttagagaggcaaataagacccctgcaagggtaggcgaggaacagacacatcatggcacgcaccaaccgcaccgcatcatcagcccaccgccgctggcgggcaagactcatcacccaagcacgcaagcaaggccaaaccgaatgcccactctgcggagccaccatcacctggaacacacatgacctgccaaccagccccgaagccgaccacatcacacccgtcagcaggggaggactcaacaccctcgacaacgggcaaatcatctgcagaacatgcaacagaagcaaaggcaatcgcagcgaaccaaacatccaattccaacaacaaaccacaaaaacgctgatcccatggtgaaaaaactgtcaacccccaccgggaccccccctgcacacccgtgcaagacctcgtacggcttagtgaaatacctcccttttgtggttttgtctgtttgtcgactttttgtgttggtggtgagtgttgtgcagcctgagcttcctggtggtcgtgagtggtgtggggagacgcgtcgttggtggcgtgtgtggggtgaggatagccgcgcatcgtatgtgtctgatgaggagtggttgtttcttatggatgctgcggtgattcatgattgtgtgtggcgtgagggcagggcggatttggtggcttcgcttcgtgctcatgtgaaggcgtttatgggcatgttggatcgttattcggttgatgtggtgtctggtggccgtggtgggggttctgcggtggcgatgattgaccggtataggaagcgcaggggggcttgagtaggtgtctggtgttgtgggttctcaggttcctcgtcatcgtgtggctgcggcgtattcggtgtctgctgggggtgatgctggggagttgggtcgtgcgtatgggttgacgcctgatccgtggcagcagcaggtgttggatgattggcttgctgtgggtggtaatggtaggcttgcttcgggtgtgtgtggggtgtttgttccgcggcagaatggcaagaatgctattttggagattgtggagttgtttaaggcgactattcagggtcgtcgtattttgcatacggctcacgagttgaagtcggctcgtaaggcgtttatgcggttgaggtcgttttttgagaatgagcggcagtttcctgatttgtatcgtatggtgaagtcgattcgtgcgacgaatggccaggaggctattgtgttgcatcatccggattgtgccacgtttgagcgtaagtgtggttgtccgggttggggttcggttgagtttgtggctcgtagccggggttctgctcgcgggtttacggttgatgatttggtgtgtgatgaggctcaggagttgtcggatgagcagttggaggctttgcttcctacggtgagcgctgccccgtctggtgatccgcagcagatttttttgggtacgccgcctggcccgttggctgacgggtctgtggtgttgcgtttgcgcgggcaagccctcggtggggggaaacgtatcgcgtggactgagttttcgattcctgacgagtctgatccggatgatgtgtcgcggcagtggcggaagcttgctggtgatactaatccggcgttggggcgtcgtctgaattttgggaccgtaagcgatgagcatgagtcgatgtctgctgccggttttgctcgggagcggcttggctggtgggatcgtggccagtctgctgcgtctgtgataccggctgataagtgggctcattctgcggtggatgaggcggctctggttggcgggaaggtttttggtgtctcgttttctcgttcgggggatcgtgtcgcgttggcgggtgctggccggactgatgctggtgtgcatgttgaggtgattgatggcctgtcggggacgattgttgatggtgtgggccagttggctgattggttggcgttgcgttggggtgacactgaaaagatcatggttgccgggtctggtgcggtgttgttgcagaaggcgttgacggatcgtggtgttccgggtcgtggcgtgattgtggctgatactggggtgtatgtggaggcgtgtcaggcttttctggagggtgttcgttcgggtgtgatcagtcatccgcgtgccgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaggctaggcggcgtgaacggtctggtaggaagcgggtgtctgtggtatgaactcggatgagttggctctgattgagggcatgtacgatcgtattcaaaggttgtcttcgtggcattgtcgcattgagggctactatgagggttctgcccgggtgcgtgatttgggggttgctattcctccggagttgcagcgtgtgcagacggtggtgtcgtggcctggtattgctgtggatgctttggaggagcgtctggattggcttggctggactaatggtgacggctacggcctggatggtgtgtatgctgcgaatcgtctatcaaccgcgtcatgcgacgtccaccttgatgcactgatttttgggttgtcgtttgttgcgatcattccccagggggatggttcggtgtctgttcgtccgcagtcgcccaagaattgtactggccggttttcggctgacgggtctcgtttggatgcgggtttggtggttcagcagacgtgtgatcctgaggtggttgaggctgagttgttgcttcctgatgtgattgttcaggtggagcggcgtgggtctcgtgagtgggttgagacgggccgtatcgagaatagtcttggtgcggttccgttggtgcctattgtgaatcgtcgccgtacttctaggattgatggccgttcggagattacgaggtctattagggcttacacggatgaggctgtgcgcacattgttgggccagtctgtgaatcgtgacttctacgcctacccgcaaaggtgggttacgggtgtgtcggctgacgagttttcgcagcctggctgggtcctgtcgatggcttctgtgtgggctgtggataaggatgatgacggcgacacaccgaatgtgggatcgtttcctgtgaattctcctacaccgtattcggatcagatgcgtttgttggcgcagttgactgcgggtgaggcggctgttccggaacgctatttcgggtttatcacgtctaacccgccttctggggaggctttggctgcggaggagtctcggcttgtgaagcgtgctgaacgccggcagacgtcgtttggtcagggctggttgtcggttggttttttggctgcccgggcgttggattcgagtgttgatgaggctgcgttttttggtgatgtgggtttgcgttggcgtgatgcttcgacgccgactcgggcggctacggcggatgctgtgacgaagcttgttggtgccggtattttgcccgcggattctcggacggtgttggagatgttgggtttggatgatgtgcaggttgaggctgtgatgcgtcatcgtgctgagtcttcggatccgttggcggcgctggctggggctatatcgcgtcaaactaacgaggtttgataggcgatggcttcgggtgctatgtcgaggcttgctgcgactgagtatcagcgtgaggcggtcaggtttgctgggaagtatgcgggctattatgccgagcttggtcgtttgtggcattccgggaagatgacagatgcgcagtatgtgcgtttgtgtgtggagttggagcgtgccggccatgatggttcggcatcgttggcgggcaggtttgtgtcggattttcgccggttgaatggtgtggatcctggtttgattgtgtatgacgagtttgatgctgccgccgcgttggctaggtcgttttcgactattaagattcttaagagtgatccggatagggtgaatgacacgattgatgcgatggctgcgggtgttaatcgggctgtcatgaatgctggccgtgacacggttgagtggtctgcgggtgcgcagggtaggtcgtggcgcagggtgacggatggtgatccgtgcgcgttttgtgccatgttggctacgaggtcggattatacgaccaaagaaagggcactcactactggtcatacgcggcgtcataagcgtggtggtaagcgtccgtttggttcgaagtatcatgatcattgtgggtgtacggtggttgaggttgttggcccttgggaaccaaatagggctgatgccgcatatcagaggacgtacgagaaggcccgtgagtgggttgatgatcatgggttgcagcagtcgcctggcaatattttgaaggctatgcgtactgttggcgacatgagatgatggtttccggttgtgtgccgccggttatcggtgcacagggttgtctcccgcacgggggtcaacaatgttgtgttgttttccgcaaggagtataggttaggctatggccgatcagagtgttgaagaacagaatgtcgacaatgatgctgttgagcccggaaagggcgaggacattgttgctgttgtgaaggatgggcaggctgccggcgatgatcatgccggtgatgtttccgtgaaggaggagtcttcttctggcacggattggaaggctgaggcccgtaagtgggagtctcgtgctaaaagtaatttcgccgagttggagaagcttcgcgcctcggatggtgatgcgggatctactattgatgagcttcgccgcaagaatgaggaactcgaagacaggatcaacgggtttgttcttgagggtgtgaagcgcgaggtggctgccgagtgtggcctgtcgggtgatgcggtcgctttcttgcacggcgacgatcgtgaagcactggtggagtctgctaaggctttgaagggtttgatcgaccatagcagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctgtcgccggagcagccgactattttcggcccggtgaagggtgccgtgtttagtggtgttcctcgcgctaagattgttggtgagggtgaggttaagccttccgcgtctgttgatgtttcggcgtttactgcgcagcctatcaaggttgtgactcagcagcgtgtctcggacgagtttatgtgggctgacgctgattaccgtttgggtgttttgcaggatctgatttcccctgctcttggtgcttcgattggtcgcgctgtggatctgattgctttccatggtattgatcctgctacgggtaagcctgctgcggctgtcaaggtgtcgctggataagacttcgaagacggttgatgcaaccgattccgctacggctgatcttgttaaggctgtcggcctgattgctggggctggtttgcaggttcctaatggtgttgctttggatccggcgttctcgtttgctctgtcgactgaggtgtatccgaaggggtctccgcttgccggtcagccgatgtatcctgcggccgggtttgccggtttggataattggcgtgggctgaatgttggtgcttcttcgactgtttctggtgccccggagatgtcgcctgcctctggtgttaaggctattgttggtgatttctctcgtgttcattggggtttccagcgtaacttcccgatcgagcttatcgagtatggtgacccggatcagactgggcgtgacctgaagggccataacgaggttatggttcgtgccgaggctgtgctgtatgtggctatcgagtcgcttgattcgtttgctgttgtgaaggagaaggctgccccgaagcctaatccgccggccgagaactgatttattgttgcggtgatgtgtcaatgtgcagggggtggtgttgatgggtatcattttgaagcctgaggatattgagcctttcgccgatatccctgaggggaagcttgaggcgatgattgctgatgtggaggctgtggctgtcagtgtcgccccctgtatcgctaaaccggatttcaaatacaaggatgccgctaaggctattctgcgtagggctttgttgcgctggaatgataccggggtttcgggtcaggtgcagtatgagtctgcgggcccgtttgctcagactacacggtcgaatactcctacgaatttgttgtggccttctgagattgctgcgttgaagaagttgtgtgagggggatggtggggctggtaaagcgttcactatcaccccaacgattaatggtcgatatgcacattctgaggtgtgttccacggtgtggggtgagggttgctcgtgcggatctgatattaacggctacgctggccctttgtgggagatatgatatgaccggttttccttacggtgaaacggttgtgatgcttcagccgactgttcgtgtcgatgatcttggtgacaaggtggaagactggtctaagcctgtcgagactgtgtactataacgtggccatctatgcttccgtttcgcaggaggatgaggccgcgggccgtgactctgactatgagcattggtcgatgcttttcaagcagcctgttgtgggtgccggttatcgttgccggtggcgtattcgtggtgttgtgtgggaggctgacgggtctcctatcgtgtggcatcaccccatgtccggttgggatgctggcacgcaggttaatgtgaagcgtaagaagggctgataggttgtggctcaggatgtgaatgtgaagctgaacttgtctggtattcgtgaggtgttgaagtcttctggggtgcagggcatgttggctgagcgtggcgagagggtgaggcgtgcggcctcggcgaatgtgggcggtaatgctttcgatagggcccagtatcgtgccgggttgtcgtcggaggtgcaggttcaccgtgttgaggctgtggcccgtattggcaccacctataagggtgggaagcgtattgaggcgaagcatggcacgctggcccggtcgattggggcggcgtcgtgatcgtctacggtgaccccaggaaatgggctaaacgcgtgctcaaggatgatggctggctgtctgatataccctgtgtggggacggtgcctgatgatttcagcggtgatctgatttggttggctcttgatggtggcccgcagttgcatgttcgtgagcgtgtttttttgcgggtgaatgtgttttctgatatgccggatcgtgctatgtcgttggcgcgtcgtgttgaggctgtgctggctgatggtgtggacggtgaccctgtggtgtactgtaggcgttctactggccctgatttgctggttgatggtgcacgttttgatgtgtattcgctttttgagcttatatgtaggcctgcggagtctgaataagcttattgtttttgttttaatgtaattgtttgatatttaatgggggttatgatggctgcaacacgtaaagcgtctaatgttcgctctgctgttactggcgacgtttatattggtgacgcgcacgcgggtgatactattaagggtgtggaggcggttccttccgggcttacagctttagggtatctgtcggatgacgggtttaagattaagcctgagcgtaaaacggatgatttgaaggcttggcagaatgcggatgttgttcgcacggttgctaccgagtcgtctatcgagatttcttttcagctgatcgagtctaagaaggaggttatcgagctgttttggcagtcgaaggttactgctggcgccgattcgggttcgtttgatatttctccaggcgccaccactggcgtgcacgctttactgatggatattgttgatggggatcaggttattcgctactatttccctgaggttgagttgatcgatcgtgacgagattaagggtaagaatggcgaggtgtatgggtatggtgtgacgttgaaggcgtatcctgcccagattaataagaagggtgatgcggtgtctggtcgggggtggatgacggctttaaaagctgatactcctccgacgcctcctccggccccggttcctccgaagcctcagccggatccgaatcctccggctggtaactgatacacgattttagggattgttgatagatgagtgacacgggtttcacgttgaagattggtgaccgtagctgggtgttggcggatgcggaggagacggcgcaggctgttcctgcccgtgtttttcgccgtgccgccaggattgcccagtcgggggagtctgcggatttcgcccaggttgaggtgatgttttctatgttggaggctgccgccccggctgatgctgtggaggccttggaggggcttcctatggttcgtgtggcggaggttttccgtcagtggatggaatacaagcctgacggtaagggtgcctcgctgggggaatagtttggctccacggcctgattgatgattatcgtggggccatcgaatatgattggaggacccggttcggttgctcggtttatgatgttggtggcccggtgatgtgttggggtgaggctgtccggctggctggcgtgttgtgtaccgatacgtctagccagttggcggcccacctgaatggttggcagcgcccgtttgagtggtgtgagtgggcggtgttggacatgttggatcattacaggtctgctaatagtgaggggcagccggagcctgtggcgaggcctacggatgagcgtagggcccggtttacgtctgggcaggtggacgatattttggcgcgtgttcgtgccggtggcggggtgtctcgcgagattaatattatggggtgaatagtgtatgtctggtgagattgcttccgcatatgtgtcgttgtatacgaagatgcctggtttgaaggctgatgttggtaaacatttgtcgggtgtgatgcctgctgagggtcagcgttctggtagcctgtttgctaagggcatgaagttggctcttggtggtgcggcgatgatgggcgctatcaatgttgctaagaagggcctcaagtctatctatgatgtgactattggtggcggtattgctagggcgatggctattgatgaggctcaggctaagttgactggtttgggtcatacgtcttctgatacgtcttcgattatgaattcggctattgaggctgtgactggtacgtcgtatgcgttgggtgatgcggcgtctacggctgcggcgttgtctgcttcgggtgtgaagtctggcgggcagatgacggatgtgttgaagactgtcgccgatgtgtcttatatttcgggtaagtcgtttcaggatacgggtgctatttttacgtcggttatggcgcgcggtaagttgcagggcgatgacatgttgcagcttacgatggcgggtgttcctgtgctgtctttgcttgccaggcagactggtaaaacgtctgctgaggtgtcgcagatggtgtcgaaggggcagattgattttaacacgtttgcggctgcgatgaagcttggcatgggtggtgctgcgcaggcgtctggtaagacgtttgagggcgctatgaagaatgttaagggcgccctgggttatttgggtgctacggctatggccccgtttttgaacggtctgcggcagatttttgttgcgttgaatccggttatcaagtctatcacggattctgtgaagcccctgtttgcgtcggtggatcaggggattcagcgggtgatgccgtctattttggcgtggattaatcgtatgccggctatgatcacgagaatgaatgcacagatgcgcgccaaggtggagcagttgaagggcgtttttgcgaggctgcatttgcctgttcctaaggtgaatttgggtgccatgtttgctggcggcaccgcggtgtttggtattgttgctgcgggtgttgggaagcttgttgcggggtttgccccgttggcggtgtcgttgaagaatctgttgccgtcgtttggtgctttgaggggtgccgccggggggcttggtggcgtgtttcgcgccctgggtggccctgttggtattgtgatcggcttgtttgctgccatgtttgctacgaatgcccagttccgtgccgctgttatgcagcttgtgggggttgttggccgggctttggggcagatcatggtcgctattcagccactgttcgggattgttgctggcgtggttgccaggttggcgccagtgttcggccagattatcggtatggttgctggtttggctgcccaattggtgcctgttattggtatgcttattgcccggctggttcctgttatcacccagattattggtatggtaacccaggttgctgcgatgattttgcctatgctgatgccggttattcaggctgttgttgctgtgatacggcaggttattggtgtgatcatgcagttggtgcctgttttgatgccggttgtgcagcagattttgggtgctgtcatgtctgttttgccgccgattgttggtttgatacggtcgctgataccggtgatcatgtcgattatgcgtgtggtggtgcaggttgttggtgccgtgctacaggtggtggcccgtattattccggttgttatgccgatttatgtttcggtgattggattcattgccaagatttatgctgcggttatcgtttttgaggctaaggttattggcgctattcttcgtactattacgtggattgtgaatcattcggtgtctggcgtgaggtctatgggcacagccatccagaatggctggaatcatatcaaatcgtttacgtctgcgtttattaacggtttcaagtcgatcatttctggcggcgttgccgcggttgtggggttttttacgcggcttggtttgtcggttgcctcccatgtgaggtccggttttaacgcggctcgtggcgctgtttcttctgcgatgggtgctatccggagtgttgtgtcttcggtggcgtctgctgttggcgggtttttcgggtcgatggcttctcgggttcgtagtggtgctgtgcgcgggtttaatggcgcccggagtgcggcttcttctgctatgcatgctatggggtccgcggtgtctaacggcgtgcatggtgtgctagggtttttccggaatctgccgggcaatattcggcgtgctctcggtaatatggggtccttgttggtgtctgctggccgtgatgtggtgtctggtttgggtaatggtatccggaatgctatgagtggcctgttggatacggtgcgtaatatgggttctcaggttgctaatgcggcgaagtcggtgttgggtattcattcgccgtcgagggtgtttcgtgacgaggttggccgtcaggttgttgctggtttggctgagggtattactggtaatgctggtttggcgttggatgcgatgtcgggtgttgcttcgcagcttccggatgctgttgatgcccggtttggtgtgcgatcgtctgtgggctcgtttaccccgtacgaccggtatcggcgtgcgagcgagaagagtgttgtggtgaatgttaacgggccgacgtatggggatccgaacgagtttgcgaagcggattgagcggcagcagcgtgacgctttgaacgcgttggcttacgtgtgataggggggtgtggttcatgtttattcctgacccgtctgatcgtgccggtttgactgtgacctggtctatgttgccgttgattggtgatgctccggagcgtgtgcttcatttgacggattatacggggtcgtctccggtgatgttgttgaatgattcgttgcgcggtttgggtgttcctgaggttgagcatttttctcagactcatgttggggtgcacggctcggagtggcgcgggtttaatgtgaagcctcgcgaggtgactttgccggtgttggtgtcgggtgttgaccctgatccggtgggcgggtttcgtgacggttttttgaaagcctatgacgagttgtggtctgcgtttcctcctggcgaggtgggggagttgtcggtgaagaccccgtctggtcgtgagcgtgtgctgcggtgccggtttgattcggtggatgacacttttacggtggatccggtgaacaggggttatgcgcgttatctgttgcatttgacggcttatgacccgttttggtatggggatgagcaaaagtttcgttttagtaacgcgaagttgcaggattggttgggtggcggccctgttaataagaagggtaccgcgtttcctgtggtgttaacaccgggtgtgggctcgggctgggataacctgtctaataagggtgatgtgcctgcgtggcctgtgattcgtgttgagggtcctttggagtcgtggtctgtgcagattgatggtttgcgtgtgtcttcggattggcctgtcgaggagtatgattggatcactattgatacggatcctcgtaagcagtctgcgttgttggacgggtttgaggatgtgatggatcgtttgacggagtgggagtttgcgcctattcctcctggcggttctcggagtgtgaatattgagatggttggtttgggtgccattgttgtgtcggtgcagtacaggtttttgagggcttggtgaatagttgatggctggtcttgttccgcatgtaacattgtttacgccggattatcgccgtgtggcgcctatcaatttttttgagtcgttgaaactgtcgttgaagtggaatggtttgtccactttggagttggtggtgtcgggggatcattctaggcttgacgggttgacgaggccgggtgcgcggctggttgttgattatggtggtggccagattttttctgggcctgtgcgtagggtgcatggtgtgggtccgtggcggtcttcccgtgtgactatcacgtgtgaggatgatattcgtctgttgtggcgtatgttgatgtggcctgtgaattatcgtcctggtttggtgggtatggagtggcgtgcggatagggattatgctcactattcgggtgcggcggagtcggtggctaagcaggtgttgggggataatgcttggcgttttcctcctggtttgtttatgaacgatgatgagagtcgtggccgctatattaaggattttcaggtgcggtttcacgtgtttgccgataaattgttgccggtgttgtcgtgggctcggatgactgtcacggtgaaccagtttgagaatgcgaagtttgatcagcgtggtttggtgtttgattgtgtgccggctgtgacccggaagcatgtgttgactgccgagtcgggttcgattgtgtcgtgggagtatgtgcgtgacgccccgaaggctacttcggtggtggttggtggccgcggcgagggcaaggatcggctgttttgcgaggatgttgattcgatggccgaggatgagtggtttgatcgtgtcgaggtgtttaaggatgcccgtaacacggattcagagcatgtgcatcttattgatgaggctgagcaggtgttgtccgagttaggggctacgtcggggtttaagatcgagttggctgagtcggatgtgttgcgttttgggccaggcaatctgatgccgggtgatttgatctatgtggatgtgggctcggggcctattgcggagattgtgcggcagattgatgtggagtgtgattcgcctggtgatgggtggacgaaggtgacacctgttgcgggggattatgaggataatccgtcggccctgttggcgcggcgtgttgccggtttggctgcgggtgtgcgggatttgcaaaaattctagaaaagatgaggggtttgttgtgggtattgtgtgtaaagggtttgatggtgtgttgaccgagtatgattgggctcaaatgtctggtctgatgggtaatatgccgtctgtgaagggcccggatgattttcgtgtgggcacgactgttcagggtgccacagtgttgtgtgaggttttgccggggcaggcttgggctcacggggtgatgtgcacgttgaatagtgttgagacggtgacagggcagctgccgggccctgggggggcccgctacgactatgtggtcctgtctcgggattggcaggagaatacggccaagttggagattgttcctggggggcgtgcggagcgtgcccgtgacgtgttgagggctgagcctggcgtgtttcatcagcagttgttggcgactttggtgttgtcgtctgacgggttgcagcagcagctggataggagggctatagcggctagggttgcgtttggcgagtctgctgcgtgtgacccgaccccggtggagggtgaccgtgtgatggttccttcgggggctgtgtgggctaatcatgctaacgagtggatgttgttgtctccgaggattgagacgggttcgaagtcgatcatgtttggcggttctgctgtgtatgcttacacgatcccgtttgatcgccagtttgctagtccgccggttgtggtggcgtctatggctacggcggctgggggcacggcacagattgatgtgaaagcctacaatattactgccaaagattttagtttggcgtttattacgaatgatggttcgaagccgaatggtgtgcctgcggttgcgaattggattgctgtcggcgtgtgaccgggttgttgttgtggcggatggtgtgatgttggggggctgtggtgtcgtggtttactcctgcactggtggcctctatttgtaccgcgttggccacggttttgggttctgttcaggcggtcacgtctaaatctcggaggcgtttgcggcggctgtcggcgcaggtggatgcgatggaagagtatacgtggggtgtgcggcgtgaggttcgccggtttaacgctgggcttccggatggggtggagccgatgcatcttcctgatgtgcctgagtttttgaaggatactgttgatggtggaggtgagtagggttgagggagttggaggaggagaagcggcagcgccgcaattttgagaaggcttcactggtgttgttgtttttgtcgcttgtgttgttggcggtggttgctgtgggtgctttgcgtttcggggcggtatcctctgagcgggattcggagcaggcgagggcccagtcgaatggtacagcggctcggggtttagccagcagtgtgaagcaggcgtgtgcttcgagtggggtggagtcggtgcggcttcaccggtctggtttgtgtgtggatgctgtgcgtgttgagcggagtgtgcagggtgtgccgggtcctgccggtgagcgcggcccgcaaggccctgcaggggttgacggccgggatggtgttaatggttcggctgggctggttggccctgttggtccgcagggttctcctggtttgaatggtgtgaagggtcctgacgggttgcctggtgtgaatggatcggatggccatgatggtgttccaggtcgtgcaggtgctgacggtgtgaacggcgctgatggtcgggatggttcgggcggtgagcgcggcgatgtgggcccttcaggtcctgtcggaccccctggtgcgcagggtgaacgggg

SEQ ID NO: 75 - DNA, 29562 bp, synthetic construct
  source 1..29562: mol_type = other DNA; note = PAC263
aatggtgtgaagggtcctgacgggttgcctggcgttaacggttcggatggccgtgatggtgttccgggtcgtgcaggtgctgacggtgtgaacggcgttgacggccgggatggtgttaatggttcggctggtgagcgcggcgaacagggcccttcaggtcctgccggcccccaaggcgaacggggtgagcgcggccccgccggtgctaacggatccgatggtaaagatggtaaagatggtaaagatggccgttctgtggtgtctgtgtactgttctgatggtcgcctggttgtgaaatatagtgacggtgtggcttccacgatatcgggctcggtagcctgccagggtgtgaaaccgtcgcctatagtgactatatcatcccacaaataaaagaggaagggtgttactgtgattgtcatgttttggggtggtgtgtggtgaggtttattcctgcggcgcatcattcttccggttcgaatagtccggtgaatagggttgtgattcacgcgacatgcccggatgtggggtttccgtccgcgtcccgtaaagggcgggcggtgtccacggcaaactatttcgcgtccccatcggcgggtgggtggggtttccgtccgcgtcccgtaaagggcgggcggtgtccacggcaaactatttcgcgtccccatcggcgggtggttcggcgcattatgtgtgtgatatttcggagactgtgcagtgcttgtcggagtctacgattgggtggcatgccccgccgaatccgcatagtttgggtatagagatttgcgcggatgggggttcgcatgcctcgttccgggtgccaggccatgcttacacgagggagcagtggcttgatccgcgggtgtggcccgcggttgagaaggcggctgtcctgtgccggcgtttgtgtgacaaatataatgttccgaagaggaaactgtctgtggccgatttgaaggccggtaaacggggtgtgtgtggccatgtggatgtgacggatgcgtggcatcagtcggatcatgacgatccggggccgtggtttccgtgggacaagtttatggccgtagtcaacggcaaagatgagagtggggagttaactgtggctgatgtgaaagccttgcatgatcagattaaacaattgtctgctcagcttagtggttcggtgaataagctgcatcacgatgttggtgtggttcaggttcagaatggtgatttgggtaaacgtgttgatgccttgtcgtgggtgaagaatcctgtgacggggaagctgtggcgcagcaaggatgctttgtggagtgtctggtattacgtgttggagtgtcgtagccgtcttgacaggctcgagtctgctgtcaacgatttgaaaaagtgatggtggtttgttgtgggtaaacagttttggttaggtgtgctggagcgggcggctaagacttttgtgcaaacgtttgttgcggtgttgggggtgacggcgggtgtcacctatacggcggagtcgtttcgcggtttgccgtgggagtctgccctgatcacggctacggtggctgcggtgctgtcggttgctacatcgtttggtagcccagcgtttgtggccggtaaacctaaaaccacgcctgtggatgcgggtttggttccaccggatgatgggggcttggttgagccgcactcggtggatgtgtcggatcctggcatgattgagcctgcagatgatgtggatcttggtgtaggctatgagcctcggcgtgctgccgagtcggaggttggcacggtagagtctactgttgcataagtgaatatatgtgtgtgccccagcggtgctgccacgatcgtgtggtggttgccgctggggcactatttttgtatattgcggtgtggctatgattcgttgctgtcgatggtgtcttcgagcatctgatacaggtggaggcaggtagagatcgtttcgctggcctggtcgagaacgttccggccgataacgtttttgtggttgtcgcggtggcggatgatagaccacatgatctcgtcggctgccgcctgcaatagttttgcctgatatgcgatcccggcgagccagtctagtgcttcctggcttgcataggggctctggtcctcgctgttgccgcgggtgttgctgttgtttgtggggtgtcctgcactgtcgcagaaccataggatttcgctgcactcgtctagcgtgtcttggtcgatagcgagatcgtcgaggctgacattgttgacggtaaggttcacgttgtcgagggagatgggtacaccgtactggttttcgacactgtcaacaatgttttgcagctggttcatgttggtgggctgttgttggatgattcggtgtaccgctgttttgagggcggtgtaggggatattggttatgttgttcatggttttatcccatccctgcgctgtcgtcttggtagtatcgactgtttgcgtaacctgtgagggtgatgagtgtttggtctgcccactgtttcacggtttgccgggtgactccgagtcgttgggctgccaccgaataggtttggtcatacccgtatacttccctgaaggctgccaggcgtgctagccgtttccgctgtttggatggctggcaggtgagggtgtagtcgtctatcgctaattgtaggtcgatcatggtgacgatgttgttgccgtggtgttggggggcggttggtgggggtggcatgcctggctccacactgggtttccatgggcctccgttccagatccattgggcggcttggatgatgtcggcggtggtgtaggttcggttcactggtcatcccctgaataggttgtcgaggttgtctgggttgctggtgttggtggtgtcgaatcgtcccacacagtggcagtagtcgtacatgagtttaataatgtgttggtggtctcccaaataggtgtttccgctgatactgtaggtggctgtgccgtctttactaatagtgtatttggcggtgatggtttcgggtgtttctgtgttggtgatgattgctgtggtggtggcgcctacggtttgtagcctggtggtttgggttccgtcgtcgaggatggtagtaaccatgagggttgtcctttagttgctggtttggttgtcggctagatgaatgatatcgggtaaaggtttcggctggtcgaggtgttgtatggttttgttggctagccgtttggctaccctgtagcacattttggtgtagtgtttgttgtctaggttgtggtattgttcccgcaccgcaatatatagtagggagtcttggtataggtcgtctgcactgattgcggggtagtgtgtggctgttttggtgcatgcccggttgagtgtgcgtagatgatggtctgtggcccacacccacgatgcggtggtggctaggtcggcttttgttggtcgtctgctcatggcactattacctggctatctggtagttgtttggtgttttgttgttgatagtgtagcacacgagtccggggttgccggtggtgcctgtgcggtgcctataccagacggattctccttccatggatgggcattggatgaaggtgcgttgtccttgctcggagatttcgaggtggtgccggtgtccggccatgaggatgtgggatgtggtgccgttgtggaattcttggccgcgccaccattcgtagtgttggttgttgcgccattggtgtccgtgggcgtgcagtatttgtgtgccggccacatcaacggtggtggtcatttcgtctcgtctggggaagtggaagtgaaggttggggtagttgttggtgagttggtaggcttcggcgatggcgcggcagcagtctacatcgaaggagtcgtcgtaggtggtgactcctttgccgaatcgtacggcttcaccgtggttgccggggatggaggtgatggtgacgttggcgcagtggtcgaacatgtggacgagttgcatcatggccatgcgggtgagcctgatttgttccgtcaagggtgtttgtgtgcgccacgcgttagagccgccttgtgacacgtatccttcgatcatgtcgccgaggaatgcgatgtggacccgttgcggctggcctgcctgttgccagtagtgttttgcgactatgagggagtgcaaatagtcgtcggcgaagtgtgctgtttctccgccggggatgcctttgccgatttggaagtcgcctgccccgatgacgaaggccgcagtgctgtagtcggtgtgggtgtcttgttcgggtttgggtggctgccattcggctagcttgtcgacgagttcgtctatagggtaggggtttgttgcgggttggtggtcgatgattttttgtatggatcggcctgtttctccgttggggagtgtccattcggagatgcgtgtgcggcgtacggtgccgttggctaggttgtcgtcgatggtgtcgatggcgttgtcgtggttggctagttgtgtgagtagccggtctatattgtctatcactgggtatcctcctcttcctcgtgtgtggtggtggcttgtttgcggcggtagtctttaatgacggtggcggagatggggtatcctgcctgggtgagcatttgggctagctgtgtggcggggatagacctgtcggcgagcacgtctgcagccttgcggccgtagcgttggatgagggtttcagttttggttgccatgatgtcccatcggttgtgtggtgggctgccatcctgtgcggcagtcgccgtcgtgtcctggtttgcgtgtgcaccacgatacggttccgtctgtgtggttgagtgttttaccgcacatgacgttttgtagatgctccggcagctcgctattgctatcgtcttgctcgtctagcaaagttttttgttgggtgaaaaactcggacacggtgccgttgtggactgggagtatccatgttttccattgttgttgtagccgggtgttccagtggaattgttttgctgcgttcgtggcttgtttgatggttttgaagtagcctacaatgatccgttgatggtcactatcgggcttgtgtggccctttccaatattgggcagctacagcgtacctgttgttgtctgtgaagcgcccccagcagtattccaccatgtgtgatagtaccttgtcgggcatgtctcgtacttggttttcgtcgagccatgcgtcgacaataatgttgcgtatggctcgcttgtctttggtggtgggtttgaatgcgatgctcacaatgcgggcctgtcgtcttgcatgaactggttgaaggtgttgttcccggcgtgttgggcttgtgtgatttgctggtcggtccagtcggggtgttgctgtttcagatagtgccagtggcacgcattgtaggtttcgtcttggagccgtgtgagatggttttcggtgatgatttgtttccacatggcccatgacacgtcgagccggttgaggatttcgagggctgggatgttgaattggttcaggaagaggatttcatgggtgtagtagtttttctcgtaggcgtcccatccgcttcggtgcctgttgggctggtttttggggtaggcttcccggcagattttgtgtaaccgtttggccatgtctttgggtagtttaatgtcggggttggcgcggatcatggatcgcatcccatcataggtggtgccccaggtgtgcatgatgcggagtgggtcttcaccatcggcccatttttctgcacagatggcgaggcgtatgcgtctcctggcggctttactggtgtcgcggcggccggggatggggcaggtgtcgaggggatccatgatgttttagtgtacctttccgtgttgtggttgtttgtctggttttattgtagcactgtgttgagggcttgtgtcaaccctgtttttccgacctgaaggtaggtgtctgtgacatcccccagggtgaggggcacatgggtggcttgggggagtgccgtctggaaggtttgggccatctggtctcctgctttgtctgggtcggaccagatgtagatgtggtcgtagccttcgaagaatttggtccaaaagttttgccacgaggttgcgccgggtagggcgacggccgaccatccgcattgttcgaggatcatggagtcgaattcgccttcgcaaatgtgtatttcggctgccgggttggccatggcggccatgttgtagatggagcctgtgtctcctgccggggttaggtatttggggtggttgtgggttttgcagtcgtgctggagtgagcagcggaaacgcatttttcttatttcggctggcccttcccaaacggggtacatgtatgggatggtgatgcactggttgtagttttcgtggcctgggatggggtcattgtcgatgtatccaaggtggtggtagcgggctgtttcttcgctgatgcctcttgctgagaggaggtcgagtatgttttcgaggtgggtttcgtagagggccgaggctttctggattcggcggcgttccgcaatgttgtatgggcgtatgctgtcgtacattcgggttttcttcttctaattgttgttgtagtttggcgaggcctcctccgataccgcatgtgtggcagtaccagacgcccttgtcgaggttgatgctcatggagggctggtggtcgtcgtggaacgggcagaggatgtgttgctcgttcttggacgggttgtaccgtatgtggtaggtgtcgaggaggcggcgggtgtcagaggtgtgggaggagctcgttgagggttgataccacataggcttcgctccagggtttgttgcgctgtttcatcactacgagtccgatagtggactggttttcgcggtttcggtgggtttcgtagttgcgtgcctcccggctggcttgtttcacgaattcggcgaggtggggctgcccggctttggcttcgataatgtaggttttgttgccggtggtgaggatgaggtcgccttcatcctctttaccgttgaggtggaggcgttctatatcatggccggtgtcgcgtagctggtggaggagtcgtgtttcccattcggctccggctcggcggtttcttgattgttgtgtcgacatgatagtcctttgtggtgttcggtcatgttccatggctgtttttcggcgagtggcccgaagaatgtgtattcggggtaggctctgagtctttcgtatcgggttccgtctgggctggatttgcctgtgcgctgtttgagtacagcgatgcgtgcctctgccggtatcgataggccgttgccgttgtcttcgccaccatacagggagactcccaatatgagttgtggtttttcggagaggccgtttttgatttcccgcctagccggggggtgttcgatgtcggagccggttttgtcggttgcgtggtgtgtgacaataatggtggagcccgtgtccctacctaatgctgtgatccattgcatggcttcttgctgggcctgatagtcactctcgcagtcttgtatgtccatcaggttgtcgataacgatgatgggtgggaaggtgttccacatttccatgtaggcttgcagttccatggtgatgtctgtccatgtgatgggtgactggaatgagaatgtgatgtgtccgccgtggtggatgctgtctcgatagtattctggcccgtagtcgtcgatgttgtgttgtatctgggcggtggtgtgttgggtgttgagtgagatgattcgtgtggaggcctcccagggggtcatgtcccctgatatgtagagggctggctggttgagcattgctgtgatgaacatggctagcccggatttttggctgccggagcgccccgcgatcatgacgagatcccctttgtggatgtgcatgtccaggttgcggtagaggggttctagctgggggatgcggggcagctcggctgcggtttgggaggctctctcgaaggatcgttggagagagagcatcgggaccttatctatctgtctatcggttggatgatgttttggtggtcagatggagtcgatgtcgatgtcagcatcagcaggggctgtggtgtcgtctagctggccgttatcgcgtttgtctacgtattcggcaaccttatcgtagatggcgtcgtcgaggggtttgagcacgaccgcgttgaagccgtttttggtgcgtacggtggcgagtttgaaggcttgttcttcgccaaggtaggcttcgaggtcgcggatcatggagtgtgggcggtcgttgctgccgcgtactttttcgatgatggcgttggggatggtttctggggtgccgttgttgaggtcgtctagggtgtggaagatggtgacatcagcgtagatgcgatcggcggtctgtccaccgtagccttcggtgttgtgttctacgtcgtggattttgaaggcgatggcggtggcgtcctggtttcgggaggggttgaagaaggtgctgttgctgttgtttcggtagtttgcgagtcccattgttgtatcctttactgttttgttggtttgtgtaggttttatcgggtgaggctgtttcgtttgctgcggaaagcctcggaaacgtcactgttactggtgatgatctttttgtactgtttgagaaggtcggctagctgtgctttgctggttgcattgttgattttgtcgatgatggtgttgtttccttctgaggcgatgttgtctacgtagtctttggcggcctggttgtagcgatcttggaggatgatggatgctgtggcgatcagtgttgccaggtcccagttccgtgccgccgaactgtttttgagtccgcctaacaggtcgatgatggcctgttttgtctgctctgctgtgtctcctcggatgaccgcccatggtgcagcatagtctccaccgtatttgagtgtgatcgtgagtcgatcattgtcgatcttgtctttatcggtcatttggtgtccttttctttattgtctgtttctggtggctgtacggtagattctaccgggtacctgtaggcgtctttcccgttgacggcccagcaggcgtcttgtacggggcagcctttacagagtgttgtgacgtgtgggacgaagatgcctgcgctgattcctttcattgcttgactgtacatggatgatacatgccggtaggtgttgttgtcaaggtcgtacagttcggtggatgtgccttgtgtcggggacttgtcgtcgttgcggctggtggccggcgtccaaaacatgccttttgttacatcgttgccgtgttggttgagcatgtaccggtaggtgtgcagctgcatactgtcggcgggtaggcgtccggttttgagatcgaggatgaaggtttcgccggtgtcggtgtcggtgaagatacggtcgatgtagccaacgatctgggtgccgtcggggagggtggtttctaccgggtattcgatgcctggtttaccgtccaggattgcggtgatgtattctgggtggttgcgtctccatgttttccagcggtccacaaaggtggggccgtacatcatccaccaattgtagtcttttttgtgtggcccgcccgattcgcacatgtttttgcatattctgccggagggtttgatttctgtgccttcggattcggcgagggctacttgtgtggcgaaaatgtttttgaaggatgcgagtttgtctggtagcgcagggtattcggcggggttgtataggtgtaggtcgtattgttcggtgatgtggtgtatggcgcttccggcgatggtggcgtaccaggtgtggtgttgggtgtggtatccgtgttggagacgccatttttcgccgcattcggcccattgtgacagtgatgagtaggagatgtggcctggatggttgatggttttcgggtattgtgctagaggcattacttgtcgcttttgttccatgggtttcgggtgtcttggccggcatcgtgttgctggtatgcgaggagtgcgaggcagtgccaggcagcatgggctagatgcggtagcccggattcataatcgaggttgttgccttgctgccatgataacaggtgccggtagagggcatcaacgctgtggctccacgggtatcctccggtccagttgttgtcgccgtatttggtggcaccgtagcctgctacgtcgccgagagcgtgaagggatgctgggtcgatgagggagagcctgcaaagtttgagttcttttcgggcaccgctgttggggtcggtgtacatgcgggttggctcatccatgagatatgtgctccttaagcgtgggttactggttagggttgtgggcgagtgctacggcgagaataatgatggcgagggtttcagcgatgatgatgggtgttgtgatcatttgctgtctcggggattgttggtgagtgttgatgcgcctaggagggtggtgagggcgcatgcggcaatgatggcgagggctgccttgtgtggggtgccggttgcgtacatccatgtgatgatgccgccttggatccaggctaggctggtgaagaacgtttcgtagctgtgtagctcaatgttgttgttgggtgtgttcatgcttgctcctgaagaatggtgttgatggttttataaatgttgtacaggtcggcttcgatggtttgtagctgtttgatttggtggtcgagattaatgtctgggttgagggtgttgatgcgggaggcaatatctgtggctgtgcgtagtgttccgccggtgtggtgaataatgtgtgccgtgtcggcgagtccggtgatgacagcgtagtgggataggagaggcatagctggggggtgctccttggcgggttactgttgcgggttgatgttgaggtcggtgacgtgcgggtggtcttctgttccggtgacgaggcagtggacggtgacgggtagtttggatgcgccgggatgtttcgcggttgcgccgtagacgatggagaaggtgtctttaccaataattttgtggagttggaggtcgatgtcggggttgccgttccagttgaggccgtgtgcggcggcctgttgttcggctttgcggttgcaggtgtgtgctgccgtgatcatggtgagtccggtggcggtttcttcaccccgtgtttgggcttgcttgtgggctttctgctgttctgcttgtagggagcggactgcggctgcctgcttggctgttttctcggctttgcgctgttggacggttttgggggtccattcggtgttggctgtggtggcttgtggggctggttgtgaggcgagtggcggattgtcgtcgggtgctgggaggaaagagcatgcggcgatgatggcggctgtgattccggcgatggtgtagccgtttttcttgttcatggctgttgtcccctttccggggtgttgttcgttgctgacatgatcaatacttccagcgaatggacctcgtgtcaagactgcgctcaaatgttctgagcgatccttgtgtggctaggggttttatcgggcgcatagggtgagtaggtggcctacgttgatgcggctcacattccagtagagttgtgtggcttcaccgccggtgagcggcttccactcgtcgtggctgaacacggtgccatcggatgcgatgaacgtgtcggggcgtagcttgtgaagttcggcttccacgctctgccggtaggtttcggcgaggccctcaaaatccatgtggtcgcaggagaggttttcgaggcgtgtcaggtcgaagggtgtggggcagtcgtagctggcgggggtgtagagctgggtgaagtggtcggcgatcttctgcatgacgggttccttttctcgtgtggtgggttgatggtttttatcgtgtggcttcggcgatgatggcgtctacatagatcatgtcgatgagatcgtggagttcctcggcctcattctcggagaggtggcgccagtcgggtggcccatatactgcgccgtcgagggtgacagtccacagtggccggatgagtcgtatggcttcttgtactttagcgtggtacatgcggcgcaccatatcgagatcgatgtcgtctgaatggtttccggtgaggctgtggaggctaagcgggtcgatttctgtctgcctgtagagggatgtgaaggatggtgtgatgagtgtgccatccatgatgggtgtgctcctttcggtggtgtaggggttgttgtggtttttatggtgtgagggttgtgatccatagtcaaggctgcgctcaatcggattgagcgtttcatggagtgtgtcgggtgtgacagatgtcactgaagcctttattgcctctctcagcgtctcaaatcttctaggggtagaaatatactagggcagccctataaatcgattctaggcccctttctgtgactctgaggggcatatgtgagtggagggtggtatgacaggtggcatggacttggaggaaggtgtccagtcgggagcgctcgatgatccggctgcacgggtgtctggaaggcttatggtctgcgtgagatatgtcacatcacctagactctaggaacactacccacacctgtagagtctattctgcagatggcaccagagccaagaatgcctctctaaggcacgtaaaggcccctctgaggctcttacaccctcaactctaggtatttgtacccccagcatattctgatcgattctagggccctttttgaggcttacgcgagaacagcacccaaagactagcccatcaacccttactctggttagctaagcctgcactatgtggacagtgtgggatgctaagagggaagaaggacacggtaaaagaaaaaagggggagtaccagccttcacgccttcaagccttaaggtcttagcactaagcacttagcaccgagccccctcaagggctcggcatcagcccgagcaggctcagccctgaaaggggtacacgccatcagggaaggcttgagagtacgaggagccttagcgacgagtactcgaaagcctgaggaaacaccatcagcactgatgggcctagcgcgttcggaaaggacacaagagtaaagtgtgacagctatccgggagtgaaacccgttctggctaggggtttcagccttaaccacctgtaaaggttacaagactctaagaaaatttaagaaacttcttaggaagaaagttgtgttgatgtcaccccaaaaacacctaaaatagccctcaaacccgcctatagagccaaacagtcaagtttgactcgtcttgacggcgtatgctaggctggacaggtagccagctggacgcaaggccagaaagtgctgacgcacttcccgaccttgcttaccatcagtctaccaaagacttaaaagtttaacagctaagcgctaagcccttaagacctaaacgcttagcaccgagccccctcaagggctcggcatcagtcctaagagcttagcccttaaggatctaaggttactataaagctttaaacactttaagtaaacttaagagcttagcacttaaagttaattaataaccttaaaggcttacacacttagcactgagcccttcaaggctcagcatcagtataaagaccttaacacctaagttaagtataaaactttaaaggcttagcgcttaaggatataaacttaacatcagtgtttaagacttaaagagttaaacacttaaagtaactataatactttaaaaatcttaagtacttaaagttaaccatcagtcttaaactttaatattataacctataagtattaaagcttataagttataaaagttttagaagagctaaggggttaacttctttacttctcttctctctttggttctttctctcttctctccttttcttcatcaggggagaagaggaacctttaccatcagcgccgatgggcttttcatcgtgtgactcgtgtgcttctggtcgcaagctcccatcgcacactccccacactcttacacccgtgcccctttcaggcttagcgtgttcggctgaaggcgtacggcgtgtcacgctcacacccttaacaccgggtgagacttaaagtgtatattatatgtagaagactttaaaacctatagagtgtttctgctgagcctgtgtcctacaccgctaggcgccaagcgctaagccttgaaacgcgaacacacacccacccccttttctctttcgtgtccttctcttttgacaccgctggggggcgatgtgatctttctcacatgccagggggtagtggagaaaacaaacaccccggcacaaacagaacaccccctcaaacgaacaaaacagcccccaggatcgactagcagggcaagggtagagtattcatacccccagacgattccaggccgttagagaggcaatgagaggctcacaggggtcatgggagatcggggaacgcgatggcacacaccaaccgcacagccagccaagcccaccgacgctggcggcaacgactcatcacccaagcccgacaacaaggccaaaccgaatgcccactctgcggagcaaccatcacctgggacacacaccagctgccaaccagccccgaagccgaccacatcacacccgtcagcaggggaggactcaacaccctcgacaacgggcaaatcatctgcagaacatgcaacagaagcaaaggcaatcgcagcgaaccaaacatcaaattccaacaacaaaccacaaaaacacttgtttcatggtgacaaacccgccaacccccaccgggcacaccccctgcacacccgtgcaagacctcgtacggcttagtgaaatacctcccttttgtggatttgtctgtttgtcgactttttgtgttggtggtgagtgtggtgcagcctgagcttcctgatggtcgtgagtggtgtggggagacgcgtcgttggtggcgtgtgtggggtgaggatagtcgcgcgcagtacgtgtctgatgaggagtggctgtttctcatggatgctgcggtgattcatgattgtgtgtggcgtgagggtcgcgcggatttggtggcttcgcttcgtgctcatgtgaaggcttttatgggtatgttggatcggtattcggttgatgtggcgtctggtggccgtggtgggggttctgcggtggcgatgattgaccggtataggaagcgcaagggggcctgattaggtgtctggtgttgttgggtctcaggttcctcgtcatcgtgtggctgcggcgtattcggtgtctgctggcggtgatgcgggtgagcttggtagggcgtatgggttgacgcctgatccgtggcagcagcaggtgttggatgattggctggctgtcggtggtaatggcaggcttgcttcgggtgtgtgtggtgtgtttgtgcctcgccagaatggcaagaatgcgatccttgaggttgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgtgcgacgaatggtcaggaggctattgtgttgcatcatccggattgtgccacttttgagaagaagtgtggctgtccgggttggggttcggttgagtttgtggcccgttctcgtggttctgctcgcgggtttacggttgatgatttggtgtgtgatgaggctcaggagttgtcggatgagcagttggaggctttgcttcctacggtgagcgctgccccgtctggtgatccgcagcagattttcctgggtacgccgcctgggccgttggcggacgggtctgtggtgttgcgtttgcgtggtcaggctttgtcgggtggtaaaaggtttgcgtggacggagttttcgattcctgacgagtctgatccggatgatgtgtcgcggcagtggcggaagttggcgggggatacgaatcctgcgttgggtcgtcgcctgaatttcgggaccgtaagcgatgagcatgagtcgatgtctgctgccggttttgctcgggagcggcttggctggtgggatcgtggccagtctgctacgtcggtgattccggctgataagtgggctcagtcggctgtggatgaggcgagtctggttggcgggaaagtgtttggtgtctcgttttctcgttctggggatcgggttgctttggctggtgccggccggactgatgctggggttcatgttgaggttattgatgggctgtctggcacgattgttgatggtgtgggccggttggctgactggttggcggttcgttggggtgatactgaccggatcatggttgccgggtctggtgcggtgttgttgcagaaggcgttgacggatcgtggtattccgggccgtggcgtggtggttgccgatactggcgtgtatgtggaggcgtgtcaagccttcctggaaggtgtaaggtctgggaatgtttctcatcctcgtgctgattctcgccgtgacatgttggatattgctgtgaggtcggctgtgcagaagcgtaaggggtctgcgtggggttggggttcctcgtttaaggatggcagtgaggtgcctttggaggctgtgtctttggcgtatcttggtgcgaagatggcgaaagcgaagcggcgtgaacggtctggtaggaagcgggtgtctgtggtatgaactcggatgagttggctctaattgagggcatgtacgatcgtatccaaaggttgtcttcgtggcattgtcgcattgagggctactatgagggctcgaatcgggtgcgtgaccttggtgtggctattccgccggagttgcagcgtgtgcagactgtggtgtcgtggcctggtatagccgtggatgctttggaggagcgtctggattggcttggctggactaatggtgacggctacggcctggatggtgtgtatgctgcgaatcggcttgctacggcgtcgtgtgatgtgcatttggatgcactaatttttgggttgtcgtttgttgcgattattcctcatggtgatgggtcggttttggttcgtccgcagtcaccaaagaattgcacaggtaagttttcggctgacggttctcgtctggaggctggccttgtggtgcagcagacgtgtgatcctgaggtggttgaggctgagcttttgttgcctgatgtgattgttcaggtggagcggcggggttcgcgtgaatgggtcgagacgggccgtattgagaatgtgttgggtgcggttccgttggtgcctattgtgaatcgtcgtcgtacttctaggattgatggccgttctgagattacgaggtctattagggcttacacggatgaggctgttcgcacactgttggggcagtctgtgaatcgtgatttttatgcgtatcctcaacgttgggtgactggcgtgtcggctgacgagttttcgcagccgggttgggtcctgtcgatggcttctgtgtgggctgtggataaggatgatgacggtgacactccgaatgtggggtcgtttcctgtcaattcgcctacaccgtattcggatcagatgagactgttggcgcagttgactgcgggtgaggcggctgttccggaacgctatttcgggtttatcacgtctaacccacctagtggggaggctttggctgccgaggaatctcggcttgtgaagcgtgctgaacgcaggcagacgtcgtttggtcagggctggttgtcggttggttttttggctgccaaggcgttggattctcgtgttgatgaggccgatttttttggtgatgttggtttgcgttggcgtgatgcttcaaccccgactcgggcggctacggctgatgctgtgacgaagcttgttggtgccggtattttgcctgctgattctcgtacggtgttggagatgctggggcttgatgatgtgcaggttgaggctgtgatgcgtcatcgtgccgaatctgcggatccgttggcggcactggctggggctatatcgcgtcaaactaacgaggcatgataggcgatggcttcgggtgctatgtcgaggcttgctgcgactgagtatcagcgtgaggcggtcaggtttgctgggaagtatgcgggctattatgccgagctgggtcgtttgtggcgtgccgggaagatgacagacgcgcagtatgtgcgtttgtgtgtggagttggagcgtgccggccatgatggttcggcatcgttggctgccaggtttgtgtcggattttcgccggttgaatggtgtggatccgggtttgattgtgtatgacgagtttgatgctgccgccgcgttggctaggtcgttttcgactatgaagattcttgagagtgacccggatagggcgaatgacacgattgatgcgatggctgcgggtgttaatcgggctgtcatgaatgctggccgtgacacggttgagtggtctgcgggtgcgcagggtaggtcgtggcgtagggttactgatggtgatccgtgtgctttttgtgccatgttggctacgaggtcggattatacgacaaaagaaagggcactcactaccggtcatacgcggcgtcataagcgtggtggtaagcgtccgtttggttcgaagtatcatgatcattgtggttgtacggtggttgaggttgttggcccttgggagccaaatagggctgatgtcgagtatcagaggacgtatgagaaggcccgtgagtgggttgatgatcatgggttgcagcagtcgcctggcaatattttgaaggctatgcgtactgttggcgatatgagataatttgatgtggtttccggttgtgcgccgccggttattggtgcacagggttgtctcccgcacgggggtcaacaatgttgtgttgttttccgcaaggagtgtagggttaggctatggccgatcagagtgttgaggaacagaatgttgacaatgatgttgtggagtccggaaaggataacggcattgttgatacagtaaaagacgatggcgggcaggaggtagccgacaatcagttgaagaatgaaggcgagggtaaatcgccggggactgattggaaggcggaggcccgtaagtgggagtctcgtgctaaaagtaatttcgccgagttggagaagcttcgcgcctcggatggtgattctggatctactattgctgagcttcgccgcaagaatgaggaactcgaagacaggatcaacgggtttgttcttgagggtgtgaagcgcgagatggcttcagagtatggtttgtccagtgatgcgatcgttttcttgtcgggtggcgataaggagtcgcttgccgagtctgcgaaagctttgaagggtttgatcgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggttgacgattttctttctgcagggaagctggagcttcctggttctatgattggtgcggttcgtgaccgtgctatcgattctggtgttttggcgaagctttcgccggagcagccgactatttttggccctgttaagggtgccgtgtttagtggtgttcctcgtgctaagattgttggtgagggcgaggttaagccttccgctagcgttgatgtttcggcgtttactgcgcagcctatcaaggttgtgactcagcagcgtgtctcggacgagtttatgtgggctgatgctgattaccgtctgggtgttttgcaggatctgatttccccggctcttggtgcttcgattggtcgcgccgtggatctgattgctttccatggtattgatcctgccactggtaaagcggctgccgctgtgcatacttcgctggataagacgaagcatattgttgatgccacggattctgctacgaccgatctggtcaaggctgtcggtcttatcgctggtgctggtttgcaggttcctaacggggttgctttggatccggcgttctcgtttgccctgtctactgaggtgtatccgaaggggtctccgcttgccggccagcctatgtatcctgccgccgggtttgctggtttggataattggcgtggcttgaatgttggttcttcttcgactgtttctggcgccccggagatgtcgcctgcctctggtgttaaggctattgttggtgatttctcgcgtgttcattggggtttccagcgtaacttcccgatcgagcttatcgagtatggtgacccggatcagactgggcgtgacctgaagggccataatgaggttatggttcgtgccgaggctgtgctgtatgtggctatcgagtcgcttgattcgtttgctgttgtgaaggagaaggctgccccgaagcctaatccgccggccgagaactgatttattgttgcggtgatgtgtcaatgtgcagggggtggtgttgatgggtatcattttgaagcctgaggatattgagccttttgccgatattcctagagagaagcttgaggcgatgattgccgatgtggaggctgtggctgtcagtgtcgccccctgtatcgctaaaccggatttcaaatacaaggatgccgctaaggctattctgcgcagggctttgttgcgctggaatgatactggcgtgtcgggtcaggtgcagtatgagtctgcgggtcctttcgctcagactacacggtctagtactcccacgaatttgttgtggccttctgagattgccgcgttgaagaagctgtgtgagggtgatggtggggctggtaaagcgttcactattacaccgaccatgaggagtagtgtgaatcattctgaggtgtgttccacggtgtggggtgagggttgctcatgcgggtcgaatattaacggctacgctggccctttgtgggagatatgatatgaccagttttccttatggtgaaacggttgtgatgcttcaaccgactgttcgtgtcgatgatcttggtgacaaggttgaggattgggggcatcctgtagaaaccgtgtaccataacgtggccatctatgcttccgtttcgcaggaggatgaggccgcggggcgtgactctgactatgagcattggtcgatgcttttcaagcagtctgttgttggtgctgattatcgttgccggtggcgtattcggggtgttgtgtggggggctgacgggtctcctatggtgtggcatcaccccatgtccggttgggatgcgggcacgcagatcaatgtgaagcgcaagaagggctgatagattgtggctcaggatgtgaatgtgaagctgaacttgccgggtattcgtgaggtgttgaagtcttctggggtgcaggctatgttggctgagcgtggcgagcgtgtcaagcgtgcggcctcggcgaatgtgggcggtaacgctttcgataaggcccaataccgtaatggtttgtcgtcggaggtgcaggttcaccgtgttgaggctgtcgctcgtataggtaccacatataagggtgggaagcgtattgaggcgaagcatggcacgctggctaggtcgattggggcggcgtcgtgatcatctacgatgaccccaggaagtgggctaaacgcgtgctcaaggatgatggctggctgtctgggataccatgcaccgggacagtgcccgatgattttacgggtgacctgatttggttggcgttggatggtggcccacagttgcatgttcgcgagcaagtttttttgcgcgtgaatgtgttttctgatacgccggatcgtgctatgtcgctagccaggcgggtggaggctgtccttgcggatggggttgatggcaaccctgtggtgtactgtaaacggtctactggtcctgatttgctggttgatggtgcacgttttgatgtgtattcgctgttcgagctgatatgtaggcctgtcgagtctgagtaaacgtatttgtttttgttttaatgtaattgtttgatatttaatgggggttgtgatggctgcaacacgtaaagcgtctaatgttcgttcagcggttactggcgacgtttatattggtgacgcgcacgcgggtgatactattaagggtgtggaggcggttcctgacggtcttaccgctttagggtatctgtcggatgacgggtttaagattaagcctgagcgtaaaacggatgatttgaaggcttggcagaatgcggatgttgttcgcacggttgctaccgagtcttctatcgagatttctttccagctgatcgagtctaagaaggaggttatcgagctgttttggcagtcgaaggttactgccggatccgattcaggttcgttcgatatttctccgggtgccacgacgggtgttcacgccctgttgatggatattgtggatggtgatcaggttattcgctactatttccctgaggttgagttgatcgatcgtgacgagatcaagggcaagaatggcgaggtgtacgggtatggtgtgacgttgaaggcgtatcctgcccagattaataagaagggtgatgcggtgtcgggtcgggggtggatgacggctttaaaagctgatactcctccggttccgccttctccgaagccgaagccggatcctaatccgccgtctgagaactgatacacgattttaggggattgttgatagatgagtgacacgggttacacgttgaagattggtgaccgtagctgggtgttggcggatgcggaggagacggctcaagctgtgcctgcccgcgtgtttcgccgtgcagctaagattgcccagtcgggggagtctgcggatttcgcccaggttgaggtgatgttttctatgttggaggctgccgccccagtggatgctgtggaggccctggaggggcttcctatggttcgtgtggccgagattttccgtgagtggatggaatataagcctgacggtaagggtgcctcgctgggggaatagtttggctccacggcctgattgatgattatcgtggggccatcgaatatgattggaggacccggttcggttgctcggtttatgatgttggtggcccgataatgtgttggggtgaggctgttcggctggctggcgtgttgtgtaccgatacgtctagccagttggcggcccacctgaatggttggcagcgcccgtttgagtggtctgagtgggcggtgttggacatgttggatcattacaggtctgctaatagtgaggggcagccggagcctgtggcgaggcctacggatgagcgtagggcccggtttacgtttgggcaggtggacgatattttggcgcgtgttcgtgccggtggcggggtgtctcgcgagattaatattatggggtgaatagtgtatgtctggtgagattgcttccgcatatgtgtcgttgtatacgaagatgcctggtttgaaggctgatgttggtaaacagttgtcgggtgttatgcctgctgagggtcagcgttcgggtagtctttttgctaagggtatgaagttggcgcttggtggtgccgcaatggtgggtgccatcaatgttgctaagaagggcctcaagtcgatttatgatgtgactattggtggcggtattgctcgcgctatggctattgatgaggctcaggctaagttgactggtttgggtcatacgtcgtctgacacgtcttcgattatgaattcggctattgaggctgtgactggtacgtcgtatgcgttgggggatgcggcttctactgcggcggcgttgtctgcttcgggtgtgaagtctggcgggcagatgacggatgtgttgaagactgtcgccgatgtgtcttatatttcgggtaagtcgtttcaggatacgggcgctatttttacgtctgtgatggcccgcggtaagttgcagggtgatgacatgttgcagcttacgatggcgggtgttcctgtactgtctttgcttgccaggcagacgggtaaaacgtcggctgaggtgtcgcagatggtgtcgaaggggcagattgattttgccacgtttgcggctgcgatgaagcttggcatgggtggtgctgcgcaggcgtctggtaagacgtttgagggcgctatgaagaatgttaagggcgctttgggctatcttggtgctacggctatggcgccgtttcttaacgggttgcggcagatttttgttgcgttgaatccggttatcaagtctatcacggattctgtgaagccgatgtttgctgccgtcgatgctggtattcagcgtatgatgccgtctattttggcgtggattaaccgtatgccgggcatgatcactcgaatgaatgcacagatgcgcgccaaggtggagcagttgaagggcatttttgcaaggttgcatttgcctgtccctaaagtgaatttgggtgccatgtttgctggcggcaccgcagtgtttggtattgttgctgccggtgtggggaagcttgtcgcggggtttgccccgttggcggtgtcggtgaagaatctactgccgtcgtttggtgctttgaagggtgccgccggcgggcttggcggcgtgtttcgcgccctgggtggccctgtcgggattgtgatcggcttgtttgctgccatgtttgctacgaacgcccagttccgtgccgctgttatgcagcttgtggctgtggttggtcaagccctggggcagattatggccgctgtgcagcctgtgtttggtttggttgcgggtctggtggcccggttggcgccagtgtttgcccagattattggtttggttgccgggctggctgcccagttgatgcctgtgattggtatgcttgttgcccggctggttcctgtgatcacccagattattggtgcggtgacgcaggtggcggccatgttgctgccggcgttgatgccggtgttgcaggctgttgttgctgtgatacggcaggttgttggcgtgatcatgcagttggtgccggtgttgatgccggtgattcagcagattttgggtgcggtcatgtctgtgctgccgccgattattggtttgatccggtcgttgatgcctgtgattgcggcggttatgcgtgtggtggtgcaggttgtttcggttgtgatacaggtggtggcccgtattcttgctgttgtggctccgatggtggctgccgtggtagggtttgttgcccgtattgttggtgctgtcgtgtcggctgttgcccgtgttattgctgctgttgcccgtgttatcgggtggattgttgctcattttgtgtcgggtttggcgcgtatgggttcggttattcaggctggctggaatcatattagggcgtttacgtctgcgtttattaacggttttaagtcggtgatttctggcggcgtgaacgctgttgtggggttttttacgcggcttggtttgtcggttgcttctcatgttcggtctggttttaacgcggctcgtggtgctgtttcttctgcgatgaatgctattcggagtgttgtgtcttcggtggcgtctgctgttggcgggtttttcagttcgatggcgtctagggttcgtagtggtgttgtgcgcgggtttaatggggccaggaatgcggcatcttccgctatgcatgctatggggtccgctgtgtctagcggcgtgcatagtgtgctagggtttttccggaatctgcctggcaatattcggcatgctctcggtaatatggggtctttgttggtgtctgctggccgtgatgtggtggccggtttgggtaacggtattaagaatgctttgagtggcctgttggatacggtgcgtaatatgggttctcaggttgctaatgctgcgaagtcggtgttgggtattcattccccgtcgagggtgtttcgtgacgaggttggccgtcaggttgttgccggtttggctgagggtattactggtaatgcgggtttggcgttggatgcgatgtctggtgtggctggtcggctgcctgatgtggtggatgcccggtttggtgtgcgatcgtctgtgggctcgtttaccccgtacgaccggtatcggcgtgcgagtgagaagagtgttgtggtgaatgttaacgggcccacgtatggtgatcctaacgagtttgcgaagcggattgagcgtcagcagcgtgacgctttgaacgctttggcttacgtgtgatagggggtgtggttcatgtttcttcctgacccgtctgatcgttctggtttgactgttacctggtctatggatccgctgtttggcgatgagcgtgtgcttcatttgacggattatacggggtcgtctccggtgatgttgttgaatgattcgttgcgcggtttgggtgttcctgaggtggagcatttttctcaaactcatgttggggtgcatggctcggagtggcgcgggtttaatgtgaagcctcgcgaggtgacgctgcctgtcctggtgtcgggtgttggtgtggatcctgtgggcgggtttcgtgacggttttttgaaagcctatgacgcgttgtggtctgcttttcctcccggggaggagggtgaactgtcggtgaagactcctgccggcaaagagcgtgtgctgaagtgccggtttgattcggctgatgacacgtttacggtggatccggtgaacaggggttatgcgcgttatctgttgcatttgacggcttatgacccgttttggtatggggatgagcagaagtttcgtttcagtaacgcgaagttgcaggattggttgggtggcggccctgtcggcaagaagggtacagcgtttcctgtggtgttgacgcctggtgttggttcgggttgggataacttgtctaataggggtgatgtgccggcgtggcctgtgattcgtgtggagggccccctggagtcgtggtctgtgcagattgatggtttgcgtgtgtcttcggattggcctgtcgaggagtatgattggatcactattgatacggatcctcgtaaacagtctgcgttgttgaacgggtttgaggatgtgatggatcgtttgaaggagtgggagtttgcgcctatcccgcctggcggttctaagagtgtgaatattgagatggttggtttgggtgccattgttgtgtcggtgcagtacaggtttttgagggcttggtgaatagttgatggctggtcttgttccgcggataacattgtttacaccggattatcaccgtgtggcgcctatcaatttttttgaatcgttgaaactgtcgttgaagtggaatggtttgtccactttggagttggtggtgtctggtgatcattctaggcttgacgggttgactaagccgggtgcacggctggttgttgattatggtggtggccagattttttctgggcctgtgcgtaaggttcatggtgtgggtccgtggcgttcttcgcgggtgactatcacgtgtgaagatgatattcgtctgttgtggcgtatgttgatgtggcctgtgaattatcgtcctggtatggttggtatggagtggcgtgccgacagggattatgcccactattcgggtgcggctgagtcggtggctaagcaggtgttgggggataatgcttggcgttttccgcctgatatatttatggtggatgataagagtcgtggccgctatattaaggattttcaggcgcggtttcacgtgtttgccgataagttgttgccggtgttgtcgtgggctcggatgactgtcacggtgaaccagtttgagaatgcgaagcaggatcagcggggtttgctgtttgattgtgtgcctgccgtgacccgtaagcatgtgttgactgccgagtctgggtctattgtgtcgtgggagtatgtgagggatgccccgaaggcgacatctgtggtggttggtggccgcggcgagggtaaggatcggctgttttgtgaggatgttgattcggcggccgaggatgactggtttgatcgtgtcgaggtgtttaaggatgcccgtaacacggattctgaacatgtgcatcttattgatgaggcggagcaggtgctgcaggagtctggggccacgtcggggtttaagatcgagttggccgagtcggatgtgttgcggtttgggccaggcaatctgatgccgggtgatttgatctatgtggatgtgggctcgggctctatcgcggagattgttcggcagattgatgtggagtgtgattcgccgggtgatggttggacgaaagtgactcctgttgcgggggattatgaggataatccgtcagcattgttggctcgccgtgttgccggtttggctgcgggtgtgcgggatttgcaaaagttttagaaggattggggtttgttgtgggtattgtgtgtaaagggtttgatggtgtgttgaccgagtatgattgggctcaaatgtctggtctgatgggtaatatgccgtcggtgaaagggccggatgattttcgtgtcggtacgactattcagggtgccacagtgttgtgtgaggtcctgccggggcaggcttgggctcacggggtgatgtgcacgtcgaatagtgttgagacggtgacggggccgcttccgggccctggcgagacccgatacgactatgtggtgttgtctcgggattgggagcagaatacggccaagttggagattgtttctggggggcgtgcggagcgtgccagggatgtgttgcgtgccgagcctggcgtgtttcatcagcagttgttggcgactttggtgttgtcgtctaacgggttgcagcagcagttggataggcgtgctatagcggctagggttgcgtttggcgagtctgctgcgtgtgatcctaccccggtggagggtgaccgggtgatggttccttcgggggctgtgtgggctaatcatgctaacgagtggatgctactgtctccgaggattgagacgggttctaagcagatccagtttggcgggtctgccgtgtatgcttacacgatcccgtttgatcgccagttcactagtgcgcctgtcgtggtggcgtctatggctacggcggctgggggcacggcacagatcgatgtgaaagcctacaatgttactgccaaggattttcggttggcgtttatcacgaatgacgggtctaagccgaatggtgtgcctgcggtggctaactggattgctgtcggcgtgtgactgtacaggtgttgtggcggatggtgtgatgttggggggctgtggtgtcgtggtttactcctgcactggtggcctctatctgtacggcgttggccacggttttgggttctgttcaggctgtcacatcccggtctaggaagcgtttacgcaggctgtctgcgcaggtggatgcgatggaagagtatacgtggggtgtgcggcgcgaggttcgaaggtttaacgccgggcttcctgacgaggtggagcctatgcatcttcctgatttgcccgagtttttgaaagatactgttgatggtggtggggggtgaattgtgagggagttggaggaagaaaaaaggcagcgccgctcgtttgagaaggcttccctgatactgttgttcctgtcgcttgtgctgttggcggtggttgctgcgggtgctttacggtacgggtctgtggcttcccagcgggattcggagcaggcgagggcccagtctaatggtacagccgctaaagggttggccagccgtgtgaagcgggtgtgtgcttcgggtgggcaggagtcggtgcggcttcaccagtctggcttgtgtgtggatgctcggcgtgttgagcggagtgtgcagggtgtgccgggtcctgcaggtgctgatggccgggatggtgttaatggttcggctgggctggttggccctgttggtccgcagggttctcctggtttg

SEQ ID NO: 76 - DNA, 83 bp, synthetic construct
  source 1..83: mol_type = other DNA; note = Cos PAC7 pIC405
aaaacccgccaacccccaccgggcacaccccctgcacacccgtgcaagacctcgtacggcttagtgaaatacctcccttttgt

SEQ ID NO: 77 - DNA, 300 bp, synthetic construct
  source 1..300: mol_type = other DNA; note = Cos PAC7 pIC400
aggcaacagaacacaaccaaacatcaaattccaacaacaaaccacaaaaacattgattccatggtgaaaaacccgccaacccccaccgggcacaccccctgcacacccgtgcaagacctcgtacggcttagtgaaatacctcccttttgttgttttatcgttttgtcgactttttgtttggtggtgtgtgtggtgcagcctgagcttcctgatagtcgtgattggtgtggggagacgcgtcggtggtggtgtgtgtggggcgaggatccgcgtgccgggtttgtgtctgatgaggagtgg

SEQ ID NO: 78 - DNA, 300 bp, synthetic construct
  source 1..300: mol_type = other DNA; note = Cos PAC7 pIC401
gaccacatcacacccgtcagccggggaggactcaacaccctcgacaacgggcaaatcatctgcagaacatgcaacagaagcaaaggcaacagaacacaaccaaacatcaaattccaacaacaaaccacaaaaacattgattccatggtgaaaaacccgccaacccccaccgggcacaccccctgcacacccgtgcaagacctcgtacggcttagtgaaatacctcccttttgttgttttatcgttttgtcgactttttgtttggtggtgtgtgtggtgcagcctgagcttcctgatagtc

SEQ ID NO: 79 - DNA, 217 bp, synthetic construct
  source 1..217: mol_type = other DNA; note = Cos PAC7 pIC402
aggcaacagaacacaaccaaacatcaaattccaacaacaaaccacaaaaacattgattccatggtgaaaaacccgccaacccccaccgggcacaccccctgcacacccgtgcaagacctcgtacggcttagtgaaatacctcccttttgttgttttatcgttttgtcgactttttgtttggtggtgtgtgtggtgcagcctgagcttcctgatagtc

SEQ ID NO: 80 - DNA, 150 bp, synthetic construct
  source 1..150: mol_type = other DNA; note = Cos PAC7 pIC403
aaaacccgccaacccccaccgggcacaccccctgcacacccgtgcaagacctcgtacggcttagtgaaatacctcccttttgttgttttatcgttttgtcgactttttgtttggtggtgtgtgtggtgcagcctgagcttcctgatagtc

SEQ ID NO: 81 - DNA, 150 bp, synthetic construct
  source 1..150: mol_type = other DNA; note = Cos PAC7 pIC404
aggcaacagaacacaaccaaacatcaaattccaacaacaaaccacaaaaacattgattccatggtgaaaaacccgccaacccccaccgggcacaccccctgcacacccgtgcaagacctcgtacggcttagtgaaatacctcccttttgt

SEQ ID NO: 82 - DNA, 25 bp, synthetic construct
  source 1..25: mol_type = other DNA; note = IC208 primer
gcttccttagcttgcgaaatctcga

SEQ ID NO: 83 - DNA, 26 bp, synthetic construct
  source 1..26: mol_type = other DNA; note = IC310 primer
gttcggctaaacccaaaagtaaaaac

SEQ ID NO: 84 - DNA, 35 bp, synthetic construct
  source 1..35: mol_type = other DNA; note = AD1541 primer
gttccagctcttccgaggaccacatcacacccgtc

SEQ ID NO: 85 - DNA, 35 bp, synthetic construct
  source 1..35: mol_type = other DNA; note = AD1542 primer
gttccagctcttcctgcccactcctcatcagacac

SEQ ID NO: 86 - DNA, 38 bp, synthetic construct
  source 1..38: mol_type = other DNA; note = IC511 primer
gttccagctcttccgagaggcaacagaacacaaccaaa

SEQ ID NO: 87 - DNA, 37 bp, synthetic construct
  source 1..37: mol_type = other DNA; note = IC512 primer
gttccagctcttcctgcgactatcaggaagctcaggc

SEQ ID NO: 88 - DNA, 37 bp, synthetic construct
  source 1..37: mol_type = other DNA; note = IC513 primer
gttccagctcttccgagaaaacccgccaacccccacc

SEQ ID NO: 89 - DNA, 38 bp, synthetic construct
  source 1..38: mol_type = other DNA; note = IC514 primer
gttccagctcttcctgcacaaaagggaggtatttcact

SEQ ID NO: 90 - DNA, 21 bp, synthetic construct
  source 1..21: mol_type = other DNA; note = AD1261 primer
cagcggcgctgctaagaactt

SEQ ID NO: 91 - DNA, 20 bp, synthetic construct
  source 1..20: mol_type = other DNA; note = AD1262 primer
ccggctggcaaatgaggcat

(19) United States
(12) Patent Application Publication
(10) Pub. No.: US 2024/0254496 A1
(43) Pub. Date: Aug. 1, 2024

(54) PHAGE-DERIVED PARTICLES FOR IN SITU DELIVERY OF DNA PAYLOAD INTO C. ACNES POPULATION

(71) Applicant: Eligo Bioscience, Paris (FR)

(72) Inventors: Aymeric LEVEAU, Paris (FR); Inès CANADAS BLASCO, Paris (FR); Aurélie MATHIEU, Paris (FR); Antoine DECRULLE, Paris (FR)

(21) Appl. No.: 18/617,782

(22) Filed: Mar. 27, 2024

Related U.S. Application Data
(63) Continuation of application No. 18/477,069, filed on Sep. 28, 2023, now Pat. No. 11,970,701.
Application No. 18/477,069 is a continuation of application No. 17/518,936, filed on Nov. 4, 2021, now Pat. No. 11,820,989.
(60) Provisional application No. 63/109,832, filed on Nov. 4, 2020.
(60) Provisional application No. 63/109,834, filed on Nov. 4, 2020.
(60) Provisional application No. 63/145,967, filed on Feb. 4, 2021.
(60) Provisional application No. 63/145,969, filed on Feb. 4, 2021.

(51) Int. Cl. C12N 15/76; A61K 39/00; A61K 39/02; A61K 39/05; A61P 17/10; C12N 9/22; C12N 9/64; C12N 15/11

(52) U.S. Cl. CPC C12N 15/76; A61K 39/02; A61K 39/05; A61P 17/10; C12N 9/22; C12N 15/11; A61K 2039/53; C12N 9/64; C12N 2310/20; C12N 2795/10343; C12N 2800/101; C12N 2800/80

(57) ABSTRACT

The invention relates to C. acnes carrying DNA vectors with a C. acnes phage packaging signal and a gene of interest. The invention encompasses a C. acnes producer cell carrying DNA vectors, with a C. acnes phage packaging signal and a gene of interest, for the production of phage-derived particles that can robustly transduce C. acnes receiver cell allowing transgene expression. The invention encompasses C. acnes phage-derived particles carrying these vectors, C. acnes containing these vectors or modified by transduction of these phage-derived particles, and methods of using these phage-derived particles.

### CROSS REFERENCE TO RELATED APPLICATIONS

This application is a continuation of U.S. application Ser. No. 18/477,069 filed Sep. 28, 2023, which is a continuation of U.S. application Ser. No. 17/518,936 filed on Nov. 4, 2021, now U.S. Pat. No. 11,820,989 issued on Nov. 21, 2023, which claims the benefit of U.S. application 63/109,832 filed Nov. 4, 2020, U.S. application 63/145,967 filed Feb. 4, 2021, U.S. application 63/109,834 filed Nov. 4, 2020, and U.S. application 63/145,969 filed Feb. 4, 2021, all of which are herein incorporated by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Sep. 27, 2023, is named EB2020-04b_USCont.xml and is 450,560 bytes in size.

### FIELD OF THE INVENTION

The present invention concerns Cutibacterium acnes phagemids and production method thereof.

### BACKGROUND OF THE INVENTION

The skin is the largest organ of the human body and the biggest interface between our body and our environment. As such it also acts as a barrier protecting us from physical (e.g., UV, wounds), chemical (e.g., acid, base) and microbial (virus, bacteria, fungi) threats. This protection is not only the result of its passive physical isolating nature made from successive layers of dense and interconnected dead cells (stratum corneum) surrounded by a lipidic matrix. It is also thanks to active mechanisms orchestrated by diverse types of skin and immune cells that secrete antimicrobial peptides (AMP), produce cytokine and chemokine to recruit lymphoid immune cells, sense skin injuries and trigger wound healing mechanisms among other processes¹.

Skin is the first organ in contact with microorganisms after our birth, it is populated with a vast amount of immune cells in close contact with a great diversity of microorganisms and thus, the skin immune system need to develop abilities to recognize beneficial microorganisms from pathogenic ones to avoid constant immune response and inflammation. Part of this education is happening early in life when specific bacterial species are colonizing the skin and modulate immune responses in order for them to be tolerated². These specific bacterial species are then able to stably colonize the skin establishing communities and becoming commensal strains.

Skin is not physiologically and spatially homogeneous throughout the body: oily (e.g cheek, back), moist (e.g., inguinal crease, interdigital web space, antecubital crease) and dry skin (e.g, volar forearm, hypothenar palm) exist depending on the body sites³. These different body sites are associated with different physiological conditions and carry distinct microbiomes with oily sites being mostly colonized with Cutibacterium acnes (formerly known as Propionibacterium acnes), whereas Staphylococcus and Corynebacterium species are more abundant in moist sites⁴. In addition to these physiological characteristics skin is also heterogeneous in space with different appendages: the sweat glands, the hair follicle, the sebaceous gland. The colonization of these appendages is only recently studied but show differences compared to skin surface (stratum corneum)⁴⁻⁶.

These skin appendages are specific anatomical places because they do not have stratum corneum. As a consequence, micro-organism inside these appendages are in contact with living keratinocytes and have access to a higher diversity of immune cells due to the dermis proximity. The hair follicle has specific immunological properties. It is able to recruit specific immune cells such as monocyte-derived Langerhans Cells precursors⁷ and actively maintain resident memory T cells (TRM)⁸ making it a potential essential place for antigen presentation. The hair follicle is also deprived of effector T cells and has a strong immunosuppressive environment making it an immune privileged area⁹.

Examples in the published literature indicate that skin-resident bacteria actively engage host immunity through an intact skin barrier, and activate specific immune cells in a species- and strain-dependent manner (Chen et al, Nature 2018; 555(7697):543). For instance, some but not all strains of S. epidermidis induce activation of S. epidermidis-specific IL-17⁺CD8⁺ T cells that protect against cutaneous infection (Naik et al, Nature 2015, 520(7545):104-108).

Due to the absence of stratum corneum, the skin appendages are also more permeable to chemicals as these will only need to cross the tight-junction barrier and not the stratum corneum which normally prevents water exchange and as a result all water-soluble substances are able to diffuse.

The pilosebaceous subunit comprising the fair follicle and the sebaceous gland is mostly colonized by C. acnes that thrive in this sebum rich and anaerobic environment. Cutibacterium acnes (formerly Propionibacterium acnes) is a gram-positive rod-shaped aerotolerant bacteria, first isolated from skin in 1897. It belongs to the order Actinomycetales, it is part of the Propionibacteriaceae family and it belongs to the genus Cutibacterium. This genus includes other human skin species such as Cutibacterium avidum, Cutibacterium granulosum and Cutibacterium humerusii¹⁰. C. acnes is one of the most prevalent and abundant bacteria on human skin where it can be found both on the skin surface (stratum corneum) and in the hair follicle. Inside the hair follicle, it is in direct contact with a large diversity of living cells such as keratinocytes, stem cells, sebaceous cells and immune cells, unlike on the stratum corneum where it is mostly in contact with the dead corneocyte. C. acnes is a commensal bacterium but has also been associated with several skin diseases such as acne vulgaris¹¹ or progressive macular hypomelanosis¹²⁻¹⁴.

In particular, new findings on C. acnes reveal that specific phylotypes might play a critical role in acne development11. Precisely, the role of C. acnes phylotype IA1 in acne is being widely underscored. Fitz-Gibbon and colleagues demonstrated that chromosomal regions, loci 1, 2 and 3, characteristic of ribotypes RT4 and RT5 (classified within the phylogroup IA1), are strongly associated with acne¹⁵. Since these chromosomal regions are absent in ribotypes that are associated with healthy skin (i.e., RT6), they represent a potential target to eliminate acne-associated C. acnes strains.

Being able to edit Cutibacterium acnes population by removing specific proinflammatory strains to prevent or cure disease such as acne vulgaris or leverage their privilege location into the pilosebaceous unit to modulate host immune response or improve wound healing are attractive therapeutic approaches. To implement such approaches, one can either genetically modify C. acnes strains in situ or provide in vitro genetically modified C. acnes. Because of the large intra-individual and inter-individual microbiome diversity both at the species and at the strain level, it appears difficult to provide a single or cocktail of engineered C. acnes strains able to colonize the skin of most patients.

Delivery of DNA in situ to the C. acnes population offers a way to circumvent such difficulties by allowing to leverage pre-establish strains potentially without disturbing the local microbiome. However, in situ delivery of genetic material to C. acnes is a challenging task for several reasons. First, there are so far no genetic elements such as plasmid able to robustly and autonomously replicate inside C. acnes. The few described genetic modifications consist in genomic insertion of synthetic DNA through homologous recombination¹⁶⁻¹⁸. This in vitro process has been shown to be of very low efficiency and rely on the use of an antibiotic selection marker to select such events. Moreover, these genetic modifications have been restricted to a few specific strains (KPA17202, one RT6 C. acnes) and might not be generalizable to all C. acnes strains. Second, in order to perform in situ genetic modification of C. acnes we need to deliver DNA into C. acnes. The only described method for introducing DNA into C. acnes is the use of electroporation^{19,20}, a method that can only be performed in vitro.

The present invention solves both the lack of replicative and stable DNA vectors and their delivery into C. acnes using phage-derived particles.

### BRIEF SUMMARY OF INVENTION

The invention encompasses Cutibacterium acnes phagemids, bacterial cells comprising these phagemids, methods for making phage-derived particles comprising these phagemids, phage-derived particles comprising these phagemids, and methods for using these phagemids, particles, and cells, particularly in treatments of Cutibacterium acnes related disorders and/or diseases.

The invention encompasses a recombinant DNA phagemid vector, phage-derived particles comprising these vectors, and Cutibacterium acnes carrying the vector, wherein the vector comprises:
a phage packaging signal allowing packaging of the DNA vector in a Cutibacterium acnes phage capsid; and
a gene of interest.

In one embodiment, the DNA vector comprises:
a phage packaging signal allowing packaging of the DNA vector in a Cutibacterium acnes phage capsid; wherein the phage packaging signal sequence is at least 80, 83, 85, 87, 90, 93, 95, 97, 98, 99, or 100% identical to the sequence SEQ ID NO: 66; and
a gene of interest.

In one embodiment, the DNA vector comprises:
a phage packaging signal allowing packaging of the DNA vector in a Cutibacterium acnes phage capsid;
a gene of interest; and
a selection marker allowing for selection of the DNA vector in Cutibacterium acnes.

In one embodiment, the DNA vector comprises:
a phage packaging signal allowing packaging of the DNA vector in a Cutibacterium acnes phage capsid; wherein the phage packaging signal sequence is at least 80, 83, 85, 87, 90, 93, 95, 97, 98, 99, or 100% identical to the sequence SEQ ID NO: 66;
a gene of interest; and
a selection marker allowing for selection of the DNA vector in Cutibacterium acnes.

In one embodiment, the DNA vector comprises:
a phage packaging signal allowing packaging of the DNA vector in a Cutibacterium acnes phage capsid;
a gene of interest;
an origin of replication allowing replication in Cutibacterium acnes; and
optionally a selection marker allowing for selection of the DNA vector in Cutibacterium acnes.

In one embodiment, the DNA vector comprises:
a phage packaging signal allowing packaging of the DNA vector in a Cutibacterium acnes phage capsid; wherein the phage packaging signal sequence is at least 80, 83, 85, 87, 90, 93, 95, 97, 98, 99, or 100% identical to the sequence SEQ ID NO: 66;
a gene of interest;
an origin of replication allowing replication in Cutibacterium acnes; and
optionally a selection marker allowing for selection of the DNA vector in Cutibacterium acnes.

In one embodiment, the phage packaging signal sequence is at least 80, 83, 85, 87, 90, 93, 95, 97, 98, 99, or 100% identical to a phage packaging signal sequence selected from the group consisting of the sequences SEQ ID NO: 76, SEQ ID NO: 77, SEQ ID NO: 78, SEQ ID NO: 79, SEQ ID NO: 80 and SEQ ID NO: 81.

In one embodiment, the DNA vector further comprises a C. acnes phage origin of replication.

In one embodiment, the DNA vector further comprises a C. acnes phage origin of replication, wherein the phage origin of replication sequence is identical at least 75, 77, 80, 83, 85, 87, 90, 93, 95, 96, 97, 98, 99 or 100% identical to the sequence SEQ ID NO: 67.

In one embodiment, the gene of interest is a DNA encoding an antigen.

The invention encompasses a Cutibacterium acnes producer cell carrying a recombinant DNA vector for the production of Cutibacterium acnes phage-derived particles that contain the recombinant DNA vector.

The DNA vector is typically packaged into proteins produced from a Cutibacterium acnes phage genome or a helper phage. The C. acnes phage genome can be introduced into the C. acnes producer cell, for instance, by transformation or transduction with a C. acnes phage whereas the helper phage can be introduced into the C. acnes producer cell, for instance, by transformation or conjugation before or after introduction of the DNA vector into the C. acnes producer cell (FIG. 1).

The Cutibacterium acnes producer cell carrying a recombinant DNA vector typically comprises a Cutibacterium acnes phage genome leading to the production of phage-derived particles carrying the DNA vector.

In one embodiment, the Cutibacterium acnes phage genome is a non-engineered/wild-type genome.

In another embodiment, the Cutibacterium acnes phage genome is engineered.

In one embodiment, the DNA vector comprises an origin of replication able to replicate only in the Cutibacterium acnes producer cell and not in the Cutibacterium acnes receiver cell.

In one embodiment, the DNA vector comprises:
a phage packaging signal allowing packaging of the DNA vector in a Cutibacterium acnes phage capsid;
at least one gene of interest;
an origin of replication allowing replication only in Cutibacterium acnes producer cell; and
optionally a selection marker allowing for selection of the DNA vector in Cutibacterium acnes.

In one embodiment, the selection marker is an auxotrophic marker and the Cutibacterium acnes producer cell growth is dependent on this auxotrophic marker.

In one embodiment, the selection marker is an antibiotic resistance marker.

In one embodiment, the DNA vector further comprises a CRISPR-Cas system.

In one embodiment, the CRISPR-Cas system targets a C. acnes chromosome locus. Preferably, the targeted locus is not present in the C. acnes producer cell. Preferably, the CRISPR array is expressing one or several crRNA targeting the chromosome locus.

In one embodiment, the CRISPR-Cas system targets several C. acnes chromosome loci. Preferably, the targeted loci are not present in the C. acnes producer cell. Preferably, the CRISPR array from the CRISPR-Cas system is expressing one or several crRNA targeting the chromosome loci.

In one embodiment, the CRISPR-Cas system targets a C. acnes plasmid locus. Preferably, the targeted locus is not present in the C. acnes producer cell. Preferably, the CRISPR array from the CRISPR-Cas system is expressing one or several crRNA targeting the plasmid locus.

In one embodiment, the CRISPR-Cas system targets several C. acnes plasmid loci. Preferably, the targeted loci are not present in the C. acnes producer cell. Preferably, the CRISPR array from the CRISPR-Cas system is expressing one or several crRNA targeting the plasmid loci.

In one embodiment, the CRISPR-Cas system is not expressed in C. acnes producer cell. Preferably the CRISPR-Cas system is repressed in C. acnes producer cell.

In one embodiment, the CRISPR-Cas system targets a proinflammatory sequence related to host disease.

In one embodiment, the CRISPR-Cas system targets a proinflammatory sequence related to acne vulgaris.

In one embodiment, the DNA vector comprises a CRISPR-Cas system targeting the DNA vector itself.

In one embodiment, the DNA vector comprises a template for homologous recombination in C. acnes phages.

In one embodiment, the DNA vector comprises a template for homologous recombination in C. acnes chromosome.

In one embodiment, the DNA vector comprises a template for homologous recombination in C. acnes endogenous plasmids.

In one embodiment, the DNA vector comprises a template for homologous recombination and a CRISPR-Cas system targeting the DNA vector itself outside of the template region.

In one embodiment, the DNA vector comprises a template for homologous recombination and a CRISPR-Cas system targeting the DNA vector itself outside of the template region wherein the RNA guide (crRNA or sgRNA) from the CRISPR-Cas system is not perfectly matching the DNA target.

In one embodiment, the DNA vector comprises an integrase gene expression cassette and a site specific recombination site allow for the integration of the DNA vector inside the chromosome.

In one embodiment, the DNA vector comprises a prime editor gene expression cassette and one or multiple pegRNAs.

In one embodiment, the DNA vector comprises a base editor gene expression cassette and one or multiple crRNAs or sgRNAs.

In one embodiment, the selection marker is catA.

In one embodiment, the selection marker is ermE.

In one embodiment, the selection marker is hygB.

The invention encompasses a C. acnes phage-derived particle comprising any of the DNA vectors of the invention.

The invention encompasses a C. acnes, in particular an engineered C. acnes, comprising any of the DNA vectors of the invention.

In a particular embodiment, the engineered C. acnes comprises at least one, two, three or more DNA vectors, in particular DNA vectors of the invention.

In a particular embodiment, the engineered C. acnes comprises a DNA vector of the invention which comprises a DNA encoding an antigen.

The invention encompasses a C. acnes engineered following transduction of any of the vectors of the invention by phage-derived particles.

The invention encompasses an engineered C. acnes whose genome is altered following the transduction by a phage-derived particle containing any of the vectors of the invention.

The invention encompasses an engineered C. acnes produced by transducing C. acnes with any of the vectors of the invention, modifying the C. acnes with a gene of interest carried by the vector, selecting for the modification.

The invention encompasses an engineered C. acnes produced by transducing C. acnes with any of the vectors of the invention, modifying the C. acnes with a gene of interest carried by the vector, selecting for the modification, and curing the engineered C. acnes of the vector.

In one embodiment, the engineered C. acnes has been modified by a CRISPR-Cas system carried by the vector and transduced by a phage-derived particle containing any vectors from the invention.

In one embodiment, the engineered C. acnes has been modified by insertion of an exogenous gene into the C. acnes chromosome.

In one embodiment, the engineered C. acnes has been modified by insertion of an exogenous gene into the C. acnes plasmid.

In one embodiment, the engineered C. acnes has been modified by deletion or mutation of an endogenous genetic sequence in the C. acnes chromosome.

In one embodiment, the engineered C. acnes has been modified by deletion, insertion or substitution of one or several nucleotides into the C. acnes chromosome.

In one embodiment, the engineered C. acnes has been modified by deletion, insertion or substitution of one or several nucleotides into the C. acnes plasmid.

The invention encompasses a method for producing C. acnes phage-derived particles that contain any vector of the inventions, comprising the introduction of any of the DNA vectors of the invention into a C. acnes producer cell and contacting the producer cell with C. acnes phage genome.

The invention encompasses a method for engineering C. acnes comprising the introduction of any of the DNA vectors of the invention into a C. acnes. The method can further comprise selecting a modified C. acnes. The method can further comprise selecting a modified C. acnes that has an insertion of an exogenous gene into the C. acnes chromosome or into an endogenous plasmid. The method can further comprise selecting a modified C. acnes that has one or several deletions, insertions or substitutions of one or several nucleotides into C. acnes chromosome or endogenous plasmids.

The invention encompasses a phage-derived particle produced by any of the methods of the invention.

The invention encompasses methods for treating a C. acnes-related disorder or disease. In one embodiment, the method comprises administering a phage-derived particle of the invention or a bacterium producing such a phage-derived particle to a subject. The invention further concerns a phage-derived particle of the invention or a bacterium producing such a phage derived particle for use in a method for treating a C. acnes-related disorder or disease.

The invention encompasses methods for modifying a C. acnes to treat a disorder or disease or skin condition or for cosmetic applications. In one embodiment, the method comprises administering a phage-derived particle of the invention or a bacterium producing such a phage-derived particle to a subject. The invention further concerns a phage-derived particle of the invention or a bacterium producing such a phage-derived particle for use in a method for treating a disorder or disease or skin condition.

In one embodiment, the method is performed ex-situ.

In one embodiment, the method is performed in-situ.

In one embodiment, the method is performed ex-situ with a C. acnes strain isolated from the subject.

### BRIEF DESCRIPTION OF DRAWINGS

In order to better understand the subject matter that is disclosed herein and to exemplify how it may be carried out in practice, embodiments will be described, by way of non-limiting example, with reference to the accompanying drawings. With specific reference to the drawings, it is stressed that the particulars shown are by way of example and for purposes of illustrative discussion of embodiments of the invention.

FIG. 1 depicts a C. acnes producer cell carrying a DNA vector with a packaging signal and a transgene which is infected by a C. acnes phage, phage-derived particles carrying the DNA vector are then produced and upon binding to C. acnes receiver cell transduce the DNA vector that replicates and leads to transgene expression. Alternatively, the C. acnes producer is not infected by a phage but carries also a helper phage that is induced to trigger phage-derived particle production.

FIG. 2A-B depicts host range determination of isolated C. acnes bacteriophages. 1 indicates strain infection with full spot lysis; 0.5 indicates lower efficiency in strain infection with single plaques observed instead of full spot lysis.

FIG. 3 depicts a gel. Individual colonies from phage-derived particles titration were streaked and a PCR on an individual colony was performed with primers IC208 (SEQ ID NO: 82)/IC310 (SEQ ID NO: 83) to confirm the presence of the phagemid. 1 and 2 refer to transductants coming from the independent production and titration of phage-derived particles carrying the same phagemid. B and W are respectively PCR on the phagemid extraction (positive control) and the ATCC 11828 strain (negative control). Presence of the plasmid after restreak confirms that transductants carry the replicative phagemid.

FIGS. 4A and 4B depict a method for C. acnes genome engineering using non-replicative vector carrying recombination template. FIG. 4A depicts a vector (pEB_HR01) containing a single homology arm (HA) to C. acnes chromosome which is conjugated into C. acnes. Because the vector is not replicative in C. acnes, only C. acnes cells that perform a single recombination event stably maintain the antibiotic marker and are able to grow on antibiotic plate. Cells that do not perform the first recombination event or cells that perform the first and the second recombination events are not able to grow on antibiotic plates (erythromycin). FIG. 4B depicts a vector (pEB_HR02) containing two homology arms to C. acnes chromosome which is conjugated into C. acnes. Selection of the final recombinant is performed using an antibiotic selection (ErmE) and a counter selection (SacB).

FIG. 5 depicts a method for C. acnes genome engineering using replicative CRISPR-Cas system selection vector carrying recombination template. A replicative CRISPR-Cas system selection vector containing a template for homologous DNA recombination with the chromosome is conjugated into C. acnes. The template contains two homology arms (LHA and RHA) leading to homologous recombination in C. acnes chromosome and removal of the target sequence of the CRISPR-Cas system. Thus only recombinants C. acnes are able to grow in the presence of erythromycin when selected for the presence of the vector expressing CRISPR-Cas system.

FIGS. 6A and 6B depict a method for C. acnes genome engineering using self-targeted replicative vector carrying CRISPR-Cas system and recombination template. FIG. 6A depicts a vector, containing an antibiotic selection marker flanked by two homology arms and a CRISPR-Cas system targeting the vector outside the homology regions, which is conjugated into C. acnes. The CRISPR-Cas system cuts the vector leading to linearization of the template and plasmid loss. Thus, only recombinant cells are able to grow in presence of antibiotic. FIG. 6B depicts a vector, containing a mutant allele flanked by two homology arms and a CRISPR-Cas system targeting the vector outside the homology regions as well as the non mutated allele of C. acnes chromosome, which is conjugated into C. acnes. The CRISPR-Cas system cuts the vector leading to linearization of the template and plasmid loss as well as the C. acnes chromosome. Thus, only recombinant cells are able to grow in the presence of erythromycin.

FIG. 7 depicts a method for sequence specific killing or plasmid curing using phage-derived particles. C. acnes phage-derived particles bind a C. acnes cell (1) allowing injection of a DNA vector encoding a CRISPR-Cas system (2). The CRISPR-Cas system is expressed (3) and cuts the chromosome (4) and/or a plasmid (5 and 6) in a sequence specific manner. Cutting of the chromosome leads to cell death (4) whereas cutting of the plasmid leads to either the plasmid loss (6) or cell death if the plasmid encodes a toxin-antitoxin system (5).

FIG. 8 depicts C. acnes transductants of phage-derived particles carrying DNA vector with phage packaging signal (cos) of different sizes. Each suspension of phage-derived particles, also containing phages, was mixed with C. acnes ATCC 11828 pseudolysogene, the mixture was incubated for 1 hour at room temperature, diluted and 4 μL of each dilution was plated on Brucella plates in presence of erythromycin (5 μg/mL). For each phage-derived particle containing a same DNA vector, two suspensions from independent productions were used (e.g pIC400.1 and pIC400.2).

FIG. 9 (A and B) depicts absorbance values from ELISAs for the presence of chicken ovalbumin (OVA) protein in different C. acnes culture supernatant diluted 1/10.

FIG. 9A and FIG. 9B represent two independent replicas. Bar graphs represent the mean of three technical replicates of the same supernatant culture. C. acnes strains ATCC 11828 (WT) was used as negative control.

FIG. 10 depicts an ovalbumin specific western blot on culture supernatant from different C. acnes strains engineered to secrete ovalbumin. From left to right: (1) Pageruler ladder, (2) supernatant from strain Ca0s22120, (3) supernatant from strain Ca0s22122, (4) supernatant from strain Ca0s22126, (5) supernatant from strain Ca0s22128, (6) supernatant from strain Ca0s22130, (7) supernatant from strain Ca0s22132, (8) supernatant from strain Ca0s16973, (9) ovalbumin.

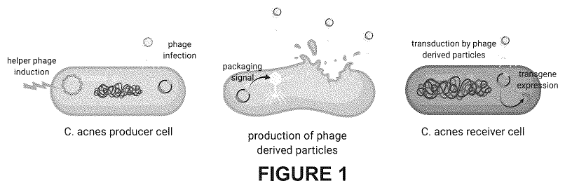

### DETAILED DESCRIPTION OF INVENTION

The inventors demonstrated, for the first time, the introduction of a recombinant replicative DNA in C. acnes by transduction, of a phage-derived particle.

The inventors also demonstrated, for the first time, the production of C. acnes phage-derived particles from a C. acnes strain, carrying a recombinant self-replicative DNA vector.

The invention relates to a C. acnes strain carrying a DNA vector comprising a phage packaging signal and a gene of interest, the production of phage-derived particles containing the DNA vector and the use of this phage-derived particles to transduce C. acnes in vitro or in situ and the subsequent expression of the gene of interest in the transduced C. acnes cell. The invention also relates to the modified C. acnes strains obtained by transduction of a DNA vector by the phage-derived particle, the modified C. acnes strains containing or not the DNA vector.

C. acnes phages are naturally present in the human skin and have been isolated numerous times since the first isolation in 1964. More recently, sequencing of C. acnes phages has revealed an unusual high level of nucleotide conservation with ˜85% identity. All C. acnes phages described so far are siphoviridae with a genome size constraint around 30 kb and a similar genome architecture. Despite their small genetic diversity, most C. acnes phages have the capacity to infect several C. acnes phylotypes and thus are considered as broad-host range. Their in-situ infectivity and their broad host range make them a relevant platform to be engineered for transgene delivery into the C. acnes population.

The inventors show for the first time that phage-derived particles can be produced from the co-occurrence of a wild-type or engineered C. acnes phage genome and a recombinant DNA vector with a packaging signal in a C. acnes cell (“producer cell”). The phage-derived particles are able to transduce the DNA vector into a “receiver” C. acnes cell and express a transgene such as an antibiotic resistance gene allowing the selection of the transductants. This widely expands the possibility to engineer C. acnes population directly on the skin, paving the way for many applications (industrial, therapeutic, cosmetic, environmental). The invention encompasses a C. acnes “producer” cell carrying DNA vectors, particularly phagemids, and methods for generating phage-derived particles and their use to modify or kill C. acnes.

### DNA Vectors

The invention encompasses recombinant DNA vectors for use in Cutibacterium acnes. Preferably, the DNA vector is a recombinant DNA vector, which is not integrated into the C. acnes chromosome. The vector allows transfer to progeny cells. The vector is preferably a phagemid. The DNA vector preferably comprises an origin of replication allowing replication in C. acnes and a phage packaging signal.

In various embodiments, the DNA vector comprises any combination of a phage packaging signal, an origin of replication allowing replication in C. acnes, a selection marker allowing for selection of the DNA vector in C. acnes, a gene of interest, and an origin of replication allowing replication in C. acnes producer cell but no replication in C. acnes receiver cell.

In one embodiment, the DNA vector comprises a phage packaging signal, an origin of replication allowing replication in C. acnes, a first selection marker allowing for selection of the DNA vector in C. acnes and a gene of interest.

In one embodiment, the DNA vector comprises a phage packaging signal, an origin of replication allowing replication in C. acnes producer cell but no replication in C. acnes receiver cell, a first selection marker allowing for selection of the DNA vector in C. acnes and a gene of interest.

Preferably, the gene of interest is exogenous to C. acnes, that is, one that is not found naturally in C. acnes.

In one embodiment, the DNA vector comprises a phage packaging signal, wherein the phage packaging signal sequence is at least 80, 83, 85, 87, 90, 93, 95, 97, 98, 99, or 100% identical to the sequence SEQ ID NO: 66; an origin of replication allowing replication in C. acnes; a selection marker allowing for selection of the DNA vector in C. acnes; and a gene of interest.

In one embodiment, the DNA vector comprises a phage packaging signal, wherein the phage packaging signal sequence is at least 80, 83, 85, 87, 90, 93, 95, 97, 98, 99, or 100% identical to the sequence SEQ ID NO: 66; an origin of replication allowing replication in C. acnes; a selection marker allowing for selection of the DNA vector in C. acnes; and a gene of interest.

In one embodiment, the DNA vector comprises a phage packaging signal, wherein the phage packaging signal sequence is at least 80, 83, 85, 87, 90, 93, 95, 97, 98, 99, or 100% identical to the sequence SEQ ID NO: 66; an origin of replication allowing replication in C. acnes; a selection marker allowing for selection of the DNA vector in C. acnes; a selection marker allowing for selection in a first bacteria wherein the first bacteria is E. coli; an origin of replication allowing replication in a first bacteria wherein the first bacteria is E. coli; and a gene of interest.

In one embodiment, the DNA vector can be efficiently introduced into and stably replicated in C. acnes producer cell using electroporation, using protoplast electroporation, using chemical transformation, using conjugation, using natural competency or using transduction.

In one embodiment, the DNA vector can be efficiently transformed into and stably replicated in C. acnes producer cell using physical methods such as electroporation of C. acnes cells or electroporation of C. acnes protoplast.

In one embodiment, the C. acnes protoplasts are generated using Mutanolysin treatment or Lysozyme treatment, Mutanolysin and Lysozyme treatment, or Mutanolysin and Lysozyme and bead-beating treatment followed by resuspension into hypotonique media.

In one embodiment, the DNA vector can be efficiently transformed into and stably replicated in C. acnes producer cell using C. acnes protoplast mix with DNA vector or DNA vector+glass beads.

In one embodiment, delivery of the DNA vector into C. acnes is by transduction. In one embodiment, the DNA vector comprises one packaging signal of a C. acnes phage selected from the group consisting of: PAC7 (typically of sequence SEQ ID NO: 68); PAC1 (typically of sequence SEQ ID NO: 69); PAC9 (typically of sequence SEQ ID NO: 70); PAC2 (typically of sequence SEQ ID NO: 71); PAC10 (typically of sequence SEQ ID NO: 72); PAC22 (typically of sequence SEQ ID NO: 73); PAC13 (typically of sequence SEQ ID NO: 74); and PAC263 (typically of sequence SEQ ID NO: 75), and is packaged into proteins expressed from the genome of a C. acnes phage selected from the group consisting of the phages: PAC7 (typically of sequence SEQ ID NO: 68); PAC1 (typically of sequence SEQ ID NO: 69); PAC9 (typically of sequence SEQ ID NO: 70); PAC2 (typically of sequence SEQ ID NO: 71); PAC10 (typically of sequence SEQ ID NO: 72); PAC22 (typically of sequence SEQ ID NO: 73); PAC13 (typically of sequence SEQ ID NO: 74); and PAC263 (typically of sequence SEQ ID NO: 75) allowing transduction of the DNA vector into C. acnes.

In one embodiment, the DNA vector comprises a packaging signal, the sequence of which is at least 80, 83, 85, 87, 90, 93, 95, 97, 98, 99, or 100% identical to the sequence of any of the above packaging signals.

In one embodiment, the phage packaging signal is of sequence at least 80, 83, 85, 87, 90, 93, 95, 97, 98, 99, or 100% identical to a phage packaging signal sequence selected from the group consisting of: SEQ ID NO: 76, SEQ ID NO: 77, SEQ ID NO: 78, SEQ ID NO: 79, SEQ ID NO: 80 and SEQ ID NO: 81.

In one embodiment, delivery of the DNA vector into C. acnes is by conjugation.

In one embodiment, the DNA vector comprises an origin of transfer selected from the group consisting of: oriT_pMRC01 (typically of sequence SEQ ID NO: 1); oriT_RSF1010 (typically of sequence SEQ ID NO: 2); oriT_pRS01 (typically of sequence SEQ ID NO: 3); oriT_pMV158 (typically of sequence SEQ ID NO: 4); oriT_pTF1 (typically of sequence SEQ ID NO: 5); oriT_pSC101 (typically of sequence SEQ ID NO: 6); oriT_pBTK445 (typically of sequence SEQ ID NO: 7); oriT_pBBR1 (typically of sequence SEQ ID NO: 8); oriT_R721 (typically of sequence SEQ ID NO: 9); oriT_pRmeGR4a (typically of sequence SEQ ID NO: 10); oriT_ColE1 (typically of sequence SEQ ID NO: 11); oriT_pTiC58 (typically of sequence SEQ ID NO: 12); oriT_pMdT1 (typically of sequence SEQ ID NO: 13); oriT_R1 (typically of sequence SEQ ID NO: 14); oriT_Tn5520 (typically of sequence SEQ ID NO: 15); oriT_QKH54 (typically of sequence SEQ ID NO: 16); oriT_R64 (typically of sequence SEQ ID NO: 17); oriT_R751 (typically of sequence SEQ ID NO: 18); oriT_RP4 (typically of sequence SEQ ID NO: 19); oriT_pKL1 (typically of sequence SEQ ID NO: 20); oriT_RK2 (typically of sequence SEQ ID NO: 21); oriT_R1162 (typically of sequence SEQ ID NO: 22); oriT_Tn4555 (typically of sequence SEQ ID NO: 23); oriT_pHT (typically of sequence SEQ ID NO: 24); oriT_Tn4399 (typically of sequence SEQ ID NO: 25); oriT_Tn916 (typically of sequence SEQ ID NO: 26); oriT_pST12 (typically of sequence SEQ ID NO: 27); oriT_pCU1 (typically of sequence SEQ ID NO: 28); oriT_pSU233 (typically of sequence SEQ ID NO: 29); oriT_F (typically of sequence SEQ ID NO: 30); oriT_pMAB01 (typically of sequence SEQ ID NO: 31); oriT_R388 (typically of sequence SEQ ID NO: 32); oriT_pS7a (typically of sequence SEQ ID NO: 33); oriT_pS7b (typically of sequence SEQ ID NO: 34); oriT_R702 (typically of sequence SEQ ID NO: 35); oriT_pMUR274 (typically of sequence SEQ ID NO: 36); oriT_R100 (typically of sequence SEQ ID NO: 37); oriT_pVCR94deltaX (typically of sequence SEQ ID NO: 38); oriT_R46 (typically of sequence SEQ ID NO: 39); oriT_pGO1 (typically of sequence SEQ ID NO: 40); and oriT_pIP501 (typically of sequence SEQ ID NO: 41).

In one embodiment, the DNA vector comprises the origin of transfer oriT_pMRC01 (typically of sequence SEQ ID NO: 1). In one embodiment, the DNA vector comprises the origin of transfer oriT_RSF1010 (typically of sequence SEQ ID NO: 2). In one embodiment, the DNA vector comprises the origin of transfer oriT_pRS01 (typically of sequence SEQ ID NO: 3). In one embodiment, the DNA vector comprises the origin of transfer oriT_pMV158 (typically of sequence SEQ ID NO: 4). In one embodiment, the DNA vector comprises the origin of transfer oriT_pTF1 (typically of sequence SEQ ID NO: 5). In one embodiment, the DNA vector comprises the origin of transfer oriT_pSC101 (typically of sequence SEQ ID NO: 6). In one embodiment, the DNA vector comprises the origin of transfer oriT_pBTK445 (typically of sequence SEQ ID NO: 7). In one embodiment, the DNA vector comprises the origin of transfer oriT_pBBR1 (typically of sequence SEQ ID NO: 8). In one embodiment, the DNA vector comprises the origin of transfer oriT_R721 (typically of sequence SEQ ID NO: 9). In one embodiment, the DNA vector comprises the origin of transfer oriT_pRmeGR4a (typically of sequence SEQ ID NO: 10). In one embodiment, the DNA vector comprises the origin of transfer oriT_ColE1 (typically of sequence SEQ ID NO: 11). In one embodiment, the DNA vector comprises the origin of transfer oriT_pTiC58 (typically of sequence SEQ ID NO: 12). In one embodiment, the DNA vector comprises the origin of transfer oriT_pMdT1 (typically of sequence SEQ ID NO: 13). In one embodiment, the DNA vector comprises the origin of transfer oriT_R1 (typically of sequence SEQ ID NO: 14). In one embodiment, the DNA vector comprises the origin of transfer oriT_Tn5520 (typically of sequence SEQ ID NO: 15). In one embodiment, the DNA vector comprises the origin of transfer oriT_QKH54 (typically of sequence SEQ ID NO: 16). In one embodiment, the DNA vector comprises the origin of transfer oriT_R64 (typically of sequence SEQ ID NO: 17). In one embodiment, the DNA vector comprises the origin of transfer oriT_R751 (typically of sequence SEQ ID NO: 18). In one embodiment, the DNA vector comprises the origin of transfer oriT_RP4 (typically of sequence SEQ ID NO: 19). In one embodiment, the DNA vector comprises the origin of transfer oriT_pKL1 (typically of sequence SEQ ID NO: 20). In one embodiment, the DNA vector comprises the origin of transfer oriT_RK2 (typically of sequence SEQ ID NO: 21). In one embodiment, the DNA vector comprises the origin of transfer oriT_R1162 (typically of sequence SEQ ID NO: 22). In one embodiment, the DNA vector comprises the origin of transfer oriT_Tn4555 (typically of sequence SEQ ID NO: 23). In one embodiment, the DNA vector comprises the origin of transfer oriT_pHT (typically of sequence SEQ ID NO: 24). In one embodiment, the DNA vector comprises the origin of transfer oriT_Tn4399 (typically of sequence SEQ ID NO: 25). In one embodiment, the DNA vector comprises the origin of transfer oriT_Tn916 (typically of sequence SEQ ID NO: 26). In one embodiment, the DNA vector comprises the origin of transfer oriT_pST12 (typically of sequence SEQ ID NO: 27). In one embodiment, the DNA vector comprises the origin of transfer oriT_pCU1 (typically of sequence SEQ ID NO: 28). In one embodiment, the DNA vector comprises the origin of transfer oriT_pSU233 (typically of sequence SEQ ID NO: 29). In one embodiment, the DNA vector comprises the origin of transfer oriT_F (typically of sequence SEQ ID NO: 30). In one embodiment, the DNA vector comprises the origin of transfer oriT_pMAB01 (typically of sequence SEQ ID NO: 31). In one embodiment, the DNA vector comprises the origin of transfer oriT_R388 (typically of sequence SEQ ID NO: 32). In one embodiment, the DNA vector comprises the origin of transfer oriT_pS7a (typically of sequence SEQ ID NO: 33). In one embodiment, the DNA vector comprises the origin of transfer oriT_pS7b (typically of sequence SEQ ID NO: 34). In one embodiment, the DNA vector comprises the origin of transfer oriT_R702 (typically of sequence SEQ ID NO: 35). In one embodiment, the DNA vector comprises the origin of transfer oriT_pMUR274 (typically of sequence SEQ ID NO: 36). In one embodiment, the DNA vector comprises the origin of transfer oriT_R100 (typically of sequence SEQ ID NO: 37). In one embodiment, the DNA vector comprises the origin of transfer oriT_pVCR94deltaX (typically of sequence SEQ ID NO: 38). In one embodiment, the DNA vector comprises the origin of transfer oriT_R46 (typically of sequence SEQ ID NO: 39). In one embodiment, the DNA vector comprises the origin of transfer oriT_pGO1 (typically of sequence SEQ ID NO: 40). In one embodiment, the DNA vector comprises the origin of transfer oriT_pIP501 (typically of sequence SEQ ID NO: 41).

In one embodiment, the DNA vector comprises an origin of transfer (oriT), the sequence of which is at least 80, 83, 85, 87, 90, 93, 95, 97, 98, 99, or 100% identical to the sequence of any of the above oriT.

In one embodiment, a donor bacterium, such as E. coli, carry a conjugative plasmid, a conjugative transposon, or an integrative and conjugative element (ICE) selected from the group consisting of: pMRC01, RSF1010, pRS01, pMV158, pTF1, pSC101, pBTK445, pBBR1, R721, pRmeGR4a, ColE1, pTiC58, pMdT1, R1, Tn5520, QKH54, R64, R751, RP4, pKL1, RK2, R1162, Tn4555, pHT, Tn4399, Tn916, pST12, pCU1, pSU233, F, pMAB01, R388, pS7a, pS7b, R702, pMUR274, R100, pVCR94deltaX, R46, pGO1 and pIP501; and is used to efficiently transfer the DNA vector into C. acnes recipient cells. In one embodiment the DNA vector contains an origin of transfer and the associated relaxase of the conjugative plasmid, conjugative transposon and integrative and conjugative element (ICE) selected from the group consisting of pMRC01; RSF1010; pRS01; pMV158; pTF1; pSC101; pBTK445; pBBR1; R721; pRmeGR4a; ColE1; pTiC58; pMdT1; R1; Tn5520; QKH54; R64; R751; RP4; pKL1; RK2; R1162; Tn4555; pHT; Tn4399; Tn916; pST12; pCU1; pSU233; F; pMAB01; R388; pS7a; pS7b; R702; pMUR274; R100; pVCR94deltaX; R46; pGO1 and pIP501.

In a preferred embodiment the DNA vector comprises an origin of transfer and the relaxase of the following conjugative plasmid, conjugative transposon and integrative and conjugative element (ICE) selected from the group consisting of: pMRC01; RSF1010; pRS01; pMV158; pTF1; pSC101; pBTK445; pBBR1; R721; pRmeGR4a; ColE1; pTiC58; pMdT1; R1; Tn5520; QKH54; R64; R751; RP4; pKL1; RK2; R1162; Tn4555; pHT; Tn4399; Tn916; pST12; pCU1; pSU233; F; pMAB01; R388; pS7a; pS7b; R702; pMUR274; R100; pVCR94deltaX; R46; pGO1 and pIP501.

In a preferred embodiment the DNA vector comprises an origin of transfer selected from the group consisting of: oriT_pMRC01 (SEQ ID NO: 1); oriT_RSF1010 (SEQ ID NO: 2); oriT_pRS01 (SEQ ID NO: 3); oriT_pMV158 (SEQ ID NO: 4); oriT_pTF1 (SEQ ID NO: 5); oriT_pSC101 (SEQ ID NO: 6); oriT_pBTK445 (SEQ ID NO: 7); oriT_pBBR1 (SEQ ID NO: 8); oriT_R721 (SEQ ID NO: 9); oriT_pRmeGR4a (SEQ ID NO: 10); oriT_ColE1 (SEQ ID NO: 11); oriT_pTiC58 (SEQ ID NO: 12); oriT_pMdT1 (SEQ ID NO: 13); oriT_R1 (SEQ ID NO: 14); oriT_Tn5520 (SEQ ID NO: 15); oriT_QKH54 (SEQ ID NO: 16); oriT_R64 (SEQ ID NO: 17); oriT_R751 (SEQ ID NO: 18); oriT_RP4 (SEQ ID NO: 19); oriT_pKL1 (SEQ ID NO: 20); oriT_RK2 (SEQ ID NO: 21); oriT_R1162 (SEQ ID NO: 22); oriT_Tn4555 (SEQ ID NO: 23); oriT_pHT (SEQ ID NO: 24); oriT_Tn4399 (SEQ ID NO: 25); oriT_Tn916 (SEQ ID NO: 26); oriT_pST12 (SEQ ID NO: 27); oriT_pCU1 (SEQ ID NO: 28); oriT_pSU233 (SEQ ID NO: 29); oriT_F (SEQ ID NO: 30); oriT_pMAB01 (SEQ ID NO: 31); oriT_R388 (SEQ ID NO: 32); oriT_pS7a (SEQ ID NO: 33); oriT_pS7b (SEQ ID NO: 34); oriT_R702 (SEQ ID NO: 35); oriT_pMUR274 (SEQ ID NO: 36); oriT_R100 (SEQ ID NO: 37); oriT_pVCR94deltaX (SEQ ID NO: 38); oriT_R46 (SEQ ID NO: 39); oriT_pGO1 (SEQ ID NO: 40) and oriT_pIP501 (SEQ ID NO: 41).

In one embodiment, the DNA vector comprises an origin of transfer (oriT) that is at least 80, 83, 85, 87, 90, 93, 95, 97, 98, 99, or 100% identical to any of these ICE.

In one embodiment, the invention encompasses a DNA vector comprising an origin of replication allowing replication in C. acnes, an oriT allowing conjugation into C. acnes, a selection marker allowing for selection in the transconjugant C. acnes, and a selection marker allowing for selection in the donor bacteria. In another embodiment, the invention encompasses a DNA vector comprising an origin of replication allowing replication in C. acnes and an oriT allowing conjugation into C. acnes as defined above.

In one embodiment, origin of replication allowing replication in C. acnes is selected from the group consisting of: R6K (typically of sequence SEQ ID NO: 42); RK2 (typically of sequence SEQ ID NO: 43); pBBR1 (typically of sequence SEQ ID NO: 44); pRO1600 (typically of sequence SEQ ID NO: 45); RSF1010 (typically of sequence SEQ ID NO: 46); pAM31 (typically of sequence SEQ ID NO: 47); pLME106 (typically of sequence SEQ ID NO: 48); pTZC1 (typically of sequence SEQ ID NO: 49); pBC1 (typically of sequence SEQ ID NO: 50); pEP2 (typically of sequence SEQ ID NO: 51); pWVO1 (typically of sequence SEQ ID NO: 52); pAP1 (typically of sequence SEQ ID NO: 53); pWKS1 (typically of sequence SEQ ID NO: 54); pLME108 (typically of sequence SEQ ID NO: 55); pLS1 (typically of sequence SEQ ID NO: 56); pUB6060 (typically of sequence SEQ ID NO: 57); p545 (typically of sequence SEQ ID NO: 58); pJD4 (typically of sequence SEQ ID NO: 59); pIJ101 (typically of sequence SEQ ID NO: 60); pSN22 (typically of sequence SEQ ID NO: 61); pGP01 (typically of sequence SEQ ID NO: 62); pIP501 (typically of sequence SEQ ID NO: 63); pCU1 (typically of sequence SEQ ID NO: 64); and pBAV1 K-T5 (typically of sequence SEQ ID NO: 65). In one embodiment, the origin of replication allowing replication in C. acnes is R6K (typically of sequence SEQ ID NO: 42). In one embodiment, the origin of replication allowing replication in C. acnes is RK2 (typically of sequence SEQ ID NO: 43). In one embodiment, the origin of replication allowing replication in C. acnes is pBBR1 (typically of sequence SEQ ID NO: 44). In one embodiment, the origin of replication allowing replication in C. acnes is pRO1600 (typically of sequence SEQ ID NO: 45). In one embodiment, the origin of replication allowing replication in C. acnes is RSF1010 (typically of sequence SEQ ID NO: 46). In one embodiment, the origin of replication allowing replication in C. acnes is pAM31 (typically of sequence SEQ ID NO: 47). In one embodiment, the origin of replication allowing replication in C. acnes is pLME106 (typically of sequence SEQ ID NO: 48). In one embodiment, the origin of replication allowing replication in C. acnes is pTZC1 (typically of sequence SEQ ID NO: 49). In one embodiment, the origin of replication allowing replication in C. acnes is pBC1 (typically of sequence SEQ ID NO: 50). In one embodiment, the origin of replication allowing replication in C. acnes is pEP2 (typically of sequence SEQ ID NO: 51). In one embodiment, the origin of replication allowing replication in C. acnes is pWVO1 (typically of sequence SEQ ID NO: 52). In one embodiment, the origin of replication allowing replication in C. acnes is pAP1 (typically of sequence SEQ ID NO: 53). In one embodiment, the origin of replication allowing replication in C. acnes is pWKS1 (typically of sequence SEQ ID NO: 54). In one embodiment, the origin of replication allowing replication in C. acnes is pLME108 (typically of sequence SEQ ID NO: 55). In one embodiment, the origin of replication allowing replication in C. acnes is pLS1 (typically of sequence SEQ ID NO: 56). In one embodiment, the origin of replication allowing replication in C. acnes is pUB6060 (typically of sequence SEQ ID NO: 57). In one embodiment, the origin of replication allowing replication in C. acnes is p545 (typically of sequence SEQ ID NO: 58). In one embodiment, the origin of replication allowing replication in C. acnes is pJD4 (typically of sequence SEQ ID NO: 59). In one embodiment, the origin of replication allowing replication in C. acnes is pIJ101 (typically of sequence SEQ ID NO: 60). In one embodiment, the origin of replication allowing replication in C. acnes is pSN22 (typically of sequence SEQ ID NO: 61). In one embodiment, the origin of replication allowing replication in C. acnes is pGP01 (typically of sequence SEQ ID NO: 62). In one embodiment, the origin of replication allowing replication in C. acnes is pIP501 (typically of sequence SEQ ID NO: 63). In one embodiment, the origin of replication allowing replication in C. acnes is pCU1 (typically of sequence SEQ ID NO: 64). In one embodiment, the origin of replication allowing replication in C. acnes is pBAV1 K-T5 (typically of sequence SEQ ID NO: 65).

In one embodiment, the DNA vector comprises an origin of replication allowing replication in C. acnes. In one embodiment, the DNA vector comprises an origin of replication selected from the group consisting of: R6K (SEQ ID NO: 42); RK2 (SEQ ID NO: 43); pBBR1 (SEQ ID NO: 44); pRO1600 (SEQ ID NO: 45); RSF1010 (SEQ ID NO: 46); pAM31 (SEQ ID NO: 47); pLME106 (SEQ ID NO: 48); pTZC1 (SEQ ID NO: 49); pBC1 (SEQ ID NO: 50); pEP2 (SEQ ID NO: 51); pWVO1 (SEQ ID NO: 52); pAP1 (SEQ ID NO: 53); pWKS1 (SEQ ID NO: 54); pLME108 (SEQ ID NO: 55); pLS1 (SEQ ID NO: 56); pUB6060 (SEQ ID NO: 57); p545 (SEQ ID NO: 58); pJD4 (SEQ ID NO: 59); pIJ101 (SEQ ID NO: 60); pSN22 (SEQ ID NO: 61); pGP01 (SEQ ID NO: 62); pIP501 (SEQ ID NO: 63); pCU1 (SEQ ID NO: 64); and pBAV1 K-T5 (SEQ ID NO: 65).

Preferably, the origin of replication is of sequence at least 80, 83, 85, 87, 90, 93, 95, 97, 98, 99, or 100% identical to the sequence of any of the above origins of replication.

In various embodiments, the selection marker is selected from ermE, catA, hygB, ermX, tetW, erm(50) and other high GC antibiotic resistance genes. In one embodiment, the selection marker is not ermE. In one embodiment, the selection marker is catA. In one embodiment, the selection marker is hygB.

In one embodiment, the DNA vector further comprises a CRISPR-Cas system. Typically, the CRISPR-Cas system comprises a CRISPR array. Typically, the CRISPR-Cas system comprises a RNA guide (crRNA or sgRNA).

In one embodiment, the CRISPR-Cas system targets a C. acnes chromosome locus. Preferably, the targeted locus is not present in the C. acnes producer cell. Preferably, the CRISPR array from the CRISPR-Cas system is expressing one or several crRNA targeting the chromosome locus.

In one embodiment, the CRISPR-Cas system targets several C. acnes chromosome loci. Preferably, the targeted loci are not present in the C. acnes producer cell. Preferably, the CRISPR array from the CRISPR-Cas system is expressing one or several crRNA targeting the chromosome loci.

In one embodiment, the CRISPR-Cas system targets a C. acnes plasmid locus. Preferably, the targeted locus is not present in the C. acnes producer cell. Preferably, the CRISPR array from the CRISPR-Cas system is expressing one or several crRNA targeting the plasmid locus.

In one embodiment, the CRISPR-Cas system targets several C. acnes plasmid loci. Preferably, the targeted loci are not present in the C. acnes producer cell. Preferably, the CRISPR array from the CRISPR-Cas system is expressing one or several crRNA targeting the plasmid loci.

In one embodiment, the CRISPR-Cas system is not expressed in C. acnes producer cell. Preferably the CRISPR-Cas system is repressed in C. acnes producer cell.

In one embodiment, the CRISPR-Cas system targets a proinflammatory sequence related to a host disease.

In one embodiment, the CRISPR-Cas system targets a proinflammatory sequence related to acne vulgaris.

In one embodiment, the DNA vector comprises a template for homologous recombination and the CRISPR-Cas system is targeting the DNA vector itself.

In one embodiment, the DNA vector comprises a template for homologous recombination in C. acnes phages.

In one embodiment, the DNA vector comprises a template for homologous recombination in C. acnes chromosome.

In one embodiment, the DNA vector comprises a template for homologous recombination in C. acnes endogenous plasmids.

In one embodiment, the DNA vector comprises a template for homologous recombination and a CRISPR-Cas system targeting the DNA vector itself outside of the template region.

In one embodiment, the DNA vector comprises a template for homologous recombination and a CRISPR-Cas system targeting the DNA vector itself outside of the template region wherein the RNA guide (crRNA or sgRNA) from the CRISPR-Cas system is not perfectly matching the DNA target.

In one embodiment, the DNA vector comprises an integrase gene expression cassette and a site specific recombination site allow for the integration of the DNA vector inside the chromosome.

In one embodiment, the DNA vector comprises a base editor gene expression cassette and one or multiple crRNAs or sgRNAs.

In one embodiment, the base editor is used to inactivate the expression of a gene by editing one or several nucleotides involved in transcription or translation of said gene. More specifically the base editor is targeting one or several nucleotides of a promoter, a RBS or a start codon.

In one embodiment, the base editor is used to introduce a premature stop codon.

In one embodiment, the base editor is used to introduce one or several rare codons.

In another embodiment, the base editor is used to modulate the expression of genes by editing one or several nucleotides involved in transcription or translation of said genes. More specifically the base editor is targeting one or several nucleotides of a promoter, a RBS or a start codon, leading to an increase or decrease of gene expression.

In another embodiment, the base editor is used to revert a mutation that leads to the inactivation, decrease or increase in activity of a gene or pathway.

In another embodiment, the base editor is used to revert a mutation that leads to an increase of pathogenicity.

In one embodiment, the base editor is used to modify the regulation of a gene by editing one or several nucleotides involved in its regulation such as nucleotides of operator sequence, transcription factor binding site, riboswitch, RNAse recognition site, protease cleavage site, methylation site or post translational modification site (phosphorylation, glycosylation, acetylation, pupylation . . . ).

In one embodiment, the DNA vector comprises a prime editor gene expression cassette and one or multiple pegRNAs.

In one embodiment, the prime editor is used to introduce one or several premature stop codon.

In one embodiment, the prime editor is used to introduce one or several rare codons.

In one embodiment, the prime editor is used to introduce or delete a nucleotide inducing a frameshift in the reading frame.

In another embodiment, the prime editor is used to modulate the expression of genes by replacing, deleting or inserting one or several nucleotides involved in transcription or translation of said genes. More specifically the prime editor is replacing, deleting or inserting one or several nucleotides in a promoter, a RBS or a start codon. leading to an increase or decrease of gene expression.

In another embodiment, the prime editor is used to revert a mutation that leads to the inactivation or decrease in activity of a gene or pathway.

In another embodiment, the prime editor is used to revert a mutation that leads to an increase of pathogenicity.

In one embodiment, the vector is a plasmid which comprises an E. coli replicon and an E. coli resistance marker allowing extraction of the plasmid from C. acnes and transformation and replication in E. coli.

In one embodiment, the vector is a plasmid which comprises an E. coli replicon and an E. coli resistance marker allowing extraction of the plasmid from E. coli and transformation and replication in C. acnes.

In one embodiment, the vector comprises 2 origins or replication, one allowing replication in C. acnes or C. acnes producer cell only, the second origin of replication allowing replication in another bacteria.

In one embodiment, the vector comprising the template DNA for homologous recombination allows expression of genes increasing recombination rate.

In one embodiment, the template for homologous recombination contains homology arms upstream and downstream of recombination points. These homology arms can be at least 50, 100, 500 or at least 1000 bp in size.

In one embodiment, the gene of interest comprised by the DNA vector can be a transgene that is exogenous to the C. acnes. Transgenes include but are not limited to:
a DNA encoding a fluorescent protein (e.g., UnaG) that leads to fluorescent C. acnes cells once a specific substrate is added;
a DNA encoding an enzymatic reporter (e.g., LacZ) that leads to the production of a chromogenic compound by C. acnes colonies;
a DNA encoding a human protein (e.g., an interleukin);
a DNA encoding an antigen (e.g. a tumor antigen, a viral antigen, a bacterial antigen, a fungal antigen, a self-antigen, an allergen or a graft-specific antigen);
a CRISPR-Cas system;
a prime-editing system; or
a base-editor system.

In a particular embodiment, the gene of interest encoded by the DNA vector is a DNA encoding an antigen, more particularly a DNA encoding an antigen selected from the group consisting of tumor antigens, viral antigens, bacterial antigens, fungal antigens, self-antigens, allergens and graft-specific antigens, as defined below.

C. acnes Strains Comprising DNA Vectors and Engineered C. acnes Strains

The invention encompasses C. acnes comprising any of the DNA vectors of the invention. The invention further encompasses C. acnes produced by any of the methods of the invention. Thus, the invention encompasses C. acnes that have been modified following transduction of any of the DNA vectors of the invention by a phage-derived particle, whether retaining the DNA vector or subsequently having that DNA vector removed (i.e., cured) from C. acnes.

Thus, the invention encompasses C. acnes produced by a method comprising producing a phage-derived particle from a C. acnes producer cell containing a DNA vector of the invention; contacting these phage-derived particles with C. acnes receiver cells leading to transduction of the DNA vector into the C. acnes receiver cell and modification of the C. acnes receiver cell with a gene of interest carried by the vector (e.g., a CRISPR-Cas system) and/or an exogenous gene inserted into the C. acnes chromosome; selecting for the modification; and curing C. acnes of the vector.

The invention encompasses an engineered C. acnes that has been modified by a CRISPR-Cas system transduced by a phage or phage-derived particle carrying a vector of the invention.

The invention encompasses an engineered C. acnes that has been modified by transduction of DNA vector and subsequent insertion of an exogenous gene into the C. acnes chromosome.

The invention encompasses an engineered C. acnes that has been modified by transduction of DNA vector and subsequent deletion or mutation of an endogenous gene into the C. acnes chromosome or C. acnes endogenous plasmid.

The invention encompasses C. acnes produced by transduction of a DNA vector of the invention.

The invention encompasses an engineered C. acnes that has been modified by delivery of a plasmid, in particular by conjugation. In a particular embodiment, said plasmid comprises a CRISPR-Cas system. In another particular embodiment, said plasmid comprises an exogenous gene. In another particular embodiment, said plasmid enables the insertion of an exogenous gene into the C. acnes chromosome. In another particular embodiment, said plasmid enables the deletion or mutation of an endogenous gene into the C. acnes chromosome or C. acnes endogenous plasmid. In a particular embodiment, said plasmid comprises an origin of replication allowing replication in C. acnes, as defined above and/or an origin of transfer as defined above.

Cutibacterium acnes, previously named Propionibacterium acnes, has been historically classified in three major phylotypes based on recA and tly sequencing: IA, IB, II and III. These phylotypes have been further subdivided using different multi-locus sequence typing (MLST) schemes into IA1, IA2, IB, II and III. More recently, Fitz-Gibbon et al (Fitz-Gibbon, S. et al. (2013) J Invest Dermatol 133, 2152-2160) have introduced a new classification based on sequence diversity of 16S rRNA gene (ribotyping) as well as a refined classification of phylotypes: IA-1, IA-2, IB-1, IB-2, IB-3, IC, II, III. The present disclosure refers to this classification but concordance between this classification and others is well-known from the skilled person and can be obtained from the following review (1.Dréno, B. et al. (2018). Journal of the European Academy of Dermatology and Venereology 32, 5-14). In a particular embodiment, C. acnes may thus be from a phylotype selected from the group consisting of phylotypes IA-1, IA-2, IB-1, IB-2, IB-3, IC, II and III.

By comparing whole genome sequences of strains isolated from acne and healthy volunteers, Fitz-Gibbon and colleagues could identify acne-associated strains (IA-2 and IB-1) and healthy-associated strains (II) in accordance with previous studies. More interestingly, they found specific loci (locus1, locus 2 and locus 3) present in acne associated strains and absent of neutral and healthy strains. Similar loci were found in a subsequent metagenomic analysis confirming the association between the presence of these loci and acne vulgaris (Barnard, E. et al. (2016) Scientific Reports 6, srep39491).

The ability of specific strain phylotypes to induce immune response has been recently investigated (Yu et al. (2016) Journal of Investigative Dermatology 136:2221-2228). Yu et al. demonstrated that the different C. acnes phylotypes induced different cytokine profiles when incubated with peripheral blood mononuclear cells (PBMC). More particularly, they showed that acne-associated phylotypes IA-2 p+ (i.e. with a large plasmid associated with acne), IB-1, and IC induced high levels of inflammatory IFN-γ and IL-17 but low levels of IL-10, suggesting that these specific phylotypes could induce both Th1 and Th17 responses. They also showed that phylotypes IB-3, II and III induced lower levels of IL-17 (and of IFN-γ for phylotype III) but higher levels of IL-10, suggesting induction of Treg responses. They further showed that phylotypes IA-1, IA-2 p- (i.e. without the large plasmid associated with acne) and IB-2 induced lower levels of IFN-γ and IL-10 and higher levels of IL-17, suggesting induction of mainly Th17 responses.

Therefore, depending on the particular immune response that is desired when using the engineered C. acnes of the invention for a particular indication, the use of a given C. acnes phylotype or strain may be advantageous. Accordingly, in a particular embodiment, C. acnes is from a phylotype selected from the group consisting of phylotypes IA-2 p+, IB-1 and IC. In another embodiment, C. acnes is from a phylotype selected from the group consisting of phylotypes IA-1, IA-2 p- and IB-2. In still another embodiment, C. acnes is from a phylotype selected from the group consisting of phylotypes IB-3, II and III.

Furthermore, a previous study showed that it was possible, in S. epidermidis, to induce different T cell responses with different strains within the same species by engineering said strains (Chen et al. (2019) “Decoding commensal-host communication through genetic engineering of Staphylococcus epidermidis” bioRxiv https://doi.org/10.1101/664656).

Therefore, in a particular embodiment, the C. acnes is a strain inducing, or engineered to induce, a given T cell response. In a particular embodiment, more particularly when the C. acnes cell is intended to be used in the prevention and/or treatment of cancer, said C. acnes is a strain inducing, or engineered to induce, increased levels of IFN-γ and/or IL-17a. In a particular embodiment, more particularly when the C. acnes cell is intended to be used in the prevention and/or treatment of an infection, said C. acnes is a strain inducing, or engineered to induce, increased levels of IFN-γ and/or IL-17. In a particular embodiment, more particularly when the C. acnes cell is intended to be used in the prevention and/or treatment of an autoimmune disease, said C. acnes is a strain inducing, or engineered to induce, increased levels of IL-10. In a particular embodiment, more particularly when the C. acnes cell is intended to be used in the prevention and/or treatment of an allergy, such as asthma, said C. acnes is a strain inducing, or engineered to induce, increased levels of IFN-g and/or IL-10. In a particular embodiment, more particularly when the C. acnes cell is intended to be used in the prevention and/or treatment of a graft rejection, said C. acnes is a strain inducing, or engineered to induce, increased levels of IL-10.

C. acnes comprising a recombinant self-replicative DNA vector of the invention (or comprising a plasmid, in particular a conjugative plasmid as defined above) can be generated for the expression of molecules of interest and modulation of C. acnes-host interaction. The molecule of interest can be carried on a self-replicative DNA vector in the C. acnes (or on a plasmid, in particular a conjugative plasmid) or can be inserted into the chromosome of the C. acnes through the action of the self-replicative DNA vector (or of the plasmid, in particular the conjugative plasmid, as defined above).

In one embodiment, the DNA vector is used for C. acnes chromosome engineering.

In one embodiment, the DNA vector is used for C. acnes plasmid engineering.

In one embodiment, the DNA vector is used for C. acnes phage engineering.

In one embodiment, the DNA vector (or the plasmid, in particular the conjugative plasmid, as defined above) is used for the expression of molecules of interest and modulation of C. acnes-host interaction. In one embodiment, the DNA vector (or the plasmid, in particular the conjugative plasmid, as defined above) is used for the expression of transgenes in C. acnes. A transgene can be cloned into the recombinant autonomously-replicating DNA vector (or in the plasmid, in particular the conjugative plasmid) under the control of a given promoter (constitutive or inducible) and followed by a given terminator. The transfer of this vector into C. acnes allows the expression of the transgene. The transgene can be, for example, a CRISPR/Cas system or can encode a human protein, such as an interleukin. In one embodiment the DNA vector (or the plasmid, in particular the conjugative plasmid) encodes several transgenes under the control of a single promoter or under the control of different promoters. The promoters can be endogenous or exogenous, inducible or constitutive.

In one embodiment, the DNA vector (or the plasmid, in particular the conjugative plasmid) is used for the modification of C. acnes genome. In one embodiment, the transfer of the vector (or of the plasmid, in particular the conjugative plasmid) into C. acnes allows the expression of a CRISPR/Cas system that cleaves the C. acnes genome (plasmid or chromosome) at a specific site, leading to modification of the C. acnes genome. In one embodiment, the vector (or the plasmid, in particular the conjugative plasmid) further comprises a gene of interest and homology with the site of cleavage to facilitate integration of the gene of interest into the C. acnes genome.

Delivery of DNA Vectors into C. acnes Strains

In one embodiment, delivery of any DNA vector of the invention into C. acnes producer cell is performed by contacting C. acnes with any DNA vector of the invention.

In one embodiment, delivery of any DNA vector of the invention into C. acnes producer cell is performed by transfection (e.g., electroporation) into C. acnes cells, where it stably replicates. In one embodiment the DNA vector transfected is purified from dam(−) E. coli cells such as ET12567 and electroporated into C. acnes cells made competent at 24° C.

In one embodiment, delivery of any DNA vector of the invention into C. acnes producer cell is performed by transfection (e.g., electroporation) into C. acnes protoplasts. In one embodiment C. acnes protoplasts are generated using Mutanolysin treatment or Lysozyme treatment, Mutanolysin and Lysozyme treatment, or Mutanolysin and Lysozyme and bead-beating treatment followed by resuspension into hypotonique media.

In one embodiment, delivery of any DNA vector of the invention into C. acnes producer cell is performed by mixing C. acnes protoplasts with the DNA vector. In one embodiment glass beads are added with the DNA vector and bead beating is performed to introduce the DNA into C. acnes protoplasts.

In one embodiment, delivery of DNA vectors of the invention into C. acnes is by transduction. In one embodiment, the DNA vector comprises one packaging signal of a C. acnes phage selected from the group consisting of the phages: PAC7 (typically of sequence SEQ ID NO: 68); PAC1 (typically of sequence SEQ ID NO: 69); PAC9 (typically of sequence SEQ ID NO: 70); PAC2 (typically of sequence SEQ ID NO: 71); PAC10 (typically of sequence SEQ ID NO: 72); PAC22 (typically of sequence SEQ ID NO: 73); PAC13 (typically of sequence SEQ ID NO: 74) and PAC263 (typically of sequence SEQ ID NO: 75) and is packaged into proteins expressed from the genome of a C. acnes phage selected from the group consisting of the phages PAC7 (typically of sequence SEQ ID NO: 68); PAC1 (typically of sequence SEQ ID NO: 69); PAC9 (typically of sequence SEQ ID NO: 70); PAC2 (typically of sequence SEQ ID NO: 71); PAC10 (typically of sequence SEQ ID NO: 72); PAC22 (typically of sequence SEQ ID NO: 73); PAC13 (typically of sequence SEQ ID NO: 74) and PAC263 (typically of sequence SEQ ID NO: 75), allowing transduction of the DNA vector into C. acnes.

In one embodiment, delivery of any DNA vector of the invention into C. acnes producer cell is by conjugation. In one embodiment, the DNA vector comprises an origin of transfer. In one embodiment, a donor bacterium, such as E. coli, is used to efficiently transfer the DNA vector into C. acnes recipient cells, where it stably replicates. In one embodiment, the DNA vector comprises an origin of transfer selected from the group consisting of: oriT_pMRC01 (typically of sequence SEQ ID NO: 1); oriT_RSF1010 (typically of sequence SEQ ID NO: 2); oriT_pRS01 (typically of sequence SEQ ID NO: 3); oriT_pMV158 (typically of sequence SEQ ID NO: 4); oriT_pTF1 (typically of sequence SEQ ID NO: 5); oriT_pSC101 (typically of sequence SEQ ID NO: 6); oriT_pBTK445 (typically of sequence SEQ ID NO: 7); oriT_pBBR1 (typically of sequence SEQ ID NO: 8); oriT_R721 (typically of sequence SEQ ID NO: 9); oriT_pRmeGR4a (typically of sequence SEQ ID NO: 10); oriT_ColE1 (typically of sequence SEQ ID NO: 11); oriT_pTiC58 (typically of sequence SEQ ID NO: 12); oriT_pMdT1 (typically of sequence SEQ ID NO: 13); oriT_R1 (typically of sequence SEQ ID NO: 14); oriT_Tn5520 (typically of sequence SEQ ID NO: 15); oriT_QKH54 (typically of sequence SEQ ID NO: 16); oriT_R64 (typically of sequence SEQ ID NO: 17); oriT_R751 (typically of sequence SEQ ID NO: 18); oriT_RP4 (typically of sequence SEQ ID NO: 19); oriT_pKL1 (typically of sequence SEQ ID NO: 20); oriT_RK2 (typically of sequence SEQ ID NO: 21); oriT_R1162 (typically of sequence SEQ ID NO: 22); oriT_Tn4555 (typically of sequence SEQ ID NO: 23); oriT_pHT (typically of sequence SEQ ID NO: 24); oriT_Tn4399 (typically of sequence SEQ ID NO: 25); oriT_Tn916 (typically of sequence SEQ ID NO: 26); oriT_pST12 (typically of sequence SEQ ID NO: 27); oriT_pCU1 (typically of sequence SEQ ID NO: 28); oriT_pSU233 (typically of sequence SEQ ID NO: 29); oriT_F (typically of sequence SEQ ID NO: 30); oriT_pMAB01 (typically of sequence SEQ ID NO: 31); oriT_R388 (typically of sequence SEQ ID NO: 32); oriT_pS7a (typically of sequence SEQ ID NO: 33); oriT_pS7b (typically of sequence SEQ ID NO: 34); oriT_R702 (typically of sequence SEQ ID NO: 35); oriT_pMUR274 (typically of sequence SEQ ID NO: 36); oriT_R100 (typically of sequence SEQ ID NO: 37); oriT_pVCR94deltaX (typically of sequence SEQ ID NO: 38); oriT_R46 (typically of sequence SEQ ID NO: 39); oriT_pGO1 (typically of sequence SEQ ID NO: 40) and oriT_pIP501 (typically of sequence SEQ ID NO: 41). In one embodiment, a donor bacterium, such as E. coli, carries a conjugative plasmid, a conjugative transposon or an integrative and conjugative element (ICE) selected from the group consisting of: pMRC01; RSF1010; pRS01; pMV158; pTF1; pSC101; pBTK445; pBBR1; R721; pRmeGR4a; ColE1; pTiC58; pMdT1; R1; Tn5520; QKH54; R64; R751; RP4; pKL1; RK2; R1162; Tn4555; pHT; Tn4399; Tn916; pST12; pCU1; pSU233; F; pMAB01; R388; pS7a; pS7b; R702; pMUR274; R100; pVCR94deltaX; R46; pGO1 and pIP501, and is used to efficiently transfer the DNA vector into C. acnes recipient cells. In one embodiment the DNA vector contains an origin of transfer and the associated relaxase of a conjugative plasmid, conjugative transposon or integrative and conjugative element (ICE) selected from the group consisting of: pMRC01; RSF1010; pRS01; pMV158; pTF1; pSC101; pBTK445; pBBR1; R721; pRmeGR4a; ColE1; pTiC58; pMdT1; R1; Tn5520; QKH54; R64; R751; RP4; pKL1; RK2; R1162; Tn4555; pHT; Tn4399; Tn916; pST12; pCU1; pSU233; F; pMAB01; R388; pS7a; pS7b; R702; pMUR274; R100; pVCR94deltaX; R46; pGO1 and pIP501.

In a preferred embodiment the DNA vector comprises the origin of transfer oriT_pMRC01 (typically of sequence SEQ ID NO: 1). In a preferred embodiment, the DNA vector comprises the origin of transfer oriT_RSF1010 (typically of sequence SEQ ID NO: 2). In a preferred embodiment, the DNA vector comprises the origin of transfer oriT_pRS01 (typically of sequence SEQ ID NO: 3). In a preferred embodiment, the DNA vector comprises the origin of transferoriT_pMV158 (typically of sequence SEQ ID NO: 4). In a preferred embodiment, the DNA vector comprises the origin of transfer oriT_pTF1 (typically of sequence SEQ ID NO: 5). In a preferred embodiment, the DNA vector comprises the origin of transfer oriT_pSC101 (typically of sequence SEQ ID NO: 6). In a preferred embodiment, the DNA vector comprises the origin of transfer oriT_pBTK445 (typically of sequence SEQ ID NO: 7). In a preferred embodiment, the DNA vector comprises the origin of transfer oriT_pBBR1 (typically of sequence SEQ ID NO: 8). In a preferred embodiment, the DNA vector comprises the origin of transfer oriT_R721 (typically of sequence SEQ ID NO: 9). In a preferred embodiment, the DNA vector comprises the origin of transfer oriT_pRmeGR4a (typically of sequence SEQ ID NO: 10). In a preferred embodiment, the DNA vector comprises the origin of transfer oriT_ColE1 (typically of sequence SEQ ID NO: 11). In a preferred embodiment, the DNA vector comprises the origin of transfer oriT_pTiC58 (typically of sequence SEQ ID NO: 12). In a preferred embodiment, the DNA vector comprises the origin of transfer oriT_pMdT1 (typically of sequence SEQ ID NO: 13). In a preferred embodiment, the DNA vector comprises the origin of transfer oriT_R1 (typically of sequence SEQ ID NO: 14). In a preferred embodiment, the DNA vector comprises the origin of transfer oriT_Tn5520 (typically of sequence SEQ ID NO: 15). In a preferred embodiment, the DNA vector comprises the origin of transfer oriT_QKH54 (typically of sequence SEQ ID NO: 16). In a preferred embodiment, the DNA vector comprises the origin of transfer oriT_R64 (typically of sequence SEQ ID NO: 17). In a preferred embodiment, the DNA vector comprises the origin of transfer oriT_R751 (typically of sequence SEQ ID NO: 18). In a preferred embodiment, the DNA vector comprises the origin of transfer oriT_RP4 (typically of sequence SEQ ID NO: 19). In a preferred embodiment, the DNA vector comprises the origin of transfer oriT_pKL1 (typically of sequence SEQ ID NO: 20). In a preferred embodiment, the DNA vector comprises the origin of transfer oriT_RK2 (typically of sequence SEQ ID NO: 21). In a preferred embodiment, the DNA vector comprises the origin of transfer oriT_R1162 (typically of sequence SEQ ID NO: 22). In a preferred embodiment, the DNA vector comprises the origin of transfer oriT_Tn4555 (typically of sequence SEQ ID NO: 23). In a preferred embodiment, the DNA vector comprises the origin of transfer oriT_pHT (typically of sequence SEQ ID NO: 24). In a preferred embodiment, the DNA vector comprises the origin of transfer oriT_Tn4399 (typically of sequence SEQ ID NO: 25). In a preferred embodiment, the DNA vector comprises the origin of transfer oriT_Tn916 (typically of sequence SEQ ID NO: 26). In a preferred embodiment, the DNA vector comprises the origin of transfer oriT_pST12 (typically of sequence SEQ ID NO: 27). In a preferred embodiment, the DNA vector comprises the origin of transfer oriT_pCU1 (typically of sequence SEQ ID NO: 28). In a preferred embodiment, the DNA vector comprises the origin of transfer oriT_pSU233 (typically of sequence SEQ ID NO: 29). In a preferred embodiment, the DNA vector comprises the origin of transfer oriT_F (typically of sequence SEQ ID NO: 30). In a preferred embodiment, the DNA vector comprises the origin of transfer oriT_pMAB01 (typically of sequence SEQ ID NO: 31). In a preferred embodiment, the DNA vector comprises the origin of transfer oriT_R388 (typically of sequence SEQ ID NO: 32). In a preferred embodiment, the DNA vector comprises the origin of transfer oriT_pS7a (typically of sequence SEQ ID NO: 33). In a preferred embodiment, the DNA vector comprises the origin of transfer oriT_pS7b (typically of sequence SEQ ID NO: 34). In a preferred embodiment, the DNA vector comprises the origin of transfer oriT_R702 (typically of sequence SEQ ID NO: 35). In a preferred embodiment, the DNA vector comprises the origin of transfer oriT_pMUR274 (typically of sequence SEQ ID NO: 36). In a preferred embodiment, the DNA vector comprises the origin of transfer oriT_R100 (typically of sequence SEQ ID NO: 37). In a preferred embodiment, the DNA vector comprises the origin of transfer oriT_pVCR94deltaX (typically of sequence SEQ ID NO: 38). In a preferred embodiment, the DNA vector comprises the origin of transfer oriT_R46 (typically of sequence SEQ ID NO: 39). In a preferred embodiment, the DNA vector comprises the origin of transfer oriT_pGO1 (typically of sequence SEQ ID NO: 40). In a preferred embodiment, the DNA vector comprises the origin of transfer oriT_pIP501 (typically of sequence SEQ ID NO: 41).

In one embodiment, a donor bacterium is selected from the group consisting of: Escherichia coli, Pseudomonas aeruginosa, Lactococcus lactis, Lactobacillus casei, Lactobacillus fermentum, Lactobacillus rhamnosus, Propionibacterium freudenreichii, Lactobacillus brevis, Staphylococcus epidermidis, Staphylococcus aureus, Cutibacterium granulosum, Cutibacterium humerusii, Enterococcus faecalis and Bacillus subtilis, carrying a conjugative plasmid, a conjugative transposon or an integrative and conjugative element (ICE).

In one embodiment the conjugation is performed growing at high density the donor bacteria, such as E. coli, harboring the mobilizable DNA vector and the conjugative machinery (ICE, plasmid, conjugative transposon). Donor cells are pelleted by centrifugation, and washed to remove antibiotics added during growth to maintain mobilizable and conjugative DNA vectors. Donor cells are then mixed in presence of C. acnes cells. The mixture donor cells—C. acnes is spotted onto Brucella agar plates and allowed to mate at 37° C. under anaerobic conditions. After mating, cells are harvested from the mating plate, re-suspended in BHI broth and plated onto Brucella agar plates that are supplemented with:
a compound killing donor cells but not C. acnes, or
an antibiotic selecting the mobilizable DNA vector.

After several days of incubation, C. acnes colonies are streaked on Brucella agar plates supplemented with the appropriate selection and the presence of the conjugated plasmid is confirmed via specific PCRs. The identity of C. acnes as well as the absence of donor cells is also confirmed by PCR analyses.

In one embodiment the conjugation is performed according to the following protocol: 2 mL of overnight cultures of E. coli donor cells harboring a mobilizable DNA vector and a conjugative machinery (ICE, plasmid, conjugative transposon) is grown in LB broth and pelleted at 6,000×g for 1 min. Supernatants are discarded and pellets are washed with 500 μL of pre-sterilized LB medium, centrifuged again using the same conditions. Pellet is then re-suspended in 200 μL of exponentially growing (OD600=0.5) C. acnes receptor BHI culture concentrated 10×. The mixture E. coli-C. acnes is spotted (50 μL/spot) onto Brucella agar plates and allowed to mate at 37° C. under anaerobic conditions for 24 hours. After that time, cells are harvested from the mating plate, re-suspended in 300 μL of BHI broth and plated onto Brucella agar plates that had been supplemented with 50 μg/mL polymyxin B and 5 μg/mL erythromycin or 3.5 μg/mL chloramphenicol depending on the selection marker present in the mobilizable DNA vector. After 7 days, C. acnes cells that grow in the presence of selection are streaked on Brucella agar plates supplemented with the appropriate selection and the presence of the conjugated plasmid confirmed via specific PCRs. The identity of C. acnes as well as the absence of E. coli donor strain are also confirmed by PCR analyses.

Methods to Modify Endogenous C. acnes Plasmids

Naturally occurring C. acnes plasmids have been described^{21,22} and some of them are able to be transferred from one C. acnes to another by conjugation²⁰. Being able to modify such plasmids is of interest to study their effect notably their pro-inflammatory role in acne vulgaris or to use them for further genetic manipulation of C. acnes. The inventors have developed methods to modify C. acnes plasmids.

In one embodiment, the method comprises, in a first step, introducing into C. acnes a replicative vector comprising:
a selection marker for C. acnes as defined above,
an origin of replication for C. acnes as defined above,
a phage packaging signal as defined above, and
a template for homologous recombination in the C. acnes endogenous plasmid.

In one embodiment, the method comprises, in a first step, introducing in C. acnes a replicative vector comprising:
a selection marker for C. acnes as defined above,
an origin of replication for C. acnes as defined above,
a phage packaging signal as defined above, and
a CRISPR-Cas system
a template for homologous recombination with the C. acnes endogenous plasmid.

Introduction can be achieved with electroporation, electroporation of protoplast, conjugation, chemical transformation or transduction. C. acnes recombinants are then preferably grown in presence of an antibiotic.

Recombinants are then typically infected with C. acnes phage to produce phage-derived particles carrying the DNA vectors.

Phage-derived particles are then typically mixed with C. acnes receiver cells containing an endogenous plasmid such as pIMPLE-HL096PA1. C. acnes transductants are then typically selected on the appropriate antibiotic.

In a second step, C. acnes transductants are grown in the presence of an antibiotic A to a high density to increase chances of a homologous recombination event occurring. Homologous recombination typically leads to introduction of a selection marker, giving resistance to an antibiotic B. In the dense culture, C. acnes strains carrying wild-type endogenous plasmid and recombinant endogenous plasmid carrying a resistance marker are typically present. The high-density culture is then preferably washed and typically put in the presence of a receiver C. acnes strain that is resistant to a third antibiotic C. Selection of transconjugant with antibiotics C and B typically leads to selection of receiver cells with the recombinant plasmid.

Other modifications enabled by the methods of the invention include the insertion of an E. coli replicon and an E. coli resistant marker on the plasmid allowing extraction of the plasmid from C. acnes and transformation and replication in E. coli.

Additionally, the plasmid carrying the template DNA for homologous recombination preferably allows the expression of genes that increase recombination rate.

The template for homologous recombination typically contains homology arms upstream and downstream recombination points. These homology arms are preferably 50, 100, 500, 1000 bp long or more.

C. acnes Genome Engineering and Engineered C. acnes Strains

The invention encompasses methods of C. acnes genome engineering and engineered C. acnes strains that have been engineered by any of the methods of the invention. An “engineered strain” is a strain that has been obtained by any of the methods of the invention to contain an alteration either found or not found in nature. For example, the engineered C. acnes strain can comprise any of the vectors or DNAs of the invention.

The invention encompasses methods for delivering DNA of interest into C. acnes strains by conjugation. The invention also encompasses methods for delivering DNA of interest into C. acnes strains via phage-derived particles. The invention encompasses methods to engineer the C. acnes chromosome with replicative and non-replicative vector methods.

In one embodiment, delivery of the DNA vector into C. acnes is by transduction. In one embodiment, the DNA vector comprises a phage packaging signal (cos) originating from C. acnes phages. In one embodiment, phage-derived particles containing the DNA vector can be generated and allow the DNA vector to be transduced into C. acnes cells.

In one embodiment, the invention encompasses replicative and non-replicative vector methods using a vector comprising at least a recombination template with one or two homology arms.

To engineer the C. acnes genome, the inventors have developed methods using replicative and non-replicative vectors.

### Non-Replicative Vector Methods

In one embodiment, non replicative vector methods use a vector comprising at least:
a phage packaging signal, as defined above;
a selection marker for C. acnes, as defined above;
a recombination template with one or two homology arms;
an origin of replication allowing replication only in Cutibacterium acnes producer cell; and
optionally a counter selection marker such as SacB.

Non replicative vector methods use vectors that carry a C. acnes replicon that replicate only in a C. acnes producer cell but not in other C. acnes cells. Thus, such vectors are able to replicate in C. acnes producer cell, get packaged into phage capsid upon contacting with phage genome leading to a phage-derived particle, and get transduced by the phage-derived particle into C. acnes receiver cell where they do not replicate.

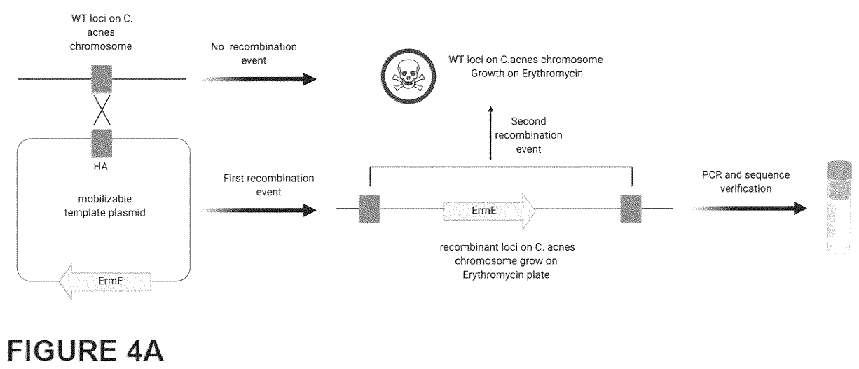

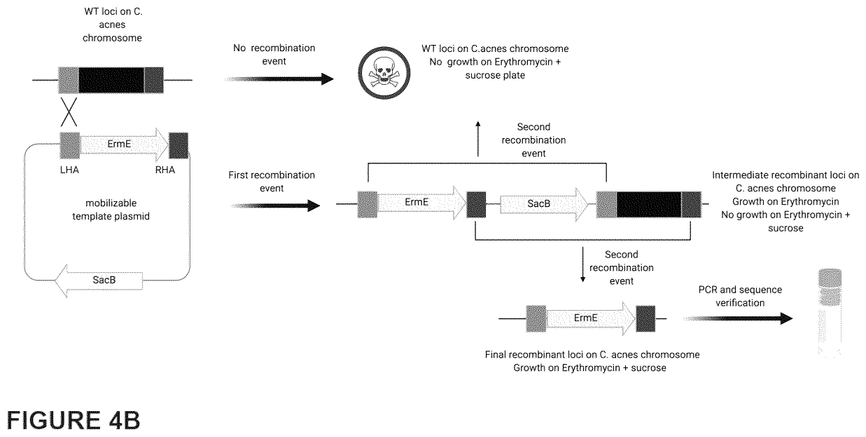

The methods comprise introducing into a C. acnes producer cell a plasmid containing a template for homologous DNA recombination inside the genome. The template can contain one (FIG. 4A) or two homologous regions (FIG. 4B) leading to homologous recombination.

In one embodiment, the method comprises a C. acnes producer cell, carrying a plasmid containing a template for homologous DNA recombination inside the chromosome where the homologous DNA is not present in the C. acnes producer cell, a phage packaging signal (cos) originating from C. acnes phages, a selection marker for C. acnes, as defined above, and an origin of replication for C. acnes producer cell but not replicating in C. acnes receiver cell. The template can contain one (FIG. 4A) or two homologous regions (FIG. 4B), leading to homologous recombination. The producer cell is typically infected by a C. acnes phage leading to production of phage-derived particles containing the DNA vector with homology arm(s). Phage-derived particles are preferably mixed with C. acnes receiver cells (e.g., ATCC 11828). Transductants can be selected on antibiotic plates, streaked on antibiotic plates and plasmid integration screened by PCR. Because the plasmid is not replicative in C. acnes, only recombinant cells that stably maintain the antibiotic marker are able to grow on antibiotic plates.

In the case where there are two homology arms present on the template DNA, a first recombination event (also called cross-over) typically leads to the full integration of the plasmid. This is typically followed by a second recombination event that removes the plasmid backbone and leads to either the modification of the chromosome or to the reconstitution of wild-type wt locus.

In one embodiment, the C. acnes producer cell carries a vector containing a left homology arm (LHA) and a right homology arm (RHA) flanking a C. acnes selection marker, for example, ermE (pEB_HR02). The two homology arms typically do not match the C. acnes producer cell chromosome. In one embodiment, the vector also contains a phage packaging signal (cos) originating from C. acnes phages, a selection marker for C. acnes and an origin of replication for C. acnes producer cell but not replicating in C. acnes receiver cell. In one embodiment the DNA vector also contains a C. acnes counter-selection marker, such as sacB, on the plasmid backbone allowing selection of the second recombination event. The C. acnes producer cell carrying pEB_HR02 is typically infected by a phage leading to production of phage-derived particles comprising pEB_HR02. The phage-derived particles are typically put in presence of C. acnes receiver cells (e.g., ATCC 11828). Transductants are typically selected on plates supplemented with the antibiotic (e.g., erythromycin), streaked onto plates supplemented with the antibiotic (e.g., erythromycin) and integration of the plasmid confirmed by PCR. Because the plasmid is not replicative in C. acnes receiver cell, C. acnes clones able to grow in the presence of the antibiotic (e.g., erythromycin), have undergone a single homologous recombination event, which has led to the integration of the full plasmid. To select for final recombinant loci, cells are typically exposed to the counter-selection (e.g., sucrose) and the antibiotic (e.g., erythromycin), which leads to cell death due to sacB activity (the full plasmid remains integrated in the chromosome). Survivors are typically screened by PCR for successful final recombinant loci presence. In one embodiment the DNA vector contains only one homology arm (pEB_HR01). In one embodiment, both pEB_HR01 and pEB_HR02 phage-derived particles are applied on the skin and no antibiotic selection is applied.

In one embodiment, the C. acnes producer cell carries a plasmid (vector) containing a left homology arm (LHA) and a right homology arm (RHA) flanking C. acnes selection marker ErmE (pEB_HR02). The vector also preferably contains an E. coli origin of replication, an E. coli selection marker, an oriT and relaxase from a conjugative plasmid and a C. acnes counter-selection marker, such as sacB. pEB_HR02 can be transformed into an E. coli donor strain (e.g. Ec0s2862). Transformants are typically selected, grown and mixed with C. acnes receiver cells (e.g., ATCC 11828). Transconjugants are typically selected on plates supplemented with the antibiotic (e.g., erythromycin), streaked onto plates supplemented with the antibiotic (e.g., erythromycin) and integration of the plasmid confirmed by PCR. Because the plasmid is not replicative in C. acnes receiver cell, C. acnes clones able to grow in the presence of the antibiotic (e.g., erythromycin), have undergone a single homologous recombination event, which has led to the integration of the full plasmid. To select for final recombinant loci, cells are typically exposed to the counter-selection (e.g., sucrose) and the antibiotic (e.g., erythromycin), which leads to cell death due to sacB activity (the full plasmid remains integrated in the chromosome). Survivors are typically screened by PCR for successful final recombinant loci presence

### Replicative CRISPR-Cas System Selection Vector Methods

The invention encompasses replicative vectors comprising an origin of replication for C. acnes.

In one embodiment, a replicative CRISPR-Cas selection vector method uses vector with at least:
a phage packaging signal (cos) originating from C. acnes phages, as defined above;
a selection marker for C. acnes, as defined above;
an origin of replication for C. acnes;
a recombination template with two homology arms; and
a CRISPR-Cas system for expression in C. acnes.

In one embodiment, a replicative CRISPR-Cas selection vector method uses a vector with at least:
a selection marker for E. coli, as defined above;
an origin of replication for E. coli;
a selection marker for C. acnes, as defined above;
a recombination template with two homology arms;
an origin of replication for C. acnes; and
a CRISPR-Cas system that is expressed in C. acnes.

Thus, such vectors are able to replicate in E. coli and are able to replicate in C. acnes. They also carry a CRISPR-Cas system able to induce double stranded breaks at the wild-type loci where recombination is wanted, leading to death of C. acnes receiver cell.

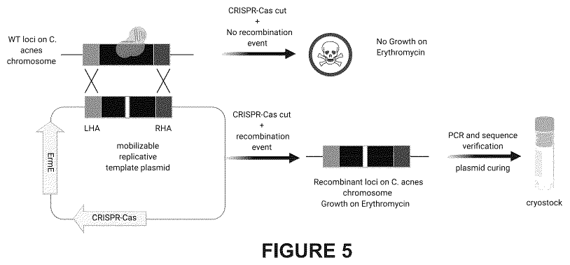

In one embodiment, the method comprises the use of a C. acnes producer cell, for example strain ATCC 6919, carrying a replicative CRISPR-Cas system selection vector containing a template for homologous DNA recombination inside the chromosome and a phage packaging signal (cos) originating from C. acnes phages. In one embodiment, the template contains two homologous regions (FIG. 5), leading to homologous recombination. The producer cell preferably does not contain the wild-type loci targeted by the CRISPR-Cas system. The C. acnes producer cell is typically infected by a C. acnes phage leading to production of phage-derived particles carrying the DNA vector. Phage-derived particles are typically put in contact with C. acnes receiver cell. After transduction into C. acnes receiver cell, cells that have recombined with the DNA template vector are not targeted by the CRISPR-Cas system because, for example, they do not have the associated PAM sequence anymore. Plating on antibiotic-containing media, e.g., erythromycin plates, typically ensures that the cells that survive have been transduced and still carry the DNA vector (e.g. plasmid) expressing the CRISPR-Cas system. Single colonies are typically streaked on antibiotic-containing media, e.g., erythromycin plates, and recombinant loci are typically confirmed by PCR and sequencing.

In one embodiment, a step of plasmid curing is performed to eliminate the plasmid.

In one embodiment, the C. acnes producer cell contains the DNA target of the CRISPR-Cas system but the CRISPR-Cas system is not expressed in the C. acnes producer cell but is expressed in C. acnes receiver cell. More preferably the CRISPR-Cas system is repressed in the C. acnes producer cell but not in C. acnes receiver cell.

Such methods can be used for scarless editing such as substitution, deletion or insertion because there is no need to introduce a selection marker to select for recombinants, the selection being done by CRISPR-Cas killing.

### Self-Targeted Replicative Vector Methods

In one embodiment, the invention encompasses self-targeted replicative vector methods. In one embodiment, the invention encompasses the use of a CRISPR-Cas system to program cutting of the DNA vector (e.g. plasmid) in one or several target sequences, leading to linearization of the recombination template that have been shown to increase recombination efficiency⁹. To be able to clone a self-targeting vector, an inducible CRISPR-Cas system can be used, for example, using an inducible promoter upstream of the gene encoding the Cas nuclease. By combining this inducible promoter with a riboswitch, even tighter inhibition of CRISPR-Cas system expression can be assured. Another strategy to generate self-targeting CRISPR-Cas system relies on promoters that are repressed in the C. acnes producer cell and not in C. acnes receiver cell. In this way, the CRISPR-Cas system will only be active once transduced in a C. acnes receiver cell.

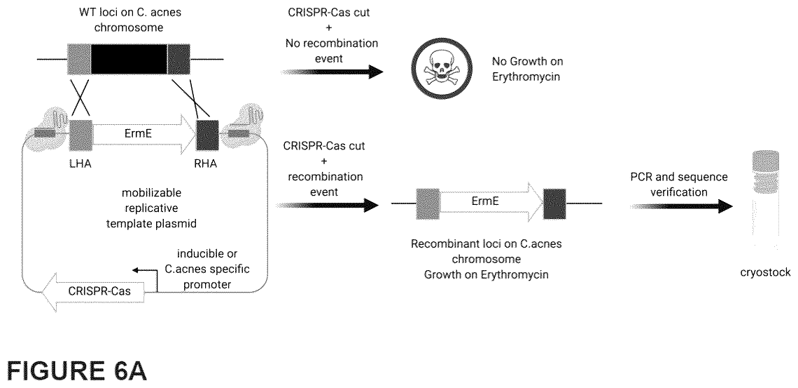

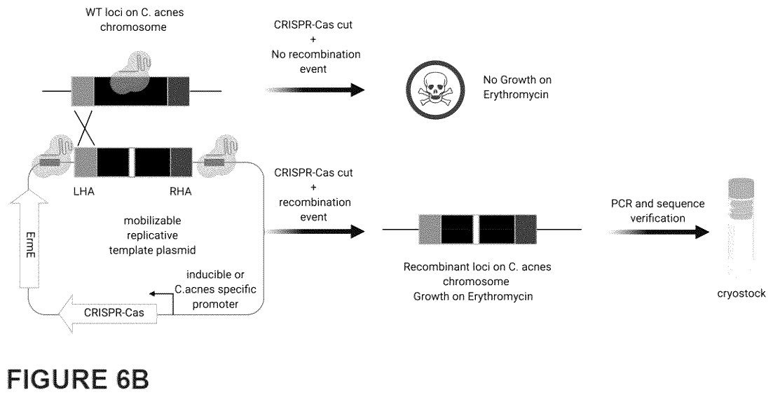

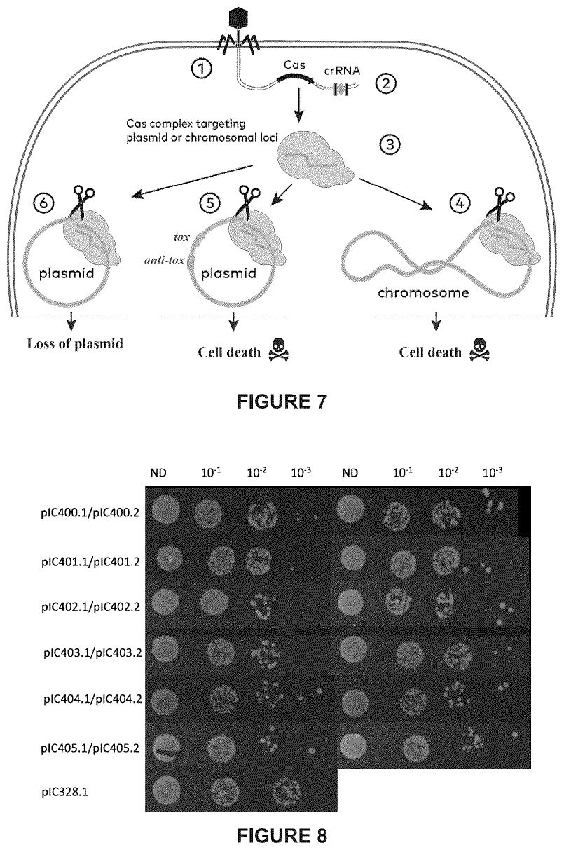

Using such strategy, for example, a gene replacement can be performed using an antibiotic marker flanked by homology arms (FIG. 6A) or by performing scarless recombination using the CRISPR-Cas system ability to kill the bacteria when targeting C. acnes chromosome (FIG. 6B).

After introduction and selection of the DNA vector (e.g. plasmid), a homologous event typically takes place leading to removal of a PAM sequence.

Additionally, the DNA vector (e.g. plasmid) carrying the template DNA for homologous recombination typically allows expression of genes increasing recombination rate.

In one embodiment, the DNA vector comprises a template for homologous recombination and a CRISPR-Cas system targeting the DNA vector itself outside of the template region wherein the RNA guide (crRNA or sgRNA) from the CRISPR-Cas system is not perfectly matching the DNA target.

In one embodiment, the invention encompasses replicative vector methods using a vector with at least:
a phage packaging signal (cos) originating from C. acnes phages, as defined above;
a selection marker for C. acnes, as defined above;
an origin of replication for C. acnes, as defined above; and
a CRISPR-Cas system for expression in C. acnes.

In one embodiment, vectors carry a CRISPR-Cas system able to induce double stranded break leading to death of most C. acnes receiver cells except C. acnes receiver cells that by spontaneous mutation or recombination do not carry anymore the CRISPR-Cas system target sequence.

Expression of Proteins by Engineered C. acnes Strains

The invention encompasses the expression of proteins by engineered C. acnes strains. By incorporating an expression cassette into the DNA vector, the protein can be expressed by the transduced C. acnes. The promoter within the expression cassette can be inducible or constitutive, allowing inducible or constitutive expression of proteins by engineered C. acnes strains. Expression of several proteins can be performed as single transcriptional unit (operon) or as separated transcriptional units. In a particular embodiment, said protein is an antigen, such as a tumor antigen, a viral antigen, a bacterial antigen, a fungal antigen, a self-antigen, an allergen or a graft-specific antigen, as defined below.

C. acnes Phage

The invention encompasses the C. acnes phage and related engineered phages, methods for producing these phages, and methods for using these phages to transduce C. acnes.

Phage-Derived Particles in C. acnes

The invention encompasses phage-derived particles comprising any DNA vector of the invention and the methods for the production of these phage-derived particles.

In one embodiment a C. acnes strain carrying any DNA vector of the invention is contacted with a C. acnes phage leading to introduction of the phage genome into the C. acnes strain and the expression of the phage proteins necessary for the assembly of a phage capsid and the packaging of the DNA vector inside the phage capsid.

In one embodiment a C. acnes strain carrying any DNA vector comprising: a selection marker for C. acnes as defined above, a C. acnes phage packaging signal (cos site) as defined above, and an origin of replication for C. acnes as defined above, is contacted with a C. acnes phage leading to introduction of the phage genome into the C. acnes strain and the expression of the phage proteins necessary for the assembly of a phage capsid and the packaging of the DNA vector inside the phage capsid.

In one embodiment, the phage genome is a wild type phage genome.

In one embodiment, the C. acnes phage is PAC7 (typically of sequence SEQ ID NO: 68).

In one embodiment, the phage genome is an engineered phage genome.

The phage-derived particles can be purified by methods known in the art. The invention encompasses purified phage-derived particles comprising a DNA vector of the invention. In one embodiment, the purified phage-derived particles are in an isolated composition or pharmaceutical composition. The composition can comprise at least 10⁴, 10⁵, 10⁶, 10⁷, 10⁸, 10⁹, 10¹⁰, 10¹¹, 10¹², 10¹³, 10¹⁴, or more purified phage-derived particles.

Sequence Specific Killing of C. acnes by Phage-Derived Particles

In one embodiment, the invention comprises specific killing of C. acnes by phage-derived particles carrying CRISPR-Cas system.

Phage-derived particles carrying a vector (e.g. plasmid) encoding CRISPR-Cas system have been recently used to perform in situ sequence specific killing of bacteria^{10,11}. The inventors have developed a method for the production of such phage-derived particles to target C. acnes, which is encompassed by the invention.

In said method, a C. acnes producer strain comprising a DNA vector of the invention is contacted with a C. acnes phage, such as PAC7 (typically of sequence SEQ ID NO: 68) to produce a high titer phage suspension.

In one embodiment, the C. acnes comprises a DNA vector comprising:
a selection marker for C. acnes, as defined above,
a C. acnes phage packaging signal (cos site), as defined above,
an origin of replication for C. acnes, as defined above, and
a CRISPR-Cas system targeting a specific C. acnes receiver cell chromosomal locus (pTarget).

High titer C. acnes phage suspensions are typically added to C. acnes. The suspensions typically contain a mix of wild-type phages and phage-derived particles carrying the plasmid. Contacting of C. acnes cells carrying the locus targeted by the pTarget CRISPR-Cas system is typically performed with phage-derived particles containing pTarget. This can be performed in vivo or in vitro. Sequence specific killing is typically observed for lysate containing phage-derived particles comprising pTarget.

In one embodiment, the phage-derived particles comprising the pTarget vector (e.g. plasmid) are not mixed with phage and allow sequence specific killing of cells carrying the DNA targeted by the CRISPR-Cas system.

C. acnes Plasmid Curing

Naturally occurring C. acnes plasmids have been described and some of them have been associated with pro-inflammatory phenotypes^{15,23} and acne vulgaris¹⁶⁻¹⁸. Being able to cure such plasmids is of interest to study their effect, notably, their pro-inflammatory role in acne vulgaris. The inventors have developed a method to cure C. acnes plasmid.

In a first step, a C. acnes producer cell carrying the DNA vector comprising:
a C. acnes phage packaging signal as defined above,
optionally a selection marker for C. acnes, as defined above,
an origin of replication that allows replication only in C. acnes producer cell, as defined above, and
a transgene, such as a CRISPR-Cas system targeting a genetic sequence of an endogenous plasmid to be cured in a target C. acnes receiver cell, the sequence being preferably in a conserved region such as the origin of replication or in loci associated with acne vulgaris,
   is infected by a C. acnes phage leading to production of phage-derived particles carrying the DNA vector.

Contacting C. acnes phage-derived particles with C. acnes receiver cell carrying an endogenous plasmid to be cured, such as pIMPLE-HL096PA1 is performed. This can be performed in vivo or in vitro.

In some embodiments, C. acnes transductants can be selected on the appropriate antibiotic. Single colonies are typically streaked on plates with media containing the antibiotic and the presence of the plasmid is typically screened by PCR. Single colonies where no positive PCR for the plasmid pIMPLE-HL096PA1 is obtained, are then cured from the vector (e.g. plasmid) comprising the CRISPR-Cas system, and typically cryostocked.

### Treatment Methods

The invention encompasses methods to treat a C. acnes related disorder or disease.

The invention encompasses the use of engineered C. acnes strains for the treatment and/or prevention of a wide range of skin diseases and disorders.

The invention encompasses methods to treat a decrease in sebum production, follicular hyperkeratinization, colonization of skin bacteria, and inflammation using engineered C. acnes strains as defined above.

The invention encompasses the use of engineered C. acnes as defined above in cosmetics and other compositions.

In one embodiment, the invention encompasses expression of therapeutic molecules by engineered C. acnes.

In one embodiment, the invention encompasses expression of non-therapeutic molecules by engineered C. acnes.

Cutibacterium acnes is one of the most prevalent and abundant bacteria on human skin, where it can be found both on the skin surface (stratum corneum) and in the hair follicle¹². Inside the hair follicle, it is in direct contact with a large diversity of living cells such as keratinocytes, stem cells, sebaceous cells and immune cells. This is not the case on the stratum corneum, where it is mostly in contact with the dead corneocyte¹³. Thus, it appears interesting to use C. acnes as a bacterial chassis for the production and delivery of therapeutic molecules in situ inside and outside the hair follicle.

Phage-derived particles and/or bacteria producing them can be delivered to the skin by dermal or other appropriate administration method to a subject.

The subject according to the invention is an animal, preferably a mammal, even more preferably a human. However, the term “subject” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, or non-mammals such as poultry, that are in need of treatment.

The human subject according to the invention may be a human at the prenatal stage, a new-born, a child, an infant, an adolescent or an adult at any age.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with an engineered C. acnes bacteria according to the invention, is preferably comprised between 1 day and 20 weeks, more preferably between 1 day and 10 weeks, still more preferably between 1 day and 4 weeks, even more preferably between 1 day and 2 weeks. In a particular embodiment, the duration of the treatment is of about 1 week. Alternatively, the treatment may last as long as the infection, disorder and/or disease persists.

The form of the pharmaceutical or veterinary compositions, the route of administration and the dose of administration of engineered C. acnes bacteria according to the invention, can be adjusted by the man skilled in the art according to the type and severity of the disease, disorder and/or infection (e.g. depending on the bacteria species involved in the disease, disorder and/or infection and its localization in the patient's or subject's body), and to the patient or subject, in particular its age, weight, sex, and general physical condition.

Particularly, the amount of engineered C. acnes bacteria according to the invention, to be administered has to be determined by standard procedure well known by those of ordinary skills in the art. Physiological data of the patient or subject (e.g. age, size, and weight) and the routes of administration have to be taken into account to determine the appropriate dosage, so as a therapeutically effective amount will be administered to the patient or subject.

Preferably, total amount of an engineered C. acnes bacteria according to the invention, for each administration is comprised between 10⁴ and 10¹⁵ bacteria.

The invention encompasses plasmids for the expression of toxins such as nuclease, more preferably CRISPR-Cas systems to kill transduced C. acnes population.

The invention encompasses plasmids for the expression of CRISPR-Cas systems where the CRISPR-Cas systems is targeted towards sequences present only in specific strains and not present in others allowing strain specific killing among the C. acnes population.

The invention encompasses modifications of C. acnes chromosome or C. acnes endogenous plasmid. Modifications such as deletion, substitution and/or insertion leading to alteration in the C. acnes-host relation are for example contemplated.

The invention encompasses vectors, e.g. plasmids, for the expression of therapeutic molecules containing one or several genes involved in the production of the therapeutic molecule.

In the case where the therapeutic molecule is not freely diffusing from C. acnes cells, such as in the case of a therapeutic protein, a fusion with a signal peptide allowing secretion or export on the cell membrane or wall of C. acnes cells is preferably encoded on the vector, e.g. plasmid. Examples of secretion systems or signal peptides include: TAT, SEC and type VII/WXG100 secretion systems. More specifically, the signal peptide can be extracted from proteins selected from the group consisting of the proteins PPA0532 (typically referenced as Q6AAD1 in the UniprotKB database as of Nov. 4, 2020); PPA0533 (typically referenced as Q6AAD0 in the UniprotKB database as of Nov. 4, 2020); PPA0534 (typically referenced as Q6AAC9 in the UniprotKB database as of Nov. 4, 2020); PPA0598 (typically referenced as Q6AA63 in the UniprotKB database as of Nov. 4, 2020); PPA0644 (typically referenced as Q6AA16 in the UniprotKB database as of Nov. 4, 2020); PPA0687 (typically referenced as Q6A9X2 in the UniprotKB database as of Nov. 4, 2020); PPA0721 (typically referenced as Q6A9T8 in the UniprotKB database as of Nov. 4, 2020); PPA0816 (typically referenced as Q6A9J4 in the UniprotKB database as of Nov. 4, 2020); PPA1310 (typically referenced as Q6A856 in the UniprotKB database as of Nov. 4, 2020); PPA1498 (typically referenced as Q6A7MO in the UniprotKB database as of Nov. 4, 2020); PPA1662 (typically referenced as Q6A771 in the UniprotKB database as of Nov. 4, 2020); PPA1715 (typically referenced as Q6A720 in the UniprotKB database as of Nov. 4, 2020); PPA1939 (typically referenced as Q6A6F6 in the UniprotKB database as of Nov. 4, 2020); PPA2097 (typically referenced as Q6A608 in the UniprotKB database as of Nov. 4, 2020); PPA2105 (typically referenced as Q6A601 in the UniprotKB database as of Nov. 4, 2020); PPA2106 (typically referenced as Q6A600 in the UniprotKB database as of Nov. 4, 2020); PPA2142 (typically referenced as Q6A5W4 in the UniprotKB database as of Nov. 4, 2020); PPA2164 (typically referenced as Q6A5U3 in the UniprotKB database as of Nov. 4, 2020); PPA2175 (typically referenced as Q6A5T2 in the UniprotKB database as of Nov. 4, 2020), PPA2152 (typically referenced as Q6A5V4 in the UniprotKB database as of Nov. 4, 2020); PPA1340 (typically referenced as Q6A826 in the UniprotKB database as of Nov. 4, 2020) and PPA2239 (typically referenced as Q6A5MO in the UniprotKB database as of Nov. 4, 2020).

In the case where secretion is not wanted or functional, a lysing module can be added to the vector, e.g. plasmid, in order to lyse the cell and release the therapeutic molecule.

In a particular embodiment, said therapeutic molecule may be displayed on the cell membrane or wall of C. acnes cells. To be displayed, a protein of interest typically requires a N-terminal secretion signal peptide such as the ones described above as well as a C-terminal LPXTG motif allowing the class F sortase from C. acnes (Girolamo, S. D. et al., Biochem J 476, 665-682 (2019)) to covalently link the protein of interest to the cell wall. Additionally a PT rich region might be integrated upstream of the LPXTG motif. Alternatively a more classical cell wall sorting sequence (CWSS) combining a LPxTG motif followed by hydrophobic amino acids and a positively charged C-terminus can be used.

In order to control expression of the therapeutic molecule, one or several of the genes, as an operon or as single isolated genes, can be put under the control of an inducible system, such as an inducible promoter, a riboswitch, a RNA-based induction method or a combination thereof. Several promoters of several transcriptional strengths might be tested and combined with different RBS strengths to optimize for in situ production of the therapeutic molecule. An RBS library approach might be used to select the best RBS variant for in vitro or in situ expression.

Examples of therapeutic molecules include but are not limited to antibodies, antibody-based drugs, Fc fusion proteins, anticoagulants, blood factors, bone morphogenetic proteins, engineered protein scaffolds, enzymes, growth factors, hormones, interferons, interleukins, and thrombolytics. Other examples include those that bind non-covalently to target (e.g., monoclonal antibodies), those that affect covalent bonds (e.g., enzymes), and those that exert activity without specific interactions (e.g., serum albumin).

Also contemplated herein are therapeutic molecules (e.g., recombinant therapeutic proteins) used to treat, for example, cancers, immune disorders, infections and/or other diseases. Engineered proteins, including bispecific mAbs and multispecific fusion proteins, and proteins with optimized pharmacokinetics are also contemplated by the present disclosure.

In some embodiments, the therapeutic proteins is Etanercept, Bevacizumab, Rituximab, Adalimumab, Infliximab, Trastuzumab, Insulin glargine, Epoetin alfa, Pegfilgrastim, Ranibizumab, Darbepoetin alfa, Interferon beta-Ia, Interferon beta-Ia. Insulin aspart, Rhu insulin, Octocog alfa, Insulin lispro, Cetuximab, Peginterferon alfa-2a, Interferon beta-Ib, Eptacog alfa, Insulin aspart, OnabotulinumtoxinA, Epoetin beta, Rec antihemophilic factor, Filgrastin, Insulin detemir, Natalizumab, Insulin (humulin) or Palivizumab.

Examples of antibodies, antibody fragments, and/or Fc fusion proteins that may be expressed in the context of the present disclosure include, without limitation, Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anifrolumab, Anrukinzumab, Apolizumab, Arcitumomab, Aselizumab, Atinumab, Atlizumab (or tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, lmgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilimumab (or tremelimumab), Tildrakizumab, Tigatuzumab, TNX-, Tocilizumab (or atlizumab), Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and Zolimomab aritox.

Other examples of Fc fusion proteins that may be expressed in the context of the present disclosure include, without limitation, Etanercept, Alefacept, Abatacept, Rilonacept, Romiplostim, Belatacept and Aflibercept.

Examples of anticoagulants and/or blood factors that may be expressed in the context of the present disclosure include, without limitation, Protein C, Protein S, and antithrombin, Factors I-VIII, prothrombinase, prothrombin, thrombin von Willebrand Factor (vWF), fibrinogen, fibrin and fibrinopeptides.

Examples of bone morphogenetic proteins (BMPs) that may be expressed in the context of the present disclosure include, without limitation, BMP1-BMP7, BMP8a, BMP8b, BMP 10, and BMP15.

Examples of enzymes that may be expressed in the context of the present disclosure include, without limitation, any of the enzymes assigned an Enzyme Commission Number (EC) number (e.g., EC1-EC6) by the International Union of Biochemistry and Molecular Biology (IUBMB) (Webb, Edwin C. Enzyme nomenclature 1992: recommendations of the Nomenclature Committee of the International Union of Biochemistry and Molecular Biology on the nomenclature and classification of enzymes. San Diego: Published for the International Union of Biochemistry and Molecular Biology by Academic Press. ISBN 0-12-227164-5 (1992), incorporated by reference herein). Other examples include: styrene monooxygenase (StyAB), toluene dioxygenase (TODC1C2AB), luciferase and lactase. In some embodiments, the enzyme is toluene dioxygenase. In some embodiments, the enzyme is styrene monoxygenase.

Examples of growth factors that may be expressed in the context of the present disclosure include, without limitation, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-a), Transforming growth factor beta (TGF-P), Tumor necrosis factor-alpha (TNF-), Vascular endothelial growth factor (VEGF), placental growth factor (P1GF), Foetal Bovine Somatotrophin (FBS) and IL-1-IL7.

Examples of peptide hormones that may be expressed in the context of the present disclosure include, without limitation, Amylin (or Islet Amyloid Polypeptide), Antimullerian hormone (or Miillerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Brain natriuretic peptide, Calcitonin, Cholecystokinin, Corticotropin-releasing hormone, Enkephalin, Endothelin, Erythropoietin, Follicle-stimulating hormone, Galanin, Gastrin, Ghrelin, Glucagon, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Lipotropin, Luteinizing hormone, Melanocyte stimulating hormone, Motilin, Orexin, Oxytocin, Pancreatic polypeptide, Parathyroid hormone, Prolactin, Prolactin releasing hormone, Relaxin, Renin, Secretin, Somatostatin, Thrombopoietin, Thyroid-stimulating hormone (or thyrotropin), and Thyrotropin-releasing hormone.

Examples of interferons (IFNs) that may be expressed in the context of the present disclosure include, without limitation, IFN-α, IFN-β, IFN-ω and IFN-γ.

Examples of interleukins that may be expressed in the context of the present disclosure include, without limitation, interleukin 1-17. In some embodiments, the interleukin is Interleukin-4, Interleukin-6, Interleukin-10, Interleukin-11 or Interleukin-13.

Other examples of therapeutic proteins that may be expressed in the context of the invention present disclosure include, without limitation, Insulin (blood glucose regulator), Pramlintide acetate (glucose control), Growth hormone GH (growth failure), Pegvisoman (growth hormone receptor antagonist), Mecasermin (IGFI, growth failure), Factor VIII (coagulation factor), Factor IX (coagulation factor, Protein C concentrate (anti-coagulation), al-proteinase inhibitor (anti-trypsin inhibitor), Erythropoietin (stimulates erythropoiesis), Filgrastim (granulocyte colony-stimulating factor, G-CSF; stimulates neutrophil proliferation), Sargramostim^{36, 37} (granulocytemacrophage colony-stimulating factor, GM-CSF), Oprelvekin (interleukin II, IL11), Human follicle-stimulating hormone (FSH), Human chorionic gonadotropin (HCG), Lutropin-a (human luteinizing hormone), Interleukin 2 (IL2), Interleukin-1 Receptor Agonist, Denileukin diftitox (fusion of IL2 and Diphtheria toxin), Interferon alfacon 1 (consensus interferon), Interferon-2a (IFNa2a), Interferon-2b (IFNa2b), Interferon-n3 (IFNan3), Interferon-pia (rIFN-β), Interferon-β Ib (rIFN-β), Interferon-ylb (IFNy, Salmon calcitonin (32-amino acid linear polypeptide hormone), Teriparatide (part of human parathyroid hormone 1-34 residues), Exenatide (Incretin mimetic with actions similar to glucagon-like peptide 1), Octreotide (octapeptide that mimics natural somatostatin), Dibotermin-a (recombinant human bone morphogenic protein 2), Recombinant human bone morphogenic protein 7, Histrelin acetate (gonadotropin-releasing hormone; GnRH), Palifermin (Keratinocyte growth factor, KGF), Becaplermin (platelet-derived growth factor, PDGF), Nesiritide (recombinant human B-type natriuretic peptide), Lepirudin (recombinant variant of hirudin, another variant is Bivalirudin), Anakinra (interleukin 1 (IL1) receptor antagonist), Enfuviritide (an HIV-1 gp41-derived peptide), p-Glucocerebrosidase (hydrolyzes to glucose and ceramide), Alglucosidase-a (degrades glycogen), Laronidase (digests glycosaminoglycans within lysosomes), Idursulfase (cleaves O-sulfate preventing GAGs accumulation), Galsulfase (cleave terminal sulphate from GAGs), Agalsidase-β (human a-galactosidase A, hydrolyzes glycosphingolipids), Lactase (digest lactose), Pancreatic enzymes (lipase, amylase, protease; digest food), Adenosine deaminase (metabolizes adenosine), Tissue plasminogen activator (tPA, serine protease involved in the breakdown of blood clots), Factor Vila (serine protease, causes blood to clot), Drotrecogin-a (serine protease, human activated protein C), Trypsin (serine protease, hydrolyzes proteins), Botulinum toxin type A (protease, inactivates SNAP-25 which is involved in synaptic vesicle fusion), Botulinum toxin type B (protease that inactivates SNAP-25 which is involved in synaptic vesicle fusion), Collagenase (endopeptidase, digest native collagen), Human deoxyribonuclease I (endonuclease, DNase I, cleaves DNA), Hyaluronidase (hydrolyzes hyaluronan), Papain (cysteine protease, hydrolyzes proteins), L-Asparaginase (catalyzes the conversion of L-asparagine to aspartic acid and ammonia), Rasburicase (urate oxidase, catalyzes the conversion of uric acid to allantoin), Streptokinase (Anistreplase is anisoylated plasminogen streptokinase activator complex (APS AC)), and Antithrombin III (serine protease inhibitor).

Other examples of therapeutic proteins that may be expressed in the context of the present disclosure include antigens, as defined below.

The invention further encompasses engineered C. acnes comprising vectors (e.g. plasmids) for the expression of antigens, such as a tumor antigen, a viral antigen, a bacterial antigen, a fungal antigen, a self-antigen, an allergen or a graft-specific antigen.

As used herein, an “antigen” refers to a molecule containing one or more epitopes (e.g., linear, conformational or both) that elicit an immunological response. The antigen may be of any type. In particular, it can be a protein, a polypeptide or a peptide, a carbohydrate, a lipid, a nucleic acid, such as DNA or RNA. In a particular embodiment, it is a protein, a polypeptide or a peptide. As intended herein, “protein” will be understood to encompass protein, polypeptide and peptide. Furthermore, for purposes of the present invention, an “antigen” encompasses a protein which includes modifications, such as deletions, additions and substitutions (generally conservative in nature), to the native sequence, so long as the protein maintains the ability to elicit an immunological response. These modifications may be deliberate, as through site-directed mutagenesis, or may be accidental, such as through mutations of hosts which produce the antigens.

In a particular embodiment, said antigen induces the activation or enhancement of an immune response, in particular specific to said antigen. In an alternative embodiment, said antigen results in tolerization or suppression of an immune response, in particular towards said antigen.

In a particular embodiment, said antigen decreases the subject inflammatory response.

In a particular embodiment, said antigen is a tumor antigen.

By “tumor antigen” is meant herein an antigenic substance produced in tumor cells. Tumor antigens can be, for example, peptide-containing tumor antigens, such as a polypeptide tumor antigen or glycoprotein tumor antigens. A tumor antigen can also be, for example, a saccharide-containing tumor antigen, such as a glycolipid tumor antigen or a ganglioside tumor antigen.

Tumor antigens include, but are not limited to, (a) polypeptide-containing tumor antigens, including polypeptides (which can range, for example, from about 8 to about 20 amino acids in length, although lengths outside this range are also common), lipopolypeptides and glycoproteins, and (b) saccharide-containing tumor antigens, including poly-saccharides, mucins, gangliosides, glycolipids and glycoproteins. Moreover, tumor antigens can be (a) full length molecules associated with cancer cells, (b) homologs and modified forms of the same, including molecules with deleted, added and/or substituted portions, and (c) fragments of the same. Tumor antigens include, for example, class I-restricted antigens recognized by CD8+ lymphocytes or class II-restricted antigens recognized by CD4+ lymphocytes.

Numerous tumor antigens are known in the art, including: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CD 4 (associated with, e.g., melanoma), MUM 1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkin's lymphoma), BCR-abl (associated with, e.g., chronic myelogenous leukemia), triosephosphate isomerase, IA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), SA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-I/Melan A, gplOO, MC1R, melanocyte-stimulating hormone receptor, tyrosinase, tyrosinase related protein-1/TRPI and tyrosinase related protein-2/TRP2 (associated with, e.g., melanoma), (e) prostate associated antigens such as PAP, PSA, PSMA, PSH-P1, PSM-P1, PSM-P2 (associated with e.g., prostate cancer), (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Lex (associated with, e.g., breast and colorectal cancer) as well as various mucins; glycoproteins may be coupled to a carrier protein (e.g., MUC-1 may be coupled to LH); (ii) lipopolypeptides (e.g., MUC-1 linked to a lipid moiety); (iii) polysaccharides (e.g., Globo H synthetic hexasaccharide), which may be coupled to a carrier protein (e.g., to KLH), (iv) gangliosides such as GM2, GM12, GD2, GD3 (associated with, e.g., brain, lung cancer, melanoma), which also may be coupled to carrier proteins (e.g., KLH).

Other tumor antigens include pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, pl85erbB2, pl80erbB-3, c-met, mn-23H I, TAG-72-4, CA 19-9, CA 72-4, CAM 17.1, NuMa, K-ras, p 16, TAGE, PSCA, CT7, 43-9F, 5T4, 791 Tgp72, beta-HCG, BCA225, BTAA, CA 125, CA 15-3 (CA 27.29BCAA), CA 195, CA 242, CA-50, CAM43, CD68KP1, CO-029, FGF-5, Ga733 (EpCAM), HTgp-175, M344, MA-50, MG7-Ag, MOV 18, NB/70K, NY-CO-1, RCAS1, SDCCAG16, TA-90 (Mac-2 binding protein\cyclophilin C-associated protein), TAAL6, TAG72, TLP, TPS, and the like.

In another embodiment, said antigen is a viral antigen.

By “viral antigen” is meant herein a protein encoded by a viral genome.

In certain embodiments, viral antigens preferably include epitopes which are exposed on the surface of the virus during at least one stage of its life cycle. Viral antigens are preferably conserved across multiple serotypes or isolates. Viral antigens suitable for use in the context of the invention include, but are not limited to, antigens derived from one or more of the viruses set forth below as well as the specific antigens examples identified below.

Orthomyxovirus: Viral antigens include, but are not limited to, those derived from an Orthomyxovirus, such as Influenza A, B and C. In certain embodiments, orthomyxovirus antigens are selected from one or more of the viral proteins, including hemagglutinin (HA), neuraminidase (NA), nucleoprotein (NP), matrix protein (MI), membrane protein (M2), one or more of the transcriptase components (PB1, PB2 and PA). In certain embodiments the viral antigen include HA and NA. In certain embodiments, the influenza antigens are derived from interpandemic (annual) flu strains, while in other embodiments, the influenza antigens are derived from strains with the potential to cause a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).

Paramyxoviridae viruses: Viral antigens include, but are not limited to, those derived from Paramyxoviridae viruses, such as Pneumoviruses (RSV), Paramyxoviruses (PIV), Metapneumovirus and Morbilliviruses (Measles).

Pneumovirus: Viral antigens include, but are not limited to, those derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. In certain embodiments, pneumovirus antigens are selected from one or more of the following proteins, including surface proteins Fusion (F), Glycoprotein (G) and Small Hydrophobic protein (SH), matrix proteins M and M2, nucleocapsid proteins N, P and L and nonstructural proteins NS1 and NS2. In other embodiments, pneumovirus antigens include F, G and M.

Paramyxovirus: Viral antigens include, but are not limited to, those derived from a Paramyxovirus, such as Parainfluenza virus types 1-4 (PIV), Mumps, Sendai viruses, Simian virus 5, Bovine parainfluenza virus, Nipahvirus, Henipavirus and Newcastle disease virus. In certain embodiments, the Paramyxovirus is PIV or Mumps. In certain embodiments, paramyxovirus antigens are selected from one or more of the following proteins: Hemagglutinin-Neuraminidase (HN), Fusion proteins F1 and F2, Nucleoprotein (NP), Phosphoprotein (P), Large protein (L), and Matrix protein (M). In other embodiments, paramyxovirus proteins include HN, F1 and F2. In other embodiments, the Paramyxovirus is Nipahvirus or Henipavirus and the antigens are selected from one or more of the following proteins: Fusion (F) protein, Glycoprotein (G) protein, Matrix (M) protein, Nucleocapsid (N) protein, Large (L) protein and Phosphoprotein (P).

Poxviridae: Viral antigens include, but are not limited to, those derived from Orthopoxvirus such as Variola vera, including but not limited to, Variola major and Variola minor.

Metapneumovirus: Viral antigens include, but are not limited to, Metapneumovirus, such as human metapneumovirus (hMPV) and avian metapneumoviruses (aMPV). In certain embodiments, metapneumovirus antigens are selected from one or more of the following proteins, including surface proteins Fusion (F), Glycoprotein (G) and Small Hydrophobic protein (SH), matrix proteins M and M2, nucleocapsid proteins N, P and L. In other embodiments, metapneumovirus antigens include F, G and M.

Morbillivirus: Viral antigens include, but are not limited to, those derived from a Morbillivirus, such as Measles. In certain embodiments, morbillivirus antigens are selected from one or more of the following proteins: hemagglutinin (H), Glycoprotein (G), Fusion factor (F), Large protein (L), Nucleoprotein (NP), Polymerase phosphoprotein (P), and Matrix (M).

Picornavirus: Viral antigens include, but are not limited to, those derived from Picornaviruses, such as Enteroviruses, Rhinoviruses, Heparnavirus, Cardioviruses and Aphthoviruses. In certain embodiments, the antigens are derived from Enteroviruses, while in other embodiments the enterovirus is Poliovirus. In still other embodiments, the antigens are derived from Rhinoviruses.

Enterovirus: Viral antigens include, but are not limited to, those derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. In certain embodiments, the antigens are derived from Enteroviruses, while in other embodiments the enterovirus is Poliovirus. In certain embodiments, the enterovirus antigens are selected from one or more of the following Capsid proteins VP0, VP1, VP2, VP3 and VP4.

Bunyavirus: Viral antigens include, but are not limited to, those derived from an Orthobunyavirus, such as California encephalitis virus, a Phlebovirus, such as Rift Valley Fever virus, or a Nairovirus, such as Crimean-Congo hemorrhagic fever virus. Rhinovirus: Viral antigens include, but are not limited to, those derived from rhinovirus. In certain embodiments, the rhinovirus antigens are selected from one or more of the following Capsid proteins: VP0, VP1, VP2, VP2 and VP4.

Heparnavirus: Viral antigens include, but are not limited to, those derived from a Heparnavirus, such as, by way of example only, Hepatitis A virus (HAV).

Togavirus: Viral antigens include, but are not limited to, those derived from a Togavirus, such as a Rubivirus, an Alphavirus, or an Arterivirus. In certain embodiments, the antigens are derived from Rubivirus, such as by way of example only, Rubella virus. In certain embodiments, the togavirus antigens are selected from E1, E2, E3, C, NSP-1, NSPO-2, NSP-3 or NSP-4. In certain embodiments, the togavirus antigens are selected from E1, E2 or E3.

Flavivirus: Viral antigens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus. In certain embodiments, the flavivirus antigens are selected from PrM, M, C, E, NS-1, NS-2a, NS2b, NS3, NS4a, NS4b, and NS5. In certain embodiments, the flavivirus antigens are selected from PrM, M and E.

Pestivirus: Viral antigens include, but are not limited to, those derived from a Pestivirus, such as Bovine viral diarrhea (BVDV), Classical swine fever (CSFV) or Border disease (BDV).

Hepadnavirus: Viral antigens include, but are not limited to, those derived from a Hepadnavirus, such as Hepatitis B virus. In certain embodiments, the hepadnavirus antigens are selected from surface antigens (L, M and S), core antigens (HBc, HBe).

Hepatitis C virus: Viral antigens include, but are not limited to, those derived from a Hepatitis C virus (HCV). In certain embodiments, the HCV antigens are selected from one or more of E1, E2, E1/E2, NS345 polyprotein, NS 345-core polyprotein, core, and/or peptides from the nonstructural regions. In certain embodiments, the Hepatitis C virus antigens include one or more of the following: HCV E1 and or E2 proteins, E1/E2 heterodimer complexes, core proteins and non-structural proteins, or fragments of these antigens, wherein the non-structural proteins can optionally be modified to remove enzymatic activity but retain immunogenicity.

Rhabdovirus: Viral antigens include, but are not limited to, those derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS).

Caliciviridae; Viral antigens include, but are not limited to, those derived from Caliciviridae, such as Norwalk virus, and Norwalk-like Viruses, such as Hawaii Virus and Snow Mountain Virus.

Coronavirus: Viral antigens include, but are not limited to, those derived from a Coronavirus, SARS, Human respiratory coronavirus, Avian infectious bronchitis (IBV), Mouse hepatitis virus (MHV), and Porcine transmissible gastroenteritis virus (TGEV). In certain embodiments, the coronavirus antigens are selected from spike (S), envelope (E), matrix (M), nucleocapsid (N), and Hemagglutinin-esterase glycoprotein (HE). In certain embodiments, the coronavirus antigen is derived from a SARS virus. In certain embodiments, the coronavirus is derived from a SARS viral antigen as described in WO 04/92360.

Retrovirus: Viral antigens include, but are not limited to, those derived from a Retrovirus, such as an Oncovirus, a Lentivirus or a Spumavirus. In certain embodiments, the oncovirus antigens are derived from HTLV-1, HTLV-2 or HTLV-5. In certain embodiments, the lentivirus antigens are derived from HIV-1 or HIV-2. In certain embodiments, the antigens are derived from HIV-1 subtypes (or clades), including, but not limited to, HIV-1 subtypes (or clades) A, B, C, D, F, G, H, J, K, O. In other embodiments, the antigens are derived from HIV-1 circulating recombinant forms (CRFs), including, but not limited to, A/B, A/E, A/G, A/G/I, etc. In certain embodiments, the retrovirus antigens are selected from gag, pol, env, tax, tat, rex, rev, nef, vif, vpu, and vpr. In certain embodiments, the HIV antigens are selected from gag (p24gag and p55gag), env (gp160 and gp41), pol, tat, nef, rev vpu, miniproteins, (preferably p55 gag and gp140v delete). In certain embodiments, the HIV antigens are derived from one or more of the following strains: HIVIIIb, HIVSF2, HIVLAV, HIVLAI, HIVMN, HIV-1CM235, HIV-1US4, HIV-1 SF162, HIV-1TV1, HIV-1MJ4. In certain embodiments, the antigens are derived from endogenous human retroviruses, including, but not limited to, HERV-K (“old” HERV-K and “new” HERV-K).

Reovirus: Viral antigens include, but are not limited to, those derived from a Reovirus, such as an Orthoreovirus, a Rotavirus, an Orbivirus, or a Coltivirus. In certain embodiments, the reovirus antigens are selected from structural proteins λ1, λ2, λ3, μ1, μ2, σ1, σ2, or σ3, or nonstructural proteins σNS, μNS, or ols. In certain embodiments, the reovirus antigens are derived from a Rotavirus. In certain embodiments, the rotavirus antigens are selected from VP1, VP2, VP3, VP4 (or the cleaved product VP5 and VP8), NSP 1, VP6, NSP3, NSP2, VP7, NSP4, or NSP5. In certain embodiments, the rotavirus antigens include VP4 (or the cleaved product VP5 and VP8), and VP7.

Parvovirus: Viral antigens include, but are not limited to, those derived from a Parvovirus, such as Parvovirus B19. In certain embodiments, the Parvovirus antigens are selected from VP-1, VP-2, VP-3, NS-1 and NS-2. In certain embodiments, the Parvovirus antigen is capsid protein VP1 or VP-2.

Delta hepatitis virus (HDV): Viral antigens include, but are not limited to, those derived from HDV, particularly δ-antigen from HDV.

Hepatitis E virus (HEV): Viral antigens include, but are not limited to, those derived from HEV.

Hepatitis G virus (HGV): Viral antigens include, but are not limited to, those derived from HGV.

Human Herpesvirus: Viral antigens include, but are not limited to, those derived from a Human Herpesvirus, such as, byway of example only, Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). In certain embodiments, the Human Herpesvirus antigens are selected from immediate early proteins (a), early proteins (β), and late proteins (γ). In certain embodiments, the HSV antigens are derived from HSV-1 or HSV-2 strains. In certain embodiments, the HSV antigens are selected from glycoproteins gB, gC, gD and gH, fusion protein (gB), or immune escape proteins (gC, gE, or gI). In certain embodiments, the VZV antigens are selected from core, nucleocapsid, tegument, or envelope proteins. A live attenuated VZV vaccine is commercially available. In certain embodiments, the EBV antigens are selected from early antigen (EA) proteins, viral capsid antigen (VCA), and glycoproteins of the membrane antigen (MA). In certain embodiments, the CMV antigens are selected from capsid proteins, envelope glycoproteins (such as gB and gH), and tegument proteins. In other embodiments, CMV antigens may be selected from one or more of the following proteins: pp65, IE1, gB, gD, gH, gL, gM, gN, gO, UL128, UL129, gUL130, UL150, UL131, UL33, UL78, US27, US28, RL5A, RL6, RL10, RL11, RL12, RL13, UL1, UL2, UL4, UL5, UL6, UL7, UL8, UL9, UL10, UL11, UL14, UL15A, UL16, UL17, UL18, UL22A, UL38, UL40, UL41A, UL42, UL116, UL119, UL120, UL121, UL124, UL132, UL147A, UL148, UL142, UL144, UL141, UL140, UL135, UL136, UL138, UL139, UL133, UL135, UL148A, UL148B, UL148C, UL148D, US2, US3, US6, US7, US8, US9, US10, US11, US12, US13, US14, US15, US16, US17, US18, US19, US20, US21, US29, US30 and US34A. CMV antigens may also be fusions of one or more CMV proteins, such as, by way of example only, pp65/IEI (Reap et al., Vaccine (2007) 25:7441-7449).

Papovaviruses: Antigens include, but are not limited to, those derived from Papovaviruses, such as Papillomaviruses and Polyomaviruses. In certain embodiments, the Papillomaviruses include HPV serotypes 1, 2, 4, 5, 6, 8, 11, 13, 16, 18, 31, 33, 35, 39, 41, 42, 47, 51, 57, 58, 63 and 65. In certain embodiments, the HPV antigens are derived from serotypes 6, 11, 16 or 18. In certain embodiments, the HPV antigens are selected from capsid proteins (L1) and (L2), or E1-E7, or fusions thereof. In certain embodiments, the HPV antigens are formulated into virus-like particles (VLPs). In certain embodiments, the Polyomavirus viruses include BK virus and JK virus. In certain embodiments, the Polyomavirus antigens are selected from VP1, VP2 or VP3.

Adenovirus: Antigens include those derived from Adenovirus. In certain embodiments, the Adenovirus antigens are derived from Adenovirus serotype 36 (Ad-36). In certain embodiments, the antigen is derived from a protein or peptide sequence encoding an Ad-36 coat protein or fragment thereof (WO 2007/120362).

In another embodiment, said antigen is a bacterial antigen.

Examples of bacterial antigens suitable for use in the context of the invention include, but are not limited to, proteins, polysaccharides and lipopolysaccharides, which are derived from a bacteria. In certain embodiments, the bacterial antigens include epitopes which are exposed on the surface of the bacteria during at least one stage of its life cycle. Bacterial antigens are preferably conserved across multiple serotypes. In certain embodiments, the bacterial antigens include antigens derived from one or more of the bacteria set forth below as well as the specific antigens examples identified below:

Neisseria meningitidis: N. meningitidis antigens include, but are not limited to, proteins, saccharides (including a polysaccharide, or lipooligosaccharide), derived from N. meningitidis serogroup such as A, C, W135, Y, X or B. A useful combination of N. meningitidis protein antigens includes one, two or three of a NHBA, a fHbp, and/or a NadA immunogen.

Streptococcus pneumoniae: Streptococcus pneumoniae antigens include, but are not limited to, a saccharide (including a polysaccharide or an oligosaccharide) and/or protein from Streptococcus pneumoniae. In certain embodiments saccharide antigens are selected from one or more of the following pneumococcal serotypes 1, 2, 3, 4, 5, 6A, 6B, 7F, 8, 9N, 9V, 10A, 11A, 12F, 14, 15B, 17F, 18C, 19A, 19F, 20, 22F, 23F, and/or 33F. A vaccine or immunogenic composition may include multiple serotypes e.g. 2, 3, 4, 5, 6, 7, 8, 9, 10, 11, 12, 13, 14, 15, 16, 17, 18, 19, 20, 21, 22, 23 or more serotypes. 7-valent, 9-valent, 10-valent, 11-valent and 13-valent conjugate combinations are already known in the art, as is a 23-valent unconjugated combination. For example, an 10-valent combination may include saccharide from serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; r 22F and 15B; A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F. etc. In certain embodiments, protein antigens may be selected from a protein identified in WO98/18931, WO98/18930, U.S. Pat. Nos. 6,699,703, 6,800,744, WO97/43303, WO97/37026, WO 02/079241, WO 02/34773, WO 00/06737, WO 00/06738, WO 00/58475, WO 2003/082183, WO 00/37105, WO 02/22167, WO 02/22168, WO 2003/104272, WO 02/08426, WO 01/12219, WO 99/53940, WO 01/81380, WO 2004/092209, WO 00/76540, WO 2007/116322, LeMieux et al., Infect. Imm. (2006) 74:2453-2456, Hoskins et al., J. Bacteriol. (2001) 183:5709-5717, Adamou et al., Infect. Immun. (2001) 69(2):949-958, Briles et al., J. Infect. Dis. (2000) 182:1694-1701, Talkington et al., Microb. Pathog. (1996) 21(1):17-22, Bethe et al., FEMS Microbiol. Lett. (2001) 205(1):99-104, Brown et al., Infect. Immun. (2001) 69:6702-6706, Whalen et al., FEMS Immunol. Med. Microbiol. (2005) 43:73-80, Jomaa et al., Vaccine (2006) 24(24):5133-5139. In other embodiments, Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Spl28, SplOI, Spl30, Spl25, Spl33, pneumococcal pilus subunits.

Streptococcus pyogenes (Group A Streptococcus): Group A Streptococcus antigens include, but are not limited to, a protein identified in WO 02/34771 or WO 2005/032582 (including GAS 40), fusions of fragments of GAS M proteins (including those described in WO 02/094851, and Dale, Vaccine (1999) 17:193-200, and Dale, Vaccine 14(10): 944-948), fibronectin binding protein (SfbI), Streptococcal heme-associated protein (Shp), and Streptolysin S (SagA).

Moraxella catarrhalis: Moraxella antigens include, but are not limited to, antigens identified in WO 02/18595 and WO 99/58562, outer membrane protein antigens (HMW-OMP), C-antigen, and/or LPS.

Bordetella pertussis: Pertussis antigens include, but are not limited to, pertussis toxoid (PT) and filamentous haemagglutinin (FHA) from B. pertussis, optionally also combination with pertactin and/or agglutinogens 2 and 3.

Burkholderia: Burkholderia antigens include, but are not limited to Burkholderia mallei, Burkholderia pseudomallei and Burkholderia cepacia.

Staphylococcus aureus: S. aureus antigens include, but are not limited to, a polysaccharide and/or protein from S. aureus. S. aureus polysaccharides include, but are not limited to, type 5 and type 8 capsular polysaccharides (CP5 and CP8) optionally conjugated to nontoxic recombinant Pseudomonas aeruginosa exotoxin A, such as StaphVAX™, type 336 polysaccharides (336PS), polysaccharide intercellular adhesions (PIA, also known as PNAG). S. aureus proteins include, but are not limited to, antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin). In certain embodiments, S. aureus antigens may be selected from a protein identified in WO 02/094868, WO 2008/019162, WO 02/059148, WO 02/102829, WO 03/011899, WO 2005/079315, WO 02/077183, WO 99/27109, WO 01/70955, WO 00/12689, WO 00/12131, WO 2006/032475, WO 2006/032472, WO 2006/032500, WO 2007/113222, WO 2007/113223, WO 2007/113224. In other embodiments, S. aureus antigens may be selected from IsdA, IsdB, IsdC, SdrC, SdrD, SdrE, CIfA, CIfB, SasF, SasD, SasH (AdsA), Spa, EsaC, EsxA, EsxB, Emp, HlaH35L, CP5, CP8, PNAG, 336PS.

Staphylococcus epidermis: S. epidermidis antigens include, but are not limited to, slime-associated antigen (SAA).

Clostridium tetani (Tetanus): Tetanus antigens include, but are not limited to, tetanus toxoid (TT).

Clostridium perfringens: Antigens include, but are not limited to, Epsilon toxin from Clostridium perfringens.

Clostridium botulinum (Botulism): Botulism antigens include, but are not limited to, those derived from C. botulinum.

Corynebacterium diphtheriae (Diphtheria): Diphtheria antigens include, but are not limited to, diphtheria toxin, preferably detoxified, such as CRM 197. In certain embodiments, the diphtheria toxoids are used as carrier proteins.

Haemophilus influenzae B (Hib): Hib antigens include, but are not limited to, a Hib saccharide antigen. The Hib antigens may be conjugated.

Pseudomonas aeruginosa: Pseudomonas antigens include, but are not limited to, endotoxin A, Wzz protein, P. aeruginosa LPS, LPS isolated from PAO1 (O5 serotype), and/or Outer Membrane Proteins, including Outer Membrane Proteins F (OprF).

Brucella: Bacterial antigens derived from Brucella, including but not limited to, B. abortus, B. canis, B. melitensis, B. neotomae, B. ovis, B. suis and B. pinnipediae.

Francisella: Bacterial antigens derived from Francisella, including but not limited to, F. novicida, F. philomiragia and F. tularensis.

Streptococcus agalactiae (Group B Streptococcus): Group B Streptococcus antigens include, but are not limited to, a protein or saccharide antigen identified in WO 02/34771, WO 03/093306, WO 04/041157, or WO 2005/002619 (including proteins GBS 80, GBS 104, GBS 276 and GBS 322, and including saccharide antigens derived from serotypes Ia, Ib, Ia/c, II, III, IV, V, VI, VII and VIII).

Neiserria gonorrhoeae: Gonorrhoeae antigens include, but are not limited to, Por (or porin) protein, such as PorB (see Zhu et al., Vaccine (2004) 22:660-669), a transferrin binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al, J Infectious Disease (2000) 182:848-855), also see, e.g., WO99/24578, WO99/36544, WO99/57280, WO02/079243).

Chlamydia trachomatis: Chlamydia trachomatis antigens include, but are not limited to, antigens derived from serotypes A, B, Ba and C (agents of trachoma, a cause of blindness), serotypes L1, L2 & L3 (associated with Lymphogranuloma venereum), and serotypes, D-K. In certain embodiments, Chlamydia trachomatis antigens include, but are not limited to, an antigen identified in WO 00/37494, WO 03/049762, WO 03/068811, or WO 05/002619, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761).

Treponema pallidum (Syphilis): Syphilis antigens include, but are not limited to, TmpA antigen.

Haemophilus ducreyi (causing chancroid): Ducreyi antigens include, but are not limited to, outer membrane protein (DsrA).

Enterococcus faecalis or Enterococcus faecium: Antigens include, but are not limited to, a trisaccharide repeat or other Enterococcus derived antigens.

Helicobacter pylori: H. pylori antigens include, but are not limited to, CagA, VacA, NAP, HopX, HopY and/or urease antigen.

Staphylococcus saprophyticus: Antigens include, but are not limited to, the 160 kDa hemagglutinin of S. saprophyticus antigen.

Yersinia enterocolitica: Antigens include, but are not limited to, LPS.

E. coli: E. coli antigens may be derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC antigens include, but are not limited to, accessory colonization factor (orf3526), orf353, bacterial Ig-like domain (group 1) protein (orf405), orf1364, NodT-family outer-membrane-factor-lipoprotein efflux transporter (orfl767), gspK (orf3515), gspJ (orf3516), tonB-dependent siderophore receptor (orf3597), fimbrial protein (orf3613), upec-948, upec-1232, A chain precursor of the type-1 fimbrial protein (upec-1875), yap H homolog (upec-2820), and hemolysin A (recp-3768).

Bacillus anthracis (anthrax): B. anthracis antigens include, but are not limited to, A-components (lethal factor (LF) and edema factor (EF)), both of which can share a common B-component known as protective antigen (PA). In certain embodiments, B. anthracis antigens are optionally detoxified.

Yersinia pestis (plague): Plague antigens include, but are not limited to, F1 capsular antigen, LPS, Yersinia pestis V antigen.

Mycobacterium tuberculosis: Tuberculosis antigens include, but are not limited to, lipoproteins, LPS, BCG antigens, a fusion protein of antigen 85B (Ag85B), ESAT-6, Mycobacterium tuberculosis (Mtb) isocitrate dehydrogenase associated antigens, and MPT51 antigens.

Rickettsia: Antigens include, but are not limited to, outer membrane proteins, including the outer membrane protein A and/or B (OmpB), LPS, and surface protein antigen (SPA).

Listeria monocytogenes: Bacterial antigens include, but are not limited to, those derived from Listeria monocytogenes.

Chlamydia pneumoniae: Antigens include, but are not limited to, those identified in WO 02/02606.

Vibrio cholerae: Antigens include, but are not limited to, proteinase antigens, LPS, particularly lipopolysaccharides of Vibrio cholerae II, O1 Inaba O-specific polysaccharides, V. cholera O139, antigens of IEM108 vaccine and Zonula occludens toxin (Zot).

Salmonella typhi (typhoid fever): Antigens include, but are not limited to, capsular polysaccharides preferably conjugates (Vi, i.e. vax-TyVi).

Borrelia burgdorferi (Lyme disease): Antigens include, but are not limited to, lipoproteins (such as OspA, OspB, Osp C and Osp D), other surface proteins such as OspE-related proteins (Erps), decorin-binding proteins (such as DbpA), and antigenically variable VI proteins, such as antigens associated with P39 and P13 (an integral membrane protein), VIsE Antigenic Variation Protein.

Porphyromonas gingivalis: Antigens include, but are not limited to, P. gingivalis outer membrane protein (OMP).

Klebsiella: Antigens include, but are not limited to, an OMP, including OMP A, or a polysaccharide optionally conjugated to tetanus toxoid.

Other bacterial antigens used in the context of the invention include, but are not limited to, capsular antigens, polysaccharide antigens, or protein antigens of any of the above. In certain embodiments, the bacterial antigens used in the context of the invention are derived from gram-negative bacteria, while in other embodiments they are derived from gram-positive bacteria. In certain embodiments, the bacterial antigens used in the context of the invention are derived from aerobic bacteria, while in other embodiments they are derived from anaerobic bacteria.

In another embodiment, said antigen is a fungal antigen.

Examples of fungal antigens used in the context of the invention include, but are not limited to, those derived from one or more of the fungi set forth below.

Fungal antigens may be derived from Dermatophytes, including: Epidermophyton floccosum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleinii, Trichophyton tonsurans, Trichophyton verrucosum, T. verrucosum var. album, var. discoides, var. ochraceum, Trichophyton violaceum, and/or Trichophyton faviforme.

Fungal antigens may also be derived from Aspergillus fumigatus, Aspergillus flavus, Aspergillus niger, Aspergillus nidulans, Aspergillus terreus, Aspergillus sydowii, Aspergillus flavarus, Aspergillus glaucus, Blastoschizomyces capitatus, Candida albicans, Candida enolase, Candida tropicalis, Candida glabrata, Candida krusei, Candida parapsilosis, Candida stellatoidea, Candida krusei, Candida parakwsei, Candida lusitaniae, Candida pseudotropicalis, Candida guilliermondii, Cladosporium carrionii, Coccidioides immitis, Blastomyces dermatitidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythium insidiosum, Pityrosporum ovale, Saccharomyces cerevisiae, Saccharomyces boulardii, Saccharomyces pombe, Scedosporium apiospermum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monilinia spp, Rhizoctonia spp, Paecilomyces spp, Pithomyces spp, and Cladosporium spp.

For example, the fungal antigen may elicit an immune response against a Candida fungus such as C. albicans.

In another embodiment, said antigen is a self-antigen.

In the context of the invention, the term “self-antigen” refers to an immunogenic antigen or epitope which is native to the subject and which may be involved in the pathogenesis of an autoimmune disease.

In some embodiments, the self-antigen is a central nervous system (CNS) antigen. In some embodiments, the self-antigen is a multiple sclerosis-associated antigen, a diabetes mellitus-associated antigen, a rheumatoid arthritis associated antigen, a myocarditis associated self-antigen, or a thyroiditis associated antigen.

Exemplary self-antigens are disclosed, for example, in US Patent Application Publication 2016/0022788, which is incorporated herein by reference in its entirety.

In some embodiments, the self-antigen is a multiple sclerosis-associated antigen. In some embodiments, the self-antigen is an antigenic peptide of or derived from myelin oligodendrocyte glycoprotein (MOG), myelin basic protein (MBP), myelin associated glycoprotein (MAG), alphaB-crystallin, S100beta, or proteolipid protein (PLP).

In some embodiments, the self-antigen is a diabetes mellitus-associated antigen. In some embodiments, the self-antigen is selected from insulin, chromogranin A, glutamic acid decarboxylase (GAD1; GAD67), glutamate decarboxylase 2 (GAD2; GAD65) and islet-specific glucose-6-phosphatase catalytic subunit-related protein and combinations thereof. Antigenic fragments and antigenic derivatives of these antigens are also contemplated. In some embodiments, the antigen can be proinsulin.

In some embodiments, the self-antigen is a rheumatoid arthritis associated antigen. In some embodiments, the rheumatoid arthritis associated self-antigen can be the peptide (Q/R)(K/R)RAA. In some embodiments, the arthritis associated self-antigen can be type II collagen or a fragment thereof.

In some embodiments, the self-antigen is a myocarditis associated self-antigen. In some embodiments, the myocarditis associated self-antigen is myosin or an antigenic fragment or antigenic derivative. In some embodiments, the antigen can be a peptide contained in human myosin. In some embodiments, the antigen can be a peptide contained within a-myosin.

In some embodiments, the self-antigen is a thyroiditis associated antigen. In some embodiments, the self-antigen is selected from thyroid peroxidase (TPO), thyroglobulin, or Pendrin.

In another embodiment, said antigen is an allergen.

An “allergen” is defined as a substance, usually a protein, which elicits the production of IgE antibodies in predisposed individuals. Similar definitions are presented in the following references: Clin. Exp. Allergy, No. 26, pp. 494-516 (1996); Mol. Biol. of Allergy and Immunology, ed. R. Bush, Immunology and Allergy Clinics of North American Series (August 1996). In a particular embodiment, the antigen is a protein allergen, i.e. any amino acid chain likely to trigger an allergic response, including short peptides of about 6 to 20 amino acids, polypeptides, or full proteins.

Non limitative examples of allergens include pollen allergens (such as tree-, herb, weed-, and grass pollen allergens), insect allergens (such as inhalant, saliva and venom allergens, e.g., cockroach and midges allergens, hymenoptera venom allergens), mite allergens, animal hair and dandruff allergens (from e.g. dog, cat, horse, rat, mouse etc.), and food allergens.

For instance, the protein allergen may be selected from the group consisting of a protein allergen of the genus Dermatophagoides; a protein allergen of the genus Felis; a protein allergen of the genus Ambrosia; a protein allergen of the genus Lolium; a protein allergen of the genus Cryptomeria; a protein allergen of the genus Alternaria; a protein allergen of the genus Alder; a protein allergen of the genus Betula; a protein allergen of the genus of Blomia; a protein allergen of the genus Quercus; a protein allergen of the genus Olea; a protein allergen of the genus Artemisia; a protein allergen of the genus Plantago; a protein allergen of the genus Parietaria; a protein allergen of the genus Canine; a protein allergen of the genus Blattella; a protein allergen of the genus Apis; a protein allergen of the genus Cupressus; a protein allergen of the genus Thuya; a protein allergen of the genus Chamaecyparis; a protein allergen of the genus Periplaneta; a protein allergen of the genus Agropyron; a protein allergen of the genus Secale; a protein allergen of the genus Triticum; a protein allergen of the genus Cynorhodon; a protein allergen of the genus Juniperus; a protein allergen of the genus Dactylis; a protein allergen of the genus Festuca; a protein allergen of the genus Poa; a protein allergen of the genus Avena; a protein allergen of the genus Holcus; a protein allergen of the genus Anthoxanthum; a protein allergen of the genus Arrhenatherum; a protein allergen of the genus Agrostis; a protein allergen of the genus Phleum; a protein allergen of the genus Phalaris; a protein allergen of the genus Paspalum; and a protein allergen of the genus Sorghum.

Examples of various known protein allergens derived from some of the above-identified genus include: Betula (verrucosa) Bet v I; Bet v II; Blomia Blo t I; Blo t III; Blo t V; Blo t XII; Cynorhodon Cyn d I; Dermatophagoides (pteronyssinus or farinae) Der p I; Der p II; Der p III; Der p VII; Der f I; Der f II; Der f III; Der f VII; Felis (domesticus) Fel d I; Ambrosia (artemiisfolia) Amb a 1.1; Amb a 1.2; Amb a 1.3; Amb a 1.4; Amb a 1l; Lollium (perenne) Lol p I; Lot p II; Lol p III; Lot p IV; Lol p IX (Lol p V or Lol p Ib); Cryptomeria (japonica) Cry j I; Cry j II; Canis (familiaris) Can f I; Can f II; Juniperus (sabinoides or virginiana) Jun s I; Jun v I; Juniperus (ashei) Jun a I; Jun a II; Dactylis (glomerata) Dac g I; Dac g V; Poa (pretensis) Poa p I; Phl p I; Phl p V; Phl p VI and Sorghum (halepensis) Sor h I.

Food allergens may originate from milk and milk products, eggs, legumes (peanuts and soy), tree nuts, cereals (such as wheat), brassicaceae (such as mustard), crustaceans, fish, and mollusks. In particular, food allergens may be ovalbumin or gluten.

The invention also encompasses vaccine and/or immunogenic and/or immunotherapeutic compositions comprising a DNA vector, as defined above, comprising a nucleic acid encoding an antigen, such as a tumor antigen, a viral antigen, a bacterial antigen, a fungal antigen, a self-antigen, an allergen or a graft-specific antigen, as defined above, or an engineered C. acnes comprising said DNA vector; and optionally an adjuvant.

Any conventional or exploratory, synthetic or biological adjuvant for vaccination, including heat-labile enterotoxin (LT), cholera-toxin (CT), cholera toxin B subunit (CTB), polymerised liposomes, mutant toxins, probiotic bacteria, oligonucleotides, RNA, siRNA, DNA, lipids can be used.

The invention also encompasses methods to prevent and/or a treat cancer in a subject in need thereof, comprising administering to said subject a therapeutically or prophylactically efficient amount of a DNA vector comprising a nucleic acid encoding a tumor antigen, as defined above, or of an engineered C. acnes comprising said DNA vector. The invention also concerns a DNA vector comprising a nucleic acid encoding a tumor antigen, as defined above, or an engineered C. acnes comprising said DNA vector for use in a method to prevent and/or treat cancer in a subject.

As used herein, the term “cancer” means a type of hyperproliferative disease that includes a malignancy characterized by deregulated or uncontrolled cell growth. Cancers of virtually every tissue are known. Examples of cancer include, but are not limited to, carcinoma, lymphoma, blastema, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, thyroid cancer, hepatic carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

The term “cancer,” is encompassed within the scope of the broader term “abnormal cellular proliferation”, which can also be referred to as “excessive cellular proliferation or “cellular proliferative disease”. Examples of diseases associated abnormal cellular proliferation include metastatic tumors, malignant tumors, benign tumors, cancers, precancers, hyperplasias, warts, and polyps, as well as non-cancerous conditions such as benign melanomas, benign chondroma, benign prostatic hyperplasia, moles, dysplastic nevi, dysplasia, hyperplasias, and other cellular growths occurring within the epidermal layers. Classes of precancers include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (high grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma.

The invention also encompasses methods to prevent and/or treat a viral infection in a subject in need thereof, comprising administering to said subject a therapeutically or prophylactically efficient amount of a DNA vector comprising a nucleic acid encoding a viral antigen, as defined above, or of an engineered C. acnes comprising said DNA vector. The invention also concerns a DNA vector comprising a nucleic acid encoding a viral antigen, as defined above, or an engineered C. acnes comprising said DNA vector for use in a method to prevent and/or treat a viral infection in a subject.

In said embodiment, said antigen preferably induces the activation or enhancement of an immune response, in particular specific to said antigen.

Particular examples of viral infections include, but are not limited to, cytomegalovirus (CMV) pneumonia, enteritis and retinitis; Epstein-Barr virus (EBV) lymphoproliferative disease; chicken pox/shingles (caused by varicella zoster virus, VZV); HSV-1 and -2 mucositis; HSV-6 encephalitis, BK-virus hemorrhagic cystitis; viral influenza; pneumonia from respiratory syncytial virus (RSV); AIDS (caused by HIV); and hepatitis A, B or C. Additional examples of viral infections include infections caused by Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arenaviridae (hemorrhagic fever viruses); Reoviridae (e.g., reoviruses, orbiviruses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class I=internally transmitted; class 2=parenterally transmitted (i.e., Hepatitis C)); Norwalk and related viruses, and astroviruses.

The invention also encompasses methods to prevent and/or treat a bacterial infection in a subject in need thereof, comprising administering to said subject a therapeutically or prophylactically efficient amount of a DNA vector comprising a nucleic acid encoding a bacterial antigen, as defined above, or of an engineered C. acnes comprising said DNA vector. The invention also concerns a DNA vector comprising a nucleic acid encoding a bacterial antigen, as defined above, or an engineered C. acnes comprising said DNA vector for use in a method to prevent and/or treat a bacterial infection in a subject.

In said embodiment, said antigen preferably induces the activation or enhancement of an immune response, in particular specific to said antigen.

Examples of bacterial infections include, but are not limited to, infections caused by Helicobacter pyloris, Borrelia burgdorferi, Legionella pneumophila, Mycobacteria sp. (such as M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, and Actinomyces israelii.

The invention also encompasses methods to prevent and/or treat a fungal infection in a subject in need thereof, comprising administering to said subject a therapeutically or prophylactically efficient amount of a DNA vector comprising a nucleic acid encoding a fungal antigen, as defined above, or of an engineered C. acnes comprising said DNA vector. The invention also concerns a DNA vector comprising a nucleic acid encoding a fungal antigen, as defined above, or an engineered C. acnes comprising said DNA vector for use in a method to prevent and/or treat a fungal infection in a subject.

In said embodiment, said antigen preferably induces the activation or enhancement of an immune response, in particular specific to said antigen.

Examples of fungal infections include but are not limited to: aspergillosis; thrush (caused by Candida albicans); cryptococcosis (caused by Cryptococcus); and histoplasmosis. Thus, examples of fungal infections include, but are not limited to, infections caused by Cryptococcus neoformans, Histoplasma capsulatum, Coccidioides immitis, Blastomyces dermatitidis, Chlamydia trachomatis, or Candida albicans.

The invention also encompasses methods to prevent and/or treat an auto-immune disease in a subject in need thereof, comprising administering to said subject a therapeutically or prophylactically efficient amount of a DNA vector comprising a nucleic acid encoding a self-antigen, as defined above, or of an engineered C. acnes comprising said DNA vector. The invention also concerns a DNA vector comprising a nucleic acid encoding a self-antigen, as defined above, or an engineered C. acnes comprising said DNA vector for use in a method to prevent and/or treat an auto-immune disease in a subject.

In said embodiment, said antigen preferably results in tolerization or suppression of an immune response, in particular towards said antigen.

Autoimmune diseases include, but are not limited to, multiple sclerosis, rheumatoid arthritis, myasthenia gravis, psoriasis, systemic lupus erythematosus, autoimmune thyroiditis (Hashimoto's thyroiditis), Graves' disease, inflammatory bowel disease, autoimmune uveoretinitis, myocarditis, polymyositis, and certain types of diabetes, including Type 1 diabetes.

The invention also encompasses methods to prevent and/or treat allergy, such as asthma in a subject in need thereof, comprising administering to said subject a therapeutically or prophylactically efficient amount of a DNA vector comprising a nucleic acid encoding an allergen, as defined above, or of an engineered C. acnes comprising said DNA vector. The invention also concerns a DNA vector comprising a nucleic acid encoding an allergen, as defined above, or an engineered C. acnes comprising said DNA vector for use in a method to prevent and/or treat allergy, such as asthma in a subject.

In said embodiment, said antigen preferably results in tolerization or suppression of an immune response, in particular towards said antigen.

In the context of the disclosure allergy relates to asthma or to the allergies due to the above-defined allergens.

The invention also encompasses methods to prevent and/or treat graft rejection in a subject in need thereof, comprising administering to said subject a therapeutically or prophylactically efficient amount of a DNA vector comprising a nucleic acid encoding a graft-specific antigen, as defined above, or of an engineered C. acnes comprising said DNA vector. The invention also concerns a DNA vector comprising a nucleic acid encoding a graft-specific antigen, as defined above, or an engineered C. acnes comprising said DNA vector for use in a method to prevent and/or treat graft rejection in a subject.

In said embodiment, said antigen preferably results in tolerization or suppression of an immune response, in particular towards said antigen.

Unless defined otherwise, all technical and scientific terms used herein have the same meaning as commonly understood by one of ordinary skill in the art to which this invention belongs.

All publications mentioned herein are incorporated herein by reference. It is understood that the present disclosure supersedes any disclosure of an incorporated publication to the extent there is a contradiction.

It must be noted that, as used in this specification and the appended claims, the singular forms “a”, “an” and “the” include plural referents unless the content clearly dictates otherwise. Thus, for example, reference to “an antigen” includes a mixture of two or more antigens, and the like.

### Definitions

### «Delivery Vehicle»

As used herein, the term «delivery vehicle» refers to any mean that allows the transfer of a payload into a bacterium.

There are several types of delivery vehicle encompassed by the present invention including, without limitation, bacteriophage scaffold, virus scaffold, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation).

Any combination of delivery vehicles is also encompassed by the present invention.

The delivery vehicle can refer to a bacteriophage derived scaffold and can be obtained from a natural, evolved or engineered capsid.

In some embodiment, the delivery vehicle is the payload as bacteria are naturally competent to take up a payload from the environment on their own.

### «Conjugation»

Conjugation is a process by which a donor bacteria actively transfers DNA to a recipient bacteria. DNA transfer involves recognition of an origin of transfer (oriT) by a protein known as the relaxase which nicks and covalently binds to the oriT DNA. The relaxase and single stranded DNA are then typically injected into a recipient cell through a type IV secretion system. During conjugation of a plasmid or ICE (Integrative and Conjugative Elements), transfer of the relaxase is coupled with rolling circle replication of the plasmid or ICE. Once in the recipient, the relaxase will recircularize the transferred strand at the oriT. Smillie et al, Microbiology and Molecular Biology Rev, 2010, P. 434-452.

Examples of conjugative plasmids are F, R388, RP4, RK2, R6K. Plasmids of the following groups are frequently conjugative and carry a type IV secretion system: IncA, IncB/O (Ind O), IncC, IncD, IncE, IncFI, IncF2, IncG, IncHM, IncHI2, Inch, IncI2, IncJ, IncK, IncL/M, IncN, IncP, IncQI, IncQ2, IncR, IncS, IncT, IncU, IncV, IncW, IncXI, IncX2, IncY, IncZ, ColE1, ColE2, ColE3, p15A, pSC101, IncP-2, IncP-5, IncP-7, IncP-8, IncP-9, Ind, Inc4, Inc7, Inc8, Inc9, Inc1 1, Inc13, Ind 4 or Ind 8.

List of type IV secretion systems can be found in public databases such as AtlasT4SS.

Conjugation is not limited to plasmids but can also occur from the chromosome of bacteria when an oriT is present. This can happen naturally through the recombination of conjugative plasmids in the chromosome or artificially by introducing an oriT at a position of interest in the chromosome. A particular class of conjugative elements are known as Integrative and Conjugative Elements (ICEs). These are not maintained in a circular plasmidic form but integrate in the host chromosome. Upon transfer, the ICE excises from the chromosome and is then transferred in a manner akin to a conjugative plasmid. Once in a recipient cell, the ICE integrates in the recipient's chromosome. Lists of ICE elements can be found in public databases such as ICEberg.

ICEs or plasmids which carry both an origin of transfer and the type IV secretion system genes are commonly referred to as mobile elements, while ICEs or plasmids that only carry the oriT can be referred to as mobilizable plasmids. Mobilizable elements can only be transferred from the donor cell to a recipient cell if a type IV secretion system is expressed in trans, either by another plasmid or from the chromosome of the host cell.

### «Payload»

As used herein, the term «payload» refers to any nucleic acid sequence or amino acid sequence, or a combination of both (such as, without limitation, peptide nucleic acid or peptide-oligonucleotide conjugate) transferred into a bacterium with a delivery vehicle.

The term «payload» may also refer to a plasmid, a vector or a cargo.

The payload can be a phagemid or phasmid obtained from natural, evolved or engineered bacteriophage genome. The payload can also be composed only in part of phagemid or phasmid obtained from natural, evolved or engineered bacteriophage genome.

In some embodiment, the payload is the delivery vehicle as bacteria are naturally competent to take up a payload from the environment on their own.

### «Nucleic Acid»

As used herein, the term “nucleic acid” refers to a sequence of at least two nucleotides covalently linked together which can be single-stranded or double-stranded or contains portion of both single-stranded and double-stranded sequence. Nucleic acids of the present invention can be naturally occurring, recombinant or synthetic. The nucleic acid can be in the form of a circular sequence or a linear sequence or a combination of both forms. The nucleic acid can be DNA, both genomic or cDNA, or RNA or a combination of both. The nucleic acid may contain any combination of deoxyribonucleotides and ribonucleotides, and any combination of bases, including uracil, adenine, thymine, cytosine, guanine, inosine, xanthine, hypoxanthine, isocytosine, 5-hydroxymethylcytosine and isoguanine. Other examples of modified bases that can be used in the present invention are detailed in Chemical Reviews 2016, 116 (20) 12655-12687. The term “nucleic acid” also encompasses any nucleic acid analogs which may contain other backbones comprising, without limitation, phosphoramide, phosphorothioate, phosphorodithioate, O-methylphophoroamidite linkage and/or deoxyribonucleotides and ribonucleotides nucleic acids. Any combination of the above features of a nucleic acid is also encompassed by the present invention.

### «Vector»

As used herein, the term “vector” refers to any construct of sequences that are capable of expression of a polypeptide in a given host cell. If a vector is used then the choice of vector is dependent upon the method that will be used to transform host bacteria as is well known to those skilled in the art. Vectors can include, without limitation, plasmid vectors and recombinant phage vectors, or any other vector known in that art suitable for delivering a polypeptide of the invention to target bacteria. The skilled artisan is well aware of the genetic elements that must be present on the vector in order to successfully transform, select and propagate host cells comprising any of the isolated nucleotides or nucleic acid sequences of the invention.

### «Phagemid»

As used herein the term “phagemid” or “phasmid” are equivalent and refer to a recombinant DNA vector comprising at least one sequence of a bacteriophage genome. A phagemid of the disclosure comprises a phage packaging site and optionally an origin of replication (ori), in particular a bacterial and/or phage origin of replication. In one embodiment, the phagemid of the disclosure does not comprise a bacterial origin of replication and thus cannot replicate by itself once injected into a bacterium. Alternatively, the phagemid comprises a plasmid origin of replication, in particular a bacterial and/or phage origin of replication.

### «Packaged Phagemid»

As used herein, the term “packaged phagemid” or “phage-derived particle” refers to a phagemid which is encapsidated in a bacteriophage scaffold, bacterial virus particle or capsid. Particularly, it refers to a bacteriophage scaffold, bacterial virus particle or capsid devoid of a bacteriophage genome. The packaged phagemid or phage-derived particle may be produced with a helper phage strategy, well known from the man skilled in the art. The helper phage comprises all the genes coding for the structural and functional proteins that are indispensable for the phagemid according to the invention to be encapsidated. The packaged phagemid or phage-derived particle may be produced with a satellite virus strategy, also known from the man skilled in the art. Satellite virus are subviral agent and are composed of nucleic acid that depends on the co-infection of a host cell with a helper virus for all the morphogenetic functions, whereas for all its episomal functions (integration and immunity, multicopy plasmid replication) the satellite is completely autonomous from the helper. In one embodiment, the satellite genes can encode proteins that promote capsid size reduction of the helper phage, as described for the P4 Sid protein that controls the P2 capsid size to fit its smaller genome.

### «Peptide»

As used herein, the term “peptide” refers both to a short chain of at least 2 amino acids linked between each other and to a part of, a subset of, or a fragment of a protein which part, subset or fragment being not expressed independently from the rest of the protein. In some instances, a peptide is a protein. In some other instances, a peptide is not a protein and peptide only refers to a part, a subset or a fragment of a protein. Preferably, the peptide is from 4, 5, 6, 7, 8, 9, 10, 11, 12, 13, 14, 15 amino acids to 4, 5, 6, 7, 8, 9, 10, 11, 12, 13, 14, 15, 20, 25, 30, 40, 50, 100, 200 amino acids in size.

### “Engineered”

As used herein, the term “engineered” means that the bacterial cells, phages, phage-derived particles, phagemids or vectors of the invention have been modified by molecular biology techniques. As will be understood by the skilled person, engineering of bacterial cells, phages, phage-derived particles, phagemids or vectors implies a deliberate action to introduce or modify a nucleic acid sequence and does not cover introduction or modification of a nucleic acid sequence through natural evolution of the bacterial cell, phage, phage-derived particle, phagemid or vector.

### “Percent of Identity”

As used herein, the percent identity is calculated in relation to polymers (e.g., polynucleotide or polypeptide) whose sequences have been aligned. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using a BLOSUM62 matrix, a BLOSUM30 matrix or a PAM250 matrix, and a gap weight of 16, 14, 12, 10, 8, 6, or 4 and a length weight of 1, 2, 3, 4, 5, or 6. In a specific embodiment the BLOSUM30 matrix is used with gap open penalty of 12 and gap extension penalty of 4.

### CRISPR-Cas System

A CRISPR-Cas system refers to DNA encoding two distinct elements, i.e. i) an endonuclease, in this case the CRISPR associated nuclease (Cas or “CRISPR associated protein”) and ii) a guide RNA. Depending on the type of CRISPR system, the guide RNA may be in the form of a chimeric RNA which consists of the combination of a CRISPR (crRNA) bacterial RNA and a tracrRNA (trans-activating RNA CRISPR) (Jinek et al., Science 2012). The guide RNA combines the targeting specificity of the crRNA corresponding to the “spacing sequences” that serve as guides to the Cas proteins, and the conformational properties of the tracrRNA in a single transcript. When the guide RNA and the Cas protein are expressed simultaneously in the cell, the target genomic sequence can be permanently interrupted (and causing disappearance of the targeted and surrounding sequences and/or cell death, depending on the location) or modified. The modification may be guided by a repair matrix.

The CRISPR-Cas system includes two main classes depending on the nuclease mechanism of action:
Class 1 is made of multi-subunit effector complexes and includes type I, III and IV
Class 2 is made of single-unit effector modules, like Cas9 nuclease, and includes type II (II-A,II-B,II-C,II-C variant), V (V-A,V-B,V-C,V-D,V-E,V-U1,V-U2,V-U3,V-U4,V-U5) and VI (VI-A,VI-B1,VI-B2,VI-C,VI-D)

The sequence of interest according to the present invention comprises a nucleic acid sequence encoding Cas protein. A variety of CRISPR enzymes are available for use as a sequence of interest on the plasmid according to the present invention. In some embodiments, the CRISPR enzyme is a Type II CRISPR enzyme, a Type II-A or Type II-B CRISPR enzyme. In another embodiment, the CRISPR enzyme is a Type I CRISPR enzyme or a Type III CRISPR enzyme. In some embodiments, the CRISPR enzyme catalyzes DNA cleavage. In some other embodiments, the CRISPR enzyme catalyzes RNA cleavage. In one embodiment, the CRISPR enzymes may be coupled to a guide RNA or single guide RNA (sgRNA). In certain embodiments, the guide RNA or sgRNA targets a gene selected from the group consisting of an antibiotic resistance gene, virulence protein or factor gene, toxin protein or factor gene, a bacterial receptor gene, a membrane protein gene, a structural protein gene, a secreted protein gene, a gene expressing resistance to a drug in general and a gene causing a deleterious effect to the host.

The sequence of interest may comprise a nucleic acid sequence encoding a guide RNA or sgRNA to guide the Cas protein endogenous to the targeted bacteria, alone or in combination with a Cas protein and/or a guide RNA encoded by the payload.

Non-limiting examples of Cas proteins as part of a multi-subunit effector or as a single-unit effector include Cas1, Cas1 B, Cas2, Cas3, Cas4, Cas5, Cas6, Cas7, Cas8, Cas9 (also known as Csn1 and Csx12), Cas10, Cas11 (SS), Cas12a (Cpf1), Cas12b (C2c1), Cas12c (C2c3), Cas12d (CasY), Cas12e (CasX), C2c4, C2c8, C2c5, C2c10, C2c9, Cas13a (C2c2), Cas13b (C2c6), Cas13c (C2c7), Cas13d, Csa5, Csc1, Csc2, Cse1, Cse2, Csy1, Csy2, Csy3, Csf1, Csf2, Csf3, Csf4, Csm1, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, SdCpf1, CmtCpf1, TsCpf1, CmaCpf1, PcCpf1, ErCpf1, FbCpf1, UbcCpf1, AsCpf1, LbCpf1, homologues thereof, orthologues thereof, variants thereof, or modified versions thereof. In some embodiments, the CRISPR enzyme cleaves both strands of the target nucleic acid at the Protospacer Adjacent Motif (PAM) site.

In a particular embodiment, the CRISPR enzyme is any Cas9 protein, for instance any naturally-occurring bacterial Cas9 as well as any variants, homologs or orthologs thereof.

By “Cas9” is meant a protein Cas9 (also called Csn1 or Csx12) or a functional protein, peptide or polypeptide fragment thereof, i.e. capable of interacting with the guide RNA(s) and of exerting the enzymatic activity (nuclease) which allows it to perform the double-strand cleavage of the DNA of the target genome. “Cas9” can thus denote a modified protein, for example truncated to remove domains of the protein that are not essential for the predefined functions of the protein, in particular the domains that are not necessary for interaction with the gRNA (s).

The sequence encoding Cas9 (the entire protein or a fragment thereof) as used in the context of the invention can be obtained from any known Cas9 protein (Fonfara et al., 2014; Koonin et al., 2017). Examples of Cas9 proteins useful in the present invention include, but are not limited to, Cas9 proteins of Streptococcus pyogenes (SpCas9), Streptococcus thermophiles (St1 Cas9, St3Cas9), Streptococcus mutans, Staphylococcus aureus (SaCas9), Campylobacter jejuni (CjCas9), Francisella novicida (FnCas9) and Neisseria meningitides (NmCas9).

The sequence encoding Cpf1 (Cas12a) (the entire protein or a fragment thereof) as used in the context of the invention can be obtained from any known Cpf1 (Cas12a) protein (Koonin et al., 2017). Examples of Cpf1(Cas12a) proteins useful in the present invention include, but are not limited to, Cpf1(Cas12a) proteins of Acidaminococcus sp, Lachnospiraceae bacteriu and Francisella novicida.

The sequence encoding Cas13a (the entire protein or a fragment thereof) as used in the context of the invention can be obtained from any known Cas13a (C2c2) protein (Abudayyeh et al., 2017). Examples of Cas13a (C2c2) proteins useful in the present invention include, but are not limited to, Cas13a (C2c2) proteins of Leptotrichia wadei(LwaCas13a).

The sequence encoding Cas13d (the entire protein or a fragment thereof) as used in the context of the invention can be obtained from any known Cas13d protein (Yan et al., 2018). Examples of Cas13d proteins useful in the present invention include, but are not limited to, Cas13d proteins of Eubacterium siraeum and Ruminococcus sp.

In a particular embodiment, the nucleic sequence of interest is a CRISPR/Cas9 system for the reduction of gene expression or inactivation a gene selected from the group consisting of an antibiotic resistance gene, virulence factor or protein gene, toxin factor or protein gene, a gene expressing a bacterial receptor, a membrane protein, a structural protein, a secreted protein, a gene expressing resistance to a drug in general and a gene causing a deleterious effect to the host.

In one embodiment, the CRISPR-Cas system is used to target and inactivate a virulence factor. A virulence factor can be any substance produced by a pathogen that alter host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, scavenging factors and factors that promote biofilm formation. For example, such targeted virulence factor gene can be E. coli virulence factor gene such as, without limitation, EHEC-HlyA, Stx1 (VT1), Stx2 (VT2), Stx2a (VT2a), Stx2b (VT2b), Stx2c (VT2c), Stx2d (VT2d), Stx2e (VT2e) and Stx2f (VT2f), Stx2h (VT2h), stx2k, fimA, fimF, fimH, neuC, kpsE, sfa, foc, iroN, aer, iha, papC, papGI, papGII, papGIII, hlyC, cnf1, hra, sat, ireA, usp ompT, ibeA, malX, fyuA, irp2, traT, afaD, ipaH, eltB, estA, bfpA, eaeA, espA, aaiC, aatA, TEM, CTX, SHV, csgA, csgB, csgC, csgD, csgE, csgF, csgG, csgH, T1SS, T2SS, T3SS, T4SS, T5SS, T6SS (secretion systems). For example, such targeted virulence factor gene can be Shigella dysenteriae virulence factor gene such as, without limitation, stx1 and stx2. For example, such targeted virulence factor gene can be Yersiniapestis virulence factor gene such as, without limitation, yscF (plasmid-borne (pCDI) T3SS external needle subunit). For example, such targeted virulence factor gene can be Francisella tularensis virulence factor gene such as, without limitation, fslA. For example, such targeted virulence factor gene can be Bacillus anthracis virulence factor gene such as, without limitation, pag (Anthrax toxin, cell-binding protective antigen). For example, such targeted virulence factor gene can be Vibrio cholera virulence factor gene such as, without limitation, ctxA and ctxB (cholera toxin), tcpA (toxin co-regulated pilus), and toxT (master virulence regulator). For example, such targeted virulence factor gene can be Pseudomonas aeruginosa virulence factor genes such as, without limitation, pyoverdine (e.g., sigma factor pvdS, biosynthetic genes pvdL, pvdI, pvdJ, pvdH, pvdA, pvdF, pvdQ, pvdN, pvdM, pvdO, pvdP, transporter genes pvdE, pvdR, pvdT, opmQ), siderophore pyochelin (e.g., pchD, pchC, pchB, pchA, pchE, pchF and pchG, and toxins (e.g., exoU, exoS and exoT). For example, such targeted virulence factor gene can be Klebsiella pneumoniae virulence factor genes such as, without limitation, fimA (adherence, type I fimbriae major subunit), and cps (capsular polysaccharide). For example, such targeted virulence factor gene can be Acinetobacter baumannii virulence factor genes such as, without limitation, ptk (capsule polymerization) and epsA (assembly). For example, such targeted virulence factor gene can be Salmonella enterica Typhi virulence factor genes such as, without limitation, MIA (invasion, SPI-1 regulator), ssrB (SPI-2 regulator), and those associated with bile tolerance, including efflux pump genes acrA, acrB and tolC. For example, such targeted virulence factor gene can be Fusobacterium nucleatum virulence factor genes such as, without limitation, FadA and TIGIT. For example, such targeted virulence factor gene can be Bacteroides fragilis virulence factor genes such as, without limitation, bft. For example, such targeted virulence factor gene can be Cutibacterium acnes porphyrins genes, CAMP-factors (CAMP1, CAMP2, CAMP3, CAMP4), Hyaluronate lyase (HYL-IB/II, HYL-IA), Lipases (GehA, GehB), Haemolysins, Sialidases, Endoglycoceramidases, Endo-β-N-acetylglucosaminidase, Dermatan sulphate adhesin (DsA1, DsA2), Proline-Threonine Repeats (PTRs) or any virulence factors included on the acne associated genomic loci 1, 2, 3(plasmid), 4 such as a tight adhesion locus (tad), Streptolysin S-associated genes (sag), nonribosomal peptide synthetases (NRPS) as described in Tomida et al.

In another embodiment, the CRISPR/Cas9 system is used to target and inactivate an antibiotic resistance gene such as, without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA β-lactamase, mecA, Omp36, OmpF, PIB, bla (blaI, blaR1) and mec (mecI, mecR1) operons, Chloramphenicol acetyltransferase (CAT), Chloramphenicol phosphotransferase, Ethambutol-resistant arabinosyltransferase (EmbB), MupA, MupB, Integral membrane protein MprF, Cfr 23S rRNA methyltransferase, Rifampin ADP-ribosyltransferase (Arr), Rifampin glycosyltransferase, Rifampin monooxygenase, Rifampin phosphotransferase, DnaA, RbpA, Rifampin-resistant beta-subunit of RNA polymerase (RpoB), Erm 23S rRNA methyltransferases, Lsa, MsrA, Vga, VgaB, Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, Lincosamide nucleotidyltransferase (Lin), EreA, EreB, GimA, Mgt, Ole, Macrolide phosphotransferases (MPH), MefA, MefE, Mel, Streptothricin acetyltransferase (sat), Sul1, Sul2, Sul3, sulfonamide-resistant FoIP, Tetracycline inactivation enzyme TetX, TetA, TetB, TetC, Tet30, Tet31, TetM, TetO, TetQ, Tet32, Tet36, MacAB-TolC, MsbA, MsrA, VgaB, EmrD, EmrAB-TolC, NorB, GepA, MepA, AdeABC, AcrD, MexAB-OprM, mtrCDE, EmrE, adeR, acrR, baeSR, mexR, phoPQ, mtrR, or any antibiotic resistance gene described in the Comprehensive Antibiotic Resistance Database (CARD https://card.mcmaster.ca/).

In another embodiment, the CRISPR/Cas9 system is used to target and inactivate a bacterial toxin gene. Bacterial toxin can be classified as either exotoxins or endotoxins. Exotoxins are generated and actively secreted; endotoxins remain part of the bacteria. The response to a bacterial toxin can involve severe inflammation and can lead to sepsis. Such toxin can be for example Botulinum neurotoxin, Tetanus toxin, Staphylococus toxins, Diphteria toxin, Anthrax toxin, Alpha toxin, Pertussis toxin, Shiga toxin, Heat-stable enterotoxin (E. coli ST), colibactin, BFT (B. fragilis toxin) or any toxin described in Henkel et al., (Toxins from Bacteria in EXS. 2010; 100: 1-29).

### Base Editing

Base editing (BE) refers to the ability to substitute a specific nucleotide base pair on a DNA or RNA molecule by another. Until recently, the only way to perform a specific substitution on DNA in vivo was using recombination of a template DNA, carrying the specific base pair change, with the locus of interest. Base editing technology relies on completely different strategies. There is no exchange of DNA, instead an enzymatic reaction converts a nucleotide to another one leading to a mismatch at the level of dsDNA that is then corrected by the cell machinery.

One of the main challenges for base editing is how to restrict activity of the enzyme performing the nucleotide conversion to the target nucleotide, for example a SNP involved in pathogenicity. This spatial restriction has been achieved recently repurposing the CRISPR-Cas system. Indeed, fusing catalytically impaired or inactive Cas nuclease to base modification enzymes that are active only on single stranded DNA, it's possible to achieve high efficiency base editing. This is possible thanks to the CRISPR-Cas ability to generate locally ssDNA bubble in an ‘R loop’ when the complex is annealed to its DNA target strand by RNA-DNA base pairing.

So far there are seven types of DNA base editors described:
Cytosine Base Editor (CBE) that convert C:G into T:A (Komor, A et al. Programmable editing of a target base in genomic DNA without double-stranded DNA cleavage. Nature 533:420-4. (2016).
Adenine Base Editor (ABE) that convert A:T into G:C (Programmable base editing of A⋅T to G⋅C in genomic DNA without DNA cleavage. Nature 551(7681) 464-471 (2017).
Cytosine Guanine Base Editor (CGBE) that convert C:G into G:C Chen, L et al. Precise and programmable C:G to G:C base editing in genomic DNA. Biorxiv (2020); Kurt, I et al. CRISPR C-to-G base editors for inducing targeted DNA transversions in human cells. Nature Biotechnology (2020).
Cytosine Adenine Base Editor (CABE) that convert C:G into A:T Zhao, D et al. New base editors change C to A in bacteria and C to G in mammalian cells. Nature Biotechnology (2020).
Adenine Cytosine Base Editor (ACBE) that convert A:T into C:G (Liu, D et al. A:T to C:G base editors and uses thereof. Patent application WO2020181180 (2020).
Adenine Thymine Base Editor (ATBE) that convert A:T into T:A (Liu, D et al. A:T to C:G base editors and uses thereof. Patent application WO2020181180 (2020).
Thymine Adenine Base Editor (TABE) that convert T:A into A:T (Liu, D et al. T:A TO A:T base editing through adenosine methylation. Patent application WO2020181193 (2020); Liu, D et al. T:A TO A:T base editing through thymine alkylation. Patent application WO2020181178 (2020); Liu, D et al. T:A TO A:T base editing through adenine excision. Patent application WO2020181195 (2020).

Base editors differ in the base modification enzymes. CBE rely on ssDNA cytidine deaminase among which: APOBEC1, rAPOBEC1, APOBEC1 mutant or evolved version (evoAPOBEC1), and APOBEC homologs (APOBEC3A (eA3A), Anc689), Cytidine deaminase 1 (CDA1), evoCDA1, FERNY, evoFERNY. ABE rely on deoxyadenosine deaminase activity of a tandem fusion TadA-TadA* where TadA* is an evolved version of TadA, an E. coli tRNA adenosine deaminase enzyme, able to convert adenosine into Inosine on ssDNA.TadA* include TadA-8a-e and TadA-7.10.

Except from base modification enzyme there has been also modifications implemented to base editor to increase editing efficacy, precision and modularity:
the addition of one or two uracil DNA glycosylase inhibitor domain (UGI) to prevent base excision repair mechanism to revert base edition
the addition of Mu-GAM that decrease insertion-deletion rate by inhibiting Non-homologous end joining mechanism in the cell (NHEJ)
the use of nickase active Cas9 (nCas9 D10A) that, by creating nicks on the non-edited strand favor its repair and consequently the fixation of the edited base
the use of divers Cas proteins from for example different organisms, mutants with different PAM motifs or different fidelity or different family (e.g. Cas12a)

Non-limiting examples of DNA based editor proteins include BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Cas12a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRY-CBE, SpG-CBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3, AncBE4.

Cytosine Guanine Base Editors (CGBE) consist of a nickase CRISPR fused to:
A cytosine deaminase (rAPOBEC) and base excision repair proteins (e.g. rXRCC1). (Precise and programmable C:G to G:C base editing in genomic DNA. Biorxiv (2020).
A rat APOBEC1 variant (R33A) protein and an E. coli-derived uracil DNA N-glycosylase (eUNG). (Kurt, I et al. CRISPR C-to-G base editors for inducing targeted DNA transversions in human cells. Nature Biotechnology (2020).
Cytosine Adenine Base Editors (CABE) consist of a Cas9 nickase, a cytidine deaminase (e.g. AID), and a uracil-DNA glycosylase (Ung). Zhao, D et al. New base editors change C to A in bacteria and C to G in mammalian cells. Nature Biotechnology (2020).
ACBE include a nucleic acid programmable DNA-binding protein and an adenine oxidase. Liu, D et al. A:T to C:G base editors and uses thereof. Patent application WO2020181180 (2020).
ATBE consist of a Cas9 nickase and one or more adenosine deaminase or an oxidase domain. Liu, D et al. A:T to T:A base editing through adenine deamination and oxidation. Patent application WO2020181202 (2020).
TABE consist of a Cas9 nickase and an adenosine methyltransferase, a thymine alkyltransferase, or an adenosine deaminase domain. (Liu, D et al. T:A TO A:T base editing through adenosine methylation. Patent application WO2020181193 (2020); Liu, D et al. T:A TO A:T base editing through thymine alkylation. Patent application WO2020181178 (2020); Liu, D et al. T:A TO A:T base editing through adenine excision. Patent application WO2020181195 (2020).

Base editor molecules can also consist of two or more of the above listed editor enzymes fused to a Cas protein (e.g. combination of an ABE and CBE). These biomolecules are named dual base editors and enable the editing of two different bases. (Grunewald, J et al. A dual-deaminase CRISPR base editor enables concurrent adenine and cytosine editing, Nature Biotechnology (2020); Li, C et al. Targeted, random mutagenesis of plant genes with dual cytosine and adenine base editors, Nature Biotechnology (2020).

In one embodiment, the base editor is used to inactivate the expression of a gene by editing one or several nucleotides involved in transcription or translation. More specifically the base editor is targeting one or several nucleotides of a promoter, a RBS, a start codon.

In one embodiment, the base editor is used to introduce a premature stop codon.

In one embodiment, the base editor is used to introduce one or several rare codons.

In another embodiment, the base editor is used to modulate the expression of genes by editing one or several nucleotides involved in transcription or translation. More specifically the base editor is targeting one or several nucleotides of a promoter, a RBS, a start codon. leading to an increase or decrease of gene expression.

In another embodiment, the base editor is used to revert a mutation that leads to the inactivation, decrease or increase in activity of a gene or pathway.

In another embodiment, the base editor is used to revert a mutation that leads to an increase of pathogenicity.

In one embodiment, the base editor is used to modify the regulation of a gene by editing one or several nucleotides involved in its regulation such as nucleotides of operator sequence, transcription factor binding site, riboswitch, RNAse recognition site, protease cleavage site, methylation site, post translational modification site (phosphorylation, glycosylation, acetylation, pupylation . . . ).

### RNA Based Editing

RNA base editing is based on the same principle as DNA base editing: an enzyme catalysing the conversion of a RNA base into another has to be brought close to the target base to perform its conversion locally. So far the only enzyme used for RNA editing is an adenosine deaminase from ADAR family that converts Adenosine into Inosine in dsRNA structure. Several seminal studies used this specificity for dsRNA and fused the ADAR deaminase domain (ADARDD) to an antisense oligo in order to program local RNA base editing. More recently the ability of some CRISPR-Cas systems to bind RNA molecules was repurposed into RNA editing. Using catalytically dead Cas13b enzyme (dPspCas13b) fused to an hyperactive mutant of ADAR2 deaminase domain (ADAR2DD-E488Q for REPAIRv1 and ADAR2DD-E488Q-T375G for REPAIRv2) Cox et al improved specificity and efficiency compare to previous RNA editing strategies.

Non-limiting examples of RNA based editor proteins include REPAIRv1, REPAIRv2

In one embodiment, the RNA base editor is used to inactivate the expression of a gene by editing one or several nucleotides involved in translation. More specifically the base editor is targeting one or several nucleotides of a 5′UTR, a RBS, a start codon.

In one embodiment, the RNA base editor is used to introduce a premature stop codon.

In one embodiment, the RNA base editor is used to introduce one or several rare codons.

In another embodiment, the RNA base editor is used to modulate the expression of genes by editing one or several nucleotides involved in translation. More specifically the base editor is targeting one or several nucleotides of a 5′UTR, a RBS, a start codon leading to an increase or decrease of gene expression.

In another embodiment, the RNA base editor is used to revert a mutation that leads to the inactivation or a decrease in activity of a gene or pathway.

In another embodiment, the base editor is used to revert a mutation that leads to an increase of pathogenicity.

### Prime Editing

Prime editors (PE), as described in Anzalone et al. (Anzalone, A. V. et al. Search-and-replace genome editing without double-strand breaks or donor DNA. Nature 576, 149-157 (2019) which is hereby incorporated by reference, consist of a nCas9 fused to a reverse transcriptase used in combination with a prime editing RNA (pegRNA; a guide RNA that includes a template region for reverse transcription).

Prime Editing allows introduction of insertions, deletions (indels) and 12 base-to-base conversions. Prime editing relies on the ability of a reverse transcriptase (RT), fused to a Cas nickase variant, to convert RNA sequence brought by a prime editing guide RNA (pegRNA) into DNA at the nick site generated by the Cas protein. The DNA flap generated from this process is then included or not in the targeted DNA sequence.

Prime editing systems include:
a Cas nickase variant such as Cas9-H840A fused to a reverse transcriptase domain such as M-MLV RT or its mutant version (M-MLV RT(D200N), M-MLV RT(D200N/L603W), M-MLV RT(D200N/L603W/T330P/T306K/W313F)
a prime editing guide RNA (pegRNA)

To favor editing the prime editing system can include the expression of an additional sgRNA targeting the Cas nickase activity towards the non-edited DNA strand ideally only after the resolution of the edited strand flap by designing the sgRNA to anneal with the edited strand but not with the original strand.

Non-limiting examples of prime editing systems include PE1, PE1-M1, PE1-M2, PE1-M3, PE1-M6, PE1-M15, PE1-M3inv, PE2, PE3, PE3b, Cas9 Retron precISe Parallel Editing via homologY (‘CRISPEY’), a retron RNA fused to the sgRNA and expressed together with Cas9 and the retron proteins including at least the reverse transcriptase (Sharon, E. et al. Functional Genetic Variants Revealed by Massively Parallel Precise Genome Editing. Cell 175, 544-557.e16 (2018).), The SCRIBE strategy: a retron system expressed in combination with a recombinase promoting the recombination of single stranded DNA, also known as single stranded annealing proteins (SSAPs)12. Such recombinases include but are not limited to phage recombinases such as lambda red, recET, Sak, Sak4, and newly described SSAPs described in Wannier et al (Wannier, T. M. et al. Improved bacterial recombineering by parallelized protein discovery. Biorxiv 2020.01.14.906594 (2020) doi:10.1101/2020.01.14.906594.), the targetron system based on group II introns described in Karberg et al. (Karberg, M. et al. Group II introns as controllable gene targeting vectors for genetic manipulation of bacteria. Nat Biotechnol 19, 1162-7 (2001) and which has been adapted to many bacterial species, Other retron based gene targeting approaches, as described in Simon et al (Simon, A. J., Ellington, A. D. & Finkelstein, I. J. Retrons and their applications in genome engineering. Nucleic Acids Res 47, 11007-11019 (2019)).

In one embodiment, the prime editing system is used to inactivate the expression of a gene by replacing, deleting, inserting one or several nucleotides involved in transcription or translation. More specifically the prime editing system is replacing, deleting, inserting one or several nucleotides in a promoter, a RBS, a coding sequence.

In one embodiment, the prime editing system is used to introduce one or several premature stop codon.

In one embodiment, the prime editing system is used to introduce one or several rare codons.

In one embodiment, the prime editing system is used to introduce, delete a nucleotide inducing a frameshift in the reading frame.

In another embodiment, the prime editing system is used to modulate the expression of genes by replacing, deleting, inserting one or several nucleotides involved in transcription or translation. More specifically the prime editing system is replacing, deleting, inserting one or several nucleotides in a promoter, a RBS, a start codon. leading to an increase or decrease of gene expression.

In another embodiment, the prime editing system is used to revert a mutation that leads to the inactivation or a decrease in activity of a gene or pathway.

In another embodiment, the prime editing system is used to revert a mutation that leads to an increase of pathogenicity.

The invention encompasses the following embodiments:
1. A C. acnes cell carrying a recombinant DNA vector comprising:
   a phage packaging signal allowing packaging of the DNA vector in a Cutibacterium acnes phage capsid, and
   a gene of interest.
2. A C. acnes producer cell carrying a recombinant DNA vector comprising:
   a phage packaging signal allowing packaging of the DNA vector in a Cutibacterium acnes phage capsid
   a gene of interest
   an origin of replication allowing replication in the producer cell, and
   a selection marker for C. acnes.
3. The DNA vector of embodiment 1, further comprising an origin of replication for C. acnes and a selection marker for C. acnes.
4. The DNA vector of any of embodiments 1-3, wherein the phage packaging signal is at least 90, 93, 95, 97, 98, 99, or 100% identical to SEQ ID NO: 66.
5. The DNA vector of any of embodiments 1-3, wherein the phage packaging signal is at least 90, 93, 95, 97, 98, 99, or 100% identical to phage packaging signal selected from the group consisting of: SEQ ID NO: 76, SEQ ID NO: 77, SEQ ID NO: 78, SEQ ID NO: 79, SEQ ID NO: 80, SEQ ID NO: 81.
6. The DNA vector of any of embodiments 1-5, wherein the DNA vector also comprises a CRISPR-Cas system.
7. The DNA vector of any of embodiments 1-6, comprising a CRISPR-Cas system targeting a C. acnes chromosome locus not present in the C. acnes producer cell strain.
8. The DNA vector of embodiment 7, wherein the targeted locus is a proinflammatory sequence related to acne vulgaris.
9. The DNA vector of any of embodiments 1-8, wherein the DNA vector comprises a template for homologous recombination in C. acnes phages.
10. The DNA vector of any of embodiments 1-9, wherein the DNA vector comprises a template for homologous recombination in C. acnes plasmids.
11. The DNA vector of embodiment 6, wherein the DNA vector comprises a template for homologous recombination and wherein the CRISPR-Cas system targets the DNA vector itself.
12. The DNA vector of any of embodiments 1-11, wherein first selection marker and second selection marker are the same.
13. The DNA vector of any of embodiments 1-11, wherein neither the first nor second selection marker is ermE.
14. The DNA vector of any of embodiments 1-11, wherein first selection marker and second selection marker is catA.
15. The DNA vector of any of embodiments 1-11, wherein first selection marker or second selection marker is catA.
16. An engineered C. acnes comprising any of the DNA vectors of embodiments 1-15.
17. An engineered C. acnes produced by modification with any of the vectors of embodiments 1-15.
18. An engineered C. acnes produced by contacting C. acnes with any of the vectors of embodiments 1-15, modifying the C. acnes with a gene of interest carried by the vector, selecting for the modification, and curing the C. acnes of the plasmid.
19. The engineered C. acnes of any of embodiments 16-18, wherein the C. acnes has been modified by a CRISPR-Cas system carried by the vector.
20. The engineered C. acnes of any of embodiments 16-19, wherein the C. acnes has been modified by insertion of an exogenous gene into the C. acnes chromosome.
21. A method for engineering a C. acnes comprising introducing the DNA vector of any of embodiments 1-15 into a C. acnes.
22. The method of embodiment 21, further comprising selecting a modified C. acnes.
23. The method of embodiment 22, comprising selecting a modified C. acnes that has an insertion of an exogenous gene into the C. acnes chromosome.
24. A method for the production of phage-derived particles comprising the transformation or the transduction of a C. acnes phage genome into the producer cell of embodiment 2.
25. A method for the production of phage-derived particles comprising the introduction of a helper phage into the producer cell of embodiment 2.
26. A phage-derived particle produced by the method of any of embodiments 24-25.
27. A recombinant DNA vector comprising:
   an origin of replication allowing replication in C. acnes;
   optionally a first selection marker allowing for selection of the DNA vector in C. acnes; and
   a gene of interest.
28. The DNA vector of embodiment 27 further comprising an oriT allowing conjugation into C. acnes; an origin of replication allowing replication in a donor bacteria and a second selection marker allowing for selection in a donor bacteria.
29. The DNA vector of embodiment 27 or 28, wherein the origin of replication allowing replication in C. acnes is R6K (typically of sequence SEQ ID NO: 42).
30. The DNA vector of embodiment 27 or 28, wherein the origin of replication allowing replication in C. acnes is RK2 (typically of sequence SEQ ID NO: 43).
31. The DNA vector of embodiment 27 or 28, wherein the origin of replication allowing replication in C. acnes is pBBR1 (typically of sequence SEQ ID NO: 44).
32. The DNA vector of embodiment 27 or 28, wherein the origin of replication allowing replication in C. acnes is pRO1600 (typically of sequence SEQ ID NO: 45).
33. The DNA vector of embodiment 27 or 28, wherein the origin of replication allowing replication in C. acnes is RSF1010 (typically of sequence SEQ ID NO: 46).
34. The DNA vector of embodiment 27 or 28, wherein the origin of replication allowing replication in C. acnes is pAM31 (typically of sequence SEQ ID NO: 47).
35. The DNA vector of embodiment 27 or 28, wherein the origin of replication allowing replication in C. acnes is pLME106 (typically of sequence SEQ ID NO: 48).
36. The DNA vector of embodiment 27 or 28, wherein the origin of replication allowing replication in C. acnes is pTZC1 (typically of sequence SEQ ID NO: 49).
37. The DNA vector of embodiment 27 or 28, wherein the origin of replication allowing replication in C. acnes is pBC1 (typically of sequence SEQ ID NO: 50).
38. The DNA vector of embodiment 27 or 28, wherein the origin of replication allowing replication in C. acnes is pEP2 (typically of sequence SEQ ID NO: 51).
39. The DNA vector of embodiment 27 or 28, wherein the origin of replication allowing replication in C. acnes is pWVO1 (typically of sequence SEQ ID NO: 52).
40. The DNA vector of embodiment 27 or 28, wherein the origin of replication allowing replication in C. acnes is pAP1 (typically of sequence SEQ ID NO: 53).
41. The DNA vector of embodiment 27 or 28, wherein the origin of replication allowing replication in C. acnes is pWKS1 (typically of sequence SEQ ID NO: 54).
42. The DNA vector of embodiment 27 or 28, wherein the origin of replication allowing replication in C. acnes is pLME108 (typically of sequence SEQ ID NO: 55).
43. The DNA vector of embodiment 27 or 28, wherein the origin of replication allowing replication in C. acnes is pLS1 (typically of sequence SEQ ID NO: 56).
44. The DNA vector of embodiment 27 or 28, wherein the origin of replication allowing replication in C. acnes is pUB6060 (typically of sequence SEQ ID NO: 57).
45. The DNA vector of embodiment 27 or 28, wherein the origin of replication allowing replication in C. acnes is p545 (typically of sequence SEQ ID NO: 58).
46. The DNA vector of embodiment 27 or 28, wherein the origin of replication allowing replication in C. acnes is pJD4 (typically of sequence SEQ ID NO: 59).
47. The DNA vector of embodiment 27 or 28, wherein the origin of replication allowing replication in C. acnes is pIJ101 (typically of sequence SEQ ID NO: 60).
48. The DNA vector of embodiment 27 or 28, wherein the origin of replication allowing replication in C. acnes is pSN22 (typically of sequence SEQ ID NO: 61).
49. The DNA vector of embodiment 27 or 28, wherein the origin of replication allowing replication in C. acnes is pGP01 (typically of sequence SEQ ID NO: 62).
50. The DNA vector of embodiment 27 or 28, wherein the origin of replication allowing replication in C. acnes is pIP501 (typically of sequence SEQ ID NO: 63).
51. The DNA vector of embodiment 27 or 28, wherein the origin of replication allowing replication in C. acnes is pCU1 (typically of sequence SEQ ID NO: 64).
52. The DNA vector of embodiment 27 or 28, wherein the origin of replication allowing replication in C. acnes is pBAV1 K-T5 (typically of sequence SEQ ID NO: 65).
53. The DNA vector of any one of embodiments 28 to 52, wherein the oriT is oriT_pMRC01 (typically of sequence SEQ ID NO: 1).
54. The DNA vector of any one of embodiments 28 to 52, wherein the oriT is oriT_RSF1010 (typically of sequence SEQ ID NO: 2).
55. The DNA vector of any one of embodiments 28 to 52, wherein the oriT is oriT_pRS01 (typically of sequence SEQ ID NO: 3).
56. The DNA vector of any one of embodiments 28 to 52, wherein the oriT is oriT_pMV158 (typically of sequence SEQ ID NO: 4).
57. The DNA vector of any one of embodiments 28 to 52, wherein the oriT is oriT_pTF1 (typically of sequence SEQ ID NO: 5).
58. The DNA vector of any one of embodiments 28 to 52, wherein the oriT is oriT_pSC101 (typically of sequence SEQ ID NO: 6).
59. The DNA vector of any one of embodiments 28 to 52, wherein the oriT is oriT_pBTK445 (typically of sequence SEQ ID NO: 7).
60. The DNA vector of any one of embodiments 28 to 52, wherein the oriT is oriT_pBBR1 (typically of sequence SEQ ID NO: 8).
61. The DNA vector of any one of embodiments 28 to 52, wherein the oriT is oriT_R721 (typically of sequence SEQ ID NO: 9).
62. The DNA vector of any one of embodiments 28 to 52, wherein the oriT is oriT_pRmeGR4a (typically of sequence SEQ ID NO: 10).
63. The DNA vector of any one of embodiments 28 to 52, wherein the oriT is oriT_ColE1 (typically of sequence SEQ ID NO: 11).
64. The DNA vector of any one of embodiments 28 to 52, wherein the oriT is oriT_pTiC58 (typically of sequence SEQ ID NO: 12).
65. The DNA vector of any one of embodiments 28 to 52, wherein the oriT is oriT_pMdT1 (typically of sequence SEQ ID NO: 13).
66. The DNA vector of any one of embodiments 28 to 52, wherein the oriT is oriT_R1 (typically of sequence SEQ ID NO: 14).
67. The DNA vector of any one of embodiments 28 to 52, wherein the oriT is oriT_Tn5520 (typically of sequence SEQ ID NO: 15).
68. The DNA vector of any one of embodiments 28 to 52, wherein the oriT is oriT_QKH54 (typically of sequence SEQ ID NO: 16).
69. The DNA vector of any one of embodiments 28 to 52, wherein the oriT is oriT_R64 (typically of sequence SEQ ID NO: 17).
70. The DNA vector of any one of embodiments 28 to 52, wherein the oriT is oriT_R751 (typically of sequence SEQ ID NO: 18).
71. The DNA vector of any one of embodiments 28 to 52, wherein the oriT is oriT_RP4 (typically of sequence SEQ ID NO: 19).
72. The DNA vector of any one of embodiments 28 to 52, wherein the oriT is oriT_pKL1 (typically of sequence SEQ ID NO: 20).
73. The DNA vector of any one of embodiments 28 to 52, wherein the oriT is oriT_RK2 (typically of sequence SEQ ID NO: 21).
74. The DNA vector of any one of embodiments 28 to 52, wherein the oriT is oriT_R1162 (typically of sequence SEQ ID NO: 22).
75. The DNA vector of any one of embodiments 28 to 52, wherein the oriT is oriT_Tn4555 (typically of sequence SEQ ID NO: 23).
76. The DNA vector of any one of embodiments 28 to 52, wherein the oriT is oriT_pHT (typically of sequence SEQ ID NO: 24).
77. The DNA vector of any one of embodiments 28 to 52, wherein the oriT is oriT_Tn4399 (typically of sequence SEQ ID NO: 25).
78. The DNA vector of any one of embodiments 28 to 52, wherein the oriT is oriT_Tn916 (typically of sequence SEQ ID NO: 26).
79. The DNA vector of any one of embodiments 28 to 52, wherein the oriT is oriT_pST12 (typically of sequence SEQ ID NO: 27).
80. The DNA vector of any one of embodiments 28 to 52, wherein the oriT is oriT_pCU1 (typically of sequence SEQ ID NO: 28).
81. The DNA vector of any one of embodiments 28 to 52, wherein the oriT is oriT_pSU233 (typically of sequence SEQ ID NO: 29).
82. The DNA vector of any one of embodiments 28 to 52, wherein the oriT is oriT_F (typically of sequence SEQ ID NO: 30).
83. The DNA vector of any one of embodiments 28 to 52, wherein the oriT is oriT_pMAB01 (typically of sequence SEQ ID NO: 31).
84. The DNA vector of any one of embodiments 28 to 52, wherein the oriT is oriT_R388 (typically of sequence SEQ ID NO: 32).
85. The DNA vector of any one of embodiments 28 to 52, wherein the oriT is oriT_pS7a (typically of sequence SEQ ID NO: 33).
86. The DNA vector of any one of embodiments 28 to 52, wherein the oriT is oriT_pS7b (typically of sequence SEQ ID NO: 34).
87. The DNA vector of any one of embodiments 28 to 52, wherein the oriT is oriT_R702 (typically of sequence SEQ ID NO: 35).
88. The DNA vector of any one of embodiments 28 to 52, wherein the oriT is oriT_pMUR274 (typically of sequence SEQ ID NO: 36).
89. The DNA vector of any one of embodiments 28 to 52, wherein the oriT is oriT_R100 (typically of sequence SEQ ID NO: 37).
90. The DNA vector of any one of embodiments 28 to 52, wherein the oriT is oriT_pVCR94deltaX (typically of sequence SEQ ID NO: 38).
91. The DNA vector of any one of embodiments 28 to 52, wherein the oriT is oriT_R46 (typically of sequence SEQ ID NO: 39).
92. The DNA vector of any one of embodiments 28 to 52, wherein the oriT is oriT_pGO1 (typically of sequence SEQ ID NO: 40).
93. The DNA vector of any one of embodiments 28 to 52, wherein the oriT is oriT_pIP501 (typically of sequence SEQ ID NO: 41).
94. The DNA vector of any one of embodiments 27 to 93, further comprising:
   a relaxase gene;
   a selection marker allowing for selection in the transconjugant C. acnes; and
   a selection marker allowing for selection in the donor bacteria wherein the donor bacteria is an E. coli strain carrying a conjugative plasmid, conjugative transposon, or integrative and conjugative element (ICE), expressing a conjugative machinery.
95. The DNA vector of embodiment 94, wherein the donor bacteria is an E. coli strain carrying the conjugative plasmid, conjugative transposon or ICE pMRC01.
96. The DNA vector of embodiment 94, wherein the donor bacteria is an E. coli strain carrying the conjugative plasmid, conjugative transposon or ICE RSF1010.
97. The DNA vector of embodiment 94, wherein the donor bacteria is an E. coli strain carrying the conjugative plasmid, conjugative transposon or ICE pRS01.
98. The DNA vector of embodiment 94, wherein the donor bacteria is an E. coli strain carrying the conjugative plasmid, conjugative transposon or ICE pMV158.
99. The DNA vector of embodiment 94, wherein the donor bacteria is an E. coli strain carrying the conjugative plasmid, conjugative transposon or ICE pTF1.
100. The DNA vector of embodiment 94, wherein the donor bacteria is an E. coli strain carrying the conjugative plasmid, conjugative transposon or ICE pSC101.
101. The DNA vector of embodiment 94, wherein the donor bacteria is an E. coli strain carrying the conjugative plasmid, conjugative transposon or ICE pBTK445.
102. The DNA vector of embodiment 94, wherein the donor bacteria is an E. coli strain carrying the conjugative plasmid, conjugative transposon or ICE pBBR1.
103. The DNA vector of embodiment 94, wherein the donor bacteria is an E. coli strain carrying the conjugative plasmid, conjugative transposon or ICE R721.
104. The DNA vector of embodiment 94, wherein the donor bacteria is an E. coli strain carrying the conjugative plasmid, conjugative transposon or ICE pRmeGR4a.
105. The DNA vector of embodiment 94, wherein the donor bacteria is an E. coli strain carrying the conjugative plasmid, conjugative transposon or ICE ColE1.
106. The DNA vector of embodiment 94, wherein the donor bacteria is an E. coli strain carrying the conjugative plasmid, conjugative transposon or ICE pTiC58.
107. The DNA vector of embodiment 94, wherein the donor bacteria is an E. coli strain carrying the conjugative plasmid, conjugative transposon or ICE pMdT1.
108. The DNA vector of embodiment 94, wherein the donor bacteria is an E. coli strain carrying the conjugative plasmid, conjugative transposon or ICE R1.
109. The DNA vector of embodiment 94, wherein the donor bacteria is an E. coli strain carrying the conjugative plasmid, conjugative transposon or ICE Tn5520.
110. The DNA vector of embodiment 94, wherein the donor bacteria is an E. coli strain carrying the conjugative plasmid, conjugative transposon or ICE QKH54.
111. The DNA vector of embodiment 94, wherein the donor bacteria is an E. coli strain carrying the conjugative plasmid, conjugative transposon or ICE R64.
112. The DNA vector of embodiment 94, wherein the donor bacteria is an E. coli strain carrying the conjugative plasmid, conjugative transposon or ICE R751.
113. The DNA vector of embodiment 94, wherein the donor bacteria is an E. coli strain carrying the conjugative plasmid, conjugative transposon or ICE RP4.
114. The DNA vector of embodiment 94, wherein the donor bacteria is an E. coli strain carrying the conjugative plasmid, conjugative transposon or ICE pKL1.
115. The DNA vector of embodiment 94, wherein the donor bacteria is an E. coli strain carrying the conjugative plasmid, conjugative transposon or ICE RK2.
116. The DNA vector of embodiment 94, wherein the donor bacteria is an E. coli strain carrying the conjugative plasmid, conjugative transposon or ICE R1162.
117. The DNA vector of embodiment 94, wherein the donor bacteria is an E. coli strain carrying the conjugative plasmid, conjugative transposon or ICE Tn4555.
118. The DNA vector of embodiment 94, wherein the donor bacteria is an E. coli strain carrying the conjugative plasmid, conjugative transposon or ICE pHT.
119. The DNA vector of embodiment 94, wherein the donor bacteria is an E. coli strain carrying the conjugative plasmid, conjugative transposon or ICE Tn4399.
120. The DNA vector of embodiment 94, wherein the donor bacteria is an E. coli strain carrying the conjugative plasmid, conjugative transposon or ICE Tn916.
121. The DNA vector of embodiment 94, wherein the donor bacteria is an E. coli strain carrying the conjugative plasmid, conjugative transposon or ICE pST12.
122. The DNA vector of embodiment 94, wherein the donor bacteria is an E. coli strain carrying the conjugative plasmid, conjugative transposon or ICE pCU1.
123. The DNA vector of embodiment 94, wherein the donor bacteria is an E. coli strain carrying the conjugative plasmid, conjugative transposon or ICE pSU233.
124. The DNA vector of embodiment 94, wherein the donor bacteria is an E. coli strain carrying the conjugative plasmid, conjugative transposon or ICE F.
125. The DNA vector of embodiment 94, wherein the donor bacteria is an E. coli strain carrying the conjugative plasmid, conjugative transposon or ICE pMAB01.
126. The DNA vector of embodiment 94, wherein the donor bacteria is an E. coli strain carrying the conjugative plasmid, conjugative transposon or ICE R388.
127. The DNA vector of embodiment 94, wherein the donor bacteria is an E. coli strain carrying the conjugative plasmid, conjugative transposon or ICE pS7a.
128. The DNA vector of embodiment 94, wherein the donor bacteria is an E. coli strain carrying the conjugative plasmid, conjugative transposon or ICE pS7b.
129. The DNA vector of embodiment 94, wherein the donor bacteria is an E. coli strain carrying the conjugative plasmid, conjugative transposon or ICE R702.
130. The DNA vector of embodiment 94, wherein the donor bacteria is an E. coli strain carrying the conjugative plasmid, conjugative transposon or ICE pMUR274.
131. The DNA vector of embodiment 94, wherein the donor bacteria is an E. coli strain carrying the conjugative plasmid, conjugative transposon or ICE R100.
132. The DNA vector of embodiment 94, wherein the donor bacteria is an E. coli strain carrying the conjugative plasmid, conjugative transposon or ICE pVCR94deltaX.
133. The DNA vector of embodiment 94, wherein the donor bacteria is an E. coli strain carrying the conjugative plasmid, conjugative transposon or ICE R46.
134. The DNA vector of embodiment 94, wherein the donor bacteria is an E. coli strain carrying the conjugative plasmid, conjugative transposon or ICE pGO1.
135. The DNA vector of embodiment 94, wherein the donor bacteria is an E. coli strain carrying the conjugative plasmid, conjugative transposon or ICE pIP501.
136. An engineered C. acnes comprising any of the DNA vectors of any one of embodiments 27 to 135.
137. An engineered C. acnes produced by contacting C. acnes with any of the vectors of any one of embodiments 27 to 135.
138. A method for engineering a C. acnes comprising introducing the DNA vector of any one of embodiments 27 to 135 into a C. acnes.
139. A recombinant DNA vector comprising:
   a phage packaging signal allowing packaging of the DNA vector in a Cutibacterium acnes phage capsid, and
   a gene of interest.
140. A C. acnes producer cell carrying a recombinant DNA vector comprising:
   a phage packaging signal allowing packaging of the DNA vector in a Cutibacterium acnes phage capsid
   a gene of interest
   an origin of replication allowing replication in the producer cell, and
   a selection marker for C. acnes.
141. The DNA vector of embodiment 139 further comprising an origin of replication for C. acnes and a selection marker for C. acnes.
142. The DNA vector of any of embodiments 139-141, wherein the phage packaging signal is at least 90, 93, 95, 97, 98, 99, or 100% identical to (SEQ ID NO: 66).
143. The DNA vector of any of embodiments 139-141, wherein the phage packaging signal is at least 90, 93, 95, 97, 98, 99, or 100% identical to phage packaging signal selected from the group consisting of: SEQ ID NO: 76, SEQ ID NO: 77, SEQ ID NO: 78, SEQ ID NO: 79, SEQ ID NO: 80, SEQ ID NO: 81.
144. The DNA vector of any of embodiments 139-143, wherein the DNA vector also comprises a CRISPR-Cas system.
145. The DNA vector of any of embodiments 139-144, comprising a CRISPR-Cas system targeting a C. acnes chromosome locus not present in the C. acnes producer cell strain.
146. The DNA vector of embodiment 145, wherein the targeted locus is a proinflammatory sequence related to acne vulgaris.
147. The DNA vector of any of embodiments 139-146, wherein the DNA vector comprises a template for homologous recombination in C. acnes phages.
148. The DNA vector of any of embodiments 139-147, wherein the DNA vector comprises a template for homologous recombination in C. acnes plasmids.
149. The DNA vector of embodiment 144, wherein the DNA vector comprises a template for homologous recombination and wherein the CRISPR-Cas system targets the DNA vector itself.
150. The DNA vector of any of embodiments 139-149, wherein first selection marker and second selection marker are the same.
151. The DNA vector of any of embodiments 139-149, wherein neither the first nor second selection marker is ermE.
152. The DNA vector of any of embodiments 139-149, wherein first selection marker and second selection marker is catA.
153. The DNA vector of any of embodiments 139-149, wherein first selection marker or second selection marker is catA.
154. The DNA vector of embodiment 139, which comprises a DNA encoding an antigen.
155. An engineered C. acnes comprising any of the DNA vectors of embodiments 139-154.
156. The engineered C. acnes according to embodiment 155, which comprises a DNA vector as defined in claim 1 which comprises a DNA encoding an antigen.
157. An engineered C. acnes produced by modification with any of the vectors of embodiments 139-154.
158. An engineered C. acnes produced by contacting C. acnes with any of the vectors of embodiments 139-154, modifying the C. acnes with a gene of interest carried by the vector, selecting for the modification, and curing the C. acnes of the plasmid.
159. The engineered C. acnes of any of embodiments 155-158, wherein the C. acnes has been modified by a CRISPR-Cas system carried by the vector.
160. The engineered C. acnes of any of embodiments 155-158, wherein the C. acnes has been modified by insertion of an exogenous gene into the C. acnes chromosome.
161. A method for engineering a C. acnes comprising introducing the DNA vector of any of embodiments 139-154 into a C. acnes.
162. The method of embodiment 161, further comprising selecting a modified C. acnes.
163. The method of embodiment 161, comprising selecting a modified C. acnes that has an insertion of an exogenous gene into the C. acnes chromosome.
164. A method for the production of phage-derived particles comprising the transformation or the transduction of a C. acnes phage genome into the producer cell of embodiment 140.
165. A method for the production of phage-derived particles comprising the introduction of a helper phage into the producer cell of embodiment 140.
166. A phage-derived particle produced by the method of any of embodiments 164-165.
167. A vaccine and/or immunogenic compositions comprising engineered C. acnes of embodiment 155 comprising a DNA vector comprising a nucleic acid encoding an antigen.
168. A method to prevent and/or treat cancer in a subject in need thereof, comprising administering to said subject a therapeutically or prophylactically efficient amount of an engineered C. acnes of embodiment 155 comprising a DNA vector comprising a nucleic acid encoding a tumor antigen.
169. A method to prevent and/or treat a viral infection in a subject in need thereof, comprising administering to said subject a therapeutically or prophylactically efficient amount of an engineered C. acnes of embodiment 155 comprising a DNA vector comprising a nucleic acid encoding a viral antigen.
170. A method to prevent and/or treat a bacterial infection in a subject in need thereof, comprising administering to said subject a therapeutically or prophylactically efficient amount of an engineered C. acnes of embodiment 155 comprising a DNA vector comprising a nucleic acid encoding a bacterial antigen.
171. A method to prevent and/or treat a fungal infection in a subject in need thereof, comprising administering to said subject a therapeutically or prophylactically efficient amount of an engineered C. acnes of embodiment 155 comprising a DNA vector comprising a nucleic acid encoding a fungal antigen.
172. A method to prevent and/or treat an autoimmune disease in a subject in need thereof, comprising administering to said subject a therapeutically or prophylactically efficient amount of an engineered C. acnes of embodiment 155 comprising a DNA vector comprising a nucleic acid encoding a self-antigen.
173. A method to prevent and/or treat an allergy in a subject in need thereof, comprising administering to said subject a therapeutically or prophylactically efficient amount of an engineered C. acnes of embodiment 155 comprising a DNA vector comprising a nucleic acid encoding an allergen.
174. A method to prevent and/or treat graft rejection in a subject in need thereof, comprising administering to said subject a therapeutically or prophylactically efficient amount of an engineered C. acnes of embodiment 155 comprising a DNA vector comprising a nucleic acid encoding a graft-specific antigen.

### EXAMPLES

### Example 1. Phage-Derived Particles for Delivery of DNA Payload into C. acnes

C. acnes phage-derived particles containing a synthetic DNA payload and able to inject it inside C. acnes were developed. It is demonstrated for the first time the stable and autonomous replication of a recombinant DNA vector that allows for transgene expression. These phage-derived particles are produced upon the co-occurrence of a C. acnes phage genome and a DNA payload inside a C. acnes producer cell. The DNA payload is introduced into the C. acnes producer cell by different methods such as electroporation, electroporation of protoplast, conjugation, chemical transformation, transduction into the C. acnes producer cell. Such phage-derived particles open possibilities to deliver DNA encoding a therapeutic molecule into all C. acnes strains in situ with high efficiency and specificity, allowing, for example, sequence specific killing due to CRISPR-Cas expression or modulation of the immune system by secretion of immunomodulators.

Being able to edit Cutibacterium acnes population by removing specific proinflammatory strains to prevent or cure disease such as acne vulgaris or leverage their privilege location into the pilosebaceous unit to modulate the immune system or improve wound healing are attractive therapeutic approaches. To implement such approaches, one can either genetically modify C. acnes strains in situ or provide in vitro genetically modified C. acnes. Because of the large intra and inter-individual microbiome diversity both at the species and strain level, it appears difficult to provide a single or cocktail of engineered C. acnes strains able to colonize the skin of most patients.

Delivery of DNA in situ to the C. acnes population offers a way to circumvent such difficulties by allowing to leverage pre-establish strains potentially without disturbing the local microbiome. However, in situ delivery of genetic material to C. acnes is a challenging task for several reasons. First, there are so far no genetic elements such as plasmid able to robustly and autonomously replicate inside C. acnes. The few described genetic modifications consist in genomic insertion of synthetic DNA through homologous recombination²⁶. This in vitro process has been shown to be very low efficiency and rely on the use of an antibiotic selection marker to select such events. Moreover, these genetic modifications have been restricted to a few specific strains (KPA17202) and might not be generalizable to all C. acnes strains. Second, in order to perform in situ genetic modification of C. acnes we need to deliver DNA. The only described method for introducing DNA into C. acnes is the use of electroporation^{26,27}, a method that can only be performed in vitro.

The present invention solves both delivery and maintenance of synthetic DNA inside C. acnes population in situ. Phage-derived particles composed of a synthetic DNA vector/payload packaged inside the phage capsid at the expense of the phage genome are used. By hijacking the phage-capsid, it was taken advantage of the ability of the phage to transduce DNA into the bacterial host. These phage-derived particles, when put in the presence of the natural bacterial host of the phage, are able to bind to the bacteria and inject the DNA vector/payload inside the bacterial cytoplasm where it can replicate and lead to expression of a protein of interest.

C. acnes phage are naturally present on the skin where they infect and replicate using C. acnes as a host. C. acnes phages have a broad host range, meaning that they can infect most of the C. acnes strain diversity isolated so far. This makes the capsid of these phages a really efficient vehicle to deliver DNA in situ into all C. acnes strains regardless of their genetic diversity. To develop phage-derived particles from C. acnes phages, several phages from the skin of volunteer individuals were first isolated by sampling nose microcomedones using Biore Deep Cleansing Pore Strips (Kao Brands Company), following manufacturer's instructions. After being removed from the nose, microcomedones were collected, homogenized in sterile water and spread onto an RCM agar plate. After incubation under anaerobic conditions at 37° C. for 7 days, plaques could be observed on the lawn of C. acnes growth. Plaques were then isolated and the phages amplified on an indicator strain. Phage DNA was extracted using the Promega wizard DNA clean-up System and sent for library preparation by mechanical random fragmentation and sequenced with an Illumina MiSeq platform. Sequencing reads were assembled using Spades. As expected from previous publications, isolated phages were genetically similar to other sequenced phages.

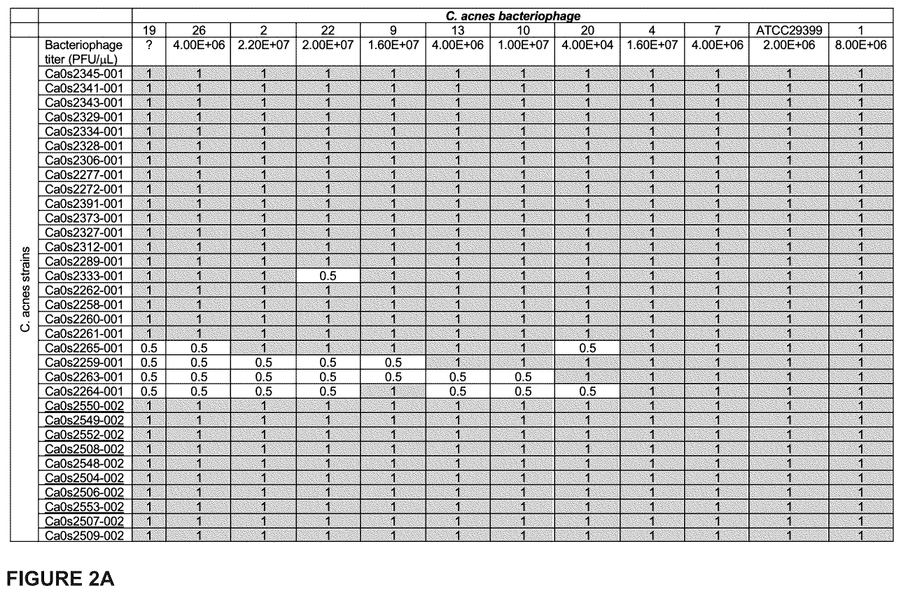

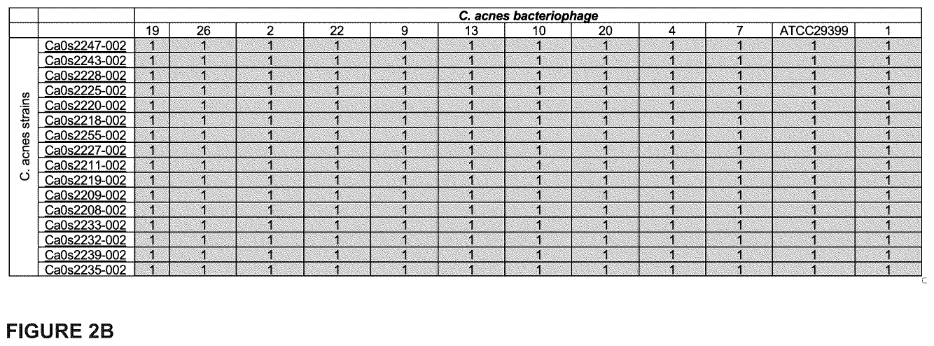

A host-range determination was performed with the different isolated phages against a collection of C. acnes strains, covering the known phylogenetic diversity. All phages were able to infect most of the C. acnes strains showing, as previously reported, a broad host-range (FIG. 2). PAC7 phage was selected for further experiments.

Genome of phage PAC7 was purified, mechanically sheared to allow for random DNA fragmentation and a PCR-free library preparation was performed prior to paired-end sequencing using illumina Mi-seq. DNA reads were assembled using Spades, a single contig was obtained and annotated. After annotation, cohesive-ends were identified and DNA fragments of different sizes, containing cohesive ends, were cloned in order to identify the packaging sequence (called cos site for phages with cohesive ends) that allow recognition by the small terminase and packaging of the phage genome into the phage capsid. Potential packaging signals from PAC7 were cloned into the pIC086 vector in two different orientations. The pIC086 vector contains:
an origin of replication allowing replication into C. acnes, and
a selection marker functional in C. acnes (here giving resistance to erythromycin).

Cos containing vectors (cosmids) were cloned into the E. coli DH10B cloning strain, sequence verified. The DNA vectors (Table 1) were introduced into the C. acnes strain ATCC 11828, and recombinants were selected on agar plates with erythromycin.

To produce phage-derived particles, a liquid culture of the different C. acnes strains carrying the DNA vector (Table 2) were grown and infected by PAC7. A strain containing a plasmid without cos PAC7 (Ca0s16973) was used as control. After infection, the supernatant was filtered and collected. Because both phage genomes and DNA vectors contain a packaging signal, they compete for packaging into the capsid, giving rise to a phage/phage-derived particle mixture.

To quantify the number of phages and phage-derived particles in the suspension, phage and phage-derived particles titration was performed. Titration of phage-derived particles was first performed with C. acnes ATCC 6919, showing high efficiency killing due to phage infection but no transductants could be observed. In these conditions, transductants are co-infected with the phage, leading to death of transduced cells and to the underestimation of phage-derived particle titers. To circumvent this, it was decided to perform titration with a C. acnes ATCC 11828 pseudolysogene strain. Indeed, C. acnes phages are not strictly temperate nor strictly lytic phages in laboratory conditions. They are able to inject their genome into cells and stay dormant in the cell without integrating into the genome. These cells carrying the phage in pseudolysogeny state are immune to phage killing. Using a pseudolysogene culture for phage/phage-derived particles titration, a higher amount of transductants were observed. However due to some residual killing of C. acnes by phages, a large variability in phage-derived particle titers can be observed in different productions from infection of the same producer cell (FIG. 8). The concentration of phage was determined by plaque assay and showed a high concentration of phage for all phage/phage-derived particle suspension with a titer of approximatively 10⁷ PFU/μL for each suspension. (Table 3). Several colonies were confirmed to be C. acnes harbouring the cosmid by PCR. Phage suspension from infection of Ca0s16973 carrying pIC086 plasmid without cos did not show any transductant, confirming that packaging, and thus, the production of phage-derived particles, was specific to cos carrying plasmids.

Titration of the phage-derived particles carrying the DNA vectors comprising phage packaging signal of different sizes shows (FIG. 8) no significant difference in number of transductants. The phage-derived particles titer was similar between all the different cosmids indicating that they are all functional and allow packaging of the DNA vector inside the phage capsid to produce phage-derived particles.

The results show, for the first time:
transduction by a phage-derived particle of a synthetic DNA vector in C. acnes
replication of the DNA vector in C. acnes
expression of a transgene (erythromycin resistance gene) carried by the replicative DNA vector.

This is a key milestone for the development of in situ DNA delivery, genetic modification and transgene expression in C. acnes.

### Materials and Methods:

Cosmids construction: Cos fragments were extracted by PCR on diluted phage PAC7 suspension, gel purified and cloned using SapI golden gate reaction and the pIC086 vector.

Introduction of cosmids in C. acnes can be performed by methods such as electroporation, protoplast electroporation, chemical transformation, using conjugation, natural competency, transduction.

C. acnes conjugation: 2 mL of overnight cultures of E. coli donor harboring the different mobilizable shuttle plasmids, grown in LB broth (Fisher Scientific), were pelleted in a benchtop centrifuged at 6,000×g for 1 min. Supernatants were discarded and pellets were washed with 500 μL of pre-sterilized LB medium and centrifuged again using the same conditions. Each pellet was then re-suspended in 200 μL of exponentially growing (OD₆₀₀=0.5) C. acnes receptor BHI culture concentrated 10× (BHI broth, Oxoid). The mixture E. coli-C. acnes was spotted (50 μL/spot) onto Brucella agar plates (Sigma-Aldrich) and allowed to mate at 37° C. under anaerobic conditions for 24 hours. After that time, cells were harvested from the mating plate, re-suspended in 300 μL of BHI broth and plated onto Brucella agar plates that had been supplemented with 50 μg/mL polymyxin B (Sigma-Aldrich) and 5 μg/mL erythromycin (Sigma-Aldrich) or 5 μg/mL chloramphenicol (Sigma-Aldrich). After 7 days, C. acnes cells that grew in the presence of selection were streaked on Brucella agar plates supplemented with the appropriate selection and the presence of the conjugated plasmid was confirmed via specific PCRs. The identity of C. acnes as well as the absence of E. coli donor strain were also confirmed by PCR analyses.

Phage/phage-derived particles production: Overnight cultures of C. acnes ATCC 11828 harbouring the different vectors (two clones per construct) were set in 10 mL BHI cultures supplemented with 5 μg/mL erythromycin. Production from phagemid pIC328 was used as a positive control. After overnight culture, once the OD600 had reached 0.8-1, 15 mL of each culture were taken and spin down at 3,000×g for 5 min. The supernatant was discarded and the pellet was re-suspended in 200 μL of PAC7 phage suspension and left on the bench at room temperature for 30 min so phages infect the cells. After one hour, 15 mL of BHI medium were added to each culture and allowed to grow/infect overnight under anaerobic conditions at 37° C. After overnight incubation, cultures were very clear, indicating that infection had taken place. Cultures were spun down at 3, 000×g for 5 min, and the supernatant was filtered through a 0.45 μm filter.

Phage titration: Serial dilutions of the phage/packaged phagemid mixture were made in MgSO₄ 5 mM and 4 μL of each dilution were spotted onto Brucella plates containing a top layer of agarose 4.5 g/L and the strain ATCC 11828. After overnight incubation under anaerobic conditions at 37° C., lysis plaques were counted.

Phage-derived particles titration: 90 μL of an overnight culture (OD₆₀₀ approx 0.8-1, concentrated ×10) of C. acnes ATCC 11828 pseudolysogene cells were mixed with 10 μL of Phage/Phage-derived particles from non-diluted to dilution 10⁻⁴ (dilution in MgSO₄ 5 mM). A control of cells with no phage was included in the assay. The cultures were incubated at room temperature for 1 hour. After this first incubation period, the cultures (bacteria+phages/phage-derived particles at different dilutions) were serially diluted up to 10⁻⁷ in BHI and incubated for 3-4 hours under anaerobic conditions at 37° C. After incubation, 4 μL of each dilution were spotted onto Brucella plates in the presence and absence of erythromycin (5 μg/mL). After 5 days of incubation at 37° C. under anaerobic conditions, colonies on BHI plates and BHI+erythromycin 5 μg/mL plates were scanned (FIG. 8).

Pseudolysogene production: strains were freshly made prior to the transduction test. PAC7 phage was added to a suspension of C. acnes ATCC 11828 cells and plated onto BHI agar plates. After 3 to 4 incubation days, cells growing on plates were recovered and either plated again to have more cells or used for titration. If successive growth on plates is needed, C. acnes phages are added to the culture in order to maintain strains in the pseudolysogene state.

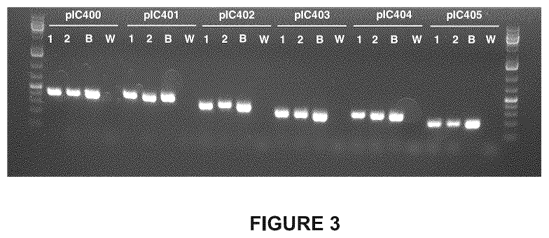

Confirmation of the phagemid transduction into C. acnes cells: colonies observed on BHI plates supplemented with erythromycin were re-isolated on BHI+erythromycin plates. Individual erythromycin resistant colonies obtained after streaking were then tested by PCR to confirm the presence of the phagemid (FIG. 3).

PCR verification of the transductant: colony PCR to check the presence of the phagemid was performed with primers IC208/IC310. A PCR performed with primers AD1261/AD1262 was also included to confirm C. acnes identity.

TABLE 1
Mobilizable DNA vectors including
packaging signal of PAC7 phage

| DNA | | | Mobi- |
|---|---|---|---|
| vector | | Primers | lisable |
| Name | Cos region | for cloning | vector |
| pIC328 | PAC7 Cos region 1 in orientation 1 | AD1542/AD1541 | pIC086 |
| | (383 bp) | | |
| pIC400 | PAC7 Cos region 1 in orientation 1 | IC511/AD1542 | pIC086 |
| | (317 bp) | | |
| pIC401 | PAC7 Cos region 1 in orientation 2 | AD1541/IC512 | pIC086 |
| | (317 bp) | | |
| pIC402 | PAC7 Cos region 2 in orientation 1 | IC511/IC512 | pIC086 |
| | (217 bp) | | |
| pIC403 | PAC7 Cos region 2 in orientation 2 | IC513/IC512 | pIC086 |
| | (167 bp) | | |
| pIC404 | PAC7 Cos region 3 in orientation 1 | IC511/IC514 | pIC086 |
| | (167 bp) | | |
| pIC405 | PAC7 Cos region 3 in orientation 2 | IC513/IC514 | pIC086 |
| | (83 bp) | | |

TABLE 2
List of C. acnes strains generated

| name | Strain description | plasmid |
|---|---|---|
| Ca0s16973 | Cutibacterium acnes ATCC 11828 | pIC086 |
| Ca0s18253 | Cutibacterium acnes ATCC 11828 | pIC328 |
| Ca0s19443 | Cutibacterium acnes ATCC 11828 | pIC400 |
| Ca0s19444 | Cutibacterium acnes ATCC 11828 | pIC401 |
| Ca0s19445 | Cutibacterium acnes ATCC 11828 | pIC402 |
| Ca0s19446 | Cutibacterium acnes ATCC 11828 | pIC403 |
| Ca0s19447 | Cutibacterium acnes ATCC 11828 | pIC404 |
| Ca0s19448 | Cutibacterium acnes ATCC 11828 | pIC405 |

TABLE 3
Results of phage titration

| strain | DNA | Phage used | Phage titer (PFU/μL) on C. acnes |
|---|---|---|---|
| infected | payload | for infection | ATCC 11828 indicator strain |
| Ca0s16973 | pIC086 | PAC7 | ~1E+8 |
| Ca0s18253 | pIC328 | PAC7 | ~1E+7 |
| Ca0s19443 | pIC400 | PAC7 | ~1E+7 |
| Ca0s19444 | pIC401 | PAC7 | ~1E+7 |
| Ca0s19445 | pIC402 | PAC7 | ~1E+7 |
| Ca0s19446 | pIC403 | PAC7 | ~1E+7 |
| Ca0s19447 | pIC404 | PAC7 | ~1E+7 |
| Ca0s19448 | pIC405 | PAC7 | ~1E+7 |

TABLE 4
Primers sequences

| Primers | |
|---|---|
| name | Primers sequence |
| AD1541 | GTTCCAGCTCTTCCGAGGACCACATCACACCCGTC |
| | (SEQ ID NO: 84) |
| AD1542 | GTTCCAGCTCTTCCTGCCCACTCCTCATCAGACAC |
| | (SEQ ID NO: 85) |
| IC511 | GTTCCAGCTCTTCCGAGAGGCAACAGAACACAACCAAA |
| | (SEQ ID NO: 86) |
| IC512 | GTTCCAGCTCTTCCTGCGACTATCAGGAAGCTCAGGC |
| | (SEQ ID NO: 87) |
| IC513 | GTTCCAGCTCTTCCGAGAAAACCCGCCAACCCCCACC |
| | (SEQ ID NO: 88) |
| IC514 | GTTCCAGCTCTTCCTGCACAAAAGGGAGGTATTTCACT |
| | (SEQ ID NO: 89) |
| AD1261 | CAGCGGCGCTGCTAAGAACTT (SEQ ID NO: 90) |
| AD1262 | CCGGCTGGCAAATGAGGCAT (SEQ ID NO: 91) |
| IC208 | GCTTCCTTAGCTTGCGAAATCTCGA |
| | (SEQ ID NO: 82) |
| IC310 | GTTCGGCTAAACCCAAAAGTAAAAAC |
| | (SEQ ID NO: 83) |

### Example 2

Effects of genetically modified C. acnes strains are tested in vitro for their effects on immune cells, in particular for their ability to induce specific cytokines or immune profiles, according to previously described protocols.

In particular, the protocol disclosed in Yu et al. (2016) Journal of Investigative Dermatology 136:2221-2228, with optional modifications and/or adaptations if needed, is implemented on said strains.

### Example 3: Secretion of Antigens by Engineered C. acnes Strains

The pilosebaceous unit (PSU) is a complex skin appendage containing a diverse set of cells such as immune cells, sebaceous cells and stem cells. It is also a highly vascularized area making it an entry point for systemic delivery of molecules. The PSU microbiota is dominated by C. acnes, therefore the ability to engineer C. acnes to secrete recombinant proteins in situ is of great interest to both modulate the activity of the cells present as well as for the delivery of molecules in the blood. The present example demonstrates the use of DNA vectors that once introduced into C. acnes lead to the secretion of recombinant proteins, here the chicken ovalbumin antigen protein. This invention opens possibilities to use engineered C. acnes strains secreting specific proteins of interest such as antigens as skin probiotics. Alternatively engineered phages or phage-derived particles can be used to deliver DNA vectors, encoding for the secretion of protein of interest, in the C. acnes population already present in the PSU.

C. acnes is one of the, if not the, most abundant and prevalent bacterial commensal of the human skin. It resides mostly in the PSU even if it can also be isolated from the skin surface. Specific strains belonging to specific phylotypes have been associated with acne vulgaris disease and are considered to be “pro-inflammatory”. In order to characterize the difference between the different C. acnes phylotypes, a few studies have been characterizing the secretome in order to identify potential proteins specific to the pro-inflammatory phenotypes. Using a subset of the identified secreted proteins, the present inventors were able to identify putative secretion signal peptides (Table 5) using signalP (Armenteros, J. et al. SignalP 5.0 improves signal peptide predictions using deep neural networks. Nat Biotechnol 37, 420-423 (2019)).

To test the ability of these secretion signal peptides to drive secretion of a recombinant protein in C. acnes, the present inventors built several replicative plasmids comprising:
a promoter driving the expression of the recombinant protein,
a signal peptide addressing the proteins to secretion systems fused to the N-terminal of a chicken ovalbumin CDS codon optimized for C. acnes,
an erythromycin selection marker for C. acnes, and
an origin of replication functional in C. acnes.

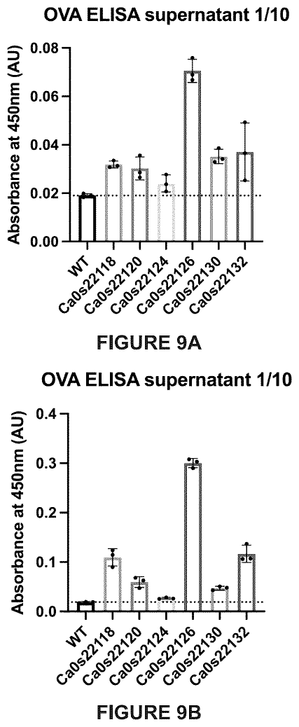

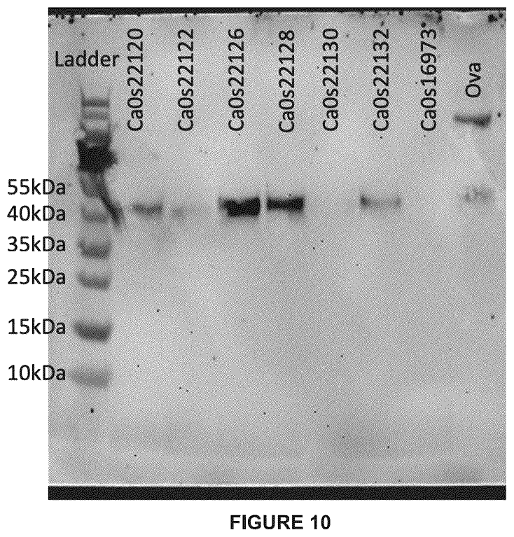

The different DNA vectors (Table 6) were introduced into C. acnes ATCC 11828 (Table 7). Introduction into C. acnes cells can be performed by different methods such as electroporation, electroporation of protoplast, conjugation, chemical transformation, transduction into the C. acnes. Presence of the DNA vectors into C. acnes was confirmed, after streaking on selective plates, by colony PCR. Secretion of chicken ovalbumin protein in the different C. acnes culture supernatants was monitored using ELISA (FIG. 9) and Western Blot (FIG. 10). As shown in FIG. 9, both replicas of ELISA experiment show a significantly higher absorbance for most engineered C. acnes strains, except Ca0s22124, compared to wild-type C. acnes (C. acnes ATCC 11828). Strain Ca0s22126 was repeatedly giving the highest signal indicating higher level of secreted ovalbumin in culture supernatant. Secretion was further confirmed by Western blot (FIG. 10). A single band just above 40 kDa was observed for culture supernatant from strains Ca0s22120, Ca0s22122, Ca0s22126, Ca0s22128 and Ca0s22132. This band corresponds to ovalbumin size (43 kDa) and to the faint band from the ovalbumin control well. No band was observed for control strain Ca0s16973 that carries the empty plasmid used for cloning the different secretion plasmids. More intense band was found for Ca0s22126 confirming the results of the ELISA.

In conclusion, the present inventors describe for the first time the use of endogenous C. acnes secretion peptide for the secretion of recombinant protein by C. acnes using replicative DNA plasmids.

### Materials and Methods:

Plasmids construction: Synthetic DNA fragments were ordered and assembled using SapI golden gate cloning in the p1047 plasmid (pIC086).

Conjugation: As described in Materials and methods of Example 1.

ELISA: The different C. acnes strains were streaked from cryostock into BHI+erythromycin plate, except for the control strain without plasmid that was streaked on BHI without antibiotic, and plates were incubated at 37° C. in anaerobic conditions for 4-7 days. When fully grown, 10 mL cultures of BHI+5 μg/mL erythromycin were inoculated with an inoculum from the corresponding streak and incubated one overnight at 37° C. in anaerobic conditions. After incubation, OD_{600 nm} was measured to control for difference in growth. 1 mL of culture was dispensed into a 1.5 mL tube and centrifuged 6 min at 6000 g. 10 μL of the supernatant was transferred to a high-binding 96 well-plate (Greiner 655061) prefiled with 90 μL of 1×PBS. Incubation of the covered plate during 2 hours at 37° C. was performed. After incubation, samples were discarded from the plate, 100 μL of PBS+5% bovine serum albumin (BSA) was added and the covered plate was incubated for 1 hour at 37° C. Three consecutive washing steps with 100 μL of PBS+0.05% Tween 20 were performed prior to the addition of 100 μL of primary antibody solution (Anti-OVA innovagen PA-0323-100 diluted 1/1000 in PBS 1×+1% BSA+0.05% Tween 20). The covered plate was incubated at RT for 1 hour. Following incubation, three consecutive washing steps with 100 μL of PBS+0.05% Tween 20 were performed prior to the addition of 100 μL of secondary antibody solution (Anti-rabbit Invitrogen A16035 antibody diluted 1/5000 in PBS 1×+1% BSA+0.05% Tween 20) and incubation at RT for 1 hour. After incubation, samples were discarded from the plate and final three consecutive washing steps with 100 μL of PBS+0.05% Tween 20 were performed. 100 μL of TMB-ELISA substrate (Thermo Scientific 34028) was added to each well and incubation was performed under light protection for 10 to 12 min at RT. 100 μL of 1 M sulfuric acid was added to each well to stop the reaction. Absorbance measurement at 450 nm was performed using an infinite reader (Tecan).

Western blot: The different C. acnes strains were streaked from cryostock into BHI+erythromycin plate, except for the control strain without plasmid that was streaked on BHI without antibiotic, and plates were incubated at 37° C. in anaerobic conditions for 4-7 days. When fully grown, 10 mL cultures of BHI+5 μg/mL erythromycin were inoculated with an inoculum from the corresponding streak and incubated one overnight at 37° C. in anaerobic conditions. After incubation, OD₆₀₀ was measured to control for difference in growth. 1 mL of culture was dispensed into a 1.5 mL tube and centrifuged 6 min at 6000 g. Filtration of the supernatant using 0.2 μm filter. 30 μL of the filtered supernatant was supplemented with 7.5 μL of LDS sample buffer (B0008 Invitrogen™) and 3 μL of Bolt™ antioxidant (BT0005 Invitrogen™) before boiling at 100° C. for 10 min. 30 μL of the mixture was loaded into a Bolt™ 4 to 12% Bis-Tris gel (NW04120 Invitrogen™). After migration, transfer on nitrocellulose membrane was performed. After the transfer, the membrane was: soaked first in 5% skim milk solution in PBS+0.05% Tween 20 for 1 h, then soaked in 20 mL 5% skim milk solution in PBS+0.05% Tween 20 containing the primary antibody (Anti-OVA innovagen PA-0323-100) diluted 1:1000 overnight at 4° C., washed three times with PBS+Tween 0.05%, soaked 1 h in 20 mL 5% skim milk solution in PBS+0.05% Tween 20 containing the secondary antibody (Anti-rabbit Invitrogen A16035 antibody) diluted 1:5000, washed three times with PBS+Tween 0.05%. Final step of revelation was performed using chemiluminescent substrate (34580 Thermofisher). Imaging was done using iBright CL1000 (Invitrogen™)

TABLE 5
Secreted proteins used to extract secretion signals

| Protein id | SignalP 5.0 prediction |
|---|---|
| YP_056615.1 | Prediction: Signal peptide (Sec/SPI) Cleavage site |
| | between pos. 23 and 24: GAA-TP. Probability: 0.4339 |
| YP_056817.1 | Prediction: Lipoprotein signal peptide (Sec/SPII) |
| | Cleavage site between pos. 20 and 21: LSA-CG. |
| | Probability: 0.9859 |
| YP_055402.1 | Prediction: Signal peptide (Sec/SPI) Cleavage site |
| | between pos. 28 and 29: AHA-VE. Probability: 0.9710 |
| YP_056047 | Prediction: Signal peptide (Sec/SPI) Cleavage site |
| | between pos. 28 and 29: AHA-AP. Probability: 0.8551 |

TABLE 6
DNA vectors encoding secretion of ovalbumin

| DNA | | signal peptide | |
|---|---|---|---|
| vector Name | Promoter | from | protein |
| p2152 | P138 | YP_056047 | chicken ovalbumin |
| p2154 | P138 | YP_055402.1 | chicken ovalbumin |
| p2156 | P138 | YP_056817.1 | chicken ovalbumin |
| p2158 | P138 | YP_056615.1 | chicken ovalbumin |
| p2160 | ProxP | YP_056047 | chicken ovalbumin |
| p2162 | ProxP | YP_055402.1 | chicken ovalbumin |
| p2164 | ProxP | YP_056817.1 | chicken ovalbumin |
| p2166 | ProxP | YP_056615.1 | chicken ovalbumin |

TABLE 7
List of C. acnes strains generated

| name | Strain description | plasmid |
|---|---|---|
| Ca0s22118 | Cutibacterium acnes ATCC 11828 | p2152 |
| Ca0s22120 | Cutibacterium acnes ATCC 11828 | p2154 |
| Ca0s22122 | Cutibacterium acnes ATCC 11828 | p2156 |
| Ca0s22124 | Cutibacterium acnes ATCC 11828 | p2158 |
| Ca0s22126 | Cutibacterium acnes ATCC 11828 | p2160 |
| Ca0s22128 | Cutibacterium acnes ATCC 11828 | p2162 |
| Ca0s22130 | Cutibacterium acnes ATCC 11828 | p2164 |
| Ca0s22132 | Cutibacterium acnes ATCC 11828 | p2166 |
| Ca0s16973 | Cutibacterium acnes ATCC 11828 | p1047 (pIC86) |

### REFERENCES

1. Pasparakis, M., Haase, I. & Nestle, F. O. Mechanisms regulating skin immunity and inflammation. Nature Reviews Immunology 14, 289-301 (2014).
2. Scharschmidt, T. C. et al. A Wave of Regulatory T Cells into Neonatal Skin Mediates Tolerance to Commensal Microbes. Immunity 43, 1011-1021 (2015).
3. Oh, J. et al. Biogeography and individuality shape function in the human skin metagenome. Nature 514, 59-64 (2014).
4. Oh, J. et al. Biogeography and individuality shape function in the human skin metagenome. Nature 514, 59-64 (2014).
5. Nakatsuji, T. et al. The microbiome extends to subepidermal compartments of normal skin. Nat Commun 4, 1431 (2013).
6. Bay, L. et al. Universal Dermal Microbiome in Human Skin. Mbio 11, (2020).
7. Nagao, K. et al. Stress-induced production of chemokines by hair follicles regulates the trafficking of dendritic cells in skin. Nat Immunol 13, 744-752 (2012).
8. Adachi, T. et al. Hair follicle-derived IL-7 and IL-15 mediate skin-resident memory T cell homeostasis and lymphoma. Nat Med 21, 1272-1279 (2015).
9. Paus, R., Ito, N., Takigawa, M. & Ito, T. The Hair Follicle and Immune Privilege. J Invest Derm Symp P 8, 188-194 (2003).
10. Scholz, C. F. & Kilian, M. The natural history of cutaneous propionibacteria, and reclassification of selected species within the genus Propionibacterium to the proposed novel genera Acidipropionibacterium gen. nov., Cutibacterium gen. nov. and Pseudopropionibacterium gen. nov. International Journal of Systematic and Evolutionary Microbiology 66, 4422-4432 (2016).
11. McLaughlin, J. et al. Propionibacterium acnes and Acne Vulgaris: New Insights from the Integration of Population Genetic, Multi-Omic, Biochemical and Host-Microbe Studies. Microorganisms 7, 128 (2019).
12. Barnard, E. et al. Strains of the Propionibacterium acnes type III lineage are associated with the skin condition progressive macular hypomelanosis. Scientific reports 6, 31968 (2016).
13. Petersen, R. L. W., Scholz, C. F. P., Jensen, A., Brüggemann, H. & Lomholt, H. B. Propionibacterium acnes phylogenetic type III is associated with progressive macular hypomelanosis. European J Microbiol Immunol 7, 37-45 (2017).
14. McDowell, A., McLaughlin, J. & Layton, A. M. Is Cutibacterium (previously Propionibacterium) acnes a potential pathogenic factor in the aetiology of the skin disease progressive macular hypomelanosis? J European Acad Dermatology Venereol Jeadv (2020) doi:10.1111/jdv.16789.
15. Fitz-Gibbon, S. et al. Propionibacterium acnes Strain Populations in the Human Skin Microbiome Associated with Acne. J Invest Dermatol 133, 2152-2160 (2013).
16. Sorensen, M. et al. Mutagenesis of Propionibacterium acnes and analysis of two CAMP factor knock-out mutants. Journal of Microbiological Methods 83, 211-216 (2010).
17. Allhorn, M., Arve, S., Brüggemann, H. & Lood, R. A novel enzyme with antioxidant capacity produced by the ubiquitous skin colonizer Propionibacterium acnes. Sci Rep-uk 6, 36412 (2016).
18. Nazipi, S., Stodkilde, K., Scavenius, C. & Brüggemann, H. The Skin Bacterium Propionibacterium acnes Employs Two Variants of Hyaluronate Lyase with Distinct Properties. Microorg 5, 57 (2017).
19. Kasimatis, G., Fitz-Gibbon, S., Tomida, S., Wong, M. & Li, H. Analysis of Complete Genomes of Propionibacterium acnes Reveals a Novel Plasmid and Increased Pseudogenes in an Acne Associated Strain. BioMed Research International 2013, 1-11 (2013).
20. Davidsson, S. et al. Prevalence of Flp Pili-Encoding Plasmids in Cutibacterium acnes Isolates Obtained from Prostatic Tissue. Frontiers in microbiology 8, 2241 (2017).
21. Aoki, S., Nakase, K., Hayashi, N. & Noguchi, N. Transconjugation of erm(X) conferring high-level resistance of clindamycin for Cutibacterium acnes. Journal of Medical Microbiology (2018) doi:10.1099/jmm.0.000875.
22. Aoki, S. et al. Transferable Multidrug-Resistance Plasmid Carrying a Novel Macrolide-Clindamycin Resistance Gene, erm (50), in Cutibacterium acnes. Antimicrob Agents Ch 64, (2019).
23. Barnard, E., Shi, B., Kang, D., Craft, N. & Li, H. The balance of metagenomic elements shapes the skin microbiome in acne and health. Scientific Reports 6, srep39491 (2016).
24. Rouet, P., Smih, F. & Jasin, M. Expression of a site-specific endonuclease stimulates homologous recombination in mammalian cells. Proc National Acad Sci 91, 6064-6068 (1994).
25. Arazoe, T. et al. Site-specific DNA double-strand break generated by I-Scel endonuclease enhances ectopic homologous recombination in Pyricularia oryzae. Fems Microbiol Lett 352, 221-229 (2014).
26. Liu, J. et al. The diversity and host interactions of Propionibacterium acnes bacteriophages on human skin. The ISME Journal 9, 2078 (2015).
27. Lood, R. & Collin, M. Characterization and genome sequencing of two Propionibacterium acnes phages displaying pseudolysogeny. BMC Genomics 12, 198 (2011).

We claim:

1. A C. acnes phage-derived particle comprising a phagemid lacking genes encoding phage structural proteins, said phagemid comprising:
a phage packaging signal allowing packaging of the phagemid in a Cutibacterium acnes phage capsid,
wherein the phage packaging signal is at least 87% identical to SEQ ID NO: 76,
a gene of interest, and
a CRISPR-Cas system.

2. The C. acnes phage-derived particle according to claim 1, wherein the CRISPR-Cas system targets a C. acnes chromosome locus.

3. The C. acnes phage-derived particle according to claim 1, wherein the CRISPR-Cas system targets a C. acnes plasmid locus.

4. The C. acnes phage-derived particle according to claim 1, wherein the gene of interest is a transgene that is exogenous to C. acnes.

5. The C. acnes phage-derived particle according to claim 4, wherein the transgene encodes a human protein.

6. The C. acnes phage-derived particle according to claim 4, wherein the transgene encodes an antigen.

7. The C. acnes phage-derived particle according to claim 4, wherein the transgene encodes an interleukin.

8. The C. acnes phage-derived particle according to claim 1, wherein the phagemid further comprises an origin of replication for C. acnes and a selection marker for C. acnes.

9. The C. acnes phage-derived particle according to claim 8, wherein the selection marker is not ermE.

10. A phagemid lacking genes encoding phage structural proteins comprising:
a phage packaging signal allowing packaging of the phagemid in a Cutibacterium acnes phage capsid,
wherein the phage packaging signal is at least 87% identical to SEQ ID NO: 76,
a gene of interest, and
a CRISPR-Cas system.

11. The phagemid according to claim 10, wherein the CRISPR-Cas system targets a C. acnes chromosome locus.

12. The phagemid according to claim 10, wherein the CRISPR-Cas system targets a C. acnes plasmid locus.

13. The phagemid according to claim 10, wherein the gene of interest is a transgene that is exogenous to C. acnes.

14. The phagemid according to claim 13, wherein the transgene encodes a human protein.

15. The phagemid according to claim 13, wherein the transgene encodes an antigen.

16. The phagemid according to claim 13, wherein the transgene encodes an interleukin.

17. The phagemid according to claim 10, wherein the phagemid further comprises an origin of replication for C. acnes and a selection marker for C. acnes.

18. The phagemid according to claim 17, wherein the selection marker is not ermE.
